SEQUENCE LISTING (54 sequences)

SEQ ID NO: 1 - protein, 6 aa, synthetic construct
  REGION 1..6: note = source = /note="Description of Artificial Sequence: Synthetic peptide"
SSYYMA

SEQ ID NO: 2 - protein, 5 aa, synthetic construct
  REGION 1..5: note = source = /note="Description of Artificial Sequence: Synthetic peptide"
DSYMS

SEQ ID NO: 3 - protein, 17 aa, synthetic construct
  REGION 1..17: note = source = /note="Description of Artificial Sequence: Synthetic peptide"
DMYPDNGDSSYNQKFRE

SEQ ID NO: 4 - protein, 8 aa, synthetic construct
  REGION 1..8: note = source = /note="Description of Artificial Sequence: Synthetic peptide"
APRWYFSV

SEQ ID NO: 5 - protein, 11 aa, synthetic construct
  REGION 1..11: note = source = /note="Description of Artificial Sequence: Synthetic peptide"
RASQDISNYLN

SEQ ID NO: 6 - protein, 7 aa, synthetic construct
  REGION 1..7: note = source = /note="Description of Artificial Sequence: Synthetic peptide"
YTSRLRS

SEQ ID NO: 7 - protein, 9 aa, synthetic construct
  REGION 1..9: note = source = /note="Description of Artificial Sequence: Synthetic peptide"
QQGHTLPPT

SEQ ID NO: 8 - protein, 117 aa, synthetic construct
  REGION 1..117: note = source = /note="Description of Artificial Sequence: Synthetic polypeptide"
EVQLVQSGAEVKKPGASVKVSCKASGYTFTDSYMSWVRQAPGQGLEWIGDMYPDNGDSSYNQKFRERVTITRDTSTSTAYLELSSLRSEDTAVYYCVLAPRWYFSVWGQGTLVTVSS

SEQ ID NO: 9 - protein, 107 aa, synthetic construct
  REGION 1..107: note = source = /note="Description of Artificial Sequence: Synthetic polypeptide"
DIQMTQSPSSLSASVGDRVTITCRASQDISNYLNWYQQKPGKAPKLLIYYTSRLRSGVPSRFSGSGSGTDFTLTISSLQPEDFATYYCQQGHTLPPTFGQGTKVEIK

SEQ ID NO: 10 - protein, 5 aa, synthetic construct
  REGION 1..5: note = source = /note="Description of Artificial Sequence: Synthetic peptide"
NYLIE

SEQ ID NO: 11 - protein, 17 aa, synthetic construct
  REGION 1..17: note = source = /note="Description of Artificial Sequence: Synthetic peptide"
VINPGSGDTYYSEKFKG

SEQ ID NO: 12 - protein, 5 aa, synthetic construct
  REGION 1..5: note = source = /note="Description of Artificial Sequence: Synthetic peptide"
DRLDY

SEQ ID NO: 13 - protein, 11 aa, synthetic construct
  REGION 1..11: note = source = /note="Description of Artificial Sequence: Synthetic peptide"
HASQDISSYIV

SEQ ID NO: 14 - protein, 7 aa, synthetic construct
  REGION 1..7: note = source = /note="Description of Artificial Sequence: Synthetic peptide"
HGTNLED

SEQ ID NO: 15 - protein, 9 aa, synthetic construct
  REGION 1..9: note = source = /note="Description of Artificial Sequence: Synthetic peptide"
VHYAQFPYT

SEQ ID NO: 16 - protein, 114 aa, synthetic construct
  REGION 1..114: note = source = /note="Description of Artificial Sequence: Synthetic polypeptide"
EVQLVQSGAEVKKPGASVKVSCKASGYAFTNYLIEWVRQAPGQGLEWIGVINPGSGDTYYSEKFKGRVTITRDTSTSTAYLELSSLRSEDTAVYYCARDRLDYWGQGTLVTVSS

SEQ ID NO: 17 - protein, 107 aa, synthetic construct
  REGION 1..107: note = source = /note="Description of Artificial Sequence: Synthetic polypeptide"
DIQMTQSPSSLSASVGDRVTITCHASQDISSYIVWYQQKPGKAPKLLIYHGTNLEDGVPSRFSGSGSGTDFTLTISSLQPEDFATYYCVHYAQFPYTFGQGTKVEIK

SEQ ID NO: 18 - protein, 215 aa, synthetic construct
  REGION 1..215: note = source = /note="Description of Artificial Sequence: Synthetic polypeptide"
DIQMTQSPSSLSASVGDRVTITCKASAAVGTYVAWYQQKPGKAPKLLIYSASYRKRGVPSRFSGSGSGTDFTLTISSLQPEDFATYYCHQYYTYPLFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 19 - protein, 214 aa, synthetic construct
  REGION 1..214: note = source = /note="Description of Artificial Sequence: Synthetic polypeptide"
QAVVTQEPSLTVSPGGTVTLTCGSSTGAVTTSNYANWVQEKPGQAFRGLIGGTNKRAPGTPARFSGSLLGGKAALTLSGAQPEDEAEYYCALWYSNLWVFGGGTKLTVLSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 20 - protein, 694 aa, synthetic construct
  REGION 1..694: note = source = /note="Description of Artificial Sequence: Synthetic polypeptide"
QVQLVQSGAEVKKPGASVKVSCKASGYTFTEFGMNWVRQAPGQGLEWMGWINTKTGEATYVEEFKGRVTFTTDTSTSTAYMELRSLRSDDTAVYYCARWDFAYYVEAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDGGGGSGGGGSEVQLLESGGGLVQPGGSLRLSCAASGFTFSTYAMNWVRQAPGKGLEWVSRIRSKYNNYATYYADSVKGRFTISRDDSKNTLYLQMNSLRAEDTAVYYCVRHGNFGNSYVSWFAYWGQGTLVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 21 - protein, 451 aa, synthetic construct
  REGION 1..451: note = source = /note="Description of Artificial Sequence: Synthetic polypeptide"
QVQLVQSGAEVKKPGASVKVSCKASGYTFTEFGMNWVRQAPGQGLEWMGWINTKTGEATYVEEFKGRVTFTTDTSTSTAYMELRSLRSDDTAVYYCARWDFAYYVEAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 22 - protein, 453 aa, synthetic construct
  REGION 1..453: note = source = /note="Description of Artificial Sequence: Synthetic polypeptide"
EVQLVESGGGLVQPGGSLRLSCAASGYDFTHYGMNWVRQAPGKGLEWVGWINTYTGEPTYAADFKRRFTFSLDTSKSTAYLQMNSLRAEDTAVYYCAKYPYYYGTSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMASRTPEVTCVVVDVSHEDPEVKFNWYVDGVEVHNAKTKPREEQYNSTYRVVSVLTVLAQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNAYTQKSLSLSPGK

SEQ ID NO: 23 - protein, 463 aa, synthetic construct
  REGION 1..463: note = source = /note="Description of Artificial Sequence: Synthetic polypeptide"
QVQLVQSGAEVKKPGASVKVSCKASGYTFTGYYMHWVRQAPGQGLEWMGWINPNSGGTNYAQKFQGRVTMTRDTSISTAYMELSRLRSDDTAVYYCARSPNPYYYDSSGYYYPGAFDIWGQGTMVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPEAAGGPSVFLFPPKPKDTLMASRTPEVTCVVVDVSHEDPEVKFNWYVDGVEVHNAKTKPREEQYNSTYRVVSVLTVLAQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNAYTQKSLSLSPGK

SEQ ID NO: 24 - protein, 214 aa, synthetic construct
  REGION 1..214: note = source = /note="Description of Artificial Sequence: Synthetic polypeptide"
DIQLTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 25 - protein, 213 aa, synthetic construct
  REGION 1..213: note = source = /note="Description of Artificial Sequence: Synthetic polypeptide"
SYVLTQPPSVSVAPGQTARITCGGNNIGSKSVHWYQQKPGQAPVLVVYDDSDRPSGIPERFSGSNSGNTATLTISRVEAGDEADYYCQVWDSSSDHWVFGGGTKLTVLSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 26 - protein, 17 aa, synthetic construct
  REGION 1..17: note = source = /note="Description of Artificial Sequence: Synthetic peptide"
AIFTGSGAEYKAEWAKG

SEQ ID NO: 27 - protein, 13 aa, synthetic construct
  REGION 1..13: note = source = /note="Description of Artificial Sequence: Synthetic peptide"
DAGYDYPTHAMHY

SEQ ID NO: 28 - protein, 11 aa, synthetic construct
  REGION 1..11: note = source = /note="Description of Artificial Sequence: Synthetic peptide"
RASQGISSSLA

SEQ ID NO: 29 - protein, 7 aa, synthetic construct
  REGION 1..7: note = source = /note="Description of Artificial Sequence: Synthetic peptide"
GASETES

SEQ ID NO: 30 - protein, 12 aa, synthetic construct
  REGION 1..12: note = source = /note="Description of Artificial Sequence: Synthetic peptide"
QNTKVGSSYGNT

SEQ ID NO: 31 - protein, 123 aa, synthetic construct [sequence omitted]

SEQ ID NO: 32 - protein, 110 aa, synthetic construct [sequence omitted]

SEQ ID NO: 33 - DNA, 19 bp, synthetic construct
  misc_feature 1..19: note = source = /note="Description of Artificial Sequence: Synthetic oligonucleotide"
  source 1..19: mol_type = other DNA
gtcctttgggcagagacag

SEQ ID NO: 34 - DNA, 19 bp, synthetic construct
  misc_feature 1..19: note = source = /note="Description of Artificial Sequence: Synthetic oligonucleotide"
  source 1..19: mol_type = other DNA
gaccagagtactccctcgt

SEQ ID NO: 35 - DNA, 20 bp, synthetic construct
  misc_feature 1..20: note = source = /note="Description of Artificial Sequence: Synthetic primer"
  source 1..20: mol_type = other DNA
ccagatttggtgaggacgat

SEQ ID NO: 36 - DNA, 19 bp, synthetic construct
  misc_feature 1..19: note = source = /note="Description of Artificial Sequence: Synthetic primer"
  source 1..19: mol_type = other DNA
gaccagagtactccctcgt

SEQ ID NO: 37 - DNA, 30 bp, unidentified
  misc_feature 1..30: note = source = /note="Description of Unknown: Pyruvate kinase muscle WT sequence"
  source 1..30: mol_type = other DNA
gaccagagtactccctcatgggcacagtgg

SEQ ID NO: 38 - DNA, 29 bp, unidentified
  misc_feature 1..29: note = source = /note="Description of Unknown: Pyruvate kinase muscle WT sequence"
  source 1..29: mol_type = other DNA
caccctgcctctgtctctgcccaaaggac

SEQ ID NO: 39 - DNA, 27 bp, synthetic construct
  misc_feature 1..27: note = source = /note="Description of Artificial Sequence: Synthetic oligonucleotide"
  source 1..27: mol_type = other DNA
caccctgcctctgcatgggcacagtgg

SEQ ID NO: 40 - DNA, 19 bp, synthetic construct
  misc_feature 1..19: note = source = /note="Description of Artificial Sequence: Synthetic oligonucleotide"
  source 1..19: mol_type = other DNA
caccctgcctctgcagtgg

SEQ ID NO: 41 - DNA, 14 bp, synthetic construct
  misc_feature 1..14: note = source = /note="Description of Artificial Sequence: Synthetic oligonucleotide"
  source 1..14: mol_type = other DNA
catgggcacagtgg

SEQ ID NO: 42 - DNA, 20 bp, synthetic construct
  misc_feature 1..20: note = source = /note="Description of Artificial Sequence: Synthetic oligonucleotide"
  source 1..20: mol_type = other DNA
cccatcacggcccgcaacac

SEQ ID NO: 43 - DNA, 20 bp, synthetic construct
  misc_feature 1..20: note = source = /note="Description of Artificial Sequence: Synthetic oligonucleotide"
  source 1..20: mol_type = other DNA
cttcttcaagacgggggatg

SEQ ID NO: 44 - DNA, 22960 bp, Cricetulus griseus
  source 1..22960: mol_type = genomic DNA
gagctgcggagggattgcggcgggtcgcagctgggataaccttgaggcccaagaggcgcagtcccgcacacccgtgactcgtcctgagactgcggacgtccgacttaggcatcgctgcaagatccggagtacgcccgaggtgagaggggagagcctacgccatctgggcccagggccgatgcctgcttctgcggagcgcaggcctgcgggcccatgcgggactaaggctcctggcatcgtgcatcccagtacgggttgggggaaaagctacactcgagcacaggtcctgagcttccaaagtcagagggcccagccagccccttcccacgcggtagttctggtccctcttctttccttctcaatctggttccaccctcttatcttactgctgccgtcctccccgccctgttctctcccactcttcgatgctcatcctcctcagccatttactagtggttgttttccctttattttaactctgtgccaccatcccccccaccttcggtgccctctttgtaatctctactcgaaataactctcctgctccccccttgcgccccggcattcatcccccatccaggtcagtatgctcttcgcgtggccctgttggctgctccgcccctccccctccattccagtcacgtccgcgccactgcatcctttcagcatccggatcctcgcccctccccctccgcgctgcttcccccgcccaccgcattctggtcacgtccgcggactcccctcaggccctcgtggtccgcggcctgtaggtagggtgcagttagtggccgtccagggagatacagccaccggttacgtaacatcatcttcccggtggtgtggcaaggagggttggcagctattccttcctattttgtgcggccgcagattcttctggttgccggcgaagcactcgtgctccgggctacccgggccctgtggggtgcctgcaagcgctcacgcggtccatcctgcggtggcgaggctgcgcgtaccgcagggctcctggaccagccagtcacactgctccaacgtgctggcccggggtgggcgatcaaccaagggcggcttccgaatgtagctgcagcccgatgcgaccagaacgctcatagcccacagagttcagtctgattagggaagcaaaatttgtcaagtcagtgctgtgaggaaggatggggctgcacgagctggtccgcacacagggattgagtaagcagtagagcacctagtctgaaggttacttaaaaatacagtcttggaaagatgcctctggatctatctcctcttccaaacccaggagattgataggataaatagagcctcataatgtgggtccagtcctgcgtggaggcagcgggggatatacacaggttttaggaatcttctgaggtggttcagcctcttagacatttgctccagactgctggagttactggcctccgtgtctgtacggacatgactaatctcaggtgaaatgaccacgagggcgtgcacatgtactttttttttgtcaatggatgaaatacagttaccttgaaggccgagtctgtcgcccagttattcctctacacattagtactaagcttggggaggcagggaggcaatgaacacccagtatcccctaggaaacgaattctctctggtccgtgtttacaatgtaactaatgttaacaattgcaactcccaagaaagccagctatctaaagggacaaatgctgcagcttttgtcttcgactgtaacggtagaaacaaaggaacaggtacccggatcagtctcttctgattttctcgtccctgatggttcatgtaagacctttctgctccctggcttgtaggaagttggatttggaagctgttgcagtaagttctgttgccacaatacttagggtggggctactttctagagtacagaagggagggccttgggtcaggattcaaggtaccattgttagcaaacaatttactgtctggtggcctgaggatgttgtaggtaaggcagtgaggctgttctgtgggaaagtgtactgtaatccaccaccgggtctgggcaagatgactcatcctttcacattagcggcattcgagaagattctcatccttgggtgagtggggaaaacttggtactcctaggtttagtaaggcccctgtttagtcagtcaaatactgagctagcgtgcatttccctaagttaaacttctatgtgtcactcagactgagtgtggtggttcagcacttggaggccaggatcgcaggttccagaggggggaaggggcagcaaaatgccggggaccaggccctttgtgctactggagtgctggaagtgtgagtggttggtcctagtctataggtccactcttaattcttgagtgataggcacagcagcttctcatggaacaaccagtatccagaatgacaatttctgtgaactcatttgagttactgcttttaagtggaactatatggatgataattaatcactttctagtacatagctgactccaaacaactcagtctcatgggggttggatataccatctgttttggcaagaaggtatgatcacaggaaaggacatcttatttcttaaagctgttgatttcttaatattgagtagaagaaacgagccatccatgggcaggaaggtagaaaaaaaacctattgcattagactcattacttgggtttttagtgtcttttgcttcagcttttctacaggaagtcaagatgagttggccaagcactaaggtggagattgaggtagaggaaaatgactgattcactttaagtttctgaatcatcccaaatacccacagccaagttaactggtaaactacaaaaatgagttgtcgcctgtgaggttttttacttcgctggtaagagtacataagtactgagtcatccttgcaaacttcactctggtacggatagaaataaatgacagactttttgattaattcctgacatgatactttcttgggtattgcaatttttcccaagagccagacttagactggatatggagtagtcggaagaagtgagcccacgtggtgagcccacgtgggtggtggcctgcaaacctctgctggcctcttaaggcttagtgtaacagctcagctcaggactgggggtggtcttacagacaccaagctgtgcagtgaggccacgcccctaccgtagtttgtgcctgtctgcacgtaggagggaagggggaaggtttgagctgtagtacctctggtggcctctgggacaccttcactgaagggtctggggagctctgaatctttcccaaagccagctaagctctaaaggaagctgcatcccttccaaacatttttttatttttattttttgttgttgttgtgtttgtttttcgagacagggtttctctgtggctttggatgctgtcctgtagaccaggctggtcttgaactcacagaaatccaactgcctcccgagtgctggaattaaaggtgtgcactaccacagcccggctccttccaaacattttcaattaacctacaggtcatattttgactttaaactactggtgttttccagcaatagtgtaaaagattttaaggcatgcccattttggaagagtagggaagcactgggtatggtggtgcacatttttaatctcagcactcaggaggcaaaggcaggcggatcttcaattcaaggacagcccagaagaagctggtaatgtagtgctcgggaaatgtttgcctgttgggcacaaagtctttccatcactagcactacataaaattgacatgcctttaattccaacattcaagatgtggtggcacgaggattggaagtttatattcagcctcagctacttgatgagctcaaggccagtcccagatacatgagaacccatatcaaacaagaaaaaaaagccaacatagggttcaatatagatatagtgattgggtatccaaaattcagggtaggggtggggacaaaggacagcgggagggttagagaagtcagagattgggagaaggaaattatttatggattgaatacaatcagaatactttttatttttggaaagtctaaaaatacaaatcattgttgaagcagagttctaagtggtagaaacacagggtttggggtccctctcccccttgggaaggaatagttgtgatcctataaaggtcactgtagacaggcactcatagtaagtaggtcagtgctcttggacagctggtactgccttccctatttaacaccccccgccccccttggttcaggacagaagctgtgcctccagttggggagaccattgtgcattctgcttacacctggttggtttcctagactcctagccagggtccagagtgggcatcattcccggtaacactggcagcttaagcctgctaaggagggcaccatgtgccagcagaacagaaactttctcccagatcatcttctggctcaaactagttgcaggagatccttggtactaaggactggagagtttctttgtccctggcattctgtcctgaaataaaatggtctttctgcaggatggtcctcataattgcatggcaggatttacagtaggactgttaaagttaaggttaaataaaagatggtttgtgcttgtttctttaattttttttttgaccccacagaaatgagccacccaaacaggtgggctcatattctgaatagccttgtaacaccagacttttctggtacagaatttgctagggttaagaagtagacctccctctggacaaggtagcaaatgccttcaatcccagcattcaggaggtaggggagatggatatcttgagttttaggatagccagggctatgtaataagaccatgtcttaaagaataaataagccaggcagtggtggtgcatggcctgacagtgccagaattagggtggcattggtcagtttttgttgttcttatgccctttgtgccagagatgggagaaggcagaaccagatgacctgagctgagcctgtgcatgcctcaacctgcagcccacactgaagcctgggctgctgtctcctttctcacagaggggcaatggctctgaggaagatactgcttcaggggctcaggctcaagagcagggctcctgtccctgagcatggtgtttaccagtgtcattggacttgccaggcttctgcagctgtctggaggttggctgtgcatgtgggtgcctcctgaggaggaggatggtggcatgggtggggccgcatgacttggcaggcactactgtacaagtagctagccaatagagcaagccctaagggcctaagaaactggggctggaactgctttcctgggccagaaaataacctcagaaagggtattctatatgttatatatagataggtggttggtttgagttgtggggctccataggggtcatgggataggaagaagagtcctgattttgaaggggggcggtgctccaggtatcaatagcctgggaagagaaacgggttagtgagctgggatccagattggtaggggacttgaaacacttgaagttttgtaggaggtctatttaattgtatcgatccattgatacatatttccacttgccactttgtatctggtagcaagataatctgcagactatagtagttctagatggggaagtgggttatatttgaagccaatgaaattttacctgttaaattgcatttccccgccctaagggcagcagcccttgtctaacaaggattgtactgtcaaccctgagaacaggcaaagcaggaaaagaaaattactggactctggcatggaactgataagtttttataatgaaaattttctatgggttatattgtacacttttctttttctttttggctttgagatcaatggatcaagttgttctccaggtcagtgaagcttccctgaggtgcacttgtgactgagggaatattagcagagtcatcctgaagggctcggctgatatatggaaattcactttacatataattcctcccatgagctttatagaatgcatagaaataaatgtgtcattttcagaaactgcgctttttttgtttgttttttggttttggtttcttttttttttttttttttccgagacagggtttctctgtggctttggaggctgtcctggaactagctcttgtagaccaggctggtctcaaacacacagagatccacctgcctctgcctcctgcgtgctgggattaaaggcgtgtgccaccactgcccggataaaggtaaggatttttttttttttttttctcaagacagggtttctctgtgtagctttggagcctatcctggcactcgctctggagaccaggctgctctcgaactcacagagatctgcctgcctctgcctcccaagtgctgggattaaaggcgtgcaccaccacaccagccacttctggttattctttgaacactccatcaggatcatacttgctagattcttggcattttgcatcaactcttgtttttgagctggggtttctttgtgtaacagtgctcccaagtgctggaattaaaggtgtgcgccaccacctggccacatcaacaattttattgataaaggatatgccactgggtgcggtgacacagcattagaaaggcagatttcttttgttttttgtttttctttctttctttttttaatagatttatttatcatgtatacagtgcatgccagaagagggcactagatcacattacagatggttgtgagccaccatgtggttgctgggaattgaactcagaacctctgaaagagcagcagtcagtgctcttaactcctgagccatctctccagccccaggaaggcagatttctgaattggagaccaatctggtctaagtagcacctccagcaggtcagggatgtatagtgagacactgtctcagagaaggaggagggagcattgtagtgaatactgccacccaggtaacaaactgttattttgacatttatcctcttaagctttttatcttaaggtggagaccaggtttttatataaacttagagaatgtggtggtgcacatctgtaacctcaaccttgaggagagaggcaatatgtagtgagttccaggccagccagagctacttagtgagaccctcttaagtaaataggggttgtagtatattacaggtttaaagactgtccaggaaggagtcagtcctagtgactcatgcctgtactcttaacacttgggaagttgagaccggaggaggatctttgtgagtgaagccagcctggggttcattaatattgtgtgtgtgtgtgtgtaacatagctgaatctatctctaagagcaatttctacctggttaaggtgattaccacaaagagctcagctttcgccttaatccttgtacagctctccaaggcaaactgtactcagctctgaacagtctttccaagtgaggccaaggagccactctgagtcaaaaaaacagcattatgtcactggaagcccaggcccagagaaccaaaggtatgacctgattatcagtggcctcaggcaggaatttgcaaggagctttaatgtcttagttggagagaagcacctggtggctcttcaggaaagtgggttcggggtctgtcccactcgtgctgacttcatagttcatagtcctatcatggtcatagccagacttttgtgtcgtgttggtttgatgtaccatctattcctttagcagccaccaaagcatggctgggaggcatgagagtttgccagccctgtttgtttcaaccatgggtagacaaaggacctgactgcctgggacttcctttgtgaggacttctcagatttggagaggggaaacaagcagaactgatcttggcagagagaccttaatgagactgagagctcatttaacaaatactacaaggtgccttagcaccttgtagaccccaaaggagaggttgaacagtgttggccaatggcagtagttagaggtctgattcctcctttaaaagttttccgttctgcagtgaaaatagtggtgcacccctggaatccagcacccagaagcccggaactcgtgggtggccagccttcttttccattgcatcactagtaggttacaggggtccccaggtctcaagaagcagagatgaattttagtgatcaagaaatccatggttgttgagtgagggtcctttactaggagtcagggcagagcaaggatgcagaggggtttattgtagatgagccccttggcctgagaactcagatgagaaggacacccagcttcattcaagcctctcaacttttgtaaaacacaaagcttctacagagttatgtcctctaaccagctcagtgagcatttagttgtgagtcggtaataaagttaaagcaaaggtcaggcggctgttgggattgtgacctgggtggttaattacctggaacagatgctcaggcttctgagagtgctctacctacctatcagacatagagagggattccagatgcttagaactggggctgggaaagccatgtctaacgtcagcacgtggaaagatcgaagagtgagttcaaggtcagcctaggttatagagtggattctaggtcatctgagactagctactgtgcaattctgtttcctaggttacagacgactccttggtgtaatgactttggagctattccagtgctcttgcgagcaaaatttgctttgtgtgtcttttaattgttgctgttgttttattgctaaagcattctgggattttgcttcctgcctagaagaactacttttttgccgcctatcatttggatcttatgctcttatgtctaataaaataagcttaggcaacgaggccagtgaaaacctagaactctggagctaaagggggcttgggacccttgtgtttgtctccttgatctgtgggaccatttgagtttgggtgaacatggtaaagaggctgttgggtaccaaagcacatacttgtagattcagtgcctcatcatggtagacttcccaaggtaccaggtgtttgtatgatgatagattagttgtaactttgggggaggatagttcttacaacttttgcaagtttcttcaagtatgtaaagtgatgtccttcctagggtggtgtgaaagctatccgtagcagctcttgtgatactcctgcttttactgccccctgggacaatatatgctttggatgggaagttacttttgttcatcaaggtctttgctgtttgctcacagtcacattgagccctttgcccaatttgaggagcacccacagaaccagcacaggccaggcctctgtgtgacttgtgcacaaagcaatcgtattaggttaatgtgtaatcgggagacagctgcctacgccaagtggctcattaagcactgcccaatctaattgcactcttcaactccccgggacccaggacctcagaaaccatgccgaaaccacacagtgaagcagggactgccttcattcagacccagcagctccatgcagctatggctgataccttcctggaacatatgtgccgcctggacattgactccgcacccatcacggcccgcaacactggcatcatttgtaccattggtgagtgagtgtggcgctcccctagatggaggcttcccacctgatgatctgctaaagagcatagcaattacaaggctaacctaggttctgagtgaggcactgtacacatttcttggaatggaggtcataagaaatggccataaagaattagtatgttttgccgggcggaagtggtggcacacagctttaatcccagcactcatgacacagaggcaggtggatctctgagttcgagctagcttagtctacagagcaagttccaggccagccagggctacacacaaagcctatttcaaaacaaaaacaaaaaagtgcactttgttttctgtgataggtagaattcaggggttcttccttagataatcgttgtaggagtatttctgagatctcaggtctttttcatatggattgtatcaccaacattcagtagttggacaacttgagggcaagggaaactgcttaattctaccctccacttccctgattatgtgggggccctaaagaatttagagagtggcagggtttccccctcgtatacgtaggttaggaaagttgactggcataatgctttgtgttcatttttggagttgattttaaaaacaattaaaggatgaagaatttgctgagaaaattacacacgttttcttgtatcatgcttttttacctgtggttttaaatatttattaataggtcctgcctcccggtctgtggagatgctgaaggagatgatcaagtctggaatgaatgtggctcgtttgaatttctctcatggaactcatgaggtaggcctcagctagagcaatttattggaaacaggaggtgtggtgataattagcgctactcccggcagatatgaaccccagttcagtgggacattgtcctagccctagcccatcctggattccctctttccctcctgtcaccttcataggcctaaacaaacaaggccagctttgtcaaggaccagggctgtcctacccttaaccttcacaaaagaaggcaggaaggatttagctggtaacccctctaggccatcaactgtcttagctctgcacttgggaacaatgcctcagggtttctctagtggctcctgtgagtctggtgaccaaaaggtcatgcagaaggttgattccggcttagttcaatcctgagatgattagctagttccatagataggggtttccacttgagcctgagtcgagtgcccaaagtctgtgatggatcatactcccctctccattggcattgtcttcctagcatcagttccctccataaggtgaggtaggaatgcatttgagctgggtctgttcattagaaattccaactgatggagaggcaatatgcaggagccagatggcagctgtgagttagaaaaacccatgtgtgccagtgctctcaagtttagaacaacccattctagcagtcgggcagataaagaaaccttaaaagtttcaaatactttcaaactggattttctactgggatcaatggaggtgggcacagacctcttcccacatcttggtttctttatgttctataaaagtattaccctgtgctctattctgccctctgacctttggaagttgtttagactaaaaatagaggaggaagagcagtccaagaaaatgaaccccctccttctcagaacatgagactcatcatagattccatagcccatattccataaacagcctggtggaggcttgcccccaaatgtcatagtacctaggatagtctagttgtacctaagcagtatatgacacctccttgctagatgtggtggtacagaactgccttgggatacatagcaggttggagacaagcctcaggaacataacaagagcctattgcaaaccaaaaagaggaagcttgttgctttccaagctctgttgccgtcggcaggaactgctaagaataaacacaagggtaagaccaactcaggcaagattggatccacttgccatggtccttgcaagtagaatctgcatggtctccgatttgcaggacagtcagaaaataaatttggggacatgacctttcatggctcttgaatcctgtaaaataataccatgcaactctgccatggcagtgaccagtgaggttcagacctgctggacaattggccagctccagcttgactttttaactataagaagcatttggagattatctgtacttttcctagattggacttgggctttattttattcatttatttgttcttgtaagtttatttttaggtttttagaatgagatgaggttaatcccagaaggttttgggaagctggtctgttaatggcatgtcgtgcacacagtttggccatgtgttaggtctcttatttcatgtgctgtctccatgaggagctggggtgtttttgctgggcttgatgtgatggttttaagttcttccggagacactgggatggcattccctccctctgacaggaacatctaccagctgcatctgatcctttcccatccttggcagtcttgtaattacacctcttgctggtcacatgggcaagatgagtattcttgctgaacaggcaatgctgtcctgctgtactgacttttaaatggcctaaatttagagcctctacaaggatggcactcatggattccagtcttcgttcaccatttggtaatgcgccagcaaagccatagctgtgacagaagacacgatctaccttccaaaagtgtaccataggttggccatacaaagaattttaataatgggccacccagtgttctaacctttgggattataatgcagttttatatacacggtgccaaatatgtgtttgtttagagcacttgtgaatgggctgaggtgtgtagctcagcaagaacaaatccctgggttcaaacctcacacatacttataaagatacattaggccagagggaaggattttaactcctataaagtagaaattattcagctgtaattcttctatgggaattaagggcaacctccaaaaaaaaactattatgagtacctctctaaaatgaatcaagcagtcagtagttactgtcagaaaagattagctctcaggccgcctgtggatgaaggacagattcaggcttccaggtgtcttaactgggcacagtggttaaaagacaaagcccttaaaatgatgaattatatatatgtcctgtgatcagtggcgtcacacagatctgcactatccacagattctatagctgtcttggctctgactcccaggtagaaaaaggaaaaggcctttgggggtactattgcaagtgaagtatagtgaacggtagttctatcagagataggccactaatcaccggttcctctccttttctaatcagacctaagaaagtgattgtgaaaggaggtgtctgcatatgaccaagcaatgagctgtttagcttggaccattgttaccttaagtcaacaactttgggtttgaattgactttgggatcgatagtggaaacagactgccagggcagacttcaaacacaaatcctgggtcctttacctgccccagttgttaggcatctttgagtcctagggatactaggacaactgaagggacacatagctgctttaagagtagggctttagtgacagtgtttcagaatgttcttgggatttgctattggagttagttctgtggtggggagaatggatgctgatgagattagtgggttggggatgaggatggaattcagacccttttctttcataagtgattggtcaactttggctcacaggtagggaatgggaactgaagtcaggtgtggggcacctgtcagtcaatctcccttctttccccagtatcacgcagagactatcaagaatgtgcgaacagccacagaaagctttgcatctgaccccatcctctaccggcctgttgctgtggctctggacacaaagggacctgagatcaggactggactcatcaaaggcgtgagtatcctaggaagtgctctgagggcaggcaaaccctccctccctggtagctttctctagtttctgactgggtaagttttagagtttaagtctaccaatgatagtgtttttattaatgcatatgggtgtgggtggtttttttgtgtggctatgcaccacatgtatgctgtgcactgcagttatggatgtctatgagacccaccatatgggtgctagatcttctgcatgaacagtaagtgctctaaaccactgcaccatctccagtcccggcatgagaatgttttttgtcattgctcttttcatctgcatagctaggaaacatgggggttggtccttaattttttttaactacttaagtctcaatggtgaacggagcttagaatttcttcaaaggaagaaggttgcttagtactccgaaccagggtttgagcatccccattaggggtgtggcttctgcagttcacacacccacgggctgtcttgaatctacagagcggcactgcagaagtagagctgaagaagggagccaccctgaagatcaccctggacaacgcctacatggagaagtgtgatgagaacatcctgtggctggactataagaacatctgtaaggtggtggaggtgggcagcaagatctacgtggatgacgggctcatttcactgcaggtgaaggagaaaggtatgcatggcgcatggtccatcgccaattcccatctccagcgctctcaagttgaatcttttttttaagattttatttatttattacgtatacaacattctgcttccatgtttatctgcacatcggaagagggcaccagatctcataacagatggttgtgagccaccatgtggttgctgggaattgaactcaggacctttggaaaggagtcagtactcttaacctctgagccatctctccagcccccaagttgaatattttaactccttttggtatagcactcgcctgcatgtatagggcctgggttcaatctccagaggatcagatttaaaaaaaaaaaaaaaaaaaactccccaaagggccaggagggtggggcgagcctttagtgtttagtcctaacacttgggagtcaatggcaggtgggtctctcaatatggcatgtagaatatttctatgtgcctttcaagtttttgttatgttcacattgttctgcctgaatgtccacctgcatgacggaagagggcaccaaatctcattacagatggttgtgagccaccatgtggttgatggcaattgaactcaggatctctgtaatagcaaccagtcagttctcttaacctctgagcaatctctccagtccctcctgtgtgcctttgaatgccagttaaatcttacctgtttaaatggtttatgtgttcgtttcattttggttttatgagagggtttttttgttgcctgtgctgggctttactatgtaactgaggccaaatgaggctgattcactagcttagctatttattttgagacaaggtctcactttgtagcgctggctgacatcactgtgtagatcaccttagtcttgaacacagatttttctgcatctgcatcttgagtgttggggtaattaacctgtgctaccacacccaaaattcaaaacatcttagcaacttgtacatgggtattgtttataggttcggaaactgacagtgtcaaattgactcagcctgaggtcttgtaatctggctttcacgtggctctagttgcctcagtgatgtgacctcctgcaagaagaggcaggtcctcatcactgatccttcacttacacaggtgctgactacctggtgacagaggtggagaacggtggctccttgggcagcaagaagggggtgaacctccccggagctgctgtggatctccctgctgtgtcagaaaaggacattcaggacctgaagtttggagtggagcagggtgtagacatggtgtttgcctctttcatccgcaaggcagaagatgtgcatgaggttaggaaggtcctgggagagaagggccagaacatcaagatcatcagcaaaatcgagaaccatgaaggcgtccgcaggtgagtcctgtggccccttttcatggtccagccatggttatgagtcctggatccctgcccagcaagtgcagcctgccaagcctggtttccgtggatgtagcattgtagtcttcacttggaaaacaatgattctgatgagtggtccatttagctcaccataggaaagaaagcatatgaagcttcgttaagttggtgttcataagaatgaggcaaagcagggtgactcgtttgacgtctgttctatgttgataagccataggtgctattggaccttggtgttacgtcagccctgtaggcactctgggcgggacacctcacctcaagggtcagcactcagaatcattagctaagtgaagccatgcgcaccttctggcctcactcttgctttgttggaagcgccaattagagaagtaagatacaagaggctataagagcaagacggaagagaagtcagcccacgcaactaaatctcatccccctcatctttcaatgaagtagtcatggtggtattgaagaagcccagcaagcaagattgctcggttgcttggatgctttgttttgttctttgcttcctattgaatatcatctttctcttctgaatcaggtttgatgagattttggaggccagtgatgggatcatggtggctcgtggtgacctgggcattgagattcctgcagagaaggtcttcttggctcagaagatgatgattggacgatgcaaccgagctgggaagcctgtcatctgtgctactcaggcatgtactgtgtactctgtttaggagctgcccatgtactgtgtactctgtttaggagaactttgggtcttgtcccatggtccctgttgtacagacatgttaacctatacatagctgtcctctggtcctacaaaatatccagggagtcatcttagcaactgcttgtcactgaacgtggcccatagtctcacacaggtctcactgaatgctcatctgtcccctcttagatgctggagagcatgatcaagaaacctcgccccacccgtgctgagagcagtgatgtggccaatgcagtcctagatggagctgactgcatcatgctgtcaggagaaacagccaagggggactaccctctggaagctgtccgcatgcagcatctggtatgttcttccgagcatggggtggaaacaagctctgccacagaggcttctgcatcctctacatggtctcttcccattctcctgttgtagtaacagtggcgtagtgtgaaagagtgcttctttatttttttttttccctgtggtgctttcactgcctctaccccagccctctttctcctaccctggaggttgccactttcattacataggccacatttgataaaagatttaagtcataggatgcagtcaaaggatgccttagacctccccctggacagtccagaagacctggtacctctgctctattctaaaagagccaagagtttgttcccagctggtctttttggtgtttggcttccaatttgatgccctactagtccctaggacagatgcttgtacccacactgagctgttttgagctggccttgcatggtgcctgtgtcctgggactgcctcccttgctgtgccacacccaaaccagggctacaaatagagctgggggtggagggagagtgttaaactcagcaggccctgggccaggaatatagaggaactgtgaagacttttctctgggaatatgttctttaagacttctaactttggtcagatatggtgatgagtcatgcctgaagtccagtattcagaggatggggcaggaggatagctaagagtttgaggctaggcaggattacaataagactctatcttatagggacaatagactccaagcagcaacgtcttctgctgcttacggatgaggacccagttcatgagccctgtgttttgccctggaacttactgagcttttacttttgactgatgaaagagtattagaaattaacttcttgagcttttctattccattttcagccagcaaaggacccctgtcctttttagccccagtccccaaatttaagccaactgaaaaaagaaagaatagcccccctctggattagtcgtcattcctttcttcccttcatctgtctgattattgagtttgcaaaaattccaaggtgaactgtattctggctgtgtgaaattttctgtggccttattatttgtgcctcctatattgaaactgtcagacaagccctgttgctgttctggttggtaatgtgggctctcatagctgcccatggcaggtagttgctttttctcccagaaagccatgtgccttcagtcaccttcacagttgctgcctttccacttttataccacatagcccttatctagttacctgcttagttgtgtcatctggccacagcgtggtcaggaacaggttaagggcatctttgaccgggtattctgaaccatgtatccacatagatgtatgtatagctgggggcaagttatatgacctcaagaccttgccctaagatgggtttgatgagatattaattatagccttagcattaaacatttactgccgtcattaaattttagataagcctctaaacacattggtgtaatcttcgtgaccccaatctgtgaatgagtcacatgatgtgataaatgtgggtttgagcccaggtttgctttctgtagtgcctgccaccggcccatttgcattatgctggaacagcagatgtggacaggtctccatgtcctacttccatgccctgttctttcctatggtcagacaccttagaacctcgaggcttgggactgcatggcgctgctcagaagatgaggcacagagtctaggttaaactggctgcctctccagggggtacaagcctttccctctgttaacacctcagcttctccccttacctgtcggctgtttcctcccttgtctaactgatgcagttcatgacctggaatttagagctaggcaccctagctatggttgttggtcctttgcactgcatgcattccattcttaacaaaaggaatacttttctgcttgtgaacctgcttatactgtcaaattagtgaaatgatttgctggttccttctcatccttcccatactaagatgccggtgattatggcattctaattttctgaaacttaaaatttcctttctgatgcctggtcttacctgaacaggtgtgtctgtaatgtaacacacttgctatcttgcaacggttttaattgctaagttttaatcagtattttgacgtgcatgcatgctttcacgggcatcattctcttcacatggttgttaagtgagccagagttttcactattttctttagaactggtatgctgtgggactttggctcttttgaccccagctttccagagctgcctcaatgtttccactgtaaacactgggctgtgtctatgtaagctgggctggaagccctgttgtagtcagacagccatgtgaagagggaagggcctattctattccttcctgtaagctgtgttgtgaggcattgtggtcagatggaagtgatttggcatccttccatgtcaaggtcctagggcctaacactttgtccctttcttagcttcgatttaaagaaaaagggaaacaaatttatagccagatattgatggtacatgcccattatcccaaagaattactttgagggcaatgggggcaagttcacacccagtaagggccattatgtaagatcttatctaaaaaactcaaatgggggcaggggggaggataaaaaaaaaataaattggagggggaaatcacaagtgggtaaataaagtctttccagaaaggaaaaaactttccttgtattagaaccaaaaaagagcacgtggactttgaccaccctgcctctgtctctgcccaaaggacagatttaactgtaccttcctctctgattaatcccctctgtcttcccatgtgttgtctctcctgttttttccttcatcccttctaccctaacccttacagatagctcgggaggctgaggcagccatgttccaccgtctgcagtttgaagaacttgtgcgagcctccagtcactccacagaccccatggaggccatggccatgggcagcgtggaggcgtcctataaatgtttagcagcagctttgatagttctgacgacatctggcaggtagggccctgagggcaggtatcgctataggataaccacttcttacttcagaaaaaaaaagccttgtgcctgagcttgggcacagcttctctccttcattcaggaaggtagccaaggaggtcatgatagggcaggaccagagtactccctcatgggcacagtggaagtcacaggcaccagtgaggatggtttctgtggagtttttgattttgctcaatttagaacagttcagtgactgttcctcctggacctttttttgcccaggacatcttcccagttgtctgtgactcatcctccctctccatttgtgacaaagctctgacaaagccctgtcccccctcccttcgtccctctggacggatattgctcccctagattgcccgtgaggcagaggccgccatctaccacttgcagttatttgaggagcttcgccgtctggcacctattaccaacgaccccacagaagctgccgccgtaggtgccgtggaggcctctttcaagtgttgcagtggggccattatcgtcctcaccaaatctggcaggtaggaggcggcagtggctccctggggatgcccacctcaattggcagccctccttagggatcccaagagtggctctgggccaagttttgctgccactaaaggactccaggcaccactcacaggtataccaaacaaaccatgtggttccaataaagttcagcttgcctcacaatgggaagcaaatacccagttttgcctagtttagctaccatgtggtcttgaaaaagggagcaagctgactcctgatctcacatgcatgtcagcagatacactaatagtgataagcactttaaaattccacttggactgggtggtggtagcgcacatgcctttactcctagcacttgggaggcaggcaggtggatatccatgagtttgaggccagcctagtctacagagagagttccagaacaggctccaaagctacacagagaaaccctgtctcaaaaataaataagtaaataagtaaataaataaatattgtgcttggaggggttggagataacatctcagtgagtaaagtatttgttgcacaaacatgagggtccgaattggaatccagcacctatggtaagaatgcatgcagacaggacagtactcggggcttcccatcaagccagtcgagtttcagactccataagagatcctgtctcaaaaaattagatgagtgtcttggcctctgaggtccctggcttacaaaggctcacaactgtctgtctagtcctagaggaatccaatgcccccttctaactccccacaggcagtacatgtatgtggtgcacaaatgcacttatggaaaatacccttacacataagtaaaaacatccagatgtgatagcccatatctgtaatcccagcactcatgaggcagaggaagaaaggtgggctacagctttgctcttaggagctctaagatataagatcaatgcagggcttttgcctcagcctctgcactttggcgaggagttcctgattacagcaactacccacttcaaggccttgtgaatgcaggccagcatgaagaagggtagtctctgctgtgttttttctggtttccacagtacttgtgtgttttcagagcttgttcagttaattcagcaaacctgagacacacctgctagaagtagacattatcacttgtcacaggtagttttgagaagggggggtcactacattcccaaaggttctagatgggctaagcattgctatataatgcctggtcactgtccggattttgtggcaccaagtgtgttttgggctctagagttaggcttctgggactgccatgggaacagcatactactgcagtccttaatattgttggctcccaggagctaggtgtatgctgcttggttgcaaatgttgctagcagttgtggatgctggtgagattggccaaagctgtctccctgctacatggaagggttcatgatatgggagggattctagccatgggccctgagggactgctgctcctattgaaggagcagttaagaaaggacagaggacaggatgtggggggtggccagtccttggaacagagtaagctctcagtgggcaaggcctgtgcctcacttgtgttagtgagctcctgctgaaggtgcttcctgtctcctgggtcttgtgacacgtttcatagtctatcctatttcccatctcaggagtgctcaccaggtggccaggtaccgcccacgtgctcccatcattgctgtgactcgaaatccccagacagctcgtcaggcccatctgtaccgtggcatcttccctgtgctgtgtaaggatgcggtacaggatgcctgggctgaggatgtagacctccgtgtgaatttggccatgaatgttggtatgtagctggaagcaagaagatggggtgctgagcaatggcttttctgggaattctgaaactgtcattctctgaagacttgggctaagcctttttgtccacaccctcccaatttggggcccaaggcaaaattaaggtgggacacacagattatgtactgctgctatagcctttgttcctctataaggttggacattggggtgggaggaggccgctggctctgtgatctccaaaggtccctacagtcttgagttcagccattgtttggtctaggaggcttgcaccaccttgtggtagaaagaagaattggttcaggcagactggtccccttatcataggcaatgggattgtaggtagctctttgggcatttgccctggaatgcccatgcctgggtggcctctgggctaggagggaagtgccaccttgtggtaggagagagtaatggttgttcaatttggggcagttaccccaagtgtttacttctattcacaacctttctattctttcaggcaaggcccgaggcttcttcaagacgggggatgtggtcattgtactgactgggtggcgccctggctctggctacaccaacaccatgcgtgtcgtgcctgtgccttgatggccctctggagcccctcttctagcccctgtcccatcccctcccccatgccatccattaggccagcaacgcttgtagtgctcactctgggccatactgtggcgctggtgggctgggacacccaggaaaatttactggctttgaaatatggactagagcccagctgtctcatggccctacccaagctagggatgaaggaggaatgcagaattgtaaaccctctgcctttatcacaaaaggg

SEQ ID NO: 45 - DNA, 32596 bp, Homo sapiens
  source 1..32596: mol_type = genomic DNA
aacccataaatctgggccctgcccaggtaggccgggacagctggggtggcctgggccgagagccaagaaaagacaccccatctggcagcccaacttggcggcaacaggtggcccggcgcccgggggtctgggaggaaagtcgctccgggggcgggccccgttgccccgccgcgtccccattggtcatcaggtttcttaaaatgtgactctgaatctgtgtccttccgccgcagaatttagtcccaccgaaagggcaacctgcccgcgcgttccgccgccgccgccgcgcttcctcctgaaggtgactgcgcccgcggggacgcagggggcggggcccgggtcgcccggagccgggattgggcagagggcggggcggcggagggattgcggcggcccgcagcgggataaccttgaggctgaggcagtggctccttgcacagcagctgcacgcgccgtggctccggatctcttcgtctttgcagcgtagcccgagtcggtcagcgccggaggtgagcggtgcaggaggctacgccatcagtccccaccaagggccagtcgcccggctagtgcggaatcccggcgcgccggccggccccgggcacgcaggcagggcggcgcaggatccagggcgtctgggatgcagtggagctcagagagaggagaacggctcctcacgcctggggcctgctcttcagaagtccccagcgccgttccttccagatcaggcgggtccgccggctcctttcccgccccagccctaccccctcattctggtcccatcctcttcctcctgccccaatcctcaatgcgcctccatcctcgcctgccttctctcggtccctcgtgattgattccacccttgcttcccctttctccgcgccgcctgttccgtgctcgttttcccctcttcctccttaagtctggtccttccaccccctcctcttcaagctgtgcgtgtcccctgattctaatgctttctgtgtaactcattgaaactgcgttctggttcccctcccgcgtccattctccattcatgcgcgaccgcccttcccgcgccccagttccctctcgccgcccctccccctgcttgctggtcacgtccgctcccccgcatccccttcctcgcctggcgtgtccgctccctccctccctctctgctctggtcgcgcccgcccacttgctccggtctccgagcgcggtcccaccccccttttccatcaccgcctcccagctcccagcaggctcgggcggtgctgaggccccgtgtccggggcgggcgggcggagggctgggctgggtgccccgcgcggcgggcgggatgcggcggcccgggggcagctggagacttacgtaacgttggcctgccccgctgccgggaggcagggcggttgcccctgcggcgcggtgccgtcccctgtggccgggattagatgggcggcctgcgagggcctgggaatggctcggggcccgagagctcgaccggcccttgccgggtggccgccagggaccacgctccatctcgccgcggccgggctgcacgtagcggccgcgcccagggcccaccccgcttcaccgggcgatggccttgggcctcgtaacgggcgggataaacctctgcaggctttgctggggcctcctggccctcgcccgttccgcgcctctccggcacgtttctttcttttcctttcttttttctttcttttttttttttttaattatccggcacattttttaacaaatgcgtcctgattgtggaacgcggaggccgcggggtggggtggggatctggttacggagggggcaggaaatctgtcgcgttcactgaacgcaaacggtgtgggtcaagggctgtttgggggtcagagttagagaccaggatgactagacgagtcatagcccaccgagctcacaatcttaaaatgtatctcctgtaatgctgggagtggggtacgagcttcctgctgtgggagggagggggacaggaagcctcgtaaggtctcaccaggtggcaagcacactggattagaagatgcaggaaagcaccttccagtctgaagatcatttaaagactcagtcatgtaagggcatcccttggcttcttaatctacgtactccagacccagggcttggacctttgctggtacgggttaaagtgaggccgacagtgaaggtccacgtggagagagacattgggaagttgtcaaaaaggcgtttagaatcaggtttgactcattaatttcctgagactctgaaggagttcagcctctacacctcagtttcccctgggaaaactgggagtaacattttcctcatgatggttgaaaggattcatgcttacgtggatgtggcacactcagtaagtgtctaatctctggcgaaatacccagaagaaaagcctgtgactgaatatattatttttgagcacaattacttagtccacaattgagcataagggccttgatccattgtcttcctactgtccccaaacgcctacagctaatcgtgaggaatcaggctctctgaataaccacgtgtctcctggaaaccagttccccaagggatctggtttaatattgacaaagtaactgataatgtaactttccaactctctgcttggagcagatgctttttggtcatctgagattgggacctgtagacaaaggcaggcaaggaggggctgagttaagcctttggtgttccgctgtaagtagaatactacctaccagaggaacattccagcccttgacttgctctgcctgctggtgagattaggattggagccagggtcagtagcccccagttggccgtcttgagggtgtggccactccctaggtcacagaggagatggaggtcaggactcccctgctgcagcgttgttagcaaataatttgccctttggtggcctgaggtgctgtgggggcaggagtgggctgtcttgtggggaagggaaatgtaacgtatcaccaaatctgtgggggagtggggctctgtggggaagatgactcaacctttcaattattctgcttttgagagaattatcttcgctgggtgagtggggaaactacattgtggttcttgcaggcttaaaaagtcccttctcccctttcagtgagtcagataatgaagcatgcatttcccctaattaaaaatgcctcaactacggagcttgcagtgagccgagatcgcgccactgcactccagcctgggcgacagagcgagactccttctcaaaaaaaaaaaaaaaaaaagccacaactaattaaaaaatgattggtccgggtcacctggttctctgtactgccttaagagcttaagtgtagactggcagagcccggtgcccagctaatgccgtctgtgcagcttcattcctggcccttttgtagcgggcacagccgcctctcctgggacttgattaatgatgacttaaagaaggttcttgaactcatttctcagttactgacttgagccaaaatgtatggatgttggtctcctgtctagcacaactgctttcattttaagcattgcattagaaataatgttgtattcatattttagcaaagagcatcattagctctttaagaaccgtacccccagtgacttagcaaatttggtggcggtgactctaaacagctagcactttatgggagttgcttctgtgccttgaatgttgtgacactgatgtggggtcacaggaaaaaccattttatttcctaaagctgtttatttcttaatattgaggcaatggaagaaatgaggaaacggccgtgggcaggaaggtagaagaaaatgcagcacttacattacaaactcactttacttgggttttagtttattttgcttcagttttttgttttttttttttttttttttttttttttttgagatagttttgctcttgttgcccagtctggagtgcaatggtgtgatctcggctcactgcaacctccacctcctgggttcaagcaattctcctgcctcagcctcccgaagtagctggaattacaggcacttgcaccacgcccagctaatatttttgtatttttagtagagatggggtttcaccatgttggccaggctggtcttgaactcctgacttaggtgatccaccggcctcggcctcccaaagtgccgggattacgggcgtgagccactgcacctggcctgcttcagcttttatgaaggcaagtcaggatgtgttatcttggccaagagctaagactgagaccggagtagaggaaaatgactcatttgacattacccttctttctgaatcactccaaaagtccacagtcaaatctcaattagctggtataacacaagaatgcgttgaccctgtgagcagagttttattgcatcttcaagggtcttcgtgagattggtttatttggtatgaggtcttaaggattttaagttgtccctttaagcatcagttctgatacagatgggaatagagctaaacaaccagatatatgaagaccatttttgataccaccatacttgcggggagcttgcatttttttaccaaggggcagactggataattgagtaatttaaatacttgggactttgttaaagaagctgttgggggaaggtgagcctgggtgagtggtggagggtgatgtgcagcatctgctgggccttcctgcctcttggtatgactgctcagctcaggagttgggtgctttagggcccctaccatgcagtgcggtgaagccccgcccctgcagtggtttgtgcttgtctgcacgtaggagggaacaagccggctgcagtaccttcggtggcctctgagactcacctcccccacttctgtccatagacaaagctcctggggagccctaagcctctttcctcatgccagcaagcagtcttggaagcaggctggggctgcagtgggagagatgccaagaccagttattcaagggcagtgaggttgccagtcctagcatttcacactgtggaccagcacagctgctggctgaatactgcagtcaagattctctgaagggagtcctatcccttccaaacatcttcagtttacttgcagataatgtttcttcattttttattttattttattttattttatttttttgagacggagtctcgcaccatgacccaggctggagtgcagtggtgcaatctcagctcactgcaagctccgcctcccgggttcatgccattctcctgcctcagcctcctgagtagctgggactacaggcaaccgcaaccacgcctggctgattttttttgtattttttagtagagacggggtttcactgtgttagccaggatggtctcagtctgctgacctcgtgatccacctgtcttggcctcccaaagtgctgggattacaggcatcagccaccgcgcctggccatgtttcttcaattttaaacaactgatatctcccttggccatgaacgaaaaagaactgcccatcagtggagtcagtcagggcacataagacacactgtgtccaccatgccatttcagaggagatttgattgagttaagcagggaaatagagatgttgtaaacgttgaaactatctgggtatccctctttggttattaacattagatgagcagaaaaacaaatgtcaccgatgggcaaacatttaaaaagtctggcagtaccaagtgtggaaaaaggtgtggaaggcagcaactcagcgttcattgatatagccactttggagagcaattcagccttatttagtaaaaatagaaataaacatactccatgacctaagacttcaatttctggatatatagaaactcacacagtacagggagacatgtactacaagagtattgattaatagaagaaacttagtttaacggggagggagagtggccaccagtaagacagtccataaataaaatggtctgttgtacagtggactagtgtaacagcttaaatgaattagctagatccatatacccactggatggatcttaaatgtgctgctgagtgaaaaacaagttactcagtgatatatacagtataccacttagggcattaagaaaaccacaatattatagggttcagtatagacatagatacactgtacatagacttcagtgaactggaaggatatagagttcatgacagtgattgggtgtcaaaatgcatgatgtggctgggagcagtggctcacacctgtaatcccagcactttgagaggctgatgcaggaggatcacttgaggccaggaggtcgagaccagcctgggcaacatcgcaagattcccatctctatttaaataaataaaatagaaaaaaaagtttaagatggaggtggaaagggttggaggagcttggggatggagaaaaaatgaactgtataaaattaaaattcttgtttttgtttttagaaagtaaagagggccgggcacagtggctcatgcctgtaatcccagcactttgggaagctgaggtgggcggatcacgaggtcaggagattgagaccatcctggctaacacggtgaaaccccgtctgtactaaaaatacaaaaaaaaaaaaaaaaaaaaattagccgggcctggtggcgggcgcctgtagtcccagctactcaggaggctgaggcaggagaatggcctgaacctgggaggtggagcttgcagtgagctgagatcgcgccagtgcactccagcctgggcaacagagcgagactccatctcaaaaaaaaaaaaaaaaagaaaaaagaaattaaagaaaatacagctcagcctttatttgtgtttttttttttttcctttttttctgagacagagtttttcactctgttgcccgggctggagggcagtggtgcgatctccgttcactgcagcctccacttcctggattcaagcaattctgtgtctcagccacccaagtagctgggattacaggtgcgcgcctggctaatttttgtatgtttagtagtgatggtgtttcaccatattagccaggctggtctcgaactcttagcctcaagtgatctgcccgcctcagcctttcaaactgttgggattacaggcgtgagccaacacagccagccatggctcagtgttaatggtcagttctgggtggtagagaggcagatgttaaaacttttttttctttaattcgtaacatagaagcaaacctataaaggctgccgtaggaagaccagtcatagtaactagttcagtgctcttggagagttggcactgcctttcctcctttatccccccgactagaatgcagggcagcccttccagtaaatgttgagccagtgcctcactttgctgaggccatcacccaccttagttgcacttaagaggaccctaaatcagggtcccaggtcccttgctgattttagagtgtggatatcatacccagaaacaccgccctacttttaatcctagtaaggaggcaccatgtcccaggacaactaatgcttcccccaaaccacctccttcaggctgaaaccagttctctgcactgagcagctgggatggaaccaggaaatcctcggcatctgaggacattgaggggtctctgacttagggcttcttcacctgaagttgagtggtctttgagggaagtaggcccatttagcatcagctgctcttccctattccacactctagttggaaataggaccttaggttcctgttgacaagtcatttactttcagccccgaagaaataaaagagccaagattttttttttttttttaaagccagggaattttactagaacctacaagtgggctcattttgttctgtgtagcctggtaacaccatactgctttctgctgtggggcctcctggggttaaagtgtgggcttaagacccaggtctcttagctagaagatatcttatcctctgtatcctgcacccatatgcaaatacattattgtcattacccttaactatagatgaagatgaacagtgcctattccagaccttactaggttctgctggcccgtcacccattttgatcatgttgctggcctagtttgattagggcaaatcttagaaactccatttccattgttgaggaagagaactagagagcaggctgacctgaatgccagcgtatcatgatgcagactttctaacggatgcaggtgttcggaagagttgtggatcgaaacgccttcatgatggcttggaggtgtaggtagcaaactgacgtcacaggaaggaacacaatcttgggtacctactggcaacgttggagggagaaagtgagcatcaggtgccatcattttatagttgatctatgtgatgaggttggtatcggagcataattggtacaaaggaaaaatgacttaggcagatgcagactcacgggccaggctattttattagggcagaatgatttggtcctttgtggaagaattggtggagtgaagcgtgaatctttcccagcacaacccaacaacagtcctggccctaagaagtggagcatgggagttgggtgtggttcgtgcctgtagtcccagctacttgggatgctgaggctggaggatcgtttgagggtgcagtgagctataatataatataatcacaccactgcactccagcctgggtgacagagtgaaaccctgtctgaaaaaaaaaaaaaagaaaaaaaaaaagttggagggaggagtgttgggtattttttatgatttttgtctcctggtttctgaagaatggccaaaaaattgtgtctgacacaaaggaaactaatataaaaagccaggagctgtctgagatgagaaaggaaagggagaatagggcacttggagctgagctgtgattgtgcctgttccaacctgtcactccagactaaggcctcttagagatggtgtctcctttctcacagaggagacatggctctgaggaagatcttactgcaggggctcgggctcaagaataaggctcctggacctgggcatggtgtgtgctgctatcagtggatacgccaggcttccactgctggctggaggttggctctgcatgtctgtgccttcctaggaggaggatgcaatagtgagtcagactggcatgggtggggccacatgtcctggcaggcactgtccaggcagctggcatgagggagaggagtctttcccagaagtccgccctgagaaaccaaggctgggctgctctcctggagccaggggagtccagtgagtgttttacatacaactctaggtacgtggttggtgggatgggcagtttgtgctgggaagaggcttgtggaggattttggagaaggcaggagagctctggccctccctgcaagggaggcttcaggtcagagcctgaggaaagacctgggcatgagatatgaggtctctggctgggtagagagcatgaaggacacatgagatctggagtctggatgaacttgctgacaagcaagctttttttgactgctaatgcgtaaatccactatagaaattttcatttgtcatttttgtacttattggcaaaaaattagagcttataggcatcagatttaatttgagatagttgaaatagggtttgtgtttgaggccaatataattttatctgctaatggcatctcgtggacttggaggcagcccttctgtaccagaacattgtcaaaagcttttactgtaaagcttgagaacaaactagtttgctggatttgggcatgtaactaacaggtttgaggcatgggattatcctgtggacttttttttttttttttttgctttgaggttctgaatgtattaaggttgaccttcttaagccgttgaaactgttctgcagtacatttgtgatgtagggcctaagacttgtatcgttttttttttttaatcacaacccagtgtacagaacttgagacatgcgtcttttctctgccaccttttaaaagcagattattcttgaagtgcatagagcagcaattgattaatggaattggtgtcttcacatttcatttacttcctcccaacaattttataggatgcatataaatatttccaaaagaggtacacataatttcttactataaaagtatttttatatttatatcagtaaatttgttaataaagaggatttttttttttctgtaatcctaccaccccaatgacgtcctattaaaatttcagtatatatcctcccaggtcttttaggtatgtttaatttggtgtcccttccccgctcccaaaaggggagggaccaggttcttgtatagaatagtggaatgttagtaaatcacaggtttaaagagacataacagtggaatctctagagcagctgtcacctggatacctggttattaaggtaatttttccattaccccaaagagctttagttacactcagctttttccttaatccttgtgcagctctccagggcacaccgtattcagctctgagcggtctttgctagtgaggccaaggagccaccctgagccaaaaggggagcattatgtcaccggaagcccaaccccagagaaccaaaggtatgacctgatattcagtggccccagccaggtctttacaggaagaccctcatatctcaggtctaagaagagccagctgatggtttttaaaaagagtggaattagttactccaacccacttattcagatcttattttgttcacaatacagtccctagattgtaggcccattggaggccacagcaaagcctttgtgttccagttggcctgatgtgccatctctcagtaatgttcccttaacagccagacttccctaagcccagctgggagctctgaaggtatgcgagccctccctcaaccatgagtgtagggaaagggaccaggggccccaggctttcctgtcagtaatgcagaagttcctcagatttagggaagggggagcagaggcataactttgattctgacaaagaggcattcagagagactgaaaggtcatttaacaaacactggaatgcttccacatactaggtgctaggagatacaaaaccatataggtcctggaagggaggattgattttttcattttggtacgtagtagatattaggggcttaggaaatacacatcgaaatgaagagtgcatttgccatgttgaaccgttagccggtatcttatttccccattttaaaagttttagaatctgtggttgaggacttgtggccatcagttttccatagccaacagactgttcactactgccttcagagctccttggacctcagcgggccttctttggagatggcagagatggatttagatgtatactctactcgagccacccagagagcccacaaagtcagagatggaacagggtaaaggagtaagggtcatatgtgtgagatgccttgatttggaactttgagatttaggatgaggtggggaagggctaaagaggagcttgttcctgagccttgcttggccgaagcatttaggctcaagcgttttagaaagagtagcccttggtctgagaactcaaggaaacagctttctgatgagacgtgtagcaagcttctggttcacatccttacctgatagttcttcaaacactgcctggtctggttcacatccttacctgatagttattcaaacactgcctggaagcttctcctgagtttttgtctctaatcagctaactaacaggctgagtgagtttagttgtaagtcattaatgaagaaagcaaaggttggggccattgtcagggttgtgacctgggctagttaattacctggaactgatggtctgtgttacagagtggtggtatacttgtcaggcttagaaaagaaatcaggatgtgtatcaaaaatcatttggggaaaagatttgaccagcaactttaatttctctatgtttgcaactatcctgttaatgtagttgtgataatttcagaattataccagtgcccttatgttatccttgctttgcaaattgcaaattgctttgcgtgtcctgacatccttctggccaacagtagatgtggttttaggtttagactcctgggatggaagcttttgcattcaggggaatgactttgggtttgggtgaggattgtaaagaggcaatatgggtgccccacgacaaagcagctatttgtagctttgtgacagcttgacatgcagagatctaggcttatcaaggcactaagctaggagtcagttgtttgtatcactggaagattggttacaacttccttcattggaagctccttcagtgcatgttaaatgatgttatttatagatagggtggtgagaaagctgtctaggtagatgtcagtcagcccagtgtaagagagacctgcttactgtgggtgcttgggactatgtggagtgggtgggaggttttaacttgttcagtaaggtcctttccattgttcacaatctggtgaaccctttttctaacatgaggagcacccacataaccagatcatgtctggcttccctgtggcttgtgtacaaagcgtgcttattgagttaatgtgtaagcaggagacagccttctgtgctaaatggtatattaaccacttctcagtcttaccactctctttcaatttgtctcgacccaggacctcagcagccatgtcgaagccccatagtgaagccgggactgccttcattcagacccagcagctgcacgcagccatggctgacacattcctggagcacatgtgccgcctggacattgattcaccacccatcacagcccggaacactggcatcatctgtaccattggtgagtgggtgtgccccttcccccaaaaaagggcttcatgggcagtgacctttctctcctgaaaagagtaactaaatgtcctaacaaacctaggtgctacatgggatactacacagattcttatgaaaggactcaggtcataggaagttgcagtaaagaattagtatgtgcataggatggcaaatacagttaataagagagtattagacatttcaaaattgctaagatggcgaggtatggtggctcccagcactttgggaggccaacgtgggaggattgcctgagcctcgaaatttgagaccagcctgagcaacttagaccctgtctctccaaaaagtgaaaaaaaaaaaaaaaaaattagctgggcatggtggcatgcacctgtagttctggctacatgggaggctgagacaaagatcacttgagtccaggagattgaagttgcagtgagccatgatcacaccactgcactccagtctaggcaacagagcgagatcctgtcttaagaaaaaaaaattgtccgggcgcagtggcacatgcctgtaatccagcacttcgggaggctgaggcaggtggatcacctgaggtcaggagttcgagaccagcctggccaacatggtgaaatcccatctctactaaaaatacaaaaaaattagccgggggtggtggcgggtacctataatcccagctacttgggaggctgaggcaggagaattgcttgaacctgggaggcggaggttgcagtgagctgagatctgaccattgcactccagccttggcaacaagaacgaaactctgcctcaaaaaaaaaaggaagaaaaaagaaaaaaacatcgctaagagtaaatttcaaatgttctcaccacaaaaatgttaagtatttgaagtcatggatatgttaactaacctgatttaattattccacattgtatccaaactgtatgtattggattacataactttgtaacccaaattataaattaccagtttataataaaaaataatttgttgcaaaaagaatccatatggtttaggttttatgctataggcaaaatttagaagatgttttccttagcaggtctttgtaggagcaacttaaagacctaggaaagatctttctaacatgttctgtgctaccaagattctgtggttggacatctggctgggtttcagtgagggtggagaaggctggccaagtcttaacctaggcttttctgatacagtgggagcctgcagaacttgaaggaaatggtcgaagtgtcccagtagatcaagaaagtaagctggcacggtagtagccttccatgcactttttaaagacttttgagctatttgggagaggaaaagttttcagggaaaaaaattctttaaacttaagcaaacttaaatgtttttccttctttgaataattaatacttgtggctttaaaacttttcctaataggcccagcttcccgatcagtggagacgttgaaggagatgattaagtctggaatgaatgtggctcgtctgaacttctctcatggaactcatgaggtgagctgtggctggaccctatggccattgtgatggcctgtaggaaacagggagggggtgcagtgttcgtttagccacagtggactagacaaggatgagtctgagtttcacagtcagtgtgaagtttgtctttactagcccatccctactctccttccctcttgtcctgacaaagcaactggctgagtctcttttagcaaaaaggaccccctttgttgctggctgtggttctcccacacacctctcctacccttagcttttacaaaggaagatatggaaaggttctactggaaaaccctctaagccttaggtgtcctggccacagcgcttgactctcctgtcccagggtttctgcttcaccttgtgttgccatggtaaaccatctagcagattgattctagcttagaaccaaaataactgggcaggtccatgagaacggtttccactattctaagttttgagggactgagcctaatgcataagcactatctggggtgtaataccccacttcctcagcactgtattctcagcctgtgccttcccaggggttctggtacattaaaataacaccagttagcactcttccccaggagcctagtaggactgtatttgtgctgggctctttattagctggctttacctatggacagaggccttgcccaggagccaggtagcagctgttgggatggctccattcctgcctccattgccagatttagaattaacccattctgaggagcttggggttccctgaggtaccatgacttatttatttttttattttataaaacaaaattttgctctgtcatccaggctggactgcagtggtatgattatggctaactggatccttgacctcccaggttcaagtgatcctcttgcctcagcctcccgagtagctgggaatacaggcatgcaccaccacacctggctaatttaaaaatttttttgggggaaatgaggtctcactatattgccttggctggtctcaaactcctgggctcaagtgattctctcaaatgttgggattacaggaatgagctaccatgctcagcctgggattgtgcctttttaaaaccttcagacttaaccataggtttcccatagatcatgggatttcgtaatggcattgataagaggaattacagaagaggcaaactttgcacctgtcttggcttctgtatttcctgttgagagtaaagaaaatgctatcctgtaaggccaattgccttacagaggttgccctctggcatttggaagttggtattaagtttggactaaaaataaagcctcaggaaatgcaatccaagagtgaattcctccttttgggaaacacaagactcttcatcatagattccctaacctgtgttcataaacagcctatggcctggctagtggctggcccttaaatgtcatggggacctgaccaagtccagcagacataccatgtaggttaagacatgtccctgtaccttttggaaaattctgtagttttccaaaagcaaggggtccttagcaggagtcaccgagaattacttgttagagaattaagtgttagcttagcttagagagagctgaagacaatgctggaggtctgttcgctgttgatccctgctgctgtagtctgccatgggctcctgcattcaggggaaggagcagaaatagatttttaagaagttgacctttaagtaggctttatggttccttcatccagtaaaataacaccacatagctctaacatggcaagggcgagtgatacctgccacacctgctggatgagagctggctccgattttggtattttaaactttaagaggcttttggagattatctctactttcactcctattcccagattataattaagatttattttttattttttatctatttattttttaaagatgtccctcttgtgtgttcattttgaagttttagaccaagatgaggttgtgtgtgggctcagcttggaaactgatctgaaattattctaatttatataatgtaatgtaaacagtttcagccttaccatacgtcagggctatcgtttcatgtgcacctttgactaggggctggggcgtacttttccagtttctgactattttaaatgctcttctgagcagaacgttgagattactgtcttccctctcactctgacagagggacatcaaatgtctgcatctgatcttttaacagctttttttttttgagacagaatcttgctctgttgcccaggctggagtgcactggcacaatctctgctcaccacaacctctgcctcccaggttcaagcaattctcatacctcagcctcctgagtagctgggattacagacctgtgccaccacgcccagctaatttttttatatttttagtagagacggggtttcgccatgttggccaggctggtcttgaacttgtgacctcaggtgatccgactgcctcggcctcctaaaggcgtgagccaccacgcccagccctcttttaacagctttggcaactagtcttcagccctcacttttggcagttcacatgggcaagatgcattcttgctgaacatgtggttccatatgccatgttttccagatttatttatttatttatttatttatttagagagggagtctcgctctgtcatccaggctggagtgcagtggcacgatcttggctcactgcaacctctgcctcctgggttcaagagattcttctgcctcagcctcctaagtatctgagattacaggcacctgccaccacacccgactaatttttgtattttagtagagacttggtttcaccttgttggccaggctgatctcgaattcctgacctcaagtgatccatccgccttggcctcccaaattgctgggattacaggcgtgagccaccacacctggcctagaaataatgacttttaaacaacctaaatgtagagccttccacaggacagcattgatggatgctttaccacataacatcccaataaagccacagctgaagtggaagactcagtacacctcccagagatgctctaagagattatgatatatgacatagatttgaataatatacctaataattggtatgtttataatatatggttttacatccccaagaccaaaaatgcatgtttgcatgaaacactcatggttacaaaaatatattaggccaccaaaaaaacccccacgtttcataaagtagaaattatacagacacattctctgataaaattttttagtggaaattaagaacaaagtcaagaaaactgaagtgtgcttactttagaaagcaaagatctcaaggtagatgaaataaatatttaactcagaacactaggggaggaaaaccctaaaaagggtgaagaaaataattttgtaagattatagctcaatgaaatgaaaataaatttgacagattaagctaagagctgattctttgtggggaaaaaatagtaaaatagaaagttctgagaagccaggtgaagaatgcgaggatgtgcaaataagagtatgaatagaaaagagaatatttcctacacattggagattttaaaagtcaagaaagacttataacttaattcctatatagagagatgactctggctattttcaaagaaaagataaatatccaaaagatagaaaatatgaatagaccagtggccataagaagttgaaaaagtgggctgggcgtgcggtggctcacacctgcaatcccagcactttgggaggccaaggcggagggatcacttgaggtcaggagttcgagaccagcctggccaacatggtgaaaccctgtctctactaaaaatacaaaaattggctgggcatggtggcacatgcctgcagtcccagctactcgggagcctgaggcaggagaatcgcttgaacctgagaggtggaggctacagtgagccaagatcgcgccactgcactccagccaaaaagttgaaaaagtgattaagatctggttcacctcagaacacttaagtccaaatgattttagtggctgaattgtctccccttcaaagttcagttacattgttaaactgttccagagcctagagaaatatagaaatcttcccactgtgttctttgaaaccaatatacgctgatactgagatcaaacaaggacagtaccaaaaccaggcaggtactaacagttagtgtgctagaccagtctcacttagatgcagaaaacaaataaaatttaataatccaaatccagtagtgattgaaaggaatgtcttgatccatgaccaagtagattttattctaggagaacaaaattctacatcgggattaagtagagttaaggttgacatttttttttttttcttctgagacggagtctcgctctgtcacccaggctggaatgcagtggcacgatctcggctcactgcaacctctgccttccgggttcacaccattctcttgcctcagcctcccgagtagctgggactacaggcgcctgctaccacgcccggctaattttgtttttgtacttttagtagagacggggtttcaccatgttagccaggatggtctcgatctcctgaccttgtgatcccccctcctcggcctcccaaagtgctgggattacaggcgtgagccactgcgcctggcctgagttaaggttgacttttaaacaacctaaatatagctaaatatagagccttccgcaggacagcattgatgtgtggaactcttatccacgtgataacatcccaacaaagccacagctgtagtggaactcagtacacctgagtcttatcattataagatgataataggtaacatttattagataattaccatgtactttgtcctaatacttcatgtattcttttactcctcacgtcaactctgaaggaaaggcaccacctatccccttaaaagaaaacaactattactattcttttttttttttttcttttagagacggaatctcactctgtctgtcgcccaggctggagtgcagtggcacgatctcggctcactgcaacttctgcctcctaggttcaagtggttctcttgcctcagcctcctgaatagctgggactacaggtgcacgccaccacgcccagctaatttttgtattttaagttgagacgaggtttcaccatgttggcctggttgttgtcaatctcttgatctcatgatccacccgccttggccttccaaagtgtttagatgacaggtgtgagccaccgcgcccagcctctattctattctattttgttctatttctattacaagccagtaagcaagaaaatatcataatttataaggaaccctataaaaaacagacaagccaagggtctgtcattaggaagtatgcctgaataagaagctgaagatttttagacacaggtttcaggcaacactgtctttagaggctaggctctggctccagctccctccagcctcctgtgaataacaggcaggcttacttgcaggtgccactttcctggacagtggtggttaaaggacaaggcccagaaagtgctgaattaggtgcccttgttaccgctaatgtcttattgatgacactatcttagagctcttttgacatcttggctctgcgtcttttttttttttttttcttgagatagggtgttgctttgttatccaggccggagtgcagtggtgtgatcatggctcactgtagccttgacctcctagacataacccacctcagcctcacaagtagctgggaccccaggcacgcaccatcatgcacagttaatttttgtgttttttgtagagacgaggtttcgccatgttgcccaggctcatctcaaactcctggcccaaactgtcctcccaccttagcctcccaaagtgtttggtttataggcatgagccactgtgcttagcctgagtccctcttttaaacaaacaaaatggtaaatggaaaggaggaaaggcttaagaaaaaagattgaagccaggatttgttgtaagcaaggagtaataaagggcagttcatttagagaaaggcatatgaccacctttccccctccaatcagaatctagaaagtgattgaggccgggcgcagtggctcacgcctgtaatcccagcactttgggaggccgaggtgggcggatcacgagatcaggagatcgagaccatcctggctaacatggtgaaaccccgtctctactaaaaatcgaagaagttagccaggcgtggtggtgggcgcctgcagtcccagctactcgggaggctgagaggcaggagaatggcgtgaacctgggaggtggagcttgcagtgagcctagatagtgccactgcactccagcctgggcgacagagcaagactctgtctcaaaaaaaaaaaaaacaaagcgattgagaaaatcaggtctgtgtgaccttagcaatgagttatttagcttgggccactgttagcttaagtcaataacttcaagtttgcgttgtagttggaatcaatagaggaaaagctctcagcattaccacatatatcagaatgtgacattgattgccagaccagccttatccaaacacaagtcctaggctttttgccctgtttatgagctttatatgctgagggtatttgatgagtcttagggaaaaaagaacagccctggggacacagctgcttttatgatgagacatgtttgcacccataccttaatgggttttggtggcaatattctgaaatttgccacctacatttcaaagatttgccctttgggtgaattagtgctgtagtagaagtgggtggaggctgaggaggttggattaagcaggtagaggatttctcagtgcatggatcgtgctgaggatggagatagagctctaagacatccacgggcctttcctgagtgatcagctttggctcctgggcaggggaattggagctggattctagtgtgggagcacgcttgtcatcttccttcttttcccccagtaccatgcggagaccatcaagaatgtgcgcacagccacggaaagctttgcttctgaccccatcctctaccggcccgttgctgtggctctagacactaaaggacctgagatccgaactgggctcatcaagggcgtgagtattctgcggagagcgaggggaaggctcagtaggcaatatgccccagagacatgtcctccaaagcgctgggttgccatgtttcttcccagtactatgaaggactgcagaggagttgaggtctacaaatgaggatttattcatcactgtaaacaatgttgatttgatctactttgctaggaaatggtaccacaaaggaaccttttttttttaccctaaaaacctaaactttaggctttctaacttggagaaccatctctttgtatctttttccccatcattaagtagcataactgaaacatattcttttcttggattatttccgtgaagtatacagagttagagaataagagcaaaaaactgtattacttttagcagtgacttgagcattgttcccgggaggaaagagcttttccattccttctgaggtgatgctgctactggtgtctccagtttggactcttgcttactctcttgtccctagagcggcactgcagaggtggagctgaagaagggagccactctcaaaatcacgctggataacgcctacatggaaaagtgtgacgagaacatcctgtggctggactacaagaacatctgcaaggtggtggaagtgggcagcaagatctacgtggatgatgggcttatttctctccaggtgaagcagaaaggtacgtatgggagctggagtccagttgtctaaaacagtcttttgtctctaaacttccttgacacaaggaagatgggaaggttggttgcctggcagtgagattgagtctgtgtgttctcaggaatcccttttataactcatttatcctcaaagataggctttaatccagcatagttacattcttctggttctggagaacacaggaacatacatacatatatatatatatatacatatatatatatatatatatatatatatatatatatatatatatttgtttcgctgtgttttgttttgttttcaagacagagtctcgctctgttgcccaggctggagtgcagtggcatgatcttggctcactgcaacctctgcctccagagttctagctattctcctacctcagcctcctgagtagctgggattacaggcacccgccaccacacccggctaatttttttgtatttttagtagagatggcgttttgccatgttggccaggctggtctcaaactcctgacctcaggtgatctgcctgccttggcctcccaaagtcagaacagtcttaattatccttatttatgggtgaggaaagtgaggtacagagaggttaaatggcttgcccaggattacacagtgtagtaggttttcaactctggtaaaacagctccagcacccataatgcaccacttcccagctcactgtccttgcgggaaaggtgcctgcttcctgttgacctgtgccctcgtgctctgcctcccctacttaccctttttcatacaggtgccgacttcctggtgacggaggtggaaaatggtggctccttgggcagcaagaagggtgtgaaccttcctggggctgctgtggacttgcctgctgtgtcggagaaggacatccaggatctgaagtttggggtcgagcaggatgttgatatggtgtttgcgtcattcatccgcaaggcatctgatgtccatgaagttaggaaggtcctgggagagaagggaaagaacatcaagattatcagcaaaatcgagaatcatgagggggttcggaggcaagtccccgttgtccctgctccagtcccagcgcagctctccgaagggcatggtccatcctgtgaatgtctgattcccagcccctagcccatcagaatgtagactcccaagccagttccaaacctgctgaatcagaatatcttaggagagtagaaggcattatgtttttttgtttttgtttttttgttttttttaaaaaaaagcttcccaggtaattgagatgctggcagcttgacattgttccctgggcctggggaccaacatttgagagaacagggtcactgctcacaggaccaggggccatgatgttctgttcctgatcagaaacactaccagtgtttgctggaatggggggaccagggggaaagatgacagcagacacttaagaaagggctctttttggcccttcctggggagccatgtggaatttcagggcctggtgtccatgttaaagcttatggcctcctggtcttcacttagaatgcagctggctcagtgatcatgctaactctggtatggtccattccactctcagaggaagatgtgtggttcttctccagtttcagattgccccaacttagcttaccccctccccaatgctcacaaagtagagcccagtgggcatggccaccatttttggcatcctgctaggaatacaactcagcacaactaagatgctagacacactcttgtggattagaagtgtgtttggggagggtgggggagcaaccctgtgcacccactgtagtggccttactgtctgagctttgtgtagatatcctctgtaccaggcaatttggggtcctcccctttgccatcctgataagccataggctagctgaacttggccctaggccaggcaaagccacattccctcttgccttcagcaggttggagtgggccacctcaaagggcagtcctcaagtgtccttgactagatgaggccatgggtctttgtggtggaagcagtcatcaggcctcaggttccctgtcttgaagtgctgattggaaaatggaggccctagagagacccctaacatgcatgggatttggagaggagaccttgggaatgagcccatttggatttgccctctcccctttcttccgtcaatgaagcatccatattggtgttgaagcccagcaggcagaattgttggcccactctgggggcctaaggtagctggactgccttgccatctgtgtgcacccatgatgatatcatggatgtctgtcctggtacaaggacatctaagttagggaatcccagggaaacttcttgtctactgccatacttgtggcctctgttctatataacctctctccccccaactttgtccatcaggtttgatgaaatcctggaggccagtgatgggatcatggtggctcgtggtgatctaggcattgagattcctgcagagaaggtcttccttgctcagaagatgatgattggacggtgcaaccgagctgggaagcctgtcatctgtgctactcaggcatgtgcccacccttccccacattctcatgtgcacactcgcatgtttgtatgggaaagctctggaggctgtctgatctcttcccatggaattgtcgcacgtaacacacagataatccccttcccccatgtacctacacaaagccatactctgtgtacctactcactatccagaggatcagcttgctgtcatttgtctctgaagacagctcaagctacatctcactaatgctctgtcccctcccagatgctggagagcatgatcaagaagccccgccccactcgggctgaaggcagtgatgtggccaatgcagtcctggatggagccgactgcatcatgctgtctggagaaacagccaaaggggactatcctctggaggctgtgcgcatgcagcacctggtgagttctggggcctgccccatcccccagggcttcggactgggcctgggatggatgcaagctctggtgcagagctttttaggtttctccatcctcttatgcacagcctttcattatcctccaagttacagcagcaagagggtgggggtggaagtggaggtggcttttttttttctcctgttctgcattcctgcccacacccccacccctccatttccttctgctctggaggcatcctccttcattggacaccacacagtttatttcacttctgacttcaaggttgtgaattcttcccatggcttaagtcctgggatacttctgcagtgaaaggaggtcttgtacctcttcctcagagtcagaagttctgagtacctttgccctattctgaaaagggctaggggctcctgctcccagctgccctcttcctttggcttccaattcagttccctctgccccgcatcctgcagacaggcgctcccgcagggggcccttgtggacctgcactggagtctgttgccttcactgagctgcctgtgctggccttgcatggtgcctgtagggggatttgctttgctgtgccattggggtacagctgctgctcttactctagaccaaaaagtcgggttgagtgactggtggcagggccacagatagagacagcggggagggtggctgaccctggcggccctggactgagcgtctggaggagtcgtggaggctctttcccttctttctcctctgagagctcgttcttcaggctcttccagcttgtcatgtcgagtgcctggccactgctcagggttggaggctcagtccctttgccctgtctgttccagctctggagctaactcagggatccctgatcagggttacataggtttggtaaaatgagtgctggaaattaactttctcccagtagtcttaggtcatgctcagtgaacttaaactttatccagatatggttttccttcagcctttctattccctttctagccagtgaaagacccgctgccctttgacctcagcccctccaagcccccaagtttaaaacgccaccccctgccaccagaaaaaacagaaaaaaaaaaaaaaaaaaaaaactaaaacacccatctggtctgggcatcttcctttcctttttcactatgtatcctgttactgggcttaaacagctttcagagaagagatgtcatttctattaaatgctctttcagtagcgaactgagttcacacttgactaaggatattttccggactgtctgtcatcagcatccttagtgggtttccccatatttaaattggtagaggccagggatggtggctcacacctgtaatctcagtactttgggaggccaaggtaggtggattgcttgagctcagaagaccagcctgggcaacctggtgaaaccctgtctctactaaaaattcaagttagctagctgggcatggtgatgcacttctgtagtcccagctacttggagagggggtgatgctggggcagcaggatcgcttgaacccaggaggttgaggttgcagtgagccaagatggtaccagcctaggtgacaaagtgacaccctgtctcaaaaaagaaaccaaacaaacataaaaaaaaaaacaaaaaaatcggtagagagtgatttctctcccaggcccacttaatgtagactgggcctggctgacacctcaccattcgtgtgatgtgattgctgttctgatgcttagatactcttggcgcagtctcacaattgccaccatggtaggaaggtgtcccaggagacggtgcaccttgaaccagtcaccactaaagtggctgcctttctgggtctctccacacatcccctctctctaatttccctacttaatcgtgtgacttcatggtctcaaaggaggaacagaggctgatcttgacttagatatactgaaccatgaaatcactgcatagaatgtggggacttgaatgtgtctttgggcaagtcatttaacctcttaagacctcatctgtaaaatggattagatatgtttaattatagccttagcattaaatattcattgctgttattattaagtgtctgataagtctctgtgtacatggatgtaatcttcctaactcccattacctccatttatagatgagggttatatggccaataaagcctgggtttgaatctaggtctactgcctccaaagccagtcttctctcctgcaacatcatgctctgtctagcaggagatgagaacaggtctccatttggagcctgtcagtggggtcagagactaagattcaggctcagggtctaaattccatatcctttcttccataccctggtgtttcctatgaacagatagatactttagggctgcaaggtttggattgcatggcactgctcagaagataagttacaggtctgggctaggctgtagctgcccctccaggtggctagacctttcctttctgtgtcaccagttaacactggccaacagttccttccattaactgttcactgctttctcctgtgtctaactgatgcagtttatgacccataactaagagcagtaccaggtatggctctgtttcctgttcatgtcccctgtcctctgggctgcatgcattccgttcttacagaaagaatacctttaacctagtacatcctgccacacatctgcttctactgtgaaattgatgagggggtattaccgattcttccctcacccatcatttactgagatgctggtgattgcattataatcctctaaagcttacattgtctttctgattcttggtcttatctgagcaagtgatctataaataactcagtggctttctcatgactgttttaattattagattttaatcaagtgtcttattaaatatatctgcatgcttccacaggcatctgtctcttcacatggctgttcagtgtgcctctcacaagttagcccacgttttctgttctcctgcttcaaactcagttgagctgccttgctttggctttgatcccagctttccagcgctgctcaatctgttgccatggcaggccattggaaaggctcagtgcatccccgtgcctgaagccaagtgagcgctcactccatgcatgcatggaggctgggcaggagcctgcctaatcaaccagccatgtgaggagggagggcctgttccttcctgtaagctatgtcatgaggcagcgtggtcaagtcctctgccagggagtggcctgggcccagcctgggcatgttttcatgccagggtgctagagcctactgccagattgtctccctccacccccaatgaaaaaatccttccagaagggaagagccaatttcccctgtattggaggggaagtggcagcacctcctgaagcagttggactttcatcaccctacctctgcatctgcctgaaggacagatttagccaattaacctaaggttaccttcctctctgataaattccccattctgtcttcccatgtgttgtgtctcgtttttttcctcctccttccctcttccttgccccctcttcccctaaaccttacagatagctcgtgaggctgaggcagccatgttccaccgcaagctgtttgaagaacttgtgcgagcctcaagtcactccacagacctcatggaagccatggccatgggcagcgtggaggcttcttataagtgtttagcagcagctttgatagttctgacggagtctggcaggtagggccctaagggcaggtaacactgttaggataaccagcctcttgctccacctgctctaggagaagacagccaggcccaacctggcatctgggcacagagcctcttctcgtctgtaggaacaccgccagggaggtcatggcagggcaggaccaaagggtcctgtggctcagtaggcacagtagatgtcacaggcacttggtgaaggactggtttctgtggagtcttgatcttggctcagctcagaatctccagtgattgggctcctcttggcctttgttcccaggaacatgttcctcaccagctgtccggtgactcttcccctccctctccttttgtgacaaagctctgacaaagctctgtccccctctcgtccctctggacggatgttgctcccctagattgcccgtgaggcagaggctgccatctaccacttgcaattatttgaggaactccgccgcctggcgcccattaccagcgaccccacagaagccaccgccgtgggtgccgtggaggcctccttcaagtgctgcagtggggccataatcgtcctcaccaagtctggcaggtaggaggcggcagcggctccctggaatgccctgctcagtggtacctcaccttgggggtcctgggagcagtccattgaacaatgctcaggtggcactgagccaaggtaagacccctctgcctgccaccttgggcctgcagggaaggattgagcagagccccttccctgggcccaaaggactctaggtagcactcataaggaatgtcagaacatttggatcaaaagcaaatttatgctggagatttattacataacagtgcacaggctgactacaaatggttatttgatattgaaaatttagtcctctaaaattgtaaaagataaccacttttgcttattccagttactatgtgctctttaaaaatttcagttgggaaatgaatttatttaaatgctgtttactgtgcctccatttggcacactagtccctgctgtttttgagccctaaagacaaattgggttccagctcaggagaggttgctgtgctatcttggctgacattctgtggggcctggcagccaggctgaggactgtgtggcctatgctgggcctccaacttgggatcccttccttggcccaggacattgagttaatgtccttcactctcctagttagggagtatgctccttgtccctgtccacagggcagcaagggtttcctggaagaggggagcaaacaggcagtgcccatgcactgaggagcagcagatgggcgtgggcagcccagagaaccaggacacaagctctgtgcagatgccctcagcagagggctccagcctcccactcttggctgaacagctccaacccgtagggttgacctttcttaaaaggtccagttcttgctgtttggctattttaagctctagtcttctggggtttcactcagctggtcctggcttcagcaattgcttccctctgaaggccttgcatagaggccaagcgtgaagtgcagggacttctctgctgtgatgtggcttaagtttccctgacacctgttgagtgtcctcataacttcccttctggtgcccctccccagctcctgagacacagctgcagctacaagtgtgcagtgtcagtgttcaagaaagtgcctggcagaggggctttagaagggtcccctgccttccaaaggagctttggcaggcagagctgctcctgcagcaacactcccatttcctgttcttgcctgctgagtagcacctagatttctaagcctcatctagatactcagatttgattctgggcctttatagcccagttgctgggactgtttcaggagctaggggccatgtggggcagggagagggcacaaaagtagagaagcctgatgttgattcccagggggctggtcagctctgctactgctccttgcagatgtcaagagtcaggtgctagtcacgtgctgcttggcttgtcactgtcattggcagcgagaggaatgggtgctggtgacattgggccagggctgcctctctgtgtcagagttcagggtgtaggaggggttctgccaaccatgggctgtgtggggtaagtgggtgaggctgatcttgctgggtcaaggtgatcctgagcccttggcctgtggaatgggggtagagggcaaatggtaacctagcatgctgtgggggatataggatgaggggctgcccgagcctcgggaggggtcctagggagcagatgttgaagaggccagagccctcagtgagctggatgagagggtgagctgtttgaacgccctgagggtacttcctggggcctcgtgtaatggtctcttctgtatgtcccccatcccatctcaggtctgctcaccaggtggccagataccgcccacgtgcccccatcattgctgtgacccggaatccccagacagctcgtcaggcccacctgtaccgtggcatcttccctgtgctgtgcaaggacccagtccaggaggcctgggctgaggacgtggacctccgggtgaactttgccatgaatgttggtacgtggctggagcaggggctagagcctagaggagcttggggatgcttgagcattggcttctgtgggaccccgaaagtttggggaatagaaaggggaacacacagaccttagtggggcaaaaggcccagcgactgttcctctcccttattgggaatgttcattctgaatctctcattctccgaagtcctaagctgagccaggagggaaaagggtcctttgagttgtagggctgagcaattcagttcctcttctcttctagtctggggctcaaagcaaaattgtccattttttggcatctgctcattactgagagttttttttgttttttgttttttttttaaataaaattggccacagctcctgtgctgtggggtggcatacacagattacgtactgatgtggccattgtccctgtataaggtagggtatcatcagatgacaggaagcagctagctctgaccctgggcaaggctttgcaccctctccaggatagtgaatgatgtccaaaggtccctgccaaccctgccatctgagtgataaggacatttcagggccttcctcctgtttgcctgggctgtgagtttggtgccaccttgtggtgtgaggaagtagtggtcagccagcctagttcagtactcaggctatggggcagctgcccaggtgcaaacctgcctggcttggcttttactcaccaacctcccttctcttcctccaggcaaggcccgaggcttcttcaagaagggagatgtggtcattgtgctgaccggatggcgccctggctccggcttcaccaacaccatgcgtgttgttcctgtgccgtgatggaccccagagcccctcctccagcccctgtcccacccccttcccccagcccatccattaggccagcaacgcttgtagaactcactctgggctgtaacgtggcactggtaggttgggacaccagggaagaagatcaacgcctcactgaaacatggctgtgtttgcagcctgctctagtgggacagcccagagcctggctgcccatcatgtggccccacccaatcaagggaagaaggaggaatgctggactggaggcccctggagccagatggcaagagggtgacagcttcctttcctgtgtgtactctgtccagttcctttagaaaaaatggatgcccagaggactcccaaccctggcttggggtcaagaaacagccagcaagagttaggggccttagggcactgggctgttgttccattgaagccgactctggccctggcccttacttgcttctctagctctctaggcctctccagtttgcacctgtccccaccctccactcagctgtcctgcagcaaacactccaccctccaccttccattttcccccactactgcagcacctccaggcctgttgctatagagcctacctgtatgtcaataaacaacagctgaagcacc

SEQ ID NO: 46 - DNA, 34218 bp, Macaca mulatta
  source 1..34218: mol_type = genomic DNA
agcatttccttctcaacaacagctaaagtgaccaggaaggcttacagaagaaagtgaaaaatttttatctcacttcaaacagaaatttacttttaggctgtcactgtctcatcccagcttatctgagctaacaggaagcgaatgaaggcctgtgattcagctgcaggcagcgggtaagcctgagcaggtcacactcatttggtctcttttgccaggaaaagaaaaaaagaacctgagagaactgtcttcacattttgaatgcgcaacattgtatctgtgaatgaaggcaagagttaacagctgtttaattgataactgctcgcatcattggttgctggctaacaattgggaaatcggaaaatgtcttgtagaaaaatgtaagaaaagttccaacaatattgacttaaattcatgacaaggtgaaaacagaaatgctgactcctgcataggttatcggctctaatgttctgacttgattatttccagatgcccagctctgcgctaatatcaacgccgtctatttgctttttactctcaggcattcgctctgcaggattccagaccctactaaattattcacatggccccaacctgtccttccttgttccgcggttctaagacaacaaatggtcctaagaggaaaagggcctcgcgctcccgctccaggcccacttcgcagtccctagttctccctactgccgctccagtgccagagcccctccgaaggcggtcgagacctccaaccacgcacaagtctgcagctctccccaacttcccgttcagctcagtctccgagggtgcgccagggcagacacccggaggagtggggagtggcagggcggggccgggagaatgctgccccggaacccataaatctgggccctgcccaggtaggccgggacagttggggtggcctgggccgagagccaagaagagacctcatctggcagcccaacttggcggcaacaggtggcccggcgcccgggggtctaggagggaagtcgctctaggggcgggatccgctgccccgccgcgttcccattggccgtcaggttctttaaactgtggctctgaatctgtgtccttccgccgcagaatttagtccccccgaaagggcaacctgcccgcgctctctgccgccgccaccgcgcttcctcctgaaggtgactgcgcccgcaaagacgcagggggcggggcccgggtcgccgggagccgagattggacagagggcggggcggcggagggattgcggcggcccgcagcgggataaccttgaggctgaggtccctgctactcgcacagcagctgcacgtccctgggatccacatctcttccaatttgcagcgtagcccgagtcggtcagcgccagaggtgagcggtgcaagaggctgcgccatctgtccccaccaagggcctgtgcacccggctagtgcggaatcccggcgcgccggccggccccgggcacgcaggcggggcagcgcaggatccggggcatgtgggacgcggtggagttcagagactggagaacggctcctcagaagtccccagcgccgttccttccagatcaggcgggtccgccggctcctttcccgtcccagccctaccccctcattctggtcccatcctcctgccccaatcctcaatgcgcctccatccttgcctgccttctctcggtccctcgtgattgattccacccttgcctcccctttctccgcgccctctgttccgtgctcgtttgttccacttcctcctcaagtccggtccttccacccactcctcttaaacctgtgcttgcctcttcgttctaatggcttccgtgtaacttactgaaactcggttctggttcccctcccgggtccatgctccattcatgcgcgaccgcccttcctgcgccccagttctctcttgccgcccctccccctgctcgctggtcacgtccgctctcccgcatccccctcctcgcctggcatgtcgcctccctccctctctgctctggtcgcgcccgcccacttgctccggtctccgagcgcggtcccgcgccccttttccatcaccgcctcccaactcccagcaggctcgggcggtgctggggccccgtgtccggggcgggcgggcggagggctgggctgggtcgcccgcgcggcaggcgggatgcggcggcccgggagcagctggaggcttacgtaacgttggcctgccccgctgcctggaggcagggcggctgcccctgcggcgcggtgctgtcccatgcgaccgggattagatgggtggcctgcgagggtctgggaatggctcgggacccgcgagctctcccggcccttgccgggtggccaccagggaccacgctccatctcgccgcggccgggctgcacgtagcggccgcgcccagagtccaccccgcttcaccgggcggtggccttgggccgcgtaacgggccgtagaaacgtctgcaggcttcgctggggcctgccggccctcgcccgctccgcgcctctccggcacgttttttttttttttcttttcgttttttcttttttctttctttctttcttttttttttttttttattatccagcaggttttttttaacacatgcgccctgattgtggaacgcggaggccgcggggtggggtgggggtgggctctggttactgagggggcaggaaatctgtggcgttcactcaacgcaaacggtgtgggtcaagggctctttgggggtcggagttagagaccagaatgactggacgagtcatagcccaccgagctcacaatcttaaaatgtatacccggtaatgctgggagtggggcacgagcttcctgctgtgggagggagggggacaggaagcctcgtaaggtctcaccaggtggcaagcacactggattagaagatgcaggaaagcaccttccagtctgaaggtcatttaaagactagtcatggaaaggcatccctgggcttcttaatctgcatactccagacccagggcttggacctttgctggtacgggttaaagtgaggccgacagtgaaggtccacgtggagagagacatcgggaagttgtcagaaaggcttttagaatcaggtttgactcattaatttcctgagactgattgaaggagttcagccgctacacctcagtttcccctgggaaaactgggagtaacattttcctcatgatggttgaaaggattcatgcctacgtggatgttgcacactcagttaagtgtctaatctctggcgaaatacccagaagaaaagcctgtgaatgaatatattattttcgagcacaattacttagtccagaattgagcataagggccttgattcattatcttactgctgtccgcaaacgcctgcagctaatcgtggggaatcaggctctctaaccacgtgtctcctggaaaccagtccccccagggatctggtttaatattgacaaagtcactgataatgtaactttccaactctctgcttggagcagatgctctttgatcatctgagattgggacctctagacaaaggcaggcaaggaggggctgagttaagccttcggtgttcccctgtaagtggaatacctcttaacaggggaacattccagcccttgacttgctctgcctgctggtgagattaggattggagccaggatcagtaacccccagttggccatcttgagggcgtggctactcccgaggccacagaggagatagaggtcaggactccccggctgcagcattgttagcaaataatttgccctttggtggcctgaggtgctgtgggggcaggagtgggctgtcttgtggggaagggaaatgtaacgcatcaccaaatctgtgggggagtggggctctgtggggaagatgactcaacctgtcaattattctgcttttgagagaattatcttcgctgggtgagtggggaaactacgtagtggttcttgcaggcttaaaaagtcccttctctttcagtgagtcaaataatgaagcatgcatttcccctaattaaaaatgcctcaacttcggagcttgcggtgagccgagatcgcgccactgcactccagcctgggcgacagagctagattccgtctcaaaaaaaaaaaaaaaagtgcctcaactaagtgaaaaatgattggtccgggtcacctggttctctgtactgccttaagaacttaggtgcagagtggcagagcccggtgcccagctaatgcagtctgtgcagcttcgttcctggcccttttgtagcgggcacagcagcctctcctgggacatgattaatgatgacttaaagaaggttcttgaactcatttctcagttactgacttgaaccaaaatgtatggatgttggtctcctgtctagcacagctgctttcattgtaagcattgcgttagaaataatgtattcatattttagcaaagagcatcattagctctttaagaactgtacccccagtgacttagcaaatttgttggctgtgactctaaacagctagcactttatgggagttgcttctgtgccttgaatgttgtgacactgatgtggggtcacaggaaaaaccattttatttcttaaagcggtttgtttcttaatattgaagcaatggaagaaatgaggaaatggccatgggcaggaagatagaaaaaaatacagcagttacattacaaactcactttacttgggttttagttcattttaaagcttcagggtttttttttttttttttgagacagttttgctcttgttgctcagtctggagtgcaatggtgcgatcttggctcactgcaacctccgcctcctgggttcaagcgattctcctgcctcagcctcctgaagtagctggaattacaggcacctgccaccacgcccagctaatatttttgtatttttagtagagatggggtttcaccatgttagccaggctggtcttgaactcctgacttaggtgatccactggcctgagcctcccaaagtgctgggattacgggcatgagccaccacgcctggcctgcttcagcttttatgaaggcaaatccggatgtgttatcttggccaaaagctaagactgagaccagagtacaggaaaatgactcatttgacattacccttctttctgaatcactccaaaagcccacagtcaaatcccaatttaactgatataccacaagaatgcattgcccctgtgagcagagttttattgcaacttcgagggtctttgtgagattggtttatttggtatgaggtcttaaggattttaagttgtccctttaagcatcagttctgatacagatgggaatagagctagacaaccagatatatgaagaccatttgtgataccaccttacttttggggagcttgcatttttttaccaaggggcagactgaataattgagtaatttgaatacttggggcattgttaaagaagctgttgggggaaggtgagcctgggtgagtggtggagggtggcgtgcagcatctgctgggctttccggcctcttggtatgactgctcagctcaggagttgggggctttagggcccccaccacccagtgcggcgaagccccgcccctgcagtggtttgtgcttgtctgcacgtaggagggagcaagccggctgcagtaccttcggtggcctctgagactcacctcccccacttctgtccgtagacaaagctcctggggagctctaagcctctttcctcatgccagcaagcagtcttggaagcaggctggggctgcggtgggagagatgccaagaccagttatacaagggcagtgaggttgccagtcctagcatttcagactgtggaccagcacagctgctggctgaacactgcagtcaagattctctgaagggagtcctatcccttccaaacatcttcagtttacttacagataatgtttattcatattttattttatttttttgagatagagtcttacactgtaacccagggtagagtgcagtggcacgatctcagctcactgcaagctccacctcccaggttcacgccattctcctgcctcagcctcctgagtagctgggactataggcgcccaccaccacgccaggctaatttttttgtattttttagtagagacggggtttcaccatgttagccaggatggtctcggtctgctgacctcgtgatccgcctgtcttggcctcccaaagtgctgggattacaggcatgagccaccgcgcccagccatgtttcttcagttttaaacaactgatgtctcccttggcaatgaataaaaaagaactactcatcaatggaatcagtcagggcacataagacccactgtgtccaccatgccatttcagaggagattcgattgagttaagcagggaaatagagatgtcgtaaacattgaaactatctcggtatccctctttggttattaacattagatgagcagaaaaacaaatgtcacccatcagatgggcaaacatttaaaaagtctggcagtaccaagggaagaaggtgtggaatgcagcaactcaacgttgattgatacagccactttggagagcaaatcagccatatttagtaaaaatagaaataaacataccccatgacctaagacttcaatttctggatatatagaaactcatacagtacagggagacatgtactacaagagtattgattaatagagaaaaactagtttaatggggagggggagtggccaccagtaagggagtccataaataaaattgtctgttgtacagtggactagtgtaacagttcaaatgaattagctagatccatatacccactggatggatcttgaatgtgctgctgagcgaaaaacaagttactcagtgatatatacagtgtaccacttagggcattaagaaaaccacaatattatagggtttcagtatagatatagatatactgtacatagacttcagtgaactggaaggatacagagttcatgacagtgattgggtgtcaaaatgcatgatgtggctgggtgcagtggctcacgcctgtaatcccagcactttgggaggctgatgcaggaggatcacttgaggccaggagatcgagaccagcctgggcaacatcacaagattcccatctctatttaaataaataaaatagaaaaaagagttcatgatggaggtggaaagggttggaggagcttggggatggggaaaaaatgaactgtataaaattataatttttttttttttaagaaagtaaagagggccaggcacagtggctgacgcctgtaatcccagcactttgggaggctgaggcgggcggatcacgaggtcaggagattgagaccatcctggctaacacggtgaaaccccgtctgtactaaaaatacacacacacacacaaattagccagacgtggtggcaggcgccaaaacgacgccccgtagacacgccgatccgatatcatcccccccgtggtggaattcccgattgcagtgaaccgagatcgcccagcagcccccccgcctgggcaacagagtgagactccatctcaaaaaaaaaaaaaaaaagaagaagaagaaagtaaagaaaatacagctcagcctttatttgtgttttttttttttttttccttttttttctgagacagagtttttcactctgttgcacaggctggagtgcggtggtgcgatctctgttcactgcaatctgcacttcttgaattcaagcaactctgtgcctcagccacccaagtagctgggattacagatgcgcgccaggctaatttttgtatgtttagtagtgacagtgtttcgccatattagccaggctggtctcgaactcctagtcccaagtgatctgcccacctcagcctttcaaactgttgggattacaggcgtgagccaccacagccggccatggctcagtgttaatggtcagttctgggtggtagagatgcagatgttaaaactttttttttctttaattcataacaaaatagaagcaaacctataaaggctattgtaggaagaccagtcatactaactagttcagtgctcttggagagttggcactgcctttcctcctttattgccccaactagaatgcagggcagcccttccattaaatgctgagccagtgcctcactttgctgaggccataacccaccttagttgcacttaagagtcctaaatcaggattcctggttctagagtgtggatatcatacccagaaacaccaccctacttttaatcctagtaaggaggcaccatgtcccaaggcagctaacgcttcccccaaaccacctccttcaggctgaaaccagttgtctgcaccgagcagctgggatagagccaggaaatactcggcatctgaggatactgagggttctctgacttaggacttcttcacctgaagttgagtggtcttttggggaagtaggcctgtttagcatcagctgctcttccctattccacactgtagctggaaataggaccttaggttcctgttgacaagtcatttacttttagccctgaagaaataaaagagccaagtgttttttatttttatttttatttttttttaaagcaagggaattttactagaacctgtaagtgggctcattttgttctgtgtagcttggtaacaccatactgctttctgctgtggggcctgctggggttaaagtgtgggcttcagacccaggtctcttagctagaagatatctcatcctctgtgtcctgcacccatatgcaaatatgctattgtcattatccttaactgtatatgaagacggacagtgcctactccagaccttactaggttctgctggcccatcacccattttgatcatgttgctggcctagtttgattagggcaaatcttagaaattccatttccattgttgaggaagagagctagagagcaggctgacccgaatgccagaatatcatgatgcaaactttctaatggatgctggtgttctgaagagttgagggttgaaaggccttcatgatggcttgaagatgtaggtagcaaactgacgtcacagcaaggaacgcaatcttgtgtacctactggcaacgttggagagagaaagtgagcatcaggtgccaccattttacagttgatctgtgtgaggctggtatcggagcataattggtacaaagtaaaaatgacttaggcagatgcagactcacgggccaggctggtttattagggcagaatgatttggtcctttgtgaaagaattggtggagtgaagcgtgaatctttcccagcgcaacccaacaacagttctggccctaagaagtggagcatgggagctgggtgtggttcgtgcctgtagtcccagctacttggggtgctgaggctggaggatcgttttgagggtgcagtgagctataatatataatgtaatataatataatataatcataccactgcactccagcctgagtgacagagtgaaagcctgtctcaaaaaaaaaaaggttggagtggggagtgttgggtattttttatgatttttgtctcctggtttctgacgaatggccgaaaaattgtgtctgacacaaaggaaactaatacaaagagccaggaactgtctgagatgagaaaggaaagggagaatagggcacctagagctgagctgtgattgtgcctgttccaacctgtaactccagactaaggcctcttagaggtggtgtctcctttctcacagaggagacatggctctgaggaagatcttactgcaggggctcgggctcaagaataaggctcctggacctgggcacggtgtgtgctgctatcagtggatacgccaggcttccactgctggctggaggttggctctgcatgtctgtgccttcctaggaggaggatgcagtagtgagtcagactggcatgggtggggccacatgtcctggcaggtactgtccgggcaactggcatgagggagaggagtctttcccagaagcctgccctgagaaaccaagactgggctgctttcctggcgccaagggagagtccagtgagtgttgtacatacaactctaggtaggtggttggtggtgtgggcagcttgtgctgggaagaggattttggagaaagcaggagagctctggccctccctgcaagggaggcttccggtcagagcctgaggaaagacctgagcttgaggtatggggtctctggctcgaagagagcattaaggatgcatgagatctgaagtctggatgaacttgctgacaggcaagcttttttttttgactgctagtgtgtaaatccactatagaaattttcatttgtcattcttgtacttattggccaaaaattagagcttataggcatcagatttaatttgagatagttgaaatagggtttgtgtttgaggccaatataattttatctgctaatggcatcttctggacttggaggcagcccttctgtaccagaacattgtcacaaagcttttactgtaaagcttgagaacagactagtttgctggatttgggcatgtaactaacagatttgaggcatgggattatcctatggacttttttttttttttttgctttgaggttctgactatattaaggttgaccactttaagccattgaaactctcctataatacatttgtgatgtagggcctaagacatgtttgtttggtttttttttttgagacatgtatctttgctgtcttttaaaagcagattattttttatttatttatttatttttttgagacggagtctcgctatgtcgcccagggtggagtgcagtggccggatctcagctcactgcaagctccgcctcccgggtttttacgccattctcctgcctcagcctcccgagtagccgggactacaggcgcccaccacctcgcccggctagttttttgtattttttagtagagacggggtttcaccgtgttagccaggatggtctcgaaccctgacctcgtgatccgcccgtatcggcctcccaaagtgctgggattacaggcttgagccaccgcgcccggccaaaaaagcagattattcttgaagtgcatagagcagcaaccgattaatggaattggtatcttcacatttcatttacttcctcccaacaattttataggatgcatataaatatttccaaaaaaggtgtacataatttcttactataaaagtatttttattttatatcagtaaatttgttaataaagaggaatttttgttgttgttgttgtaatcctaccaccccaatgacatcctattaaaatatcagtatatatcctcccaggtcttttaggtatgtttaatttggtgtcccttccccactcccaaaaggggagggaccaggttcttgtatagaatagtggaatgttagtaaatcacgtgtttaaagagacataacagctgaatctgtagagcagctgccacctggatacctggttattaaggtaatttttccattaccccaaagagctttagttacactcagcttttaccttaatccttgtacagctctccagggcacactttattcagctctgagcggtctttgcaagtgaggccaaggagccaacctgagccaaaaggagagcattatgtcaccggaagcccaagcccagagaaccaaaggtatgacctgatattcagtggccccagccaggtctttacaagaagaccctgatatctcaggtctaagaagagccagctgatggtttttaaaaagagtggaattagttactctgacccacttactcagatcttattttgttcacaatgtagtccccagattgtaggcccattggaggccacagcaaggcctttgtgttccaactggcctgctgtgccatctctcagtaatgttcccttaacagccagacttccctaagcccagctgggagctctgaaggtatgccagcccccccttaaccatgagtgtagggagagggaccagggcgccccaggctttcctgtcagtaatgtagaagtttctcagatttagggacgtgggagcagaggcataactttgattctgacaaagaggcattcagagagactgaaaggtcatttaacaaacactagaatgcttccacttactaggtgctaggaaatacaaaaccatataggtcctggaagagaggatacatagtagatattagaggcttaggaaatacagttgaaatgaagaatggatttgccacgttgaatagttagttggtatcttatttccccattttaaaagttttagaatctgtggttgaggacttgtggccatcagttttccatagccgacagactgttcactattgccttcagagctctttggaccttaataggccttctttggagatggcagagatgggtttagatgcatactctgctggagccacccagagagcccacaaagtcagggatggaacagggtaaaggagtaagggtcatatgtgtgagatgccttgatttggaactttgagatttaggaagagatggagaagggctaaagagggacttgtttctgagccttgcttggctgaagcatttaggctaaagcgttttagaaagagtagcccttggtgtgagaactcaaggaaacaactttctgatgagaaatgtagcaagcttctggttcacatccttacctgatagttcttcaaacactgcctggaagcttctcctgagcctttgtctctaatcagctaactaagaggctgagcgagtttagttgtaagtcattaatgaagaaagcaaaggtcggggccattgtcagggttgtgacctgggctagttaattacctggaactgatggtctgtgttacagagtggtggtatacttgtcaggcttagaaaagaaaacaggatgtgtatcaaaaatcttttggggaaaagatttgaccagcaactttaatttctctatgttgcagctatcctgttaatgtagttgtgataattttagaattataccagtgcccttatgttatccttgctttgcaaattgctttacgtgtcctaacgtccttctggccaacagtagatgtggttttaggtttagcctcctaggatggaagcttttgcattcaggggaatgactttgggttgggtgaggattgtaaagaggcaatgtgggtgccccacgacaaagcagctatttgtagctttgtgacaacttgacatacagatatctaggcttatctaggcactaagctaggagttagttgtttgtatcactggaagattggttataacttccttcgttggaagctccttcattgcatgttaaatgatgttatttatagatagggtggtgggaaagctgtctaagtagatgtcagcctagtgtaagagagacctgcttactgtgggtgcttgggactgtcagcagtaggtgggaggttttaacttgttcagtaaggtccatttccattgttcaccatcttgtgaaccctttgtctaacatgaggagcaaccacataaccacagcatgtctggcttccctgtggcttgtgtacaaagcgtgcttattgagttaatgtataagcaggagacaccctcctgtgataaatggtatattaaccacttctcagtcttaccactctgtttcaatttgtctggacccaggacctcagcagccatgtcgaagccccatagtgaagccgggactgccttcattcagacccagcagctgcatgcagccatggctgacacattcctggagcacatgtgccgcctggacattgactcaccacccatcacagcccggaacactggcatcatctgtaccattggtgagtgggtgtgccccttccccaaaaaaagggcttcatggaaagtgaccctttctctcctgaaaagacttaactaaatgtcctaacaaacctaggtgctacatgggatgctacacagattcttataaaaggactcaggtcataggaagttgcagtaacgaattagtatgtgcacaggatggcaaatacagttaataagagagtattagacatttcgaaattgctaagatggccaggtatggtggctcccagcactttgggaggccaaggtgggaggattgtctgaggctagaaatttgagaccagcctgagcaacttagaccctatctctccaaaaagtaaaaacaaaagaaaaaaaaaattagctgggcatggtggcatgcacctgtagttctgactacatgggaggctgagaaaaagatcacctgagtccaggagattgaagttgcagtgagtcatgatcacaccactgcactccagtctgggaaacagagcgagatcctgtcttaggaaaaaaaaaaaattgtctgggtgtggtggcacatgcctgtaatcccagcactttgggaggctgaggtgggcggatcacctgaggtcaggagaaccagcctggccaacatagtgaaatcccatctctactaaaaataaaaaaaaatagccgggcgtggtggcaggtgtctataatcccagctacttgggaggctgaggcaggagaatcgcttgaacctgggaggtagaggttgcagtgaaccgagacctgaccattgcactccagccttggcaacaagaatgaaactctgtctcaaaaaaaaagaaagaaaaaagaaaaaaacattgctaagagtaaatttcaaatgttctcaccacaaaaatgttaagtacttgaagtcatggatatgttaactaacctgatttaattattccacattgtatccaaactgtatgtattggattacataactttgtaacccaaattataaattaccagtttataataaaaaacaatttgttgcaaaaagaatccatatgctttaggttttacgctataggcaaaatttagagggtgttttccttagcaggtctttgtaggagcaacttacagacctaggaaagatctttttaatatgttctgtgcaaccaagattctgtggttggacatctggctgggtttcagtgagggcggagaaggctcgccaagtcttaacctaggcttttctgatacagtgggagcctgcagaatttgaaggaaatggtcgaagtgtcccagtaaatcaaaaaagtaagctggcacggtggtagccttccatacactttttaaagacttttgagctatttgggagaggaaaagttttcagggaaaaaaattctttaaacttaagcaaacttaaatgtttttccttctttgaataattaatacttgtggctttaaaacttttcctaataggcccagcttcccgatcagtggagacgttgaaggagatgattaagtctggaatgaatgtggctcgtctgaacttctctcatggaactcatgaggtgagctgtagctggaccctgtggccattgtgatggcctgtaggaaacagggagggggtgcagtgttcatttagccacaacagattaggcagggatgagtctgagtttcacagccagtgtgaagtttgtctttactagcccatccctactctccttccctcttgtcctggcgaagaaactggctaagtctcttttagcaaagaagaccccttttgtcactggccatgtttctctcacacacctctcctaccgttagctttcacaaaggaagatatggaaaggttctcctggaaaaccctctaagccttagatgtcctggccacagcgcttgactctcctgtccccaggtttctgctacagcctgtattgccatggtaaaccatctagcagatcgattctggcctatcttagaaccaaaataactgggcaggtccatgagaagagtttccattattctaagttttgaatgactgagcctaatgcacaagcactcgctggcgtataatacccctcttcctcagcactatattcccagcctgtaccttcccaggagttctgtacattaaaataacaccagttagcactcttcctcaggagcctagtaggactgtatttgtgatgggctctttattacctggctttacctatggacagaggccttgcccaggagccaggtagcagctgtcaggatggctccattcctgcttctgttgccagatttagaataaacccattctgaggagtttggggttccctgaggtaccatgacttatttatttttttattttataagacaaaattttgctctgtcatccaggctggactacagtggtatgatcatggctcactggatccttgacctcccaggttcatgtgatcctcttgcctcagcctcccaagtaactggtaatacaggcatccaccaccgtacctggctaatttaaaaaaaaaattttgtgtaaatgaggtctcactatattgcctaggctggtctcaaactcctgggctcaagtgattctctcaaatgttggaattagaggaatgagctactacccccagcctgggattgtgcctttttaaaaccttcagacttaaccatagatttcccatagatcatgggatttcgtaatggcattgataagaggaatcacagaagaggcagactttgtccctgtcttggcttctgtatttcctgttgagagtaaagaaaatgctaccctgtaaggccaagtgccttatagaggttgccctctggcatttggaagttgatattaagtttggactaaaaataaagcctcaggaaatgcagtccaagagtgaattcctccttttgggaaacttgagactcttcatcatagattccctaacctgtgttcataaacagcctatggcctggctagtggctggcccctaaatgtcatggggacctgaccaagtcctgcagacataccacgcaggttaagacatgtccctgtaccttttggaaaattctgtagttttccaaaagcaaggggtccttagcaggagtcaccgagaattactacaagtgttagcttagcttaaagagagagagctgaagacaatggtggaggtcagttcactgttgatccctgctgctgtagtctgccgtgggctcctgcgttcagggaaaggagcagaaatagatttttaagaagttgacctttaagtaggctttgtggttccttcatccagtaaaataacaccacatagctcttacatggcaagggggagtgatatctgctgcacctgctggatgagagctggctccgattttggttttttaaactttaagaggctttttggagattatctctgctttcactcctactcccagattataattaagattttttttaaattttttaatttattttttaaagatgtccctcttgtgggtttattttgaagttttagaccaagatgaggttgtctctgggctcaatttggaaactgatttgaagttattctaatttgtgtaatgtaacataaacagtttcaaccttaccgtttgtcagggctgttgtttcatatgcacctttgactaggggctgggatgtacttttctagttcctgactattttaaaggctcttctgagcagaacattgagattactgtcttccctctcactctgacagaggaacatcaaatgtctgcatctgatcttttaacagctttctttttttgagacagagtcttgctctgttgcccaggctggagtgccctggcacgatctcggctcaccacaacctctgcctcccaggttcaagcaattctcatgcttcagcctcctgcatagctgggattacagacctgtgccaccatgcccagctaatttttaaatatttttagtagagacagggtttcgccatgttggccaggctggtcttgaactcgtgacctcgggtgatccaactgcctcggcctcccgaagtgttgggattacaggcgtgagccacacgcccagctctcttttgacagctttggcaactagtcttcagccctcacttttggcagttcacatgggcaaggtgcattcttgctgaacatgtggttccatatgccgtgttttccagatttatttatttatttagagagagagtcttgctgtgtcacccaggctggagtgcaatggcacgatcttggctcatcttggctcactgcaacctccacctcctgggttcaagagattcttctgtctcagcctcctaagtagctgagattacaggcatctgctaccatgcccggctaatttttgtattttagtagagatggggtttcaccttgttggccaggctgatctcgaattcctgacctcaagtgatatgtctgccttggccttccaaagtgctgggattacaggtgtgagccactgtgcctggcctagaaaaaatgactttcaaacaacctaaatgtagagccttccacaggacagcattgatggatgaaactctttaccatgtaacatcccaacaaagccacagctatagtagaagactcagtacacctcccagaaatgctctaagagattataatatgtaacatagatttgaataatacacctaataattggtatgtttataatacatagttttacatccccaagaccaaaaatgcatgtttgtgtgaaacactcatggttacaaatatatattaggccaccaaaaaaaacccccaagtttcataaagtagaaattatacagacacattctctgctaaaaattttttaatggaaattaagaacaaagtcaagaaaactgaagtgtgcttactttagaaagcaaagatctcaaggtagatggaataaatatttaactcagaacactaggggaggaaaaccctaaaaagggtgaagaaaaataattttgtaagattatagctcaatgaaatgaaaataaatttgacaaattaagctaagagctgattatttgtgggtaaaaaatagtaaaatagaaagttctgggaagccaggtgaagaatgcaaggatgtacaaataagagtatgaatagaaaagggaatatttcctacacattggagattttaaaagtcaagaaagacttataacttaattcctatagagagagatgacactggctattttccaagaaaagattaaatgccaaaaagatagaaaatatgaatagaccagtggccataagaagttgaaaaagtgggctgggctcacacctgcaatcccagcactttgggaagccaaggaagagggatcacttgaggtcaggagttcgagaccagcctggccaacatggtgaaaccctgtctctactaaaaatacaaaactggcggggcatggtggcacatgcctgcagtcccagctactcaggagcctgaggcaggagaattgctagaacctggggaggtggaggctacagtgaactgagattgcaccactgcactccagccaaaaagttgaaaaagtggctaagatctggttcatctcagaacatttaagtccaaacgattttagtggctgaattgtctccccttcaaaattcagttacattgttaaactgttccagagcctagagaaatacagaaatcttcccactgtgttctttgaaaccaacatactctgatactgagatcagacaaggacagtaccaaaaccaggcaggtactaacagcgtgctagaccagtctcacttagatgcagaaaacaaataaaatttaataatccaaatccagtagtgattgaaaggaatgtcttgatccatgaccaagtagattttattctaggagcacaaaattctacattaagattaagtagagttaaggttgacttttttttttttttttttttttttttttttttgagacggagtcttgctctgtcacccaggctggagtgcagtggccggatctcagctcactgcaagctccgcctcccgggtttacgccattctcctgcctcagcctcccgagtagctgggactacaggcgcccgccacctcgcccggctatttttttgtattttttagtagagacggggtttcaccgtgttagccgggatcgtctctcgatctcctgacctcgtgatccgcccgtctcggcctcccaaagtgctgggattacaggcttgagccaccgcgcccggccgacttttttttttttttttaagacggagtctcactctgtcaaccaggctggaatgcagtggcacgatctcggctcactgcaagctgtgcctcccgggttcacaccattctctgcctcaggctcccgagtagctgggactacaggcacctgccaccaacgcctagctaattttgtttttgtatttttagtagagattgggtttcaccgtgttagccaggatggtctcgatctcctgaccttgtgatcccccctacctcggcctcccaaagtgctgggattacaggcgtgagccactgcgcccggcctgagttaaggatgacttttaaacaacctaaatgtagctaaatatagagccttctgcaggacagcattgatgggtggaactctttaccacatgataacatcccaacaaagccacagttatggtggaactcagtccacctgagtcttaccattataagatgataataataggtaacatttgttaaataattaccatgtactttgtcctaatacttaatgtattcctttactgctcacatcaactctgaaggaaaggtaccacatccccttaaaagaaaacaactatttctattctattcttttattttttttagagacagaatctcactctgtcacccaggctggagtgcagtggcacgatctaggctcactgcaacttctgcctcctaggttcaagtggttctcttgcctcagcctcccgagtagctgggtctacaggtgcatgccaccattcccagctaatttttgtattttaagtcaagatggggtttcaccatattggcctggatggtcttgatctcttgatgtcatgatccacctgccttggcctcccgaagtgttgggattataggcgtgagccaccgcacccagcctctattcaattctattttgttctatttctattacaagccagtaaggaagaaaatatcataatttataaggaaccctataaaaaacaaacaagccaacggtccctcattagggagtatgcctgaataagaagctgaagattttcagatacaggcttcaggcaacattgtctttagaggctaagctctggctccagctccctccagcatcctgtgaataacaggcaggcttacttgcaggtgccgctttcctggacagtggtggttaaaggacaaggcccagaaagtgctgaattaggtgcccttgttatcagcaatgtcatattgatcacgctgtcttagagctctttcgacatcttggctctgcatctttttttttttttttttttaaatcttttttcttgagatagggtgttgctttggatagtagggtatccaggccagagtgcagtggtatgatcatggctcactgtagccttgacctcctagacataagtgatcctcccacctcagcctcacaagtagctgggaccccatgcacccaccatcatgcacagttaatttttgtgttttttgtagagatggggtttcgccgtgttgcccaggctcatctcaaactcctgggcccaagctgtcctcccaccttagcctcccaaagtgtttggtttacaggcatgagccactgtgcttagcctgagtccctcttttaaacaaacaaaatggtaaatggaaaggaggaaaggcttaagaaaaaagattgaagcacagatttgttgcaagcaaggaataataaagggcagttcatttagagaaaggcatatgaccacctttccctgtctccagtcagaatctagaaagtgattgaggctgggcgcagtggctcacccctgtaatcccagcacttttgggaggccaaggtgggcggatcacgaggtcaggagatcgagaccatcctggataacatggtgaaacccagtccctactaaaaatccaaaaaattagccaggcctgctggtgggcgcctgcagtcccagactgaggcaggagaatggtgtgaacctgggaggcggagcttgcagtgagctgagattgtgccattgcactccagcctgggcggcacagtgagactgtattcccccaaaaaaaaagtgattgagaaaatcaggtctgtgtgaccttagcaatgagttgtttagcttgggccactgttagcttaagtcaataacttcaagtttgcgttgtagttggaatcaatagaggaaaagctctcagcattaccacatatatcagaatgtgacattgattgccagatcagccttatccaaacacaagttctaggctttttgccctgtttatcagctttatttgctgagggtatttgatgagtcttagggaaaaaagaacagccctgggaacacagctgcttttatgatgagacatgtttgcacccataccttaatgggttttggtggcaatattctgaaatttgccaccaacgtttcaaagatttgcccttagggtgaattactgctgtagtagaagtgggtggaggctgaggagattggattaagcaggtagaggagttctcagtgcatggatcgttctgaggatggagatagagctctaagacatccacaggcctttcctgagtgatcagctttggctcctgggcagggaaattgagctggatcctagtgtgggagcacgcttgtcatcttccttctttttccccagtaccacgcggagaccatcaagaatgtgcgcacagccacggaaagctttgcttctgaccccatcctctaccggcccgttgctgtggctctggacactaaaggacctgagatccgaactgggctcatcaagggcgtgagtattctgtggagagcgagggaaagactcagtaggcaatatgccccagagacatgtcctccaaagcgttgagatgccgtgtttcatcccagcactatgaaggactgcagaggagttgaggtctacaaatgagggtttattcatcactgtaaacaacgttgatttgatttactttgctaggaaatggtaccacagaggaaccctttttttaccctaaaaacctaaactttaggctttctaacttggggaaccatctctttgtatctttttccccatcattaagtagcataactgaaacattatattctattttcttagattatttctgtgaagtgtactgagttagagaataagagcaaaaaactgtattatttttagcggtgacttgagcattgttccctggaggaaagagcttttccattgcttctgcggtgatgctgctactggtgtctccagtttggactctggcttactctcttgcttactccctagagcggcactgcagaggtggagctgaagaagggagccactctcaaaatcacgctggataacgcctacatggaaaagtgtgacgagaacatcctatggctggactacaagaacatctgcaaggtggtggaagtgggcagcaagatctacgtggatgatgggcttatttctctccaggtgaagcagaaaggtacgtatgggagctggaatccaattgtctaaaacccatcttttgtctctaaagttccttgacacaaggaagatgggaaggttggttgcctggcagtgagattgagtctgtgtgttctcaggaatcccttttataactcatttatcctcaaagacaggctttaatccagcatagttacattcttctggttccggagaacataggaacatatatatatacacacatatatttcttttttgagacagagtctcgctctgttgcccaggctggagtgcagtggcacgatcttggctcactgcaacctctgcccccagggttctagcgattctcctacctcagcctcctgagtagctgggtttacaggcacccgccaccacgcctggctagtttttttgtatttttagtagagacggggtttcaccatgttggccaggctggtctcaaactcctgacctcaagtgatctgcctgccttggcctcccaaagtcaaaacaatcttaattctccttttttatgggtgtggaaagtgaggtacagagaggttaaatggcttgcctaggattacacagttagttgtagtagggtttcaactctggtaaaacagctccagcacccatgatgcaccacttcccagctcactgtgcttgggggaaaggtgcctgcttcctgttgagctgtgccctcggtgctctgcctcccctgcttaccccttttcaaaacacaggtgctgacttcctggtgacggaggtggaaaatggtggctccttgggcagcaagaagggtgtgaaccttcctggggctgctgtggacttgcctgctgtgtcggaaaaggacatccaggatctgaagtttggggtcgagcaggatgtcgatatggtgtttgcgtcgttcatccgcaaggcagctgatgtccatgaagttaggaaggtcctgggagagaagggaaagaacatcaagataatcagcaaaatcgagaatcatgagggggttcggaggcaagtccccattgtccctgctccagtcccagcgcagctctctaaagggcatggtgcatcctgtgaatatctgattcccatcagaatgtagactcccaaacctgttccaaacctgctgaatcacaatatcttagtagagtagaaggcattgtgtgtgtgtgggttttgtttttttgttttgttttgttttgtttttaaaaagcttcctaggtaattgagatgctggcagcttgacattgttccctgggcctggggaccaacatttgagagaacagggtcactgctcatagtaccaggggccatgatgctctgttcctgatcagaaacactaccagtgtttgctggaatgtggggaccgggggaaagatgacagcagacacttaagaaggggctcgttttggcccttcctggggagccatgtggaatttcaggggctggtgtccatgctaaaacatatggcctcctggtcttcacttagaacgcagctggctcagtgatcatgctaactctggtatggtccattccactctcagagtaagatgtgtggttcttctccggggtcagattgcctcaacttagcttaccccctccacagtgctcatgaggtggagcccagtgggcatggccaccatttttggcatcctgctaggaatacaacttagcactactaagatgctagacctacgcttgtggattaggagtgtgtttggggagggtgggggaacaaccctctgcacccactgtagtggccttactgtcttagctttgtgtagataccctctgtaccaggcaatttggggccctcccctttgccatcctgataagctgaacttggcgctaggccaggcaaagccacattccctcttgccttcagcaggttggagtgggccacctcacagggcagtcttcaagtgtccttgactagataaggccatggggctttgtggtggaagcagtcagcaggccttgggttccctgtcttgaagtgctgattggagaatggaggccctagagagacacctaacatgcatgggatttggagaggagaccttgggaatgagcccatttggatttgccctctcccctttcttctgtcaatgaagcatccatattggtgttgaagcccagcaggcagaattgttggcccgctctgggggactaaggtagctggactgccttgccatctgtgtgcatccatgatgatgtcatggatgtctgtcctggtatgaggacatctaagttagggaatcccagggaaacttcttgtctaccggcatacttgtggcccctgtctgtataacctctctccccccaactttgtccatcaggtttgatgaaatcctggaggccagtgatgggatcatggtggctcgtggtgatctaggcattgagattcctgcggagaaggtcttccttgctcagaagatgatgattgggcggtgcaaccgagctgggaagcctgtcatctgtgctactcaggcatgtgaccacccctccccacattctcctgggcacactcacatgtgtgtatgggaaagctctggaggctgtctgatcttttcccatggaattgtcccatgtaacacacagataatccccttcccccatgcacctacacaaagccatgctctgtgcacctactcactatccagaggatcagcgtgctgtcatttgtctctgaaaacagcccaagccacatctcactaatgctctgttcctcccagatgctggagagcatgatcaagaagccccgccccacccgggctgaaggcagtgatgtggccaatgcagttctggatggagccgactgcatcatgctgtctggagaaacagccaaaggggactatcctctggaggctgtgcgcatgcagcacctggtgagttctggggcctgccccatcccctgggctgtggactgggcctgggttggatgcaagctgtggtgcagagctttttaggtttccatatcctcttaggcacagccgttcattatcctccaagttacagcagcaagagggtgggggtggaggtggaggtggctttttttttttttttttttaattctgtttttcattcctgccgacacccccacccctccatttccttctgctctggaagcatcctccttcactggacaccatacagttcatttcacttctgacttcaaggtggtgaatccttcccatggcttaagtcctgggatacttctgcagtgaagggaggtcttgtacctcttcctcagagtcagaagttttgagtacctttgccctattctgaaaagggctcggggctcctgctcccaactgccctcttcctttggcttccaattcagtcccccgacccccacatcccgcagacaggcgctcccccagggagcccctgtggacctgcactggagtctgttgcctgcactgagctgcctgtgctggccttgcatggtgcctataggaggatttgctttgctgtgccattggggtacagctgctgctcttacaacagaccaaaaagtcaggttgagtgactggtggcagggccacagatagagacagcagggaggggggctgaccctggtggccctggactgagcgtctggaggagtcgtggaggctctttcccttctttctcctctgagagctccttcttcaggctcttccagcttgtcatgttgagtgcctggccactgctcagggttggagactcagtccctttgccctgtctgttccagctctggagctaacttggggatccctgatcagggttacataggtttggtaaaatgagtgctggaaattaactttctcccagtagtcttaggtcaggctcagtgaacttcaactttacccagatatggtttttcttcagccattctattccctttctagccagtgaaagacccgctgccctttgacctcagcccctccaagcccccaagtttaaaacgccaccccccgccaccaaaaaaaaaaaacaactaaaacacccacctcgtctgggcatcttcctttcctttttcactatgtgtcctgttactgggcttaaacagctttcagagaagagatgtcatttctattaaatgctctttcagtagcgaactgagttcagacttgactaaggatattttctgggctgtcatcagcatccttagtgggtttccccatattggaattggtagaggccggggacggtggctcacacctgtaatctcagtactttgggaggccaaggtaggcggatcgcttgagctcagaagaccagcctaggcaacatggtgaaaccctatctctactaaaaattcaaaagttagctagctgggcgtggtgatacacttctgtagtcccagctacttggagagggggtggtgctggggcagcaggatcactgaacccaggaggttgaggttgcagtgagccaagatggtaccagcctaggtgacaaagtgacaccctctctcaaaaaagaaaccaaacaatcataaaaaaaggaaacacaaaaaatcagtagagagtgatttctctcccaggcccacttaacgtagcctgggcctggctgacacctcaccgtttgtgcgatgtgattgctgttctgatgctgagatcctcttggcgcagtctcacaattgctccatggtaggaaggtgtcccagagacagtgcaccttcaaccagtcaccactaaagtgactgcctttctgggtctctccacacgtcccctctgtctaattcccttacttaattgtgtaacttcatggcctcaaaggtgggacagaggctgatcttgacttagatttactgaaccatgaaatcactgcatagaatgtggagacttgaatgtgtcttttggcaagtcatttaacttctcaagaccttatctgtaaaatggattagatatgtttaattatagccttagcattaaatattcattgctcttattattaagtgtctgataagtctctgtgtatatggatgtaatcttcctaactcccattacctccatttatagccgagggttatatggccaataaagcctgggtttgaatctaggtctgctgactccaaagccagtcttctctcctgcaacatcatgctcttaggtctagcaggagatgagaacaggtctccatttggagcctgtcagtggggtcggagactaagaatcaggctcagcttctaaatttcatgtcctttcctccataccctagtgtttcctatgaacagacagataccttagagctgcaaggcttggattgcatggcagtgctcagaagataagttacaggtctgggccaggctgtagctgcccctccaggtggctagacctttcctttctgtgtcaccagttaacactggccaacagttccttcccttcactgttgattgctttctctgtgtctaactgatgcagttaatgacgcataactaagagcagtagcaggtatggctctgtttcgtgttcctgttccctgtcctctgggttgcatgcattccattctgataaaaagaatacctttaacctagtacatcctgccacacatctgcttctactgtgaaattcatgaggggctattaccgattcctccctcacccatcatttacttagatgctggtgattgcattataatcctctaaagcttacattgtctttctgattcttggtcttatctgagcaagtgatctgtaaataacccagtggctttctcatgactgttttaatcattagattttaatcaagtgtcttattaaacatatctgcatgcttccacagacatctgtctcttcacatggctgttcagtgtgtctctcacgagttagcccaagttttctgttctcctgcttcaaactcagttgagctgccttgctttggcttggatcccagctttccagcgctgctcaatctgtttccatggcaggccattggaaaggctcagtgcatccctgtgcctgaagccaagtgagcgctcactgcgtgcatgcatggaggctgggcaggagtctgcctaaccagacagccatgtgaggagggagggcctgttcctttctgtaagctatgtcgtgaggcagcgtgatcaagtcctctgccagggagtggtgtgggcccagcctgggcatgttttcatgccagagtgctagagcctactgccagattgtctccctccatccccaatcaaatccttccagaacggaagagccaatttcccctgtgttggaggggaagtggcagcacctcctgaagcagttggactttcatcaccctacctccgcatctgcctgaaggacagatttagccaattaacctaaggttaccttcctctctgattaattccccattctgtcttcccatgtgttgtgtcttgtttttttcctcctccttccctcttccttttcccctcttcccctaaaccttacagatagctcgtgaggctgaggcagccatgttccaccgcaagctgtttgaagaacttgtgcgagcctcaagtcactccacagacctcatggaagccatggccatgggcagcgtggaggcttcttataagtgtttagcagcagctttgatagttctgacggagtctggcaggtagggccctaagggcaggtaacactgctaggataaccagcctcttgcttcagctgttccaggagaagacagccaggcccaacctggcacctgggcacagagcctcttcttgtctataggaacaccgccagggaggtcatggcagggcaggaccaaagggtcctgtggctcagtaggcacagcagatgtcacaggcacttggtgaaggactggtttctgtggagtcttgaaattggctcagctcagcatctccagtgactgggctactcttggcctttgtccctaggaacatgttcctcaccagctgtccgggtgactcttcccctccctctccttctgtgacaaagctctgacaaagctctgtccccctctcgtccctctggacggatgttgctcccctagattgcccgtgaggcagaggccgccatctaccacttgcaattatttgaggaactccgccgcctggcgcccattaccagcgaccccacagaagccaccgccgtgggtgccgtggaggcctccttcaagtgctgcagtggggccataatcgtcctcaccaagtctggcaggtaggaggtggcagcggctccctgggatgccccgctcagtggcacctctccttgggggtcctgggagcagtgcattgaacaatgctcaggtggcactgagccaaggtaagacccctctgcctgccaccttgggcctgcagagaaggattgagcagagccccttccctgggcccagaggactctaggcagcacacataaggaatgtcagaacatttggatcaaaagcaagtttatgctggagatttattacataatagtgcacaggctgactacaaatggttatttgatattgaaaatttagtagccaggcgctgtggctcactcctgtaggccgggcgcggtgacttgtgcctgtaatcccagcattttgggaggccgaggtgggcggatcacgaggtcaggagattgagaccatcctggctaacacggtgaaaccccatctctactaacaatacaaaaaattagctgggcatggcggcgggcgcctgtagtcccagctactctggaggctgaggcaggagaatggcgtgaacccaggaggtggagcttgcagtgagccgagatcgcaccactgcactccagcctgggcgacagagtgagattccgtctcaaaaaaaaaaaaaaaaatttagtcctctaaaatagtaaaagataaccacttttgcttattccagttactatgtgctctttaaaaatttcagttgggaaaatgaatttatttaaatgctgtttactgtgcctccatttggcgcactagtccctgctgttttcgagccctaaagacaaattggtttccagctcaggagaggttgctgtgctatcttggctgacattctgtggggccaggcagccaggctgaggactgtgtggcctatgctgagcctccaacttgggatcccttccttagcccaggacattgagttaatgtccttcactctcctagttagggattatgctccttgtccctgcccacagggcagcgagggtttcctggagaagagcaaacaggcagtgcccatgcactgaggagcagcaggtgggcatgggcagcccagagaaccaggacacaagctctgtgcagatgccctcagcagagggctgcagcctcccactcttggctgaacagcttcaaccagtagggttgacctttcttaaaaggtccagttcttgctgtttggctattttaagctctagtcttctggggtttcactcagctggtcctagcttcagcagttgcttccctctgatggccttgcatataggccaagcgtgaagtgcagggacttctctgctatgatatggcttaagtttccctgacacctgttgagtgtcctcataacttcccttctggtgcccctccccagctcctgagacacagctgcacctacaagtgtgcagtgtcagtgttcaagaaagcgcctggcacaggggcttcagaagggtcccctgccttccaaaggagctttggcaggcagagctgctcctgcaccaacactcccatttcctgttcttgcctgctgagtagcacttagatttctaagcctcacctagatactcagatttgactctgggcctttatagcccagttgctggactgtttcaggagccaggggccatgtgggacagggaagagggcacaaaagtagagaagcctgatgttgattcccatggggctggtcagctctgctactgctctacatgctacttggcttgtaggtgtcaccggcagcgagaggaatgggtgctggtgacactgggccagggctgcctgtctgtgtcagagtgtaggaggggttctgccaaccatgggctgtgtgagataagtgggtgaggctgaccttgggtcaagataatcctgggtcaggataatcctgggcccttggcctgtggagtgggggtagagggcagatggtagcctagcctgctatggaggatataggatgaggggctgccctagccttgggaggggtcctagggagcagatgttgaagaggccagagccatcagtgagctgggtgagaggatgagctgtttgaataccctgaggtacttcctggggccttgtgtgatggtctcttctgtatgtcccccatcccatctcaggtctgctcatcaggtggccagataccgcccacgtgcccccatcattgctgtgacccggaatccccagacagctcgtcaggcccacctgtaccgtggcatcttccctgtgctgtgcaaggacccagtccaggaggcctgggctgaggacgtggacctccgggtgaactttgccatgaatgttggtacgtggctggagcaggggctagagcctagaggggtttggggatgcttgagcattggcttctgtgggaccctgaaagtttggggaatagaaagggaaacatagaggggcaaaaggcccagctgactgttctctccctcattgggaatgttctttctgaatctgtcattcccggaagtcctaagctgagcccaggagagaaaagggtcctttgagttgtagggttgagcagttcctttcctcttctcttctagtctgggactcaaagcacaattgtccattctttggcatctgctcattactgagggttttgtttttgtttttgttttttaaataaaattggctacagctcctgtgctgtggggtggcatacacagattatgtactgatgtggccattgtccctgtataaggtagggtatcaccagatgacaggaagcagctagctcttgaccctgggcaaggctttgcaccctctggaggatgaagtgaaagatgtccaaaggtccctgccaaccctgccaaccctgccatctgagtgataaggacatttcagggcctccctcctgtttgcctgggctgtaagtttggtgccaccttgtggtgtgaggaagtagtggtcagccagcctagttcagtgctcaggctatggggcagctgcccaggtgcacacctgcctggcttggcttttacttaccaatctcccttctcttcctccaggcaaggcccgaggcttcttcaagaagggagatgtggtcattgtgctgaccggatggcgccctggctccggcttcaccaacaccatgcgtgttgttcctgtgccgtgatgaacctgagagcccttcctccagcccctgtcccacccccttcccccaacccatccattaggccagcaacgcttgtagaactcactctgggctgtaatgtggcactggtgggctgggacaccagggaagaagattagtgcctcgctgaaacatggctgtgtttgcagcctgctctagtgcgacagcccagagcctggctgcccatcatgtggccccacccaatcaagggaagaaggaggaatgctggactggaggcccctggagccagatggcaagagggtgacagctttctttcctgtgtatactccgtccagttccttgagaaaaaatggatgcccagaggactcccaaccctggcctggggtcaggaaacagctagagagttaggggccttagggcaaggggctgttgtccccttgaagctgactctggccctggcccttacttgcttccccagccccctgggcctccccagttcgcacctgtccccgccctccactcagctgtcctgcagcaaacactccaccctccaccctccaccttccattttcccccactactgcagcgactactgcagcgcctccaggcctgttgctatagagcctacctgtatgtcaataaacaacagctgaagcacc

SEQ ID NO: 47 - DNA, 31207 bp, Chlorocebus sabaeus
  source 1..31207: mol_type = genomic DNA
atgaaggcctgtgattcagctgcaggcagcgggtaagcctgagcaggtcacactcatttggtctcttttgccaggaaaagaaaaaagaacctaagagaacggtcttcgcattttgaatgcgcaacattgtatctgtgaatgaaggcaagagttaacagctgtttaattgataactgctcacatcattggttgctggctaacaattgggaaatcggaaaatgtcttgtagaaaaatgtaagaaaagttccaacaatattgatttaaattcataacaagggtgaaaacagaaatgctgactcctgcataggttatcggctctaatgttctgacttgatatttccagatgccgagctctgcgctaatatcaacaccgtctatttgctttttactctcaggcattcgctctgcaggattccagaccctactaaattattcacatggccccaacctgtccttccttgttccgcggttctaagacaacaaatggtcctaagaggaaaagggcctcgcgctcccgctccaggttcacgtcgcagtccctagttctccctactgccgctccagtgccagagcccctccgaaggcggtcgggacctccaaccacgcacaagtctgcagctctcctcaacttcccgttcagctcagtctccgagggtgcgccagggcagacacccagaggagtggagagtggcagggcggggccgggagaatgctgccccggaacccataaatctgggccctgcccaggtaggccgggacagttggggtggcctgggccgagagccaagaaaagagaccccatccggcagcccaacttggcggcaacaggtggcccggcgcccgggggtctaggagggaagtcgctctaggggcgggctccgctgccccgccgcgttcccattggccgtcaggtttcttaaactgtgactctgaatctgtgtccttccgccgcagaatttagttcccccgaaagggcaacctgcccgcgcgttccgccgccgccaccgcgcttcctcctgaaggtgactgcgcccgcgaagacgcagggggcggggtccgggtcgccgggagccgggattggacagagggcggggcggcggagggattgcggcggcccgcagcgggataaccttgaggctgaggcccctgctactcgcactgcagctgcacgtccctgggatccacatctcttccagtttgcagcgtagcccgagtcggtcagcgccggaggtgagcggtgcaagaggctgcgccatctgtcctcaccaagggcccgtgcacccggctagtgcggaatcccggcgtgccggcgggcccgcgcaggatccggggcgtctgggacgcggtggagctcagagacgggagaatggctcctcagaagtccccagcgccgttccttccagatcaggcgggtccgccggctcctttcccgtcccagccctaccccctcattctggtcccatcctcctgccccagtcctcaatgcgcctccatccttgcctgccttctctcggtccttcgtgattgattccacccttgcctcccctttctccgcgccctctgctccgtgctcgtttgttccacttcctcaagtcctgtccttccacccactcctcttaaacctgtgcgtgccccttcattctattggcttccgtgtaacttactgaaactccgttcttgttcccctcccaggtccatcctccattcatgcgcgaccgcccttcccgcgccccagtcccctcttgtcgcccctccccctgcttgctggtcacgtccgctctcccgcatccccctcctcgcctggcatgtcgcctccctccctctctgctctggtcgcgcccgcccacttgctccggtctccgagcgcggtcccgcgccccttttccatcaccgcctcccagctcccagcaggttcgggcggtgctggggccccgtgtccggggcgggcgggcggagggctgggctgggtgccccgcgcggcgggcgggatgcggcggcccgggagcagctggaggcttacgtaacgttggcctgccccgctgccgggaggcagggcggctgcccctgcggcgcggtgctgtcccatgcgaccgggattagatgggtggcccgcgagggcctgggaatggctcgggacccgcgagctcacccggcccttgccgggtggccaccagggaccacgctccatctcgccgcggccgggctgcacgtagcggccgcgcccagggtccaccccgcttcaccgggcggtggccttgggccgcgtaacgggccgtggaaacgtctgcaggcttcgctggggcctgccggccctcgcccgctccgcgcctctccggcacgtttttttttttttcttttcgttttttctttttttttttttattatccagcacgtttttttaacaaatgcgccctgattgtggaacgcggaggccgcggggtggggtgggggtgggctctggttacggagggggcaggaaatctgtggcgttcactcaacgcaaacggtgtgggtcaagggctgtttgggggtcggagttagagaccagaatgactggacgagtcatagcccaccgagctcacaatcttaaaatgtataccccgtaatgctgggagtggggcacgagcttcctgctgtgggagggagggggacaggaagcctcgtaaggtctcaccaggtggcaagcacactggattagaagatgcaggaaagcaccttccagtctgaaggtcatttaaagactagtcaaggaaaggcatccctgggcttcttaatctgcatactccagacccagggcttggacctttgctggtacgggttaaagtgaggcctacagtgaaggtccacgtggagagagacatcgggaagttgtcagaaaggcttttagaatcaggtttgactcattaatttcctgagactgattgaaggagttcagctgctacacctcagtttcccctgggaaaactgggagtaacattttcctcatgatggttgaaagggttcatgcctacgtggatgttgcacactcagttaagtgtctaatctctggcgaaatacccagaagaaaagcctgtgaatgaatatattattttcgagcacaattacttagtccagaattgagcataagggccttgattcattatcttactgctgtctgcaaacgcctgcagctaatcgtggggaatcaggctctctaaccacgtgtctcctggaaaccagtccccccagggatctggtttaatattgacaaagtcactgatgatgtaactttccaactctctgcttggagcagatgctctttgatcatctgagattgggacctctagaggggctgagttaagccttcggtgttcccctgtaagtggaatacctcttaacaggggaacattccagcccttgatttgctctgcctgctggtgagattaggattggagcctggatcagtaacccccagttggccatcttgagggcgtggctactcccgaggccacagaggagataaaggtcaggactccctggctgcagcattgttagcaaataatttgccctttggtggcctgaggtgctgtgggggcaggagtgggctgtcttgtggggaagggaaatgtaacgcatcaccaaatctgtgggggagtggggctctgtggggaagatgactcaacctgtcaattattctgcttttgagagaattatcttcgctgggtgagtggggaaactacgtagtggttcttgcaggcttaaaaagtcccttctcccctttcagtgagtcaaataatgaagcatgcatttcccctaattaaaaatgcctcagcttcggagcttgcggtgagccgagatcgcgccactgcactccagcctgggcgacagagctaggttccgtctcaaaaaaaaaaaaaaaagtgcctcaactaagtgaaaaatgattggttcgggtcacctggttctctgtactgccttaagaacttaggtgcagagtggcagagcccggtgcccagctaatgcagtctgtgcagcttcattcctggcccttttgtagcgggcacagcagcctctcctgggacatgattaatgatgacttaaagaaagttcttttttttttttttttttgagatggagtctcgctctgtcgcccgggcgggagtgcagtggccagatctcggctcactgcaagctccgcctcccggattcaagcgattctcctgcctcagcctcccgtgtagctgggactacaggcgcccgccactttgcccggctatttttttgtattttttagtagagacggggtttcaccgtgttagccaggatggtcttgatctcctgacctcgtgatccgcccgtctcggcctcccaaagtgctgggattacaggcttgagccaccgcgcccggccttaaagaaagttcttgaactcatttctcagttactgacttgagccaaaatgtatggatgttggtctcctgtctagcacagctgctttcattgtaagcattgcgttagaaataatgtattcatattttagcaaagagcatcattagctctttaagaactgtacccccagtgacttagcaaatttggttgctgtgactctaaacagctagcactttatggtagttgcttctgtgccttgaatgttgtgacactgatgtggggtcacaggaaaaaccattttatttcttaaagctgtttcttaatattgaagcaatggaagaaatgaggaaatggccatgggcaggaagatagaaaaaaatgcagcacttacattacaaactcactttacttgggttttagtttattttaaagcttcagggtttttttgttttgtttttgtttttttttttttttttgagacagttttgctgttgttactcagtctggagtgcaatggtacgatcttggctcactgcaacctccgcctcctgggttcaagcgattctcctgcctcaacctcctgaagtagctggaattacaggcacctgccaccacgcccagctaatatttttgtatttttagtagagatggggtttcaccatgttggccaggctggtcttgaactcctgacttaggtgatccactggcctgagcctcccaaagtgctgggattaccggcatgagccaccacgcctggcctgcttcagcttttatgaaggcaaatccggatgtgttatcttggccaagagctaagactgagaccagagtacaggaaaatgactcatttgacattacccttctttctgaatcactccaaaagcccacagtcaaatcccaatttaactggtataccacaagaatgcgttgcccctgtgagcagagttttattgcaacttcgagggtctttgtgagattggtttatttggtatgaggtcttaaggattttaagttgtccctttaagcatcagttctgatacagatgggaatagaactagacaaccagatatatgaagaccatttgtgataccaccttacttttggggagcttgcattttttttaccaaggggcagactgaataattgagtaatttgaatacttggggcattgttaaagaagctgttgggggaaggtgagcctgggtgagtggtggagggtggcgtgcagcatctgctgggctttccggcctcttggtatgactgctcagctcaggagttgggggctttagggcccccaccacccagtgtggcgaagccccgcccctgcagtggtttgtgcttgtctgcacgtagaagggagcaagcccgctgcagtaccttcggtggcctctgagactcacctcccccacttctgtccatagacaaagctcctggggagctctaagcctctttcctcatgccagcaagcagtcttggaagcaggctggggctgtggtgggagagatgccaagaccagttattcaagggcagtgaggttgccagtcctagcatttcagactgtggaccagcacagctgccggctgaacactgcagtcaagattctctgaagggagtcctatcccttccaaacatcttcagtttacttacagataatgtttcttcatattttattttatttttttgagatagagtcttgcactgtaacccagggtagagtgcagtggcacgatctcagctcactgcaagctctgcctcccgggttcacgccattctcctgcctcagcctcctgagtagctgggactataggcgcccaccaccatgccaggctaatttttttgtattttttagtagagacagggtttcaccatgttagccaggatggtctcggtctgctgaccttgtgatccgcctgtcttggcctcccaaagtgctgggattacaggcttgagccaccgtatccggccttgtttcttcagttttaaacaactgatatctcccttggcaatgaatgaaaaagaactactcatcggtggaatcagtcagggcacataagacccactgtgtccaccatgccatttcaggggagattcgattgagttaagcagggaaatagagatgtcgtaaacgttgaaactatctcggtatccctctttggttattaacattagatgagcagaaaaacaaatgtcacccatcagatgggcaaacatttaaaaagtctggcagtaccaagtgtggaaaaaggtgtgcaatgcagcaactcaacgttgactgatgcagccactttggagagcaattcagccatatttagtaaaaatagaaataaacataccccatgacctaagacttcaatttctggatatatagaaactcatacagtacagggagacatgtactacaagagtagagtgttgattaatgaggaaaactagtttaatggggagggggagtggctaccagtaagggagtccataaataaaattgtctgttgtacagtggactagtgtaacagttcaaatgaattagctagatccatatacccactggatggatcttgaatgtgctgctgagtgaaaaacaagttactcagtgatatatacagtgtaccacttagggcattaagaaaaccacaatatttatagggtttcagtatagatatagatacactgtacatagacttaagtgaactggaaggatacggagttcatgacagtgattgggtgtcaaaatgcatgatgtggctgggtgcagtggctcacgcctgtaatcccagcactttgggaggctgatgcaggaggatcacttgaggccaggagatcgagaccagcctgggcaacatcacaagattcccatctctatttaaataaataaaatagaaaaaaaagttcatgatggaggtggaaagggttggaggagcttggggatggagaaaaaatgaactgtataaaaatataattttttttatttttagaaagtaaagagggccaggcgcagtggctgacgcctgtaatcccagcactttgggaggctgaggcgggcagatcacgaggtcaggagattgagaccatcctggctaacacggtgaaactccgtctttactaaaaatacacacatacacacaaaaattagccaggcatggtggcaggcacctgtagttccagctacttgagaggctgaggcaggagaatggcatgaacctgggaggtggagcttgcagcgagctgaggtcatgccactgcactccagcctgggcaacagagtgagactccatctcaacaacaacaacaaaaaggagcagaagaaagtaaagaaaatacagctcagcctttattatttgtgggttttttttttttccttttttttctgagacagagtttttcactctgttgcacaggctggagtgcggtggtgcgatctctgttcactgcaatctgcacttcttggattcaagcaactctgtgcctcagccacccaagtagctgggattacaggtgcgcgccaggctaatttttttatgtttagtagtgacagtgttttgccatattagccaggctggtctcgaactcctagtcccaagtgatctgcccacctcagcctttcaaactgttgggattacaggcgtgagccaccacagccggccatggcttagtgttaatggtcagttctgggtggtagagatgcagatgttaaaacttttttttttctttaattcataacaaaatagaaggaaacctataaaggctattgtaggaagaccagtcatactaactagttcagtgctcttggagagttggcactgcctttcctcctttattgccccaactagaatgcaggacagcccttccattaaatgctgagccagtgcctcactttgctgaggccataacccaccttagttgcacttaaggggaccctaaatcaggattcctggttctagagtgtggatatcatacccagaaacaccaccctacttttaatcctagtaaggaggcaccatgtcccaaggcaactaacgcttcccccaaaccacctccttcaggctgaaaccagttgtctgcactgagcagctgggatagagccaggaaatactcggcatctgaggatactgagggttctctgacttaggacttcttcacctgaagttgagtggtcttttggggaagtaggcccatttagcatcagctgctcttccctattccacactgtagctggaaataggaccttaggttcctgttgacaagtcatttacttttagccctgaagaaataaaagagccaagtgtttttgtttgtttgtttgtttgttttttaaagcaagggaattttactagaacctgtaagtgggctcattttgttctgtgtagcttggtaacaccatactgctttctgctttggggcctgctggggttaaagtgtgggcttcagacccaggtctcttagctagaagatatctcatcctctgtatcctgcacccatatgcaaatatgctattgtcattacccttaactgtatatgaagacagacagtgcctaccccagaccttactaggttctgctggcccatcacccattttgatcatgttgctggcctagtttgattagggcaaatcttagaaattccatttccgttgttgaggaagacagctagagagcaggctgacccgaatgccagaatatcatgatgcaaactttctaatggatgctggtgttctaaagagttgaggattgaaaggccttcatgatggcttgaagatgtaggtagcaaactgatgtcacagcaaggaacgcaatcttgtgtacttactggcaacgttggagagagaaagtgagcatcaggtgccactattttacagttgatctgtgtgaggttggtatcggagcataattggtacaaagtaaaaatgacttaggcagatgcagactcacgggccaggctggtttattagggcagaatgatttggtcctttgtgaaagaattggtggagtgaagcgtgaatctttcccagcgcaacccaacaacagtcctggccctaagaagtggagcatgggagctgggtgtggttcatgcctgtagtcccagctacttgggatgctgaggctggaggatcatttgagggtgcagtgagctataaaatatactatactatactatactatactatactatactatactataatcataccactgcactccagcctgggtgacagagtgaaagcctgtctcaaaaaaaaaaaaggttggagtggggagtgttgggtattttttatgatttttgtctcctagtttctgaagaatggccgaaaaattgtgtctgatacaaaggaaactaatacaaagagccaggagctgtctgagatgagaaaggaaagggagaatagggcacctggagctgagctgtgattgtgcctgttccaacctgtaactccagactaaggcctcttagagatggtgtctcctttctcacagaggagacatggctctgaggaagatcttactgcaggggctcgggctcaagaataaggctcctggacctgggcacggtgtgtgctgctatcagtggatacgccaggcttccactgctggctggaggttggctctgcatgtctgtgccttcctaggaggaggatgcaatagtgagtcagactggcatgggtggggccacatgtcctggcaggcactgtccgggcaactggcatgagggagaggagtctttcccagaagcccgccctgagaaaccaagactgggctgctttcctggatccaagggagagtccagtgagtgttttacatacaactctaggtaggtggttggtgggatgggcagcttgtgctgggaagaggattttggagagagcaggggagctctggccctccctgcaagggaggcttcaggtcagagcctgaggaaagacctgagcttgaggtatggggtctctggctcaaagagagcattaaggatgcatgagatctgaagtctggatgaacttgctgacaggcaagcttttttttttgactgctagtgtgtaaatccactatagaaattttcatttgtcattcttgtacttattggcaaaaaattagagcttataggcatcagatttaatttgagatagttgaaatagggtttgtgtttgaggccaatataattttatctgctaatggcatcttctggacttggaggcagcccttctgtaccagaacattgtcacaaagcttttactgtaaagcttgagaacagactagtttgctggatttgggcatgtaactaacagatttgaggcatggaattatcctatggacttttttgtttttttgctttgaggttctgaatatattaaggttgaccaccttaagccattgaaactctcctataatacatttgtgatgtagggcttaagacatgtttgtttggttttttttttttttgacacgtgtatctttgctaccttttaaaagcagattattcttgaagtgcatagagcagcaactgattaatggaattggtatcttcacatttcatttacttccaacaattttataggatgcatataaatatttccaaaaaaggtatacataatttcttactataaaagtatttttattttatatcagtaaatttgttaataaagaggaatttttgttgttgttgttgtaatcctaccaccccaatgacatcctattaaaattttagtatatatcctcccaggtcttttaggtatgtttaatttggtgtcccttccccactcccaaaaggggagggaccaggttcttgtatagaatagtggaatgttagtaaatcatgtgtttaaagagacataacagctgaatctgtagagcatctgccacctggatacctggtaattaaggtaatttttccattaccccaaagagctttagttacactcagcttttaccttaatccttgtacagctctccagggcacactttattcagctctgagcggtctttgcaagtgaggccaaggagccaacctgagccaaaaggagagcattatgtcaccggaagcccaagcccagagaaccaaaggtatgacctgatattcagtggccccagccaggtctttacaagaagaccctgatatctcaggtctaagaagagccagctgatggtttttaaaaagagtggaattagttactctgacccacttattcagatcttattttgttcacaatgtagtccccagattgtaggcccattggaggccacagcaaggcctttgtgttccaattggcctgctgtgccatctctcagtaatgttcccttaacagccagacttccctaagcccagctgggagctctgaaggtatgcgagccctccctaaaccatgagtgtagggagagggaccagggcgccccaggctttcctgtcagtaatgcagaagtttctcagattgagggacgggggagcagaggcataactttgattctgacaaagaggcattcagagagactgaaaggtcatttaacaaacactagaatgcttccacttactaggtgctaggaaatacaaaaccatataggtcctggaagagaggatacatagtagatattagaggcttaggaaatacaattgaagaatggatttgccacgttgaatagttagttggtatcttatttccccattttaaaagttttagaatctgtggttgaggacttgtggccatcagttttctgtagccgacagactgttcactattgccttcagagctctttggaccttaacaggccttctttggagatggcagagatgggtttagatgcatactctgctggagtcacccagagagcccacaaagtcagggatggaacagggtaaaggagtaagggtcatatgtgtgagatgccttgatttggaactttgagatttaggaagagatggggaagggctaaagagggacttgtttctgagccttgcttggctgaagcatttaggctaaagcgttttagaaagagtagcccttggtctgagaactcaaggaaacaactttctgatgagaaatgtagcaagcttctggttcacatccttacctgatagttcttcaaacactgcctggaagcttcccctgagtctttgtctctaatcagctaactaagaggctgagcgagtttagttgtaagtcattaatgaagaaagcaaaggtcggggccattgtcaggattgtgacctgggctagttaattacctggaactgatggtctgtgttacagagtggtggtatacttgtcaggcttagaaaagaaaacaggatgtgtatcaaaaatcttttggggaaaagatttgaccagcaactttaatttctctatgttgcagctatgctgttaatgtagttgtgataattttagaattataccagtgcccttatgttatccttgctttgcaaattgcaaattgctttacatgtcctaacgtccttctggccaacggtagatgtggttttaggtttagactcctgggatggaagctttcgcattcaggggaatgactttgggttgggtgaggattgtaaagaggcaatgtgggtgccccacgacaaagcagctatttgtagctttgtgacaacttgacatacagagatctaggtttatctaggcactaagctaggagtcagttgtttgtatcactggaagattggttataacttccttcgttggaagctccttcattgcatgttaaatgatgttatttatagatagggtggtgggaaagctgtctaagtagatgtcagcccagtgtaagagagacctgcttactgtgggtgcttgggactatcagcagtaggtgggaggttttaacttgttcagtaaggtccatttccattgttcaccatcttgtgaaccctttgtctaacatgaggagcaaccacataaccacagcatgtctggcttccctgtggcttgtgtacaaagcatacttattgagttaatgtataagcaggagacaccctcctgtgataaatggtatattaaccacttatcagtcttaccactctctttcaatttttctggacccaggacctcagcagccatgtcgaagccccatagtgaagccgggactgccttcattcagacccagcagctgcatgcagccatggctgacacattcctggagcacatgtgccgcctggacattgactcaccacccatcacagcccggaacactggcatcatctgtaccattggtgagtgagtgtgccccttccccaaaaaaagggcttcatagaaagtgaccctttctctcctgaaaagactgaactaaatgtcctaacaaacctaggtgctacatgggatgctacacagattcttataaaaggactcaggtcataggaagttgcagtaaagaattagtatgtgcacaggatggcaaatacagttaataagagagtattagacatttcacaattgctaagatggccaggtatggtggctcctagcactttgtgaggccaaggtgggaggattgtctgaggctagaaatttgagaccagcctgagcaacttagaccctctctctccaaaaagtaaaaacaaaagaaaaaaaagaattagttgggcatggtggcatgcacctgtagttctgactacatgggaggctgagaaaaagatcacctgagtccaggagattgaagttgcagtgagccatgatcacaccactgcactccagtctgggaaacagagcgagatcctgtcttaggaaaaaaaaaattgggtgtggtggcacatgcctgtaatcccagcactttgggaggctgaggtgggcggatcacctgaggtcaggagaaccagcctgaccaacatagtgaaatcccgtctctactaaaaataaaaaaaaaatagccgggcgtggtggcgggtgtctgttatcccagctacttgggaggctgaggcaggagaattgcttgaacctgggaggtggaggttacagtgaaccgagacctgaccattgcactccagccttggcaacaagaacgaaactctgtctcaaaaaaaaagaaagaaagataaaagaaaaaaacattgctaagagtaaatttcaaatgttctcaccacaaaaatgttaagtacttgaagtcatggatatgttaactaacctgatttaattattccacattgtatccaaactgtatgtattggattacataactttgtaacccaaattataaattaccagtttataataaaaaacaatttgttgcaaaaagaatccatatgctttaggttttatgctataggcaaaatctagagggtgttttccttagcaggtctttgtaggagcaacttacagacctaggaaagatctttttaatatgttctgtgcaaccaagattctgtggttggatatctggctgggtttcagtgagggcggagaaggctggccaagtcttaacctaggcttttctgatacagtgggagcctgcagaatttgaaggaaatggtcgaagtgtcccagtaaatcaaaaaagtaagctggcacagtggtagccttccatacactttttaaagacttttgagctatttgggagaggaaaagttttcagggaaaaaaattatttaaacttaagcaaacttaaatgtttttccttctttgaataattaatacttgtggctttaaaacttttcctaataggcccagcttcccgatcagtggagacgttgaaggagatgattaagtctggaatgaatgtggctcgtctgaacttctctcatggaactcatgaggtgagctgtagctggaccctttaggaaacagggagtgggtgcagtgttcatttagccacaacggattaggcaaggatgagtctgagtttcacagccaatgtgaagtttgtctttactagcccatccctactctccttccctcttgtcatggcgaagaaactggctaagtctcttttagcaaagaagaccccttttgtcgctggccatgtttctctcatacacctctcctaccattagctttcacaaaggaagatgtggaaaggttctcctggaaaaccctctaagccttagatgtcctggccacagcgcttgactctcctgtccccgggtttctgctacagcctgtattgccatggtaaaaccatctagcagatcgattctggcctatcttagaaccaaaataactgggcaggtccatgagaagagtttccattattctaagttttgaatgactgagcctaatgcacaagcactcgctggcgtataatacccctcttcctcagcactatattcccagcctgtgccttcccaggagttctgtacattaaaataacaccagttagcactcttcctcaggagcctagtaggactgtatttgtgatgggctctttattacctggctttacctatggacagaggccttgcccaggagccgggtagcagctgtcaggatggctccattcctgcctctgttgccagatttagaataaacccattctgaggagtttggggttccctgaggttccatgacttatttatttttttattttataagacaaaattttgctctgtcatccaggctggactacagtggtatgatcatggctcactggatccttgacctcccaggttcatgtgatcctcttgcctcagcctcccaagtaactggtaatacaggcatccaccaccgtacctggctaattaaaaaaaatttttttgtgtaaatgaggtcttactatattgcctaggctggtctcaaactcctgggctcaagtgattctctcaaatgttggaattagaggaatgagctactacccccagcctgggattgtgcctttttaaaaccttcagacttaaccatagatttcccatagatcatgggatttcgtaatggcattgataagaggaatcacagaagaggcagactttgtccctgtcttggcttctgtatttcctgttgagagtaaagaaaatgctaccctgtaaggccaagtgccttatagtggttgccctctggcatttggaagttggtattaagtttggactaaaaataaagcctcaggaaatgcagtccaagagtgaattcctccttttgggaaacttgagactcttcatcatagattccctaacctgtgttcataaacagcctatggcctggctagtggctggcccctaaatgtcatggggacctgaccaagtccagcagacataccacgcaggttaagacatgtccctgtaccttttggaaaattctgtagttttccaaaagcaaggggtccttagtaggagtcaccgagaattactacaagtgttagcttagcttaaagagagagagctgaagacaatggtggaggtctgttcactgttgatccctgctgctgtagtctgccgtgggctcctgcgttcagggaaaggagcagaaatagatttttaagaagttgacctttcagtaggctttgtggttccttcatccagtaaaataacaccacatagctcttacatggcaagggggagtgatatctgctgcacctgctgcatgagagctggctccgattttggttttttaaactttaagaggctttttggagattatctctgctttcactcctactcccagcttataattaagattttttaaaaatttttttatttattttttaaagatgtccctcttgtgggtttattttgaagttttagaccaagatgaggttgtctctgggctcaatttggaaactgatttgaagttattctaatttgtataatgtaaggtaaacagtttcaaccttaccgtttgtcagggctgttgtttcatatgcacctttgactaggggctgggatgtacttttctagtttctgactattttaaatgctcttctgagcagaacgttgagattactgtcttccctcttactctgacagaggaacatcaaatgtctgcatctgatcttttaacagctttctttttttgagacagagtcttgctctgttgcctaggctggagtgcactggcacgatctcggctcaccacaacctctgcctcccaggttcaagcaattctcatgcttcagcctcctgaatagctgggattacagacctgtgccaccatgcccagctaatttttaaatatttttagtagagacagggtttcgccatgttggccaggctggtcttgaactcgtgacatcgggtgatccaactgccttggccccccaaagtactgggattacaggcgtgagccacacgcccagctctcttttgacagctttggcaactagtcttcagcccttacttttggcagttcacatgggcaagatgcattcttgctgaacatgtggttccatatgccgtgttttccagatttatttatttatttagagaggcagtcttgctgtgtcacccaggctggagtgcagtggcacgatcttggctcactgcaacctccacctcctgggttcaagagattcttctgtctcagcctcctaagcagctgagattacaggcatctgctaccatgcccggctaatttttgtattttagtagagatggggtttcaccttgttggccaggctgatctcgaattcctgacctcaagtgatatgtctaccttggcctcccaaagtgctaggattacaggtgtgagccactgtgcctggcctagaaaaaatgactttcaaacaacctaaatgtagagccttccacaggacagcattgatggatgaaactctttaccatgtaacatcccaacaaagccacagctgtagtagaagactcagtacacctcccagaaatgctctaagagattataatatataacatagatttgaataatacacctaataattggtatgtttataatatatagttttacatccccaagaccaaaaatgtatgtttgtgtgaaacactcatggttacaaatatatattaggccaccaaaaaaaaccccaagtttcataaagtagaaattatacagacacattctctgataaaaaattttttaatgcaaattaagaacaaagtcaagaaaactgaagtgtgcttactttagaaagcaaagatctcaaggtagatggaataaatatttaactcagaacactaggggaggaaaaccctaaaaagggtgaagaaaaataattttgtaagattatagctcaatgaaatgaaaataaatttgacaaattaagctaagagctgattatttgtgggtaaaaaatagtaaaatagaaagttctgggaagccaggtgaagaatgcaaggatgtacaaataagagtatgaatagaaaagggaatattttctacacattggagattttaaaagtcaagaaagacttataacttaattcctatagagagagatgacactggctattttccaagaaaagattaaatagccaaaaagatagaaaatatgaatagaccagtggccataagaagttgaaaaagtgggctgggctcacacctgcaatctcagcactttgggaggccaaggcggagggatcacttgaggtcaggagttcgagaccagccaacatggtgaaaccctgtctctactaaaaatacaaaactggcggggcatggtggcacatgcctgcagtcccagctactcaggagcctgaggcaggagaatcgctagaacctggagaggtggaggctgcagtgaactgagattgtaccactgcactccagccaaaaagttgaaaaagtggctaagatctggttcacctcagaacatttaagtccaaatgattttagtggccgaattgtctccccttcaaaattcagttacattgttaaactgttccagagcctagagaaatacagaaatcttcccactgtgttctttgaaaccaacatactctgatactgagatcagacaaggacagtaccaaaaccaggcaggtactaacagcatgttagaccagtctcacttagatgcagaaaacaaataaaatttaataatccaaatccagtagtgattgaaaggaatgtcttgatccatgaccaagtagagtttattctaggagcacaaaattctacattaagattaagtagagttaaggttgactttttttttttttttttaaagacggagtgtcactctgtcaaccaggctggaatgcagtggcacgatctcggctcactgcaagctgtgcctcccgggttcacaccattctctgcctcaggctcccgagtagctgggactacaggcgcctgccaccaacgcctagctaattttgtttttgtatttttagtagagattgggtttcaccatgttagccaggatggtctcgatctcctgaccttgtgatcccccctacctcggcctcccaaagtgctgggattataggcgtgagccactgcgcctggcctgagttaaggatgacttttaaacaacctaaatgtagctaaatatagagccttctgcaggacagcattgatgggtggaactctttaccacatgataacatcccaacaaagccacagctatggtggaactcagtccacctgagtcttaccgttataagataataataggtaacatttattaaataattaccatgtactttgtcctaatacttaatatattcttttactgctcacatcaactctgaaggaaaggtaccacatccccttaaaagaaaacaactatttctattctattcttttatttttttttagagacagaatctcactctgtcttccaggctggagtgcagtggcacgatctcggctcactgcaacttctgcctcctaggttcaagtggttctcttgcctcagcctcccgagtagctgggtctacaggtgcacgccaccattcccagctaatttttgtattttaagtcaagatggggtttcaccatattggcctggatggtctcgatctcttgatgtcgtgatccgcccgcctcagcctcccgaagtgttgggattacaggcgtgagccaccgcgcccagcctctattcaattctattttgttctatttctattacaagccagtaagcaagaaaatatcataatttataaggaaccctataaaaaacaaacaagccaacggtccctcattagggagtatgtctgaataagaagctgaagattttcagatacaggcttcaggcaacattgtctttagaggctaagctctgtctccagctccctccagcatcctgtgaataaccggcagacttacttgcaggtgccgctttcctggacagtggtggttaaaggacaaggcccagaaagtgctgaattaggtgcccttgttatcagcaatgtcatattgatcatgctgtcttagagctcttttgacatcttggctctgcatctttttttttttttttttttttaaatcttttttcttgagatagggtgttgctttggatagtagggtatctaggccagagtgcagtggtatgatcatggctcactgtagccttgacctcctagacaaaagtgatcctcccacctcagcctcacaagtagctgggaccccatgcacacaccatcatgcacagttaatttttgtgttttttgtagagatggggtttcgccatgttgcccaggctcatctcaaactcctgggcccaagctatcctcccaccttagcctcccaaagtgtttggtttacaggcatgagccattgtgcttagcctgagtccctcttttaaacaaacaaaatagtaaatgaaaaggaggaaaggcttaagaaaaaagattgaagcacagatttgttgcaagcaaggaataataaagggcagttcatttagagaaaggcatatgaccacctttccccctctccagtcagaatctagaaagtgattgaggctgggcgcagtggctcacccctgtaatcccagcactttttgggaggccaaggtgggcggatcaccaggtcaggagatcgagaccatcctggataacatggtgaaacccagtccccactaaaaatccaaaaaattagccaggcctgctggtgggcgcctgcagtccccgactgaggcaggagaatggtgtgaacctgggaggcggagcttgcagtgagctgggatagtgccattgcactccagcctgggcggccagtgagactatctcaaaaaaaaaaagtgattgagaaaatcaggtctgtgtgaccttagcaatgagttgtttagcttgggccactgttagcttaagtcaataacttcaagtttgcgttgtagttggaatcaatagaggaaaagctctcagcattaccacatatatcagaatgtgacattgattgccagatcagccttatccaaacacaagttctaggctttttgccctgtttatcagctttatttgctgagggtatttgatgagtcttagggaaaaaagaacagccctggggacacagctgcttttatgatgagacatgtttgcacccataccttaatgggttttggtggcaatattctgaaatttgccaccaacgtttcaaagatttgcccttagggtgaattactgctgtagtagaagtgggtggaggctgaggagattggattaagcaggtagaggagttctcagtgcatggatcgttctgaggatggagatagagctctaagacatccacaggcctttcctgagtgatcagctttggctcctgggcagggaaattgagctggatcctagtgtgggagcacgcttgtcatcttccttctttttccccagtaccacgcggagaccatcaagaatgtgcgcacagccacggaaagctttgcttctgaccccatcctctaccggcccgttgctgtggctctggacactaaaggacctgagatccgaactgggctcatcaagggcgtgagtattctgtggagagcgagggaaagactcagtaggcattatgccccagagacatgtcctccaaagcgttgagatgccatgtttcatcccagcactatgaaggactgcagaggagttgaggtctacaaatgaggatttattcatcactgtaaacaatgttgatttgatttactttgctaggaaatggtaccacagaggaaccctttttttaccctaaaaacctaaactttaggctttctaacttggggaaccatctctttgtatctttttccccatcattaagtagcctaactgaaacattatattctattttcttagattatttctgtgaagtgtactgagttagagaataagagcaaaaaactgtattatttttagcggtgacttgagcattgttccctggaggaaagggcttttccattgcttctgaggtgatgctgctactggtgtcttcagtttggactctggcttactctcttgcttactccctagagcggcactgcagaggtggagctgaagaagggagccactctcaaaatcacgctggataacgcctacatggaaaagtgtgatgagaacatcctatggctggactacaagaacatctgcaaggtggtggaagtgggcagcaagatctacgtggatgatgggcttatttctctccaggtgaagcagaaaggtacgtatgggtgctggaatccaattgtctaaaaccatcttttgtctctaaagttccttgacacaaggaagatgggaaggttggttgcctggcagtgagattgagtctgtgtgttctcaagaatcccttaatattttttttttttttttttttgagacggagtctcgctctgtggcccagactggagtgcagtggccggatctcagctcactgcaagctccgcctcccgggtttacgccattctcctgcctcagcctcccgagtagctgggactacaggtgcccgccacctcgcctggctagttttttgtattttttagtagagacggggtttcaccgtgttagccagaatagtctcgatctcctgacctcgtgatccgcctgtcttggcctcccaaagtgctgggattacaggcttgagccaccgcgcccggccaggaatcccttttataactcatttatcctcaaagacaggctttaatccagcatatttacattcttctggttccggagaacataggaacatatatatatatacacacatatatttcttttttgagtgagagtctcgctctgttgcccaggctggagtgcagtggcacgatcttggctcactgcaacctctgcccccagggttctagcgattctcctacctcagcctcctgagtagctgggtttacaggcacccgccaccatgcctggctagtttttttgtatttttagtagagacggggtttcaccatgttggccaggctggtctcaaactcctgacctcaagtgatctgcctgcctcggcctcccaaagtcaaaacaatcttaattctccttttttatgggtgtggaaagtgaggtacagagaggttaaatggcttgcctaggattacacagttacttgtagtagggtttcaactctggtaaaacagctccagcacccataatgcaccacttcccagctcactgtacttgggggaaaggtgcctgcttcctgttgagctgtgccctcggtgctctgcctcccctgcttaccccttttcaaaacacaggtgctgacttcctggtgacggaggtggaaaatggtggctccttgggcagcaagaagggtgtgaaccttcctggggctgctgtggacttgcctgctgtgtcggaaaaggacatccaggatctgaagtttggggtcgagcaggatgttgatatggtgtttgcgtcgttcatccgcaaggcagctgatgtccatgaagttaggaaggtcctgggagagaagggaaagaacatcaagataatcagcaaaattgagaatcatgagggggttcggaggcaagtccccattgtccctgctccagtcccagcacaactctctaaagggcatggtgcatcctgtgaatatctgattcccagccccctagcccatcagaatgtagactcccaaacctgttccaaacctgctgaatcacaatatcttagtagagtagaaggcattgtgtgtgtgtgggttttgtttttttgttttgttttgttttgtttttaaaaagcttcctaggtaattgagatgctggcagcttgacattgttccctgggcctggggaccaacatttgaggaacagggtcactgctcatagtaccaggggccatgatgctctgttcctgatcagaaacactaccagtgtttgctggaatgtggggaccagggggaaagatgacagcagacacttaagaaggggctcgttttggcccttcctggggagccatgtggaatttcaggggctggtgtccatgctaaaacttatggccttctggtcttcacttagaacgcagctggctcagtgatcatgctaactctggtatggtccattccactctcagagtaagatgtgtggttcttctccaggttcagattgcctcaacttagcttaccccctgcgcagtgctcacgaggtagagcccagtgggcatggccaccatttttggcatcctgctaggaatacaacttagcactactaagatgctagacctacgcttgtggattaggactgtgtttggggagggtgggggaacaaccctctgcacccactgtagtggccttactgtcttagctttgtgtagatatcctctgtaccaggcaatttggggccctcccctttgccatcctgataagctgaacttggcgctaggccaggcaaagccacattccctcttgccttcagcaggttggagtgggccacctcacagggcagtcttcaagtgtccttgactagataaggccatgggtctttgtggtggaagcagtcagcaggcctcgggttccctgtcttgaagtgctgattggagaatggaggccctagagagacacctaacatgcatgggatttggagaggagaccttgagaatgagcccatttggatttgccctctcccctttcttctgtcaatgaagcatccatattggtgttgaagcccagcaggcagaattgttggcccgctctgggggactaagctagctggactgccttgccatctgtgtgcatccatgatgatatcatggatgtctgtcctggtacgaggacatctaagttagggaatcccagggaaacttcttgtcttccggcatacttgtggcccctgtctgtataacctctctccccccaactttgtccatcaggtttgatgaaatcctggaggccagtgatgggatcatggtggctcgtggtgatctaggcattgagattcctgcggagaaggtcttccttgcccagaagatgatgattgggcggtgcaaccgagctgggaagcctgtcatttgtgctactcaggcatgtgaccacccttccccacattctcctgggcacactcacatgtgtgtatgggaaagctctggaggctgtctgatctcttcccatggaattgtcccatgtaacacacagataatccccttccaccatgcacctacacaaagccatgctctgtgcacctactcactatccagaggatcagcgtgctgtcatttgtctctgaaaacagcccaagccacatctcactaatgctctgttcctcccagatgctggagagcatgatcaagaagccccgccccacccgggctgaaggcagtgatgtggccaatgcagttctggatggagccgactgcatcatgctgtctggagaaacagccaaaggggactatcctctggaggctgtgcgcatgcagcacctggtgagttctggggcctgcccccatcccctgggctttggactgggcctgggatggatgcaagctctggtgcagagctttttaagtttccatatcctcttaggcacagccattcattatcctccaagttacagcagcaagagggtgggggtggaggtggaggtggcttttttttttttttaattctcctgtttttcattcctgccgacacccccacccctccatttccttctgctctggaggcatcctccttcactggaatcatacagttcatttcacttctgacttcaaggtggtaaatccttcccatggcttaagtcctgggatacttccgcagtgaagggaggtctcgtacctcttcctcagagtcagaagttttgagtacctttgccctattctgaaaagggctcggggctcctgctcccaactgccctcttcctttggcttccaattcagtcccccgacccccccatcccgcagacaggcgctcccccagggagcccctgtggacctgcactggagtctgttgcctgtactgagctgcctgtgctggccttgcatggtgcctataggaggatttgctttgctgtgccattggggtacagctgctgctcttacaacagaccaaaaagtcaggttgagtgactggtggcagggccacagatagagacagcagggagggtggctgaccctggtggccctggactgagcgtctggaggagtcgtggaggccctttcccttctttctcctctgagagctcattcttcaggctcttccagcttgtcatgttgagtgcctggccactgctcagggttggagactcagtccctttgccctgtctgttccagctctggagctaacttggggatccctgatcagggttacataggtttggtaaaatgagtgctggaaattaactttctcccagtagtcttaggtcaggctcagtgaacttcaactttatccagatatggtttttcttcagccattctattccctttctagccagtgaaagacccgctgccctttgacctcagcccctccaagcccccaagtttaaaacgccaccccccgccaccaaaaaaaaaacaacaaacaactaaaacacccacctcgtctgggcatcttcctttcctttttcactatgtgtcctgttactgggcttaaacagctttcagagaagagatgtcatttctattaaatgctctttcagtagcgaactgagttcataatttgactaaggatattttctgggctgtcatcagcatccttagtgggtttccctatattggaattggtagaggccagggacggtgccttacacctgtaatctcagtactttgggaggccaaggtaggcagattgcttgagctcagaagaccagcctaggcaacatggcgaaaccctatctctactaaaaattcaaaagttagctagctgggcgtggtgatacacttctctagtcccagctacttggagagggggtggtgctggggcaggaggatcactgaacccaggaggttgaggttgcagtgagccaagatggtaccagcctaggtgacaaagtgacaccctctctcaaaaaagaaaccacacaatcataaaaaaaggaaacacaaaaaaatcagtagagagtgatttctctcccaggcccacttaacgtagcctgggcctggctgacacctcaccgtttgtgcgacgtgattgctgttctgatgctgagatcctcttggcgcagtctcacaattgcccccatggtaggaaggtgtcccaggagacagtgcaatttcaaccagtcaccactaaagtgactgcctttctgggtctctccacacgtcccctctctctaattcccttacttaattgtgtaacttcatggcctcaaaggtgggacagaggctgatcttgacttagatttactgaaccatgaaatcactgcatagaatatggagacttgaatgtgtcttttggcaagtcatttaacttctcaagaccttatctgtaaaatggattagatatgtttatagccttagcattaaatattcattgctcttattattaagtgtctgataagtctctgtgtatatggatgtaatcttcctaactcccattacctccatttatagataagggttatatggccaataaagcctgggtttgaatctaggtctgctgactccaaagccagtcttctctcctgcaacatcatgctcttaggtctagcaggagatgagaacaggtctccatttggagtctgtcagtggggtcagagactaagaatcaggctcaacttctaaatttcatgtcctttcctccataccctagtgtttcctatgaacagacagataccttagagctgcaaggcttggattgcatggcagtgctcagaagataagttacaggtctgggccaggctgtagctgcccctccaggtggctagacctttcctttctgtgtcaccagttaacactggccaacagttccttcccttcactgttgattgctttctcctgtgtctaactgatgcagttaatgacgcataactaagagcagtagcaggtatggctctgtttcctgttcctgttccctgtcctctgggttgcatgcattccattctgataaaaagaatacctttaacttagtacatcctgccacacatctgcttctactgtgaaattcatgaggggctattaccgattcctccctcacccatcatttacttagatgctggtgattgcattataatcctctaaagcttacattgtctttctgattcttggtcttatctgagcaagtgatctgtaaataactcagtggctttctcatgactgttttaatcattagattttaatcaagtgtcttattaaacatatctgcatgcttccacagacatctgtctcttcacatggctgttcagtgtgtctctcacaagttagcccaagttttctgttctcctgcttcaaactcagttgagctgccttgctttggcttggatcccagctttccagcgctgctcaatctgtttccatggcaggccattggaaaggctcagtgcatccctgtgcctgaagccaagtgagcgctcactccgtgcatgcatggaggctgggcaggagtctgcctaaccagacagccatgtgaggagggagggcctgttcctttctgtaagctatgtcgtgaggcagcgtgatcaagtcctctgccagggagtggtgtgggcccagcctgggcatgttttcatgccagagtgctagagcctactgccagattgtctccctccatccccaatcaaatccttccagaacggaagagccaatttgccctgtattggaggggaagtggcagcacctcctgaagcagttggactttcatcaccctacctccgcatctgcctgaaggacagatttagccaattaacctaaggttaccttcctctctgattaattccccattctgtcttcccatgtgttgtgtcttgtttttttcctcctccttccctcttccttgtcccctcttcccctaaaccttacagatagctcgtgaggctgaggcagccatgttccaccgcaagctgtttgaagaacttgtgcgagcctcaagtcactccacagacctcatggaagccatggccatgggcagcgtggaggcttcttataagtgtttagcagcagctttgatagttctgacggagtctggcaggtagggccctaagggcaggtaacactgctaggataaccagcctcttgcttcagctgttctaggagaagacagccaggcccaacctggcacctgggcacagagcctcctcttgtctataggaacaccgccagggaggtcatggcagggcaggaccaaagggtcctgtggctcagtaggcacagcagatgtcacaggcacttggtgaaggactggtttctgtggagtcttgaaattggctcagctcagcatctccagtgactgggctactcttggcctttgtccctaggaacatgttcctcaccagctgtctgggtgactcttcccctccctctccttctgtgacaaagctctgacaaggctctgtcctcctctcgtccctctggacggatgttgctcccctagattgcccgtgaggcagaggccgccatctaccacttgcaattatttgaggaactccgccgcctggcgcccattaccagcgaccccacagaagccaccgccgtgggtgcnnnnnnnnnnggatgaagcaaaagatgtccaaaagtccctgccaaccctgccatgtgagtgataaggacatttcagggcctcaatcctttttgccttggctgtaagtctggtgccacattgtggtgtcaggaaggagtgttcagtcagcctagttcagtgctcgggctatggggcagctgcccaggtgcacacctgcctggcttggcttttacttaccaatctcccttctcttcctccaggcaaggcccgaggcttcttcaagaagggagatgtggtcattgtgctgaccggatggcgccctggctccggcttcaccaacaccatgcgtgttgttcctgtgccgtgatgaacctgagagcccttcctccagcctctgtcccacctccttcccccaacccatccattaggccagcaacgcttgtagaactcactctgggctgtaatgtggcactggtgggctgggacaccagggaagaagattagtgcctcgctgaaacatggctgtgtttgcagcctgctctagtgggacagcccagagcctggctgcccatcatgtggccccacccaatcaagggaagaaggaggaatgctggactggaggtccctggagccagatggcaagagggtgacagcttcctttcctgtgtatactccgtccagttccttgagaaaaaaatggatgcccagaggactcccaaccctggcctggggtaaacagctagagagttaggggccttagggcacggggctgttgttcccttgaagctgactctggccctggcccttacttgcttccccagccccctgggcctccccagttcacacctgtccccgccctccactcagctggcctgcagcaaacactccaccctccaccttccattttcccccacttctgcagcgactactgcagcgccttcaggcctgttgctatagagcctacctgtatgtcaataaacaacagctgaagca

SEQ ID NO: 48 - DNA, 24791 bp, Canis lupus
  source 1..24791: mol_type = genomic DNA
gaaatgcagtggggctgtcctgaggggaagggtatgtagcccatcaccaggtttgtggatgagtggggaggcctctgggggaagatgactcaacctttcaacctctctgcttttgagagaattctctttgttgggtgagtgggaaactgcattgtggtcttcacaggtttaaaaagtcccttctcttcatttagtgagtcaaatagtgaaacacgcattttccccagttaagtgaaaagtggttgtccaggttgtcccggctctctgtactcccctaagagcttaggtgcagcgtgacacaggcctgtgcccaagtaacgtagtctatgcacctgcattcctgagacttccccaggggacacggcagtcctttgtgggaggggggcctgttatggttatgaataatgatgactgaaagaatgtccttgaactcacctctgacttgagtcaaaatagatggacattagtctcttccagtcttagtttagctgctctcattctaagcactgcgttaggagtaaataattttgatgttgaattcatagtttaacagcatttttgggtgtctaataatttgccagtgacctggccaatttggtaggggggactccagacagaactgggaccttgtacaagttgtttctgtgccttggatgttgtgacattaatggggtggggggtgtcacgggaaaagccattttatttcttaaagctgtgtgtttcttaatgttgaggcagtggaagaaatgaggaaatgactgtgagctaggaaaaacccaactcttgcattatcaactcacactaccctggttctaatttatgttgcttcagcttgtatgaaggcatgtcaggatgtgttattttggccaagagctaagactgagctcaaggtaggggaaaatgactcatttgacattttatttctgaatcattccaaacgtctacagtcaaatctcagttaggtggtaccccacaaggatgagtcgcccgtttgagcaaagttttcttgcagccttgttgaggacgttgggaaatgtagaagtggtttactcggtgggagatcctgtaggaggggagcctggctggctcagccagtagaaagggcgactcttgatcttggggtcgggagtttcagcctcatgttgggtacagagattactttaaaaaagaaacattttttaaaagttctccaggactgtgagtcatcactttaaacctcagttctggtccagctaggactagagctaaaggaagagcgttcttaatgcgagcatgctctgggaacttgggtttttcaccacgagcccgtctcagactagaccaatgaatagtctgcttgtttggggctttgttaaggaagctgtttgggggaaggtgagccagggtggctagtggatgggggtgtgcaacatctggtgggcctctctctcggcctcccggtgtgactgctcagctcaggggagggggtgttggcagagccaccgagtggtgtggcgcccgcccctgcagcggtctgtgcagttctgcacgtaggaaggagggcctgcaggaaggagggccagccgggctgcagtacctccggcggcctctgagacttacctcccccacttctgtccatagcaaagcttctggggagctctaagcctctcttccctgacgccagcaagctatgtttggaagttaggcaggggccgtggtgggggagatgccaagccaagctgtgacagtgccagcttagctgatatagttcgaaaaggtctctggaaaggcgcccattccttccgagcctttgaagtcgacctacagctagtacttctttaacttcaggtaattagtgtcttccgtgtttccagcgacgagggcaaaagaattaacccggtcagtgaatcagtcacggtataggagacaaaaccacgggcaacttgaaggagccctgggagttttaatctcgttagaggtagggaagtagaggcttttgataactggtatctctcttttgttatgacattagaaaacaggttaatgtcccctatccagagggtctacattttaaagctctgacaatattgaatgctggcagaagtgttagagggcaagaactcacattgctggatggttgtaaatggatataacctcgttagggcccttcagcaatatttagcaaaatagaaaataaacataccctgtgacttaggaatttcatttctggatatatatagaagctcacacttgcacatagagccgtgtaccaggagaattgttgattgcattgaataatagggggaaaattgattgggttgccaaccagtaagggaatgcgtaaataaccgatgtagtcgtgcagcggatcggcatagtcgttcgtatgaattaactagatctgtgtacccgcaggatagatcttacatgtgctgaaaaaaagcaagatgtttatttgtacagtattgtactgagggcattttagaaaaccggtgtcataggaatcagtatagatatagatgcttaaatggattagaaggatacaaaattcctgacagtggctgggtgtcaaagttcgtcgaaaggttggagggccctggggatggagggaaaataaactacaaaattgtaatggttaagaaaaaaaagtctacaggaattctggctaaatgttagtagtcagttctgggcggtagaaatacggatgttaatattcctttttttttttcttcaattaaaaataaagcaaaatagaagcagatctgtgaaagtcactttaggaagaccagttgtactaactaggtcagtgttcttggagagctgggactgcccttcctcctttcatcctccccttctctccctccagctatggtgcaggacaacccctcctttaaatgctgagctagaacacctcactttgcggaggctgtcacccatcctggttgtgtatcggactttgaagggccctaacctagagtcccaggtcccttgctacttccaaactatgcatatcatgccctaaaacactgcctcactattttaggcctagtaaggaggcgtggtaacacaggacaacaacagtttcctccaaacttccttcttcaggctgaaaccacttctctgctttgagcttctgtggtcaaacaaggacatctgaggtttctctgacttgggacttcctgctctggagtggagtggttctttggaggctgggcccgtgttgtatctacttttgtgccctaatcccactttgaaatatgacttgagactcctgcttgtaaagccacgtccctgaataaataaaagcaagggaattttacttgaacctgcaagtgggctcattttgtttcctgtaagcaggtgagctagactttgctgtctgctagggagcatgctgagggttggaacctcagctccctcctccgtatctcgctccatatataaatgcacaatatcattactctaactgtttatcagggcaaacagtcattcggtgcagaccttactaggttctgtgggtggcctggcacctgttttgatcgtgttgtcagattggggtcagtcttgggaattgggaattctattgctccatcatctaggaactctctagagagcacactggcccgaacgccttgtctgttctgaagattagaggcctgaaaggccttcatcatgctagcttggagacttggaggtgtaggtagcatattgatgacagagccaggaccacagttgtgggcatcaggtgccatcatcgttgggttactctgtgtggcgaggtcagcgtcagaggaaacttggtgcggagagccctggctggttaaaaattggcttgtgtgcatacagctcacgggccaggctggtttactagggcagaatgatttgggcctttgtgaaagaatcagtggagtgaagcgtgaatctttcccggcacaaacttgccaacagtactggccctaagaagtggcacgtcgtatcttttatgtgattccctcccactgtggtttctggagcgtggccgaagacctgtgtctggcacaaaggaaactatagttttggagaaccaggagctgcttaagatgagaaaggaaaggtgggccggggacctggggagctcaggtgtgaccgtgcccaccccatccccttggcctggtagggcttctcacggtgtctcctttctcacagaggagaaatggctctgaggaagatgttactgcaggggctcaggctcaagaataaggttgccgtacctgggcatggcatttgccaccatcagtggatacgccgggcttctactgtaggctggaggttggctctgcatgtctgtgcctcccgggaggagggtgcaagagtgagtcagcggggcacccgctggctgcgtgtcccggcaggcgctgtccagacggccagcatgagggagggagctcttcgcagaggcccgcgggaaaccagggctttggctgctgccctggggccaggggagagtcctggggggttttggcaagaacttgaggtagtggtggttggcaggacgggaaactggtgctcaggaggacctgagaggcaggcagcagagccctgacttccagcaaggggggctcagaggctgggagctgagtgctctggtttggtagacgtgtctaagggcactttgagatcttgtaggagtttggacaagcaagcttttttttctttttttttctttttttctgattactggtgtggaaatccactcaaatgccagataagtgttcattattattattattttttacatttcataggctgggtgagcagaaaatcagcttgcaaaacctcagatttaaattgagatggtaaaatagggttgttgtttgagcgcaatataattgtatttgctaatggcatctcctggccttggagacagcccttctgtaccagaatatccttgtcacaaagattatgctgagaagtttgaggacagactaaacaggcaaagaagtttgctggatttggatgcataactaacaggcttggagcatagaattgtcctgtggtctatgtcaactttttttttttttttccttttctttctttttttttttttttttaatttttatttatttatgatagtcacagagagagagagagagagaggcagagacacaggcagagggagaagcaggctccatgcaccgggagcctgacgtgggattcgatcccgggtctccaggatcgcgccctgggccaaaggcaggcgccaaaccactgcgccacccagggatcccttttttttccttttctttctttggggttctaagtggatcagagttggccttcttaagccagtggaatggtctggcaatgcttttgtgatctggggcctgagacgtgttatctcgcctctgccacatttaagcagagtattcctgaagtgcatagagaggcggttgattaatgaaaccagaagttaagcttcatttttgttttgattccaacatttttaccaggaaacttataaatgtatcatttttggaagaagtacatttgtgatttttcttaatacaaaaatatttccattagtaaatttattagtaaagcgaagaaaacattttgctgtaatcctaccacctaggtgacctgccattactttatgggtaaatagacttccaggcctattatgcatgtttagtttagtagcccttctccctcaaaaactgggaattgaccaggttcttgtatagaatagtgaaatgttaattaaccacatatttaaagggaatttagagcgaaaataatctgggtattcaggaggagataaagcatcatagagctagcaagaatttcctttacctgcaaattttctgttaccattttcttggtatttccttggaggtatctgtgtgtgtgtgtgtgtgtgtgtgtgtgtgtgttttaagattttatttatttattcgagagagagagagaggcagagggagaagcaggctccatgcagggagtctgatgtgggacttgatcccgggcccctgggatcatgccctgagctgaacgcaggcgcttaaccgctgagccacccaggagtcccatatctatgtttttctctgtaacatagcagagtctccgggagcagctaccatctggttacctagttactaaggtcatatttccattgccccaaagaacttcagttacactcagcttctaccttgatccttgtacagctctgcagagcaaaccttactcagctctgagcggtctttgcaagtggggccaaggagccaccctgagccacaaaaaggacattatgtcaccggaagcccaagccccgagaacacaaggtatgacccaacaacctgtggccccggccaggtctttgtttgcaaggaaacactcttgtctcgggtctagagaagagatagctgactatgtttttgtgttttttatgtttctgtttttggtttttgtcaggtgtttttttttttttttttttattttaaattttatttattcaggacagacacagacagacagagagaggcagagacacaggcagagggagaagcaggctccatgcagggagcccgatgtgagactcgatctcgggtctccaggatcacgccctgggctggaggcggtgccaaaccgctgggctatcagggctgctccagctggtgatttttaagaagatgcaggttaattattccaatccactttttgagatcttgttttattcatgatgacagtcctcagactggatgatgattccacttggaggccacagctaggcctttgtgttccagttggctgggtgtgccacctggtgccatctcttcagtcatgttctcttaacaggcagacttacctcagccctgccatgtgctccgaaggcacaggagccctgactccacgcgagtgccaataaagaaaccctggctgccgaggcaggccctctagtaattcataaattcttcagatttggggaaggagggaagagaggtataactttggttttgacaaagaagctttgaggtacacggaaaggtcgttcagcaaacactggaacactccccagtactaggtgctaacagagaacaggtaggtcacagaggagaggacctccaaacattttcgttttggtgcctggtgtgtattagggagttacgaaacacacgttgaaaggagcagcgcatttgccgtgtggaatagttagccaacatcttatttccccatttttgaagtatcagcctctgtagctgagcatgggtagccatccgttttcactagctgacaagttttgctcacaactggtcacggaacttcttgggcgtgtatcagaattcttgaaggtagcagatatgggctgagatgcaagctaccccgagaggccccaaagtcaagggcggggggaggaggaagtaaaagtggcatgtatggcgtgcctggattcagagccctgagagttaggaggagctgggacaggcctgaggagaggcttgttcaggagcctgggctgaagccttgaagctcccagggtgttaggagtaacccttggtccaagaacttgaagaagggacagccttgtgatgaggagggtcgagcttctgactcacatccttacctgatggtccttcagaccagcctggaagcttcttaggtctctataatcagcaaactaaccggctgagtgagtttagctgtcagtagtgaaggaagcaaaggtcgggacagttgtcaggattgtgaccgggtctgtttaattacctgtaacaggtggtctttgcggtgtgcttgtcagaaagggaatcgggatggtgtatcagaaaatcaggtgggggagagggtttggccagcagtctggatggatttcctcaggtgtaactgtcctgttaaagtaggtgtaatacgtctagaacctgaccagtgccattggggttatctggttttatgcaaattgctttgcacgtccctaaagtcctgctgaccacaagtagctgtggttttcaggtgcagactcctggggtgaaagtgcgctcgggattttgggtcagctctgcttcctgtccaaggagtggttcttactatctgtcagtgaactgggatcggctctcctgccagagcaagccaaggaatgaacttacatgttgctctggcttagagaaaatctaggattccagaggtcaaaggactgcctggatgatgattgtctttacaaccaggaaaatcactttgagtttgggtggggattattaaaaaccactatggatgccccaccccatagcggacgtttatagcttgagatagcttttcatacaaagatttatatttgccaagatactaaactagcagttagtgctttctgtcgctggaaggttggtcacgccttcctttggaggtagcttccttcgatgcctgtgagatgttgtttggaggaggacgaggaaagctgtcaaagcggatactagcccgttgtaagggagatctgcttacagcggatgcttgggaagcacgtgtgttggggacgaggttttaaattggtctttgaggtcctttccacttgctcatggtcacgtgaaccctttgcccgacttgaggagtgcccacgccaccagagcatggtggggctccctgtggctcgtgcagagtgtgcctattgagttaatgtgtaagcaggaaacagcctcttgtgctaagagatctattaaccacctgggcttctcagactgatgactctctcttcaattcccctggatccaggacctcagcagccatgtcaaagccccatagtgacgtcgggaccgccttcattcagacccagcagctgcacgcggccatggctgacacgttcttggaacacatgtgccgcctggacatcgactccccacccatcacggcccggaacactggcatcatctgtaccattggtgcgtgggtgtgtcccccgctgcgccccccccccccccaaaaaaaaaacaggggcttcatatgaccgtgatcttccctgaaaagacaaaagaaatgtagcaaacctgggtgctgagcgagatgctacacaggttctttgggaactctggactccggtcctaggaagttgcaataaggaattttgcgtgctctgtgtttgctgctaaaggcagaatatagacagtgtgttgttccatagcacatcttttaggagcatctcagaaatctagggaagatcctttttttttttttttttttaagattttatttattcatgagagacacagagggaggcagtgacacaggcagagggagaagcaggctccatgcagggagcccagtgtgggactcaatcctgggaccccaggatcacgccctgagcctaaggccgacgctcaaccactgagccacccggggatccctagggaagatctttttgacttgctgtgtgcaaccaagattctgtggttggacatctggctaggcttcagggccaagtcttacttaggccttcttagttagatgggggcctgtaggatttcaagggcatagcaaaagcatccccttagctcagggaagtgggtaaacataatattagtcatgtatatgcttttgaaagaattttgagctgtttctgggaggaaaatttaagggaaaaggatatcaagagttcttcaaacttgaaagtgttattccacctttgaatatttaacactttggctttaaaactttttttccccctccactaggaccagcttcccgatcggtggagacgttgaaggagatgattaagtctggaatgaatgtggctcgtctgaacttctctcatggaactcacgaggtaagcctcagctggacagcctggccataggggcagcctgtaggaaattgggtggcagtgtggttcatttagtcatagtgaattaggcaagaggaagtctggggtttacagccagcgggacgagtgtcttcactggcctatcctatcatcctttccccatctcctgccatcccagttggccaagtcttcttacccaaagatgacctatttggttattggccatggttttctcacactcgtgtactgactaaagtgtggaaggtttggggcacctggctggctgaggcagaggagcgtgcaactcttgatctcagaggtgaacatttggggcccacactgagtgtagagcttgctgggaaaataactttaaaaaaagagtgtggaagggttctactggaaaaccctgtaagccgttaggtagcatggcctcagtgcttgacacctcctacccctgggtttctatagctgttcatattgcagtgatgaaccaaaaagtcaccctgcagattgatttctggccgaatgtagacccaagataactgagcaggtccaagagaagagtttctataccattgcaagttaaggaccgagtccgatgtaatcacactatccagcatacagtaccccacttcatcaacactgttcctagcctctctcttcccatgggttctagcatgttaaaatagagtcagcacactccccagcagcctggtaggaaaggatctgtactgaggctgagtatgagctgacttgacctgtggacagagacttgacccagggaccaggtggcaactcttgggacatgctccatcccccctcctgttcccaaactaattttgagggaggagtggaggttccctgagggacaattccttttaaaacccatcgggttccatcataagctgtgggatttcaaagtggtatcgataaagaatgaaggcgccagacttagttccttctttcgacttctgatactttctgttcagaataaagaaaaagctgccctgtaaggacaagtggtgaggggttgccctctggcatttggaagctgctgtttagactaaaaataaagcctcaggaagtacagcccaagaggtgaacttctccttctggaaacgtgagactctccagcatgggttccctaacccagtttcataaacagcctatggcctggcaggcagctggcctccaaatgtcatgggggcctgactgagttcagaggagctgggctcagccggcacttcaggcaggttaagacacgttcgtggtgacattctgcaattttccaaagtcggggtccttggcaagagtccccaggaattcttgcaagggttagctcagcccagagagagagctaaagatatcagtggggatcagttcaccattgatccctgctgccggaccatgccagggtctgctgggtgcagcagaagcagcagaaatagatgtgaagaagttgacctttaacaaggcttgatatggcctctcccatccagtaaattaacaccacagcagctccaaaatggcaaggctgtgtggtgtccgctacacgtgctggacaggggggctggcttggggatttggactttttaaaacaagagccatttggagataatctctgcttaaatacttattcccagattagaattgggatttggggaaaaaaaacaaaaacacccaagttcctgttgtttgaggtttttgactagaataaagttgtctctgggatcaagggtttaaaaactcctctgaaattgttctgatatcctataatgtcacataagcagtttgaccgcatatccgagctcttacttaatacccacctttggttagtggctacaatctgctttcccagctcttgacaatttttaaatactcttctaaacagagcattgaaatagttttctctccctcacactgaagggacgtctattgtctgcatctgatcttttctgacagctttgcctgccacactgtccctaacccccttgggcagttcatatgtgcaagaggtattcttactcaatgtgtggtcccagagacacagacctttttctttttaagattttatttattcatgagagagactgacaggcagagaaacaggcagagggaggaacaggctccatgcagggagcccagtgcgagacttgatcccggaactctgagatcacggcctgagccaaaggcaaacgctcaactgctgagccacccaggcgtccggagacactgacttttaaacagcctaaatttagaaccttccacagggacagtattgatggatggaactctttaccatttggtaacatccaacaaaaccacagccatagtggaagactcagtacacttcccagaaatgcaccaaaagatgacagtatagaggatctgaatgatgtaagacctaataatctcatttgtttatacatagccgatgattttatgccccccccaagaacaaaaacacgtgttgatttagaatgtttatgattaagtaagttattaggccaccaaaacccctcaagttccataaaatggaaattatatagaccatattttctgggatatctttttatgcaaattttcagtctccggaaaactttcaggggcatgggcacacagtaggttaatcatcttactcttgatttcagctcaggtcatgacctcagggttgtgagatcaagccccacatcagactccgtgctgggcatggagttttcttaggatcctccctttccctcagtcactcccctacttcatccacaaacaaaacttacaggcttaaatgtactttttggaaaacaaaagtcttaatgaaagttaatgaaatacttaaaatactaggagccaaataatctaaaagggataaaagaaaagcactgatcttcaagcataacttgataaaacaaaatgaatttgacaaaccgaaaataacttcctgtgaaacaaacttctggaaagtctggtcaagaatgaggatgcataaaaagaccatgaaaagggactatttcccatatattaggagagagtttgaaagtcataaaaagacttctctaatttctgtaagttgagatgaagttgactatcctccaagaaaaggtaagtccaagaggtagaaagtgtagattaacagcctgaagaaatcggaaaggtgggcagcccaggtggctcagcggtttggcaccgccttcagcccagggtgtgatcctggagacccgggatcgagtcctacgttgggctctctgcatggagcctgcttctccctctgcctgtgtctctgcctctctctctctctctctgtgtgtctctctcatgaataaataaaaaaaaaaaaaaagaaaaaaagaactcgaaaaggtgactgtgggctctagctccatcccaggtcaagaggacttgatggcttacttctgtctcccctcaaaggaactttgtcatgctgttccagagcctagaagtcttcccaatatgttctttgaagccaacacacaccctgatagtgaagtgagatgaggacagtaccgcaaccagacaggttagctcttggggcctgtctctcacttagaagcagaaagcaagggtaaaattcaacagtccacatccactcatctgttaaaggaatttcttcccccgtgaccatgtgaattttattctaggaacataaagtccacactgggatgaaatagagtggccagcatgaggtgatggtgataactaggtgctcacctctgttctaaggacttcgtggattcttttacttttcacatcagctctgaggaaaaggcatctgacaacaacgaaacacctactcctttaaaataaattacctctatattctgttctatacagaatatttttattctgtttcacaagctagcaatagaaagacatattttcataactcctagtacggacatctgcccccagagaacaagccatggtcctcatcagagactataatagaagctgaagattctcagacacagcttcaggcagcaccatccttggagatgagactctggttccagctcccccctcagctgccttatgggtgctgctctcccgggcaggctgaaggacagacaaggcctgtcaagtgctgaactgggtgcccctgttaccagcaatgtcctggtggctacactaagaacttggtgacgtcttggctttgagtccctttttcagtcgtcagtcgtggcgagtggaaagggaaaaggctggaagccagggcgcgtggttcagagagaggcgcattttcctgttccctagaccactccagaaagtgattgtgaaaaccaggtctgtgggcccctaggcgatggcttatttagcttgagctattgtaagcttaagtaaataactctggagtttgaattgtagttggaatcaataggggaaaagctcagaactgccaactctcccaccggactctgacactgattgcccggccagtatcacccacacacacgttagtgcaaggtctttttctcccgcggtctgctttatttgccaagggagcatgagcgtaagggagacagacttctttcatgctgacaccgtgtttacatcatccctgtagtgggctttggtggccgtattctcgggtttctaagctttggccttgagtgagtgagtgagtgagtgccacggaaggagtgggtggcaacgaggcgattgaaatgagcaagtggggggtgctcagtgagtggatgaccctgacgccagacacaggagggtagaggtctggaacatcacaggcctttgcggagtgatctgcttcagccgcggctccagatcttgggtgggagcacagctgtcctcttccttcccccagtaccacgcagagaccatcaagaacgtgcgtgcagccacagaaagcttcgcttctgaccccatcctctaccggccagtggctgtggccctggacaccaaaggccctgagatccgaactggcctcatcaaaggcgtgagtatcaggggccagtgggagggaaggtgcaggaggcagtgggccctagagagccgtcctcggtggcgtgtgtcccggttggaggccgtgttcctcacagtgcagatctggaaattctattcatcatggctaaaaaagttgatttggtgtactttgctaaaaaatggtacaaagaagaatcttttttttactttactctgagaacctccttccaagtctgtccgttgcttttcctctatcgttacatagcgagacgatgatgttgtaggacgttgtaagtctttcttgggtcacttccttgagaaaagattgtttatttccaaagccagaagctttattgttcctcatggctgggattggagcatgttccctggaggaaagagcttttcttttccttcggagcgggcccggctcctgtggtttggactcgtggcttattctcttgtctgtagagcgggactgcagaggtagagctgaagaaaggagccactctcaagatcaccctggacaatgcctacatggaaaaatgtgacgagaacatcctgtggctggactacaagaacatctgcaaggtggtggaagtgggcagcaagatctacgtggacgatgggcttatttctctgcaggtgaagcagaaaggtatgtactggaggcaatgtctcggtgtctagaaccagccctaaatttcctttgacccaaggaaagtgggaagtttggctgacagcaaggttaaggctggtttttctcaggatttctttctgtaattccttgtcctttacaatagactttggctcaacttaatcaccttcttggggagcataggagaagatttctttttccccatcctgtttgtgtctttgtggttttcttccttccatcgctcagtgtgatacgtgtcaggcactatcccaagtattttacatggtaatttgttcacgttttataacaaccttatgagtgggtattatgatctctgacaggtggggaaacaggcacagagaggttagttgacctttcccagactttacgattagtgagttaggatctgagttctggacatagtggctccagcacccacaatgtgctgctttccagttcaccctgctggggagagcctgcttcctgttggacattgtgcttccatccctccatccccatttcgcacaggtgctgacttcctggtgacggaggtggagaacggtggctccttggggagcaagaagggtgtgaacctccccggggctgctgtggacctgcctgctgtgtcagagaaggacatccaggatctgaaattcggggtggagcaggacgtagatatggtgtttgcgtctttcatccgcaaggcagctgatgtccatgaggtcaggaaggtcctgggagagaaagggaagaacatcaagataatcagcaaaattgagaatcatgagggagttcggaggtgggtgtcctgaccgccccctgcccctcagccccagcccggcattccagaaagcagactgtatcccgtggatacccaggtcccaaacctggcgacctacccatcagagtgcaaactcccaagcttaggctagacccactggggaggagaaggcattggcatttttaattatttacataagggttttttttttttttttaatttgatgtatttattcatgagagacacaggcagaaggagaagcaggctccatgcagggagcccaatgtgggacttgatcctgggattccaggatctcgccctgagtcaaaggcagatgctcaaccactgagccacgcaggtgcccctatttacttagttttttgttgaattttatttaagtaatctctatacccgatgtggggcttgaacccacaatcctgagatgaagagtcacatgttctttcaagtgggccagccaggggccccaaggaatgggtatttttaagaaaatacatatatcatatatatcttcccaggtgattggcatggcgatgcaccccagaagtgactgacattgttctgtaggcccaggcccagtgttgggagtggatggggagggtcactgctcacaggggccctcatgctgtcctcctgatgagcgctacctgtgtttgctggaataagaggacgtttccccccatcctacacctccaccccatttttcccagtggaaggaagaagatggcaggctgttaggaaggtgctcttgtttggcccctcacttgggagataggtggaagctcatggtgtccttgtcttcacttaaaaggcagttggcctgctgactgggccggttctggcatgttcccagcttcctggaggaaagcatgtggtcctagctgtacttcacctccttcccagtatccccaagtagagctaaaagagtatggccagcttggtgacatctgttcctctgcagctccctgctaggaacataagtcaccagaactaaaaccccagacctacttgggtggattaggcctgtgcttggagagggcagaggggcagccctacttgatcttgccatccttagctttgtgcgggtggcctttgtaccaggcaatatggagtctaccttctgtcctgataaaccatgggtgatagctgtccttggccataggccaggccaagccacatgtcctcatgccttcagcaggttggaggggcccccctcaaggaacagccctcaggtgtccttggctggaaggccacagacctttgtgtcaggagcagtcagctaaccttggaagtgctggcggcagtgggggtggtagagaggtgccttaggcggggcctcatgacctgcagggggagaggaaacaaacccagggggcttcggaggaaaccttgtcttttcccttaccccttggtggaacctccatacgagtgctaaagcagcccagcaagcagtactgttagcccaccttgggagaccaggtagctggaacaccccatcccctttgtgcccacggcgaggtcctggacagctgtcctggtgaagggctgctgagtcagagtccaggagaacctctgtgcgtcactgcacttgcggccttgttccatagaacctctctctccctgccctctttgtcaggtttgatgagatcctggaagccagcgatggcatcatggtggctcgaggtgatctaggcattgagattcctgcagagaaggtcttccttgctcagaagatgatgattgggcggtgcaaccgagcgggcaagcctgtcatctgtgcgacacaggcatgtgcctctccccacactcacacactcacaggcgggtgctgcttggtctcttcccaggaccctgccccacgcctctcaaacacacgtgcaccctcccttcccctcgtgcatgcacacaaagccttactctgtgcacgaacacaggatccagggaatcagcctgtagtccttcctttctggagaccgcccaggccacatctcactaatgctctgtccccgcgcagatgctggagagcatgatcaagaagcctcgtcccacccgggctgagggcagtgatgtggccaatgcagtgctggatggagctgactgcatcatgctgtctggagagaccgccaaaggggattaccccctggaggctgttcgcatgcagcacctggtgagttcctcgggcctccccgccgatgtctgtaggctcgggctgggtggaagcaggctctggtacgaggcaacacctttcaggcttctgtgcccacaaccttttatgatcctctaataagtcacagcaagactgcggaggtgtgtgctggggaggagagggtgtcttggttttcgttctcttctgtcttcactgtccacgctccatctcctgtctcctcctgtcctggaggcatcctccttcatcagcctacacggttcacttctggcctcgaggttgtgaatctgtcccatgattcaagtactgaggtacttgtccctcttcctctggacagcccagaggacccagaagaggccgtgggctcctgctccctgccagcctgtctggcttccgtttgcatccccacctggccacatcaggtgtctccccccgcccccaggggccccgggcctgcactggagactgtcgtgtgtgtgaggggaccgagctggccttgcatggcgcctgcagcggggcctgcttcgctgagctgctgaacggtggggtttagtgactggcagcaagtccacaagtagaggcaggtggaaggcgactgagcctgctggggcgtggagggagagcctccggaagagtcctggaggcagctttttcccaccctggcagctagctagctctgacgctcttgggcttgtccacgtgcccctgcctggctccttctcagttccttggccctgtctggtcctgctctagacacacggaagacttcacccagggttcacttaccagggctcctgaggcctgggaggcagaggcctgcacattcactttcttaacagtagtccagtgtcagcacttaacgtgtgtggagctctgcaaacttgcctctgatcctgaagtggtttttccttgacctattccttttcccatcagcagagggaccctgtgccctttgcgtgatgccgcctcccccgaactctcccagatgtaaccccccaatacacaaagtctcttcctttcctcgttcactttttgtgtgtccagttattagcctgtgtagattgatgtaatctttgtatatcccctgtgtgatagatgtcaccccaatttacggatgaggattacgtgatgataaagccttagtttgaacccagaactgctggctgcagggccagtgttgtcctgtatgtagtgttctgtctagcaggcgataaggccacgtctccatcaggggcctgtactgggctcagagattaagactctccctctggcctctgaattccagctatttttccataaccagtttctcctctggacagacagacacaccttagagagaaccccagggcttggatggcactgctcagaagataagtgatgaggtgtaggtgaggtggcctctcctcacgtccccagttaacgccgagcttctcttaaccgttcactgctttctctcgtgtgttgtgtaaatgaggcaattaaccacccataactcagggctaggccgtctcaggtacgtcccgtttcctgctcggtctcctgtcctttggattgcatgcactccgttcttagagaagaaagaaaacctttaacctagtgaatcctgtacctgcctttaccgtcaaattaatggaacgttctgatttttctcgcacccatcattctctaagattccaatcttccgaagcttaaattttctttctacttcctgatgttatccgagcaggtagtctgtgactataacggaacggctttctcatgactgtgataatccttaatgttgtaatcaagcgtctctgttaaactcacccgtgtgcttccacaggcttctgtcccttcacatggctgttgagtgtgcccctcgcaagtttgccatctccccacctttgtgcttggacctcggttaagctgtgtggctttggcttcatctgaccccagctttccagcgctgctccgcgcgtttccatggcaggccatcggaaaggctcctagcagccgcatgcggaggccaagcaagtgctccatgtgtgcatggaagccaggcaagacgccccgtggtaaccagacagccatgtgaggagggagggcctgttccttcctgtaagctgtgtcttgaggcagcatggtcaggtcctgccagggagcggcgtgggcccagcctgggcatgtttctatgccaaggcgccggtgcctgccgccatcttgtctccctctacccccccacccccaatcagaaaatccttccagaatggaaaaccccatttttcctcttttggagaggaaacagcagcagttgatgctggaaattctccagccatccctgagcagttgggctttcatcatgctgcctcttcgcatctgcatgaaggacagatttagccaattaacctaaggttaccttcctctgattaattccccattctgtctttccatgtgttgtctctcgtttttctccctcctccttccctcttcttgcttctcctcccccctccaaaccttacagatagctcgtgaggctgaggcagccatgttccaccgcaagctgtttgaagaacttgtgcgaggctcaagccactccacagacctcatggaagccatggccatgggcagcgtggaggcttcttataagtgtttagcagcagctttgatagttctgacggagtctggcaggtagggccccaagggcaggtaactccgtaggataaccagcctcttgctccagctgatctagaagacggctggggcgcagccctgcagctggggcccggctccagtcctctcctcgtctgcaggaagccagccagggagtcagggcagggcaggaccgcaagggcctccggctcggtaggcacagtggctgccacaggcacctggtgatgggccagttcccatgaaccttggtcttgcgcgggtctgcggctccagttgtccgccgcctcttggcctcctgtccggggacacgtccctcaccagctgtctgtgagcctcttcccctccgtctcccgtgcgacaccgctctgacagtcctgtccccctcctgtccctctggacggatgttgctcccctagattgcccgtgaggcagaggccgccatctaccacttgcaattatttgaggaactccgccgcctggcgcccattaccagcgaccccacagaagccgccgccgtgggcgccgtggaggcctccttcaagtgttgcagcggggccataatcgtgctcaccaagtctggcaggtaggaggtggcagcggctcccagggcacgccctgctcggggcaccttccctgggcgtcccgagagccgtgcgttgatcggggctcagatggccctgagcccaggtaagcccctctgcctgccacctgggcctgaggacccgaggttagactagagacttcgttgccgaacgacagactgcacatgctcacttgagtatgtgaaagttcatcttttaagaaagtaattgatgaccagttttgttcattccagccactacatgccctttaaaagttcagtttgggagattagtgctgttcactgacgtgctgttgggttggtgcacctgtctctgtctgtctttgagccccaaaggcaagcagattcaggcctaggaaaggtcaccacgtcatcttgctggcaccctagtgtcgggccgccgagctgaggctgctgattggccttggcttgggatctcctcctcatctggacatggagtcagcctctgtcattgccccagtctgggagtgtgctcctcatccctgcctgcagggccacaggaattcctggaaggggaagggatggggtcaggctggtggtccccgagcatggaggtgcggcaggtggggttgcgtgtagaaccgggacatgagatctgtgcagattacctcagcagagggctccggataaacagctccaaccatcagggttgacctttgtggaaaggtccagcctttgctctttggctatttttagctccagtcttctgggatttcactctaaccagtcccgacttcagcacttgcttccctctgaagccttgtatgggggccaggcctttagcagggcagcctctgctgtgacatggcttaagtttccctgacacctgtcggtgtccttagggcttgccttccgacgcccagtgcctcctgcaagcaagcccctccccagctcctgagacccgtctacagccacaagtgtacaatgttcaaggaagcatgtggcactggggcccaggggggccctctaccctccaggggactctgcacagacagagcctcccacattaaccttcccatctcctggtctagcctcctgggtaatacccaggtttggctctagagctgggttgctaggactgttttagggacttgggggccatgtaggctgggggagagggcacagcagtagagggctggctttgtaggttccctggggggctggtgagctctgctgctgttctttgcagatgccaagagtcaggtgctattcacatgctgcttggcttgtagctgctgctggcagcaggaggaatgggtgctggtgaggttgggcagggctgtatctgtgccaggatgggaaagttcagaggaggggcttctgccaactgagtcgtgtggggtgagtgggtgagactgaccgggccgggtcggtgtgatcctggggccctgggctgtggggaggggggtaagaagccaagtggacagatagcataggaagaagagtcggattctgttcctcggaaggtggaggacgaggggcagccctggcatggggagcagatgctaaagtggccagagtctgatgctggcagaatgagttggattagaggctgagttgttgctgaaggccctgtggggggctctgggaggctctggaacactgtttaatggtctcttctgcttggccgtcatcctgtcgcaggtccgctcaccaggtggctagataccgcccccgcgcccccatcatcgctgtgacccggaaccaccagactgctcgccaggcccacctgtaccgcggcatcttccctgtggtgtgtaaggacccagtgcaggaggcctgggctgaggacgtggacctccgggtgaacttggccatgaatgttggtgcgtggctgggagcaaagggctagcacctgcctggaggggtggggacggtgcttgggcactgatctccctgggatcctagagaaaggggaacatgctgaccttggagtgtgcaagagtcctagctgtcttcccagggaacgttctctgactcctccttcccattccctgaagttctgggctaagcccaggagggaggaggagccttggagttctggggctgagctgcccacttctctgtcttcccatccggggcccaaagcaaaggatcctggatagtcaggaatgtcccttcttgggcatctgctccttgttgagggttaagaaaagattagagcgttacatcatggggtgtcctacacacagatcacgtactcatggcctttgtccctaataaaagtaggatgtccgtgcatgagagggactactccaccctctagagtataaaatgatgtccaaaggtccccggcagctgccatttcaattgatgaggaagagtcggggcctccctggtccctgggctgggcggcttgcgccaccttgtggtgggaggcagtaagggtcacccagtctggctacctcggcgggcgggtcagacaggtgctctcctgcctggcttgctttttctcaccgacctttcttctcttctttcaggcaaggcccgaggcttcttcaagaagggagatgtggtcattgtgctgaccgggtggcgccctggctccggcttcaccaataccatgcgtgtggtgcctgtgccgtgacgggccccagagtccctcctccagcccctgtcccacccccttccccaacccatccattaggccagcaatgcttgtagtgctcactcgggggttgtcatgtggcactggtgggctgggatgccagggaagatgaacgcctctgtaaaacatgacttttttaggaccctgttcagtgcaggtggcccagagctgggctgcacatcaggtgacctcaccgcagcaacggacgagggaagggtgcaggactggaagccccagagctttaacacggagggcaacagctcctgcttctcctcctctgtgtactccattcggttcctatagaaaatggatacgcagagaactcccaaccctggcctggctgggtctagagaccaccgcaagaatcgcagcccagggcagtggttccagttacactggctcaggcccttactcgcttctccacccccctgtctcccccactcccacctgcccttctgttcctaggctcagctgcaggcaaacactccaccctccatttcccccactcctgcagctgcctccaggcctgttgctatagagcctacctgtgtgtcaataaacaacaattgaagcaccca

SEQ ID NO: 49 - DNA, 23008 bp, Mus musculus
  source 1..23008: mol_type = genomic DNA
cgatgtccttccgccacggaaggtagtcctcctcaaaagggcaacctgcttgtcccgcctaccctgagcttctctcaggaggtgcgggcgccccgttgagaggcggggcgccgccggccgcagcccggattgggcgaggggcggggctgcggagggattgcggcggcccgcagctgtgataaccttgaggcccagtcggcgcagccccgcacagcagcgactcgtcttcacttgactgacgtccgctctaggtatcgcagcaggaaccgaagtacgcccgaggtgagcggggagaacctaagccatctgtccccagagccgatgcccactggtgtgtaacgcaggcctgcctgcccacgcgggactcaggcgccaggcatcgagcatcccactccgggttggggaggagccacacttgagtccaagtcttaatcctccaaattgagggtggccggctagctcctttcccacggtctcattctgctctctcttccatctcaatctggttccagcctcttatcttactattgctatcctcgccaccctgttctcgccctgctccgatgctcgtcctcctacatccactacgacttacgaccttcctcttagctctgtgccctcataattcggtgccctcctcttaatctctacttgaaataattcccctactcaccccacccccaccccccattcagcctccgcgttcatcctcctttcaggtcagcgttctcttcccgcgccagtgttcgcagctcttcgtcgcccctcccccctccgatccggtcacgtccgcgccgcagcatcctttcaacctccatatcctcgcccctccccctcgcagcttttcccctccccccgcattctagtcacgtccgcggactcctcgtggtccggtgcatggaggtaggatgcagtcactagcggtccttggtaatgcagctacaagttacgtaacttttgtattcccggtggcctggcaagaaaggttggccgctattgctgcctgttttgtgtgcccgcagatttttggtggtgggcacgggccctcattgctctcgcgctccatcctgcggcggaggggcggcacgtactgcgatgcgtctgattaatcggtcatgctgctcccacgtgctggcgagggggagggtgggcgatcaacccatacccgttcccgacagaaactggagcccgacgctaccagaacacacatagcccacagagctcggtctaattagggaagcagattttgtcaagtcagtgctatgaggaagatggggctgcatgcacaaggtggccggcatactggttttgaaaaagccgggcatcttctagtctgaaggctataaagacccagtaaatacccagtcatagtaagataagagctcgaccactttttccaaatccaggacactggtagtttggaaaaagtgaaccttatagtgaaaatccagtcgtgcatggaggcagctaggttttggaatccggtgtgatctattaatattcttaggtttggcctctggtcctgtgtcactccaggctgctaaattactggtctccttgtaagtacgggccatcctcgatgtagtgactaacctcgggtgaaatatccagaagggcatgcattatatccttttggtcaatgaatgaagtagttaccttgaaggctaccaacttgattctgtctccttactatttttatgcacattaggggtaagcttggggaggcaggtaggctctgaatacccagtatgcccatggaaacaaattctctctggaatcccgtcagtgtttacaaagtaattaatgttacattgcagctctcctgagaaagagctatctaaagggagaaatctgtgcagcctttgtctttggctgtgagggtagaaacaaagaaacaggtacccagatagaagccttccggtcctctttgttcaagtaagccttttatgccttctggcttgtaggaagtttggtactgttgccccaacagtggggtgggggggctcagatcaagactcaaggaagcattgttagcaaataatttgccctctgatggcctgattatgctgtaggaaaggcagtgagggtgttctttgggaaagtagtgtaaaccatcaccgggtctgggcaagatgactcaacctttcaactttgcggcttttgagaaaattttcttccttgggtgagtgaggggaccacacttgttctctgaagtgttgtaaggttccctcttttttagtgagtcaaatagcgtgcattttccctgaattaaacatgtctcactcagactgagtgtggtggttcagctttaatcccagtacttgggaggaaggcctgtgagcttgagaacatggagcgggtgataccctgtttccagaggtggcgggtatcacctaaatgaaggtgaccataagagtggaaatgtggagtggctgcttctagacggcatattttcattcttgaatgattggcacagcagcttttcatagaacaaccagtatccagattggcagtttctctcgaggcatttctgaattattgcttaagccaaactatatgaatactaatcacttatttagtacatagctattttcattttaagcattatattttaaattatttaatttgtagggaaatttgttaagaactgtttgccagcactcaggaggcagaggcatgcagatctgagtttgaggccagcctactctacaaagtaccaagacatccagagctacacagaaaccctattttgggagtgggctttggtggaaatgtatctcaaccaggtataatggcccttgatccctttattcccggtgttctggaggcaaactggcaaatgactttgagtttagtccagccagtgagtgacttggcacatttcatagctgactccaaacagatttatgggggctgggtatgccatctgtgttatatgaacgtgttggtcacaggaaacgctattttatttcttaaagctgttaattgcttaatattgagtggaagggatgaggaagcggctatggacagaaaggtagaaaaagcgtttcttgccttaaactcacattgggtttttagcatctttagcttttttgcagcaagtcgacacgagttgggcaagaactaagctggagattgaggtagaggaaaatgactcatttgtctttcagtttctcaatcatctcaaatacccacaggcaagttaactggtgagtttttttttttttttttttttttactgcttctggtaaaggaccgagtcatctctgtaaactgcactcatggagactataataggagtagatgactgtatgacgaagaacattcttcctgccctggaactttcgtgcgtcttgaaatttttaaccaagagccagacttactttggctatgtagtaatcaggagaggtgagccccacgtgcatcgtggcctgcaaaactgctggatcttaaggcctagtgtagctactcaggtttggaggtggtcccagggcaccaccagctgtgctgtgaggccacacccctactgtggtttgtgcctgtctgcacgtaggagagaggtctgggctgcagtaccttaagtggcctctagggagctctgagcatctcctgaggcctgcctccaaaggaagctgtattccttctaaatgtgtgaataacctttaggccatattttggccttaaagtactggtgtctccagtgtaaaagactttaccctttgccatgcccactttggaggagtcaaaggtttgatttgatgtagagaagggctgggtgtggtggaatgtatttttaatctcagagagacaggacagtcaggatttgtagagtccttgtctcaaaaaaacaaagtagggaaggggctggcaatgtagatctgatacaaagccatggtccatccctactactacataaaatgcacatagtagcacgtgtctgtaatcatactgagcaaaggacagtgggattggagaagttgtgagttggaaaaaacaaaatggactatgaactgaataaagtcagaatgctatttaattaagttttggaaactataaagataaaaagtcagtgttgatgctaagttctgggtgggagaaatggagtctttagatcccactccttggggaaaaggcggtcgtgcaagcctgcgggcagaagtagtaagtaggtcagtgctcttggacagctggtgctgccttccttccgactttaatcaccctgcccccaactgtggctccgggctgagccggtgcctcctgttgctgagaccattgtgcattctgcttacacctggttctctgggcttctgagccagggtccagagcatgggcagcattcccggaaacactggccttgctaaggaagaccgcatgtgccaaccggacagaaacttctgcaaagtcacctccttcctgctctggctagttctccgctctgcacagctgagctagaagccaggaaatcccttgctttgaggaaagtaagaccttgtcttaaaaatcaaataagctgggccgtgaggcaggcagatctctcagtttgaggccagcctggtctattgagttccaggacagccagggctacacagagaaaccctgtctggaatctgactctcaacccccaaaatctctgctcaagtgatctgtacccttgataccgcacatttaattttgttgtatgctgggtactgtgctccaggccttaccactttctggggatagaactgtcacctgctttgaccctggtgttggcttctcattagggtcactgatagctggacagcagcctttctgatggatgaagtgagatgatcttgggtactggggaaagtaggcttggggtgacatcagagaaaggttgctacaaagccctgttcctaggccaggctgctttattagggcagcaatgatttggacctacgtgacgatgtcattggtggggtgaggcatgaccttccaagagtgttggctctaagaagcagggtggcaccaggcatttttggtttggggattttttttttttaatgacctttgttcctgacacgagggagatgaatgtgaagggccgaggagaactgcctgagacaggaaagggcagaaccaggtaatcagagccaagctgggcctgtgcatgctccaacctacagcccaactgaggcctgggctggtgtctcctttctcacagaggggcaatggctctgaagaacatcctgcttcaggggctcaggttcaagagtaaggcttctgccccagggcatggtatctactgctgtcgtgggacacaccgggtttctgctgccggctggaggttggctgtgcatgtgggtgcctcctgaggaggaggatgctgtagtgagttggctggcatgggtggggccgcatgtcctggcagtcactgctatctatacaagtatttggccaataaggcaagtcctgcccaggggcccaccttaccctaaggaacaaggctggagctgctgtcctgggccagaaaagacttctaaaagggtgttctatttatactatactatagataggtggttggctggagttgtggggctctggaggccatgcaatgggcagaagagctgtgggagggagggaggggctccaggtcagagccttgggagtgaaaatagctgggttctaaggatgaggttggtaggggacttgaaacccctgtaaggtttgttagaagtctgtttaattgtgtccatccattggaacacatgtattttcatttgtcactttgtatatggtagcaaggtaagtctactctgcagactttaggtttaattctagatagagaaatgggttgtgtgtgaaattttacctgctaaattgcatcctgagccctggagggcagcagtgcttgtctgttggaacatccttgttgcacaacaaagttctgtcaagcctgagaccggtcaaaacaggagaagaaaattgcatggaactgataggttgtggtcatgaaatttttccatggattagccacacacttttgggaaggtgacttttaaagtcagtggattaagttgcccttcctgggtcagtgaagctgccctgctgagcacttttgagggcatagagaacacacatctatcactatattttagcagggtaattctgaaggacacggctgatgtcacagtgagctttatagggtgcatagagataaatgtgtcattttcaaagactacttaattaacagttttattaataaaggacacgctgggcagacagaagtgtagtaagatgctgtctcagaagagtagggcagtaacgtgatacaggctatcatccaaggtggagcagcgagggtcagtgagatagcttggaaacacacacaggagcttgcttctgatggctccagtgcttctgtccccagaacctgagtggtaggaggggagagcacagttgtggtcctctgatccctgcaaggtgcccaccaggtaccccctaaaactaaatagtgaaatttgtgtattgggggaataaagtacttcaagggatccagtaaagggtcacttttaccactgggtttttttatttatatcttctccactgtatatctgttttaatgtagttaacagcaactttttgccttgttattaaggtcgttaccacaaagagcttagtcatattcagctttcaccttaatccttgtagctcttcagggcaaagcatactccactctgagcgatctttccaagtgaggccaccaagccacactgagtcaacaaaacagcaatatgccatcagaagcccagacccagagaaccaaaggtatgacctgatcactgggcctcactcaggcagggatttgcgagggcacactcgtgtctcagatgggaagaacggcctggtggctggctttttcagtagttttcagacactgtcctggtgtctgtcccgctcacttgtcccatctcggacacagcaaggcctttgtgtctcattggcttggtgtaccatctgtactgtcccttagcggccacttatcctaagcctggctgggagccgtgagtttgccagttgtgtttgtttcggccatgagtagaccaaggacctggctgcctagggacttctcacatttggggagagggaaaaccaggcagaattgatcttggcagagacccggagctcatttaacaaacaaggtgctgggaggcaaggagctaagactctggagaagttgacatcttggtgtctgatgtgtcttgtagagttaggaatgaacaatgttggccactggtttcttccttcctttaaaggttctcaattctgcaaagaatgtagtggttgcacccctgtaatcccagcacccaagagctggaacatgagactgtcatcagttcaagaccagcctgtgctacatatgataactttgtcttaaagtttttcatatctgcatttgaagacttacaggtgtctggtacttggtcactagtgggtggctgaagtctcaggttgcagagacagacttaggtacatactctccaggcgtccagaaaagccagagttccgggaagagtgggggtcctcagctgaggagacaggggagagcaaggatatggagatactgtggagcacaggctgaagactttatgttcaggagtttgagaggagccttgtggcctgaaaactcagatgagaaagaatgtcagcttcattcaagtctcagatagtctgcgagctatagcccagagctctcccgagagttctgtcctctaatcagctgactatccagctcagtgagcacttagttgtaatgaagtaaaagcaaaggtcaggacagctgttggaattgtgacctgggtggttaattacctggaacagatgttcagactactgggtgctatacctatcagacagagaaagaggctccaaatagccagacctcgggctggggaagctaggtctaatgtcagcccttggaaagattaaagatggtaacttcaaggtcagcctaggttgcacagtggatttcaggccagtgtagagtagctgctgtgattccatctcctaggttacagaagactccttggtgaggttataatgactttggggccatttcaatgcccttgtgcaaaatttgctttgtatgtcttgtaggtggtactgtgtttttgtttttgcttttgtttattttttgctaaagaattatggacttttgcttcctgcccagaagaactgttttgttccctgtaatttggaccttactcttgtgtctgataaaagctaaagaatacatttagatagacctgggactttgggagtaaaagaaaggctttgggacagggtttctcttgtttgcaggaccactttaggtttgggtgaatgagagagtgagaccatgctgagtgcctaccaaagccaatactggagattggacatcatgataggcttcccaagatgtttgtgtgctgacatactagttagaacatgcgctggggggtgtttcttactgtaactttggaggtttccttgtgtggggaaagtgatgttctttctagggttgatgagaaagctcttaagacaaggtgacagcccatgtgagaagcctgcttaaactggctcccagggtgtgtgcagtgggtagggagttactcttgttcaccaaggtctttgctgtttgccaatagtcacaagagccctttgcccaatttgagtagcacccacataaccagcacgtgccaggcttttatgctgcttgtgcacaaagcaatcttattgggttaatgtgtaagcaggagacagcttcctatgccaagtgcctcattaagcagtgcctaatctaattgcactcttcaaatctccgggacccaggacttcaggaaccatgccgaagccacacagtgaagcagggactgccttcattcagacccagcagctccatgcagccatggctgacaccttcctggaacacatgtgccgcctggacattgactctgcccccatcacggcccgcaacactggcatcatttgtaccattggtgagtgtggccctccttcctctaaatggaggcttctacctgatttgaaaggcatagtaaccattgcagagctagcctaggttctgagtgaggcacggtccacatttctaggggagtagaggtcttgggaattggccatcaagaagaattagtgtgctttttctgtagttggtgaggttcaggggtggtctttttcatgtgctgtcaccaacagcattcggtagacagacatcttgaaggcagagaaaactgttgccctccacttttctggctgaatgaggccctgcagaatttgtagagaatgttagtgttcccacataattaagaaagtgactagtacaatagtcttttctgtactttggaaagacctttctcttaatttgtgaaggtaattaaaagcaaaggatgaagagttgtttaaatgttgagcaaatttcagacattttcctacctgagtcatgattttcttcctgtggatctaaatgtttcttgatagggcctgcttcccgatctgtggagatgctgaaggagatgattaagtctggaatgaatgtggctcggctgaatttctctcatggaacccatgaggtgagcgtcaacgagatccaggagactcagcgattccttaacagtcgtactgcaggcaggtgtgagtccaggggtcccagtgaacggaacattgccgtttctctcttctaacttcactggaatagaaacctggcctgctttgtcacccaccgaccagggttagccctaccgtcaacctttatgaaagaaggcacgtaagggtttagctggaaaccctaggccatcagatgtcttggcccccatgcttcagggtttttacagtggttcctgtgtgtgaaccaaaaggttctgagcagatggatagctggagtcattttaagatctaccttttaaatacttctctctccccctccctccctctcgacagggtttctctgtatagccctggctgtcctggaactcactttgtagaccaggctggcctcgaattcagaaatctacctgcctctgcctcccgagtgctgggattaaaggcttgtgccaccaccgcccagctttttaaatactttctaacttgactgtggatttcctactggtattggtgaaggagggaaggagactcctctctgcctcttggtttctgtgtcctatttagagtaaaagcattaaccctgtgctgttttgccctctgacctttggaagttgtttggactaaaaatagatggagaagaatggtccaagaagtgaaccccagaacatgagaatcttcatagattccctaacccatattccataaatagcttggaggctagtgcccaaatgtcatggaacctgagatagttcctcaggcacctaagcaatatttgacacattcttgctgggtgtggtggtacaagattgtggttttgaggccagaatggggactatcctcagtaacatagtaagatcttattgcaaaccaggagaggtccttattttccaaactctggctttccacaggagctcccaggaagaaggtgagcctagttcagagacagaactagggccacttgccatggtccttgcgagtagtgtgtttagtcaggcaaaaatagatttggaggtgctgacctttagggctcttgagtccagtaaaataacaccatgcagcaggtctaagagggcaggggacagtgagactgtccagaccactgggcaggccagctctgccttgatttcagactaaggcattagagattggctgtactttgaacctttttatatcacaatataaagcttcacaagtcagggctcttattccatgtgcaccttcagtgaggctctgggtgatggtggtgctggtcttggtgattccctggagaccgtggaaaccaagctccttccctctgacaggaacatcagccacctagctgcacctgatcttgacagctttggctgtgtctctaattcccatctcttgcttttcacatattcaagatgtgtcattcttgctgaacaggcagtactgtactcccacactggcttttaaacagcctaaatttagagcctctacaaggatagcactgatggctgccagtcttccccatttggttacatgccagaaaaaccacagctgtgataatggatagcacgacccagctcccagcagtgttaccatgcagagaaggctaattcaccaccagatactccaatcacaatgcagctttatatatacgaagctgaagagtgtttattatgctcgtgaatgtgctgaggtgtgtaactcagtgtgtacagccataattcttcagtggaaattaagggagaaatccaaacttctagaggatctctaaagcaaatgaaggcagtcggtagtcaatattttgggatatctggagctgggttaccgggtggtggtcagcctttgggcagcttcggtccttgtggatgaatggtggttccagctctgccctaacaaacaggcttgcaggtgttttgctgtgctcagtggttcaaggaccagactcataaagtgctgaattgaatggtccattgtcaccagtgtcacaaggatatgcactggcaacaaactattttgctatcttggctctgagtcccagataggaaagggaaaaggtttggggaaactttattacaagtgaagaaagcaatggcggttgcaccggggcaggctcctggtcggaggaagtggttatgaaagcagggtctgcgtgactagagctgtttagctcggccattgctcactaagtcaacagctttgagtttgaattgcagttgggatcgatagtgaaaacagatagaggctgccagggcagaaatcttcaaacacaaatcctgggtctttgcttgtcctgattagcatcccttggtgagtcctagggacactgggacaacagaagggtcccacaggatgggtttatagtcttccctttaattgattttggtggcagtattctggaactgtatgagagttggagttgatgctgttgtgtagagggaagaatggatattgctaaggttaggagatgggtgaaggtcaggagtcagacatacttttttttttttatttgctaattgatcatctttagctccagggtggggattggaaactggacagggacacctcacctgccaatctgcctttctttctccagtaccatgcagagaccatcaagaatgtccgtgaagccacagaaagctttgcatctgatcccattctctaccgtcctgttgcggtggctctggatacaaagggacctgagatccggactggactcatcaagggcgtgagtatctagaatagcctggtagggggtcacacttttgctatgtaaataacctatttagtctcactctgggaaacgggtattttgtttgttttatttcttcctcaatatacaaattcaggctttatagaaaggtgagaggtttctttggactttgagccagagttgagcgcccccatcaggggcattggcttcttcagttcacactcccatttcctgctttaatccatagagcggcaccgctgaggtggagctgaagaagggagccactctgaagatcaccctggacaacgcttacatggagaagtgtgacgagaacatcctgtggctggactacaagaacatctgcaaggtggtggaggtgggcagcaagatctacgtggacgatgggctcatctcactgcaggtgaaggagaaaggtatgtctggtacacagtccgtggccaatgccaactccaatccccagagctctggcaagcacagacctcgaatgtatgaagatctgggtttaatctccagaggatcaaaagtctaaggttattgttggtctgcgtccctgacctgtctgaaatactgtctcagaaaaaaggcagatggggctggagagatggctcagtactgactgatcttccgaagatcctgagttcaaatatcccagtaaccacatggttgctcacaaccatctataatggttgtgatgccctcttctggtgtctaaagagagctacagcgtgtataaaaggtctttgggccggagcaagtgggggatcctaaattcaattcccagtagccacatgatggctcacaaaccatctctacagatacagtgtacagataaaacacattaagtaaataaataaataaataaataaatataaaaggtctttgggccggagcaagtgggggatcctaaattcaattcccagtagccacatgatggctcacaaaccatctctacagatacagtgtacagataaaatacattaagtaagtaaataaataaataaataaataaataaataaatttttttaaaaaagaaaagggcaaataacccacaaaggtccaggtacctttagtcctccgtcctagcgttcgggaatcaggaaggtggagatgtctcggtgcagcatatgttagactactattatatgccttagaatgagagttaaagttacttattctaaatactttgtgacagtttgagagggtttcctatagctagccttgaactcttgattcttctgtttccacctcccaaatgctcacattaagaatatacaccaccagctgggcatggtggcgcacgcctttagtcccagtactcgggaggcagagacaggcagatttctgagttcgaggccagcctggtctacaaagtgagttacaggacagccagggctatacagagaaaccctgtctcgaaaaccaaaaaagaatgtacaccacctcgtgtggctatttatttgtttattcattaatttgaggcaaggtttccctttgtagccctgcctgacttggaattaactgtgtgtgtagaccaggctggtcttgaactcaaaggtctgtttgcatttgcctcttgtgctaccatacctaaaactcaaattttcttagcagtttgtaagtaagtatttataggtgagaaaactgacttggctttcctgaagtgttttgtttggtttggtttttgttttgtgtgtgttggagtcttaattatggctttagagtcctcctccctctgcttcttgtaaattgaggtggtcttctgtgatccctttcacacaggcgctgacttcctggtgacggaggtggagaatggtggctccttgggcagcaagaagggcgtgaacctgccgggcgctgctgtggatctccccgctgtgtcggaaaaggacatccaggacctgaagtttggggtggagcaggatgtggacatggtgtttgcatctttcatccgcaaggcagccgacgtgcatgaagtcaggaaggtgctgggagagaagggcaagaacatcaagatcatcagcaaaatcgagaaccatgaaggcgtccgcaggtgagtcctgagacccttccattgcccagcccttgagaggggtgtggccatggtgtgtcctggatacctgctcagcaaaatacagcctgctgggattggtccaggcggacatctgaatcagcattagggaggccaagtatttttagtcatcattttgggacccggctggatactcaagggcctcagatgtccatgctaaagcttgaagccttagaaatcttctggtctgataatggtgctgatgaggagtggcccattcagcttcccatagagaagcatgatgcctacgtaaatggaaattaattaaggtggcattcataaggatgagtggtttattgacaaatgttcatacttggctttctccctactgcttcccctagaactgcttctgtgggttacagtgggcttggctctgtgtcctttgtactgggcaactgggagtctctttctatcttgataagccataggtgctgatggccttggtatttggggctggggagtgggtcagctcaaagggcagcagtcagtgcccttagctaaatgatgatccactttgtagaagatccactggcctcattctgtctttgaagtgtcgattagggaagtacaaaacggggtggggggtgagatgcagaccaaaacctccctgaaatatttattatggtgtttaagaagtccagccagtaaaattgttgtgctgtgagtattatcatactgtgctgtggatgccccctcgcctgtgtgcccctgggtgtgacctttgaaagcatctctgtcgggatcccaggtactttggttgtgcttcctgtcctctattatctttctcttctttaccaggtttgatgagatcttggaggccagtgatgggatcatggtggctcgtggtgacctgggcattgagattcctgcagagaaggtcttcctggctcagaagatgatgatcgggcgatgcaaccgagctgggaagcctgtcatctgtgccacacaggcatgtgctatttcattccttctgcattctccacctaggagacctggccttgtcctgtcctttgggcacacatagctgtgatctgtgcacctgcacaatcttaagggaattatcttggcaattatcactgaagatggcctaggatctcatttagtgatggtcttttaccgagagcccttgtctgtcccctcctagatgctggagagcatgatcaagaagccacgccccacccgtgctgaaggcagtgatgtggccaatgcagtcctggatggagcagactgcatcatgctgtctggagaaacagccaagggggactaccctctggaggctgttcgcatgcagcacctggtaagtcctccaagcctaccaccaaggcctctgcatcacccagtcttttacctccctccgaccacggccagaagagtgaggtgtgtggagcatgctctgcttcttgattttcacgttgtgctctcgctgcctgcgccccaccacgttgtcctgctctggcgattaccttttccattacgtaggccacatctggctaaaatattaaagtcctaggacttagtcaagggataccttcctccctcctgaacacccagacggcggggcggcctctattctaaaggagccaagagtgtgtattcttggctgttcgcctggtttggcttctaatttgatctcttgatggtcccatgagcagatgcttctctgcactgcaggctgtagccatactaagctgctttgagctggccttgcatggtgcctgtcacatgggacgtctcttgctatgccaaacccaatgtagggctagaaatagctctgggcgtggggaatgggtgctgaatttagcaggttctggactggagattataaagactttctctgggcaaatctatgctctttttgactaagtcttctggtttcagtaagatagggtctggaggtccagcattcagagcctgaggcaggaagatagcttgaggctaggctgggctagaataaggcattatcttacagaaacaacagacttccagctgacctgactcctgctgactgtgatgggtgaggacccagaacttcctgagcagagcagttagctagggcgccagttaggacctttccttgcctcatgaaagcattgttggctaactttcttggagcttttctattcccttttcggccagcaaagaaccactgttctttttgtgtctccagtttccaataagcccccaaactgaaagaaaaaaaagcccccttcaggattagacatcttaccttgcttcatttgtgtacagctgttaagtagattccatgatctacccatggtttatctgaattgtagctgtagccagatgtgtgcctatattgaaacagaccagccgttttgtagaagcttgaactcagcttgtctcagctggtcacctcctgatctgattggtattgggagctgatcttcacagcttctcagtagctagcatgcagacattccttctccagcagtctgtgtgccttcctgatctacaccaaacccccttcttttctagtcacctgcttagttgtcttatcacctcagagtggtcaggaacaagaccaggtagtctaaaccatgcagtcacatacatggatttatctttgtatagctctgggtgacttatatgaccgcaagaccttgcccaaggtggcatttgatgagattaattataattaattagtcataacattaaacaatttactgccataatgaaatgttagataaccctctgggctcattgatgtaatctttgcattctcattttctttttaaagggaatcacatgtgatagtgtgtttgagcacagatttgctatcctcatagggccagccagctgtctgtttgcaccctgctgtagcaggtgtgggcagagtaggagttagttctcatgtcctgccctctctcatgccctgccctttcctatgaacagacaccttagaacctcgaggctgggattgcatggccctgctcagaagatgagtcacagagtccgggttagactgtggctgccccttcagggggtgcaagcttctctctcatcagttaacactcaggatagcttctccccttcatctgttcgctgcctcctcctctgtctaactgatatagttcatgacctgtaattaagagctagacatcccagctatggtcgtttcctgttcatgtcctttgggctgcatgcattccatttatttgtaactaaaagaatactttccacttgcaaatctgctaatactaccaataaatgtgagttattggttttacatcttctctatactaaaatacttgggattgcactctctaaaacttagattttcattctaatgcctggttttacttgaacagatagtctatatataacacatttgctgttttgtaacagttttaattgctaagttttaattggtgtcttaaggcatgcatgctttctcaggcatctgcctcttcacacggctgtccactgtgttcaagtgagccagagttggccactgttctgtttagaactggcgcaccatgtaactttggctcttttgacctttgaccccagctttcagagctgcccagatgtttctattataaaccaggtgcaaggactcgctcttgtatgtaggctaagctagatgtcttgtaaccacacagccgtgtgtggaggggaggcctagttcttcctgtaagctgtgtcatgaggcagtgtggtcaagtggaagtgtggttggctccaccttggcatctttccatgccaaggtcctagggcctaacaatatgtccctgtcttagcttcaatcaaaaacaaaagaaattgatggtgcctgcctgttatcctagcacttgggaggctgaggcaaagaaaatagtgatttttgaggataactgtggcaagttcaaggctgataggggctatggtaagatcccatctcaaacacatgggggtgagtcccatctcataaacacatggggatggggttttttttaagaaacaaaggggaaagtcccaaaaggataatatctttctagaacggaaggaactttccttgtatttgaacagtaaggggaaaaggagcagcccaaaatcccacgcaaccattccaggagcatatgggctttgaccaccctgcctctgcatctgcctctgcatgaagaaaagattaaacctaaacctaagggtgccttccttcctctctgatgtagttccctgtctttccatgtgttgtctctcttgtttttgcctttatccctcttccttatccctcctaccctaaaccttacagatagctcgggaggctgaggcagccatgttccaccgtctgctgtttgaagagcttgtgcgagcctccagtcactccacagacctcatggaggccatggccatgggcagcgtggaggcctcttataagtgtttagcagcagctttgatagttctcacggagtctggcaggtagggccctaagggcaggtatcattataggataaccagcttctcgcgcaactaggtccgctatgtgcctgagcctaggcacagcctctctccttcaggaagacagccaaggtcaccatagggcaggaccaaaggattcccttgggcacagtggaagtcacagcacctggtgcaggatggttcctgtggagtttctaatcttgctcagttcagaacatggagtggctcaccttctcctggccatttttgtgcccagggacatgttccttcccagttgtctgtgactcctttcctccctctccatttgtgacaaagctctgacaaagccctgtcccccgtcctcgtccctctggacggatgttgctcccctagattgcccgagaggcagaggctgccatctaccacttgcagctattcgaggaactccgccgcctggcgcccattaccagcgaccccacagaagctgccgccgtgggtgccgtggaggcctccttcaagtgctgcagtggggccattatcgtgctcaccaagtctggcaggtaggaggcggcagtggctccctggggatgcccacgctcagttgacacctctccttgaggatgccaagagtgagtggctctgggccagtttaaggcccctggctgccactagaggattccaggcagcactcacagatagaccaaaccaactggctggctccagtgcacgttcaaagcctgcctcacagagagctggaacaaacacccagtttcaccgtgattagtactgtggggccttttgacagaacactgcgtggcagctgggcatggcggagcatgcctctaatctcagcacttgggccaaagaggtaggcgaatgtctggatttgtagcctgtctagtctacaaagtgagttccatccaggacagagccctactcacaaaatagctcagctggtaaagttgcttgctacacaagcttgacccatatttggtccccagaaaccatggaggacggagaaggctggccctgggtatgcgcaagggtacacacttggaggggtcttggtggataaaggatttgcacaatcatgactatctgaatttgaatctggcaccttaaaggttttttattattactttttatttttttaaagtgcacacagaggtcaccaggaaaggtggtctggcctttaggagcactgtcagttctttcagaggcccaacacctgcctatatatggcagctcacaactgtctgactccagtcctggggaaatctaatgcatgtggtcagaatacccaggcagctgtagtgaatgtacggtggagggagagagtgaggtgcccagtgtctatctatctatatagcacaatagatagataaaaaatacccaagtgtggtggtgtgtgcctgtagccccagtgctcatggtgcagaggaagaaagagcaagttgtatcacagcctagctacagaaagccaaacacatgtaaaatcagtgtggaggactaggcactggtctgtctccctaaggcagtgttcatgaactaagtagcagaaagctacttaggcctgggctgaggatggtggcctctgtgtaagcttggccatgaatgttggtatgtagctggaagccagggatgatggggtactgagaaatggggacactaaaactatcatttttagtcctggagttttgaaagctctaaaatacaaggtctatgctaattctggggtttctctgaagagttcctggttccagcagctacctccttccttcaaagcctatgtatgcaggctagcatgaagctctgctgtggaattcctcagtcccccgtgcctagctaattgagtaatctgatagagatagacactatcatttgttacaggttgagaataggggttccctacattcccaggggatcttgaatggccagatattcctcttaccacacctgatcaccacccagatttctttttctttcttttttttttaaatttatttatttatatgagtacacgtacttcagacataccagaagaggacattggtatcggatcccattacagatggttgctgggatcccatgtggttgctgggaattgaactcaggccctctggaagagcaatcagtgctcttaactgctgagctatctctccagcccccagatttctttttctttctttttttttttaaagattgatttattattatatctaagtacactgtagctgtctgcagatgcaccagaagagggtgtcagatttctttatggatagttgtgagccaccatgtggttgccgggacctgaactcaggaccttctgaagagcagtcagtgctcccaaccactgagctatctctccagccctaccacccagatttctaaaaccatagaaattctgaggtttcttttaacattagctgctaggactcccataggagaacagtatagtgttatggtgaacattgttggcttccagggcctggtaactctgctgctgttctttgcagaagaagtcaggagctaggcacatggtacttggattgtaaaagttgctggcagctacaggagtgggttctgctgagattgggccaaagctgcctcactgccagatggaagggttcatttgtgggaagaattctaccagccatgctcctataggactgcccatactgagagcaggataatcatcttagaaaagacaggacaggtctgaggggcaggccagaccttgaaacagttgtcagtgggcaaagcctgtggtccagagttgaattagagggtattacttttggcttaggcttactgaaagggtcttatgacatgtttaacggtcagtctttccaacctgtttccatctcaggagtgctcaccaagtggccaggtaccgccctcgggctcctatcattgccgtgactcgaaatccccagactgctcgccaggcccatctgtaccgtggcatcttccctgtgctgtgtaaggatgccgtgctgaatgcctgggctgaggatgtcgaccttcgtgtaaacttggccatggatgttggtatgtagctggaaacaagggaatgatgaagtgctaataaatggggaccctaaaactaccacttcctgaagtcatgggctgggcctatctgttttatcacccagttgtaagattagctggagctactgtcctgagcagggtggggttagagggtggggaacacaagcttttgtggccttattcctatatagagacaaggaggcagctgaccctgactctctagagtataaactgaatggtgtccaaaggtctctgcattctgagttcagcctcagcccttgtttaggctaggaggcttgcaccatcttgtggtgagaagaagtaactgtcaacctgctcccctacccagacgaggattattagggcagttatctgcacctgccttgttggatttgtgtctgggttgggaaaagtgccactttgtgtgatcaattaggactgtggggctttgcagttttcctcaagtgtatacctctactcaccaacctccttttccccccaggcaaggcccgaggcttcttcaagaagggagatgtggtcattgtgctgaccgggtggcgccctggctctggattcaccaacaccatgcgtgtagtgcctgtaccttgatggccctctggagcccctcttctagcccctgtcccttcccctcccctatcctttccattaggccagcaacgcttgtagtgctcactctgggccatagtgtggcgctggtgggctgggacaccagggaaaattaatgcctctaaaacatgcaatagagaccagctattattcagggccctacctgagccaggggtggaggaggaatgcaggactggaaaccctgactttatcacagaagggcggcagcatctctgggctttgcttctgtagaaagttgtcagaattcccagccctagcctggagtcaggagacagcaaaagagtaggggctgagggtgtggggcccagggtcccagtgtagatgacgacttctggccctggccctgacctgctttcccaacagctttggcctccccacttcttgtgcactccacttctgtcactgcagacactccactctccaccttgtattctgcagagtctccaggcctgttgctatagtgcccacctgaatgtcaataaacagcagctgaagcacctgt

SEQ ID NO: 50 - DNA, 21995 bp, Rattus norvegicus
  source 1..21995: mol_type = genomic DNA
aatgtccttccgccacggaaggtagtccccctcaaaagggcaacctgcttgtcccgcctaccctgcgactctctcagaaggtgcgggtgcctgttgagaggcggggctctgctagctgctgcccggattgggcgaggggcggggctgcggagggattgcggcggcccgcagcagtgataaccttgaggcccagtctgcgcagccccgcacagcagcgacccgtcctaagtcgacagacgtcctctttaggtattgcaacaggatctgaagtacgcccgaggtgagcggggagaacctttgccatctgtgcccagagccaatgcccactagtgcgcaacgcaggccttcttgcttgcccacgctggacccaggcgcctggcatcgagcatcccattccgggttgggggagaagctacacttgagtctaagtactaatcttccaaattgaggcgcggggtggggggcggctagctcctttcccacgtactcgttctgctctcctttctcaatctggttccaacttcttattttattattgctatcctcgccaccttgttccaatgctcatcctcctacattcactagtactttcgaccttcatcttagctctgtgcccttatcattcggtgctctccttgtaatctctccttgaaataactcccctgctcacccccctcccgcttcagcaccgacgttcatcctcctttcaggtcagcgtgctcttcccgcgcctgggttagcagctccttgtcgcccctcccccactcctttctggtcacgtccgcgccatagcatcctttcagcctccctatcctcgcccctccccacagcttttcccctccccctcattctagtcacgtccgcgaactcccctctggccctcgtggtccgttgcatgtaggaagaatgcaatcactagcaatcctaggtgatgcagctacaaattatgtaatttttgtaattccggtggccaggcaagaaaggttggccctattgctgcctattttgtgcgcccgcagatttctgtggtcgtgggctaggctcctcgcgcaccggggtcctgcgtgctcccgtgctccatcctgcggcggcgggcctgcacgtactgcggggcgtcggctcccacgtgctggagaggggaaggtgggcggtcaactcatacctgctccctacagaagctggagccggacgcgacccgaacgcacacagcccgcagagctcagtctaattagggaagcagaatttgtcaagtcagtgctatgaggaagatggagctgcatgcacaaggtggccggcatactaggtttgaaggagccgggcatcttctagtctggaggctatataaagacccattcatagtaagatatctctcgatcaccttttccaaatccaggacattggtagttcggaaaaagtgaacctaatagtgaaaattcatttgtatgtggcggcagctgcgttttagaatccgctgtcatttattaatatctgaggtttttggtcctgtgtcatgagtggaaacatttactccaggctgctaaattacttgtctccttgtgtgtacgacatcctcgacctactttctggtgaaatgcccaaaagggaatgcatatatcctttttgtcaatgaagtagttacattgaagaccaccaacttcagtctgtctctgctatttttacgcaggttaaggttatgcttggggaagcccgtaggctccgaatacttattcacatggaaacaaaatctctctggaaccccatcggtgtttacaaagtaactaatgttacattgcaactctcctgagacaaccatctaaaaggagaaatctgtgcagcctttgtctttggctgtgagggtagaaacaaagaaacaggtatccagatagccttccgagtctggtctctgttcatgtaagactttcatgctttctggcttgcaggaagttacatttggtactactgttgcagtactgttgcctccaaattcagggtgtggctacattctagggtacagaggggaggccttaggtcaacactgaaggtagcattgttagcaaataatttgccctctgctggcttgagtatgttgtagggaaggcaatgagaatgttctgtgggaaagtgtaaaccatcaccgggtctgggcaagatgactcaacctttcaacttagaggcttttgagagaattttcttccttgggtgagtggggaaaccacacttgttctccaaggtgttgtaagatcccctcttaagtgagtcaaatactatagttcatttccctaaattaaacatgtctcaccgggactgagtgtggtgtgcatttggctttaatcccagcactgaggaagaagggctagacctgtgagcttgagaccatgtaggtggtgggtatcaagtaaatgaaggtgacctttgtgctgccataagagtggaagtgtggagtggctgcttttagtctgcatatcttcatttttgagtgacacagcagcttctcagaaccaccagtatccagaatggcagtttctgggaatgcgtttctgagttattgcctaagccaaactctgaatgctagtcacttatttagtacatagctattttcattttaagccttatattttaaattagttgtagggaaatttgttctataatgaggagatctttgccagcaatcaggacgtagaggcatgcagatctgagtttaaggccagcctagtctacacagccagagctacacaggaaccttgttaaagctgttgattgcttaatattgagtggaagaaatgaggaagcagctatggacagaggggtagaaaaacttttttttttttagcatctttagcttttctgcagcaagtcgagatgagttgggcaagaactaagctggagattggggtagaggaaaatgactcatgtctttcagtctctgaatcattccaaatacccagagccaagttaacttgttgtccccccccccctaccacttctggtaaaagaccgagtcatctctgtaaactgcgctcatagaaactataataggagtaaatgacagactatatgacaaagaacattcttcctgctgtggaactctcgtgagtcttgaaatttttaccaagaggcagacttagtagtgggagaggggagcctacatggtcatggcctgcaaacctctgcctgatcttaaggcctggtgtaacagctgaggattggggagtggtcttagggccaccactagctatgcagtgaggccacacccctgctgtggtttgtgcctgtctgcacgtaggagggaggtctgggctgcagtaccttcagtggcctctcgggagctctgagcatctcctgaggcctccaaaggaagcagtattccaaatgtgtgactaacctgcagtcatattttggctttaaactactggtgtctccagcaatgagtgtaaaagattttaccccataccatgcccattttggaggagtcaaagttttgaccttaagtaaagaagggctgagcttggtggcatgtgtttttaatctcagagacaggaggaactgagttcgaggacagtccgggctatgtagagagtccttgtctcaaaaaaaaagtagggaaggagctggtaatctctatggaattccattctgatggaatgtttgcctatggtgcataaagccttgctccatccctagtactacatagtatgctcatagtagcacgtgtctgtaatcaagctgagggtgggagcaaaggacaatgggattggagaagttatgagttggaaaaaaacaaatttgtgtgtgaatttattaaaatcagaatcctttttcgtttttggaaaatgtaaaaacaaatctgtgataatgctaagttctaggtgggagaaataggttttggattccactcctccttgggaaaaaggcaaccctgtaggcagaagtagtaagtaggtcagtgctcttggacagctggtgctgtcttctttaatcacccccacccccaactgtggctcagggcattttaaatgctgaaccagtgcctcctgttgctgaggccattgtgcattctgcttacacctggtcctccagggtctggaggagcacaggcatcattcccagaaacactggtccttctaactgctaaggaagacagcatgtgccaacagaacagagacttctgcaaatcccctccttcctgctctagctagttctctgctctgcacagctgagctagaagccaggaaatccctctctttgaggaaagagtttaaagtagaactgttcaggttaaggttaaataaaagttggtttttgcttatttttctctctagttgttttgaccacaggagtttaacttgacttcaccaggtggtagctagcctagtaagccaggcctttctgagagaatatactcttctttggacatggtagcatatgcctttaatcacagcactcaggaggcagaggctaaccagggctagatagtaagacctggtcttaaaaaatcataatctgggccgtagtgacacacgcctggaatcccagcacctagcagatctctgagttaaaggccagccttgtctattgaattccaggacaaccaaggctacacagagaaaccctgtctagaatctgacccccaacctaatctctgcttaagtgatctgtacccccaataccatatatgacttaattttgttgtattctgggtactgtgttccagaccataccaatttctgggggtggaattgtcagctgcttggaccctggtgtagcctgtgggttagggtcacttctgtagatctgtgctccagaggaagctcctagagagcagggtgaccagcatgttgcgtgtcatcttgcagcctttctgatggatgaaactaggtgatcttgggtactggaaaaagtaggcttcagatgatatcaaaaaaatggtggctacaaagccccgtccctaggcagcagtggttcggggcccgtccctaggcagcagcagttcgggccccattcgtccctaggcagcagtggttcgggccccgtccgtccctaggcagcagcggttcgggcccacatgaagatgtagttgttgtcggagtgaggcatgaccttacaagagttggctctataaaaagcagggtggcactgggcatttggggttttaagtgacctttgttcctgacacgagggaaatgaatgtgaagggccaaggaactgcctgagacaggaaagggcagaaccaggtaatcagccaagccaggcctgtgcatgctccaacctgcagcctgcactgaggcctgggctggtgtctcctttctcacagaggggcaatggctctgaggaacatcctgctgcagggcctcaggttcaagagtaaggctcctgcctctgggcatggtgtctgctgctgccgtgggacacaccgggcttctgctgctggttggaggttggctgtgcatgtgggtgcctcctaaggaggaggatgctgtagtgagttgcgctggcatgggtggggccgcatgtcctggcagtcacagctatcaatctatgcaggtaattagccgatagtgcgagccctgcccagagggccccctactctaagagacaagggtggaccaggaaaagacaccaagagtagggtgttctatttttatactatactataggtaagtgattggctggagttggggctctggaggccatgggacaggcagaagagctatgggtgggtgagagtggggggtcagagccttgagtgaaaagaactgggttctagggataaggttagtaggagagttggaacgcttgtgagttttgttagaggtctattttgtatcagtccattgaaacacatgtatttccatttgtcactttttatctggtagcaggataagtccactctgcagacttcaggtttaattctagagagagaaatgggttgtatatttgagattttacctgctagattgtgccctgagccctggaggcagcagtgcttgtctatcagaacattcttgtcagcacaacaaagattgttctgtcaagcctgagaccagtcaaaacaggaaaagaaaattgctggactctggcatggaactgataggttgtggtcatgaaatttttccatggattagacacactttggggaaggtggctttttaaatcaatggattaagttgcccttcccaggtgaatgaagctgcccagcttgagcacttgggagggcaaagagaacacatctgttactgaatcttagggtagtttcgaaagacacagggctgatgtgtggaacttcgcaatgagtttataggatgcatggaaataaatgtgcatttccaaagactgttcttaatttcacatcaacagtttctttattaataaggatatgctggttggattttgctggagaccactctggcctatgttgtaccttacataataagaaacagtctcagggggttggggatttagctcagtggtagagcacttgcctaagaagcgcaaggccctgggttcagtccccagctccgaaaaaaagaaccaaaaaaaaaaaaaagaaacagtctcagaaggggggagggaatgtcataatacatgctaccactcaggtaatgaagaagggggaaacaaagcaacgagggtcagtgagacagatggctgggaggacacggaaacttgcttctgatgactcaggtacttctatccccagaacctgagagatagaaggaatgagaagacttgtggtcctctgatctctatacggcgtccaccatgtaccccctaaaactaaatagtaaattttaggtgttgggaataaagtacttcaggggatccagtgaggggtcacttaccaccgggtttttgtcttctccagtatacatctattttaatgtagttgaatctctaaaagcaacttttaccttgtcattaaggtcattttcacaaagagcttagtcacattcagcttttcccttaatccttgtagctcttcagggcagagcatactcagctctgagctgtctttccaagtggggccatcaagccaatctgggtcaaaaaaacggcaatatgtcaacagaagcccagacccagagatccaaaggtatgacctgatcacctggccctgagcaggaatttgcaaggatacacttgtctccggtggagagaagggcctagtggctggcttttcagtacttttcagacactcttctaatgtctgtcccactcatttggacttcatagttcatagtcccatcgtggtcacagcaaggcctttgtgtctctttggctttgtgtaccatctgtactgtcccttagtggccacttaacctaagcctggctgggagccatgagggtttgccaaccatgagtttgtttcaaccatgagtagaccaaggacctggctgcctaggatatcctgttgtatacaggacttcctcagatttggggagagggaaaactagtagaccctggagaagaggttgacttgacgtcttggtacctaatgtgtcttgtagagttaggaatgaccaatgttggccgctggtttcttcttcctttaaaagttctcaattctgcaaggaatgtagtggtgtacctctaatcccagcacccaagagctggaatatcaggactgtcatgagttcaaggccagcctgggttgtatatgagaacttgcctcaaagtttttcatctccaggcctctgatttttgatcactagtgggtggctgcaatgtcaggtggcagagccaggcttgggtgcatgttctttaggtatccagaaaagccgatgtccagggtagagtgcgggtctttgggaaggcagggaagagcaaggagatgtggagtgcaggctgtgtgctcaggagtttgaaaggagcctcgtggcctgaaaattgattcaagtctctggtattctgtgaggtacatcccaggttctcgcttactcagccaactatccagctcagtgagtgcttagctgtaagtcgtaataaagtaaaagcaaaggtcaggacagctgttgggattgtgacctgggtgattaattgcctggaacagatgttcagactactgtgtgtggtccctatcagacagagaaggagactctagatagccagaactggggctgggaaagctgggtctaatgtcagcccttgggaagagtagtgactttcaaggtcaacctcggttgcacagtggatttcaggccagcatagactagctactgtgtgtgattccatctcctaggttactgaagactccttggtgaggttgtaatgactttggagccatttcaatgtccttgtgcaaaatttgctttgtgtgtcttgtacatggtgctgttttttgcttcttgtccagaaaaaaacttgttgcctgtaatttggaccttattcctgtgtcggatagaggctaaggaatacatttaggtgatacctcaaacctaggactttaaaaagaaaggacttgggacagggttcgtctcttggcagcaggatcgctttaggtcatggcgaacatgataaagaggctatgtcagtgccaaagcaggtttgacattgtcgtgataggcttcccagatgttcgtgtgctgacacattagctacgtgctctgcaggagtggggaggtttccttgtgtgggaaggtgatgttctctctagggttggtgtgaaagctcttcaaatagatggcagccgttgtaagaagcctgcttaaactggcttccagggtgtgtatgtgcactgggaaggaagctacttttgttcaccaaggtctttgctgttcgttcatagtcacaggagccctttgcccaatttgagtagtacccacataaccagcacgtgccaggcctctgagtgacttgtgcacaaagcagtcttattgggttaatgtgtaagcgggagacagtctcctatgccaaatggttcattaagcactgcctagtctaattgaactcttcaaatctccgggatccaggatctcagaaaccatgcccaagccagacagcgaagcagggactgccttcattcagacccagcagctccatgcagccatggctgacaccttcctggaacacatgtgccgcctggacattgactccgcacccatcacggcccgcaacactgggatcatctgtaccattggtgagtgagtgtggcccccttcgcagggcttctgcctggtttgaaaggcgtaataaccattgcagggctaacctaggttctgagagacactgtccacacttttagaggaagtattggtgtacttcttctgcagttggcgaggttcagggtggtcttagtgggcctttgcaggagtagttcagggacttcagatctttttcctgtgctgtcactccactttctgcagaatttatagaaaatgatagtgtccttccttacatataaattaagagagtccttccttacatataaatcaagagagttgactagcacagtagtctttctggactttggaaagacctttctctaaatttgtggaggtgattaaaaacaaaggacaaggagttgctttgatagtgagcagatttcatatactttcctacctgagaatcatgattttcttcctgtggctctaaatgtttcttggtaggccctgcttcccgatctgtggagatgctgaaggagatgattaagtctgggatgaatgtggctcggctgaatttctctcatggaacccatgaggtgagtggcagcttgatccaggaggttcgggacttgctgctgctgtgctccaggcttctcagtgaatggagcgttgctctagctagcatttacatccctctctccctcttcccctctggccaacttcattggcatcaacaacctggcctgctttgtcgccggccagggttatccctaccatcaagctttgtgaaggaaaccctttatgtaagggtttggctggaaactctaggccatcatgtgtcttgactgcacttggcacccacgcctcgggctttctacagtggctcctgtgagtccggtgaaccaaaaggtcatgcagaagacttagttcaggccccagatggctaggtaggtgcagatggttttcccgtgagaccgagtctaacgcacaaatgcagtgcggtctggggatatcccacttactcctgctgcactgtctgcccatcatcagtgcccctccctaggagtgaggtagggatgacctgggctgtttattattaggtggccccactgatggagaggctgtgcctaggaagcaggtggcagctgttagaagggtccattcctgcccctgctcactttgagaacaacccgctctgagcagatgggtaggtgggggcgtccaacgatccgccttctgactttctaacttgacagcctttcctactggcactgatgaaggagggagagactccttcctgcctcttggtttctctgtctatttagagtataagtattaaccctgtgctattttgccttctgacctttgaagttgttttgactaaaaatagatggagaagaacagtccaagaagcgaacccttccttcccagaacataaggatcttcatagattccctaacccatattctataaatagcttgtagactagcccccaaatgtcatgggagtgcaatggtccctcaggcacctaagcagtacttgatacattcttgcttgggtgtggtgctacagaatgtgggttcaaggccagcgtgaggtaacataccaagatcctattgcaaaccaagagaggtcgttgctttccgggctctgttgctctccatcagagctcccaggactaaacgcaaaggtaagcatagctcagagtcagaactagatccatttgccatggtcctcaggtctgttcagccaggcaaaaatagatttggagacactgacctttagggctcttagatccagtaaaataacaccgtgcagcagcccggacatgacaggggacagtgagacagttcagaccactgggcagctcagctctgccttgattctaactctaaggcattagagattagctgtcctttgaacctttttatgtcacataattaaagcttcaccagggctttcttattctatgtgcaccttcgatgaggggctgtggctgtctgtcttggatgaggggctgtggctgtctgtcttggttttaaccattcctggagacggtggaatgaatcgagctccttcgagctccttccctctgacgggaccatcaattagtcagctgcacctgatccctgggcagttttggcagtgtctctagttcccgtctcctgcttttcacatggtcaggatgtgtcccttttgctgaacaggcagtgctgcactcccacactgacttttaagcagcctaaatttagagcctctacaaggacagcaccgatggctacccgtcttcaccatttggttgtgtgccaggaaaaccattgctgtgatagtggaaagcacggtctacctcccagaagtgttcccgtgtagagaaggttgctggaccatcagacactcgaatcttttttttttttaaagatttattcatttattatatataagtacactgtagttgtcttcagatacactagaagagggcatcagatctctctacagatggttgtgagccaccatgtggttgctgggaattgaactcatgacctctggaagagcagccgggtgctcttaaccactgagccatctctccagccccagacactcgaatcttaatgcagttttacatacatgcggtagaaagtttgtcattgctgaattgaatggtctgctgtcagctgtgtcacttggaggcacattggcaacagactattttgccatcgtggctctgtgtcccaaagaggaaagggaaaaggttttgtttgggggaactttattgcaagtgaagaatagcaaaggcagttccactggggcacgctactggtcagagaaagtgattgtgaaagcaatgtttgcacgaccagagcaatgggctcttcagctccggccattgttcaaagtcaacagctttgagtttgaattgaggttgggatcgatagtgaaaacagatacagacagccagggcgcaaatcttcaaacacaagtcctaggtctttgcctgtcctggttgttagcattctttggtgagtcctagggacactagaacaacagaagacacacagtaggacaggcttaatactcttcgagttggagttgatgctgttatgtggaggggagaatggataaggtgaggttcaggacttggacatacttttcttggctaattgatcatctttggctccagggtggctcagggcatgggaactggacttgggtgtggagaacacctacctgtcaatctccccttctttctctccagtaccatgcagagactatcaagaatgtccgtgcagccacagaaagctttgcatctgatcccattctctaccgacctgttgcggtggctctggatacaaagggacctgagatccggactggactcatcaagggcgtgagtatccaggagtttaggtctttaaatgagaatatttttcatctgcctggtaggaattattatagatgcacatttttggtatgtgaataacatacttaagtctcactctggggacctggtttgttgtttgttttgtttgtttccctcaataaacaaattcaggatttacagaaaggtgatcggtttcttggggctttgagccagagtttgagcgccgccatcagggtgttggcgtccacagtcacacgcctctgctgtctttaatctagagcggcaccgcagaggtggagctgaagaagggagccacactgaagatcaccctggacaacgcctacatggagaagtgcgacgagaacatcctgtggctggactataagaacatctgcaaggtggtggaggtgggcagcaagatctacgtggacgatgggctcatctccctgcaggtgaaggagaaaggtatgtgtggtgtacagtccacggcccaatgccactcccatccccagaactctggtaagcacttaacctagcatgtatgaattggtctcccaaggatcaaaagtttaaggtggttgttggtctgcatccctggcctgtctgaaacactgcctgagaaaaaaaagacaaataacctacaaaggcctatgtgtacacctctaccctttagttccagcactcgggaatcagcaggtgtgtgagttctcatgtgtaagactactcctgtatgccttagaatgatagttaaaatttaccgaatctaaacactttgatagtctaagacagcgttttcagtagctagccttggactcctgcttctccggtttccacctctcaaatgctagcattaaaaatacacatcaccattcatggctgtttgtttattcatttattttgaggcaaggtttcactcttttgacttggaattaactgtgtgggccaagctggttttgaacttgatctgtttgcgtgtgcctcttgtgctaccaccccaaaaactcagattttcttagcagcctgtgggcaggggtttataagtgaggaaactgatttattggcttgcttaatcatggctctagagttctcttggcttctcacaaactgagatagatggtcttgatccctttcacacaggtgctgactacctggtgacagaagtggaaaatggtggctccttgggcagcaagaagggcgtgaacctgcctggtgctgctgtggacctccctgctgtgtcagaaaaggacatccaggacctgaagtttggggtggagcaggacgtggacatggtgtttgcgtctttcatccgcaaggcggctgacgtgcatgaggttaggaaggtcctgggagagaagggcaagaacatcaagatcatcagcaaaatcgagaaccatgaaggtgtccgcaggtgagtcctggtggagacctttttcacattacccagccatcgagagagagagaaaggtgtgtctgtgctgtgtcctcagcaaaataaagcctgccaggactggtccagaatatctgggtcagcatcggaaaggcaaagtagtgttagtcattattttggaaactcgggcctggtgtccacctagatagagccttggaagtcgtctggcctgacaatggtgctgaggaagactggcccattcagcttcccatagggacgcatgctttagttaagatgtcattcataaggatgagtggtttagtgatacatacttggcattctctcacttcccctagacctacttctgtgggttacgagtgggcttggaagagttagggcggtaattgttaacacacacacacacttgtccttggctctgtgtccttttatactgggcaatttttccctcttgataagccataggtgctggtggccttggtatttgttcagccctggaggaggtggggagtggaacagctcaaaggacagcactaaggaccctagtaaggacccttagctaaatggagcatgggcctttgtaggtctgtgggcctcaatttgtctttgaagtgctgattagggaagtatgatacaaggggttgtgagatgtagaccgaagaacccatggaactgaacctcatccccttgacgtatccccgcaagtaaaatggttattgtgctgtgggtatcatcaaggtggtgggtgcccactccctcaccgtgtgcctgggggtgtggccttggagggcacctctgtcagggccccaggaaagctctgtccattctttggttgtacttcctgttctatagcatctttctttttatcaggtttgatgagattttggaggccagcgatggaatcatggtagctcgtggtgacctgggcattgagattccggcagagaaggtcttcctagctcagaagatgatgattggacgatgcaaccgagctgggaagccagtcatctgcgccacccaggcatgtgctatcccttccttctgtgttctccacctaggagacctggtcttgacctggcctttaggtacacgtacccgcacatagctatgacctgcgtacctgtgcaagcttcggggaattgccctggcaatcatcactgaagatgtcctgctcttccattatttagtgactttcatttagcggtggtctcttacttaataaaaacccttgtttgtcccctcctagatgctggagagcatgatcaagaagccacgccccacccgtgctgaaggcagtgacgtggccaatgcagtcctagatggagctgactgcatcatgctgtccggagaaacagccaaaggggactaccctctggaggctgttcgcatgcagcacctggtgagtaagtcctcagagcctggggtagaagcagctctgctggagaggcctctgtccagtcttgttacattgctcccgtcacagcaaggagagtgaggtttgtggaggtgtgcttgagtttcattgtgctttcactgcctgcacctgcccctttgtcctgctctggggattacataggccacatctggctaaaatatcaaggtcctaggatgcagtcaagggatgccttccttgtggacacccagagggcctgggtacctctattctaaaggagccaagagtttgttcagctattctccttgtttagcttctcatttggtctcctggtggtctgtcccatgggcagatgcttctctccactgcaggctgtagccatactgagctgctttaagctggccttgcatggtgcctgtgacatgggacttcctcccttgctgtgccagacccaactcggggctacaaatagctctgggggtggggaatgggtgctgaatttagcaggttctgtctggtctaggaattataaagacttctcaggcatattatgtgctcttttgattaagtcttctggtttcagtaagatagggtctggcgcaagcttgtaattccaacaatcaaatcagcctgcggcaggaagatggcttgagattaggctggactagaatgagacatcttgtagaagcaacaggcgtcagctgacctgtccctgcggactttccacccaggcctccgtcttctgtgttctgctccagaacttcttccggagcagaacaattatctggggcatcagttagggaccgttgcctgataaaggcatggtggattagctttttgggggttttctattcccttttcagccaacaaagaaccacagtatcttttgtggctccagtttcccacaagcccccccaaacttaagacaagtgaaagaaaagaaggaagccccttctgggttaggcatcttaccttgctttatatgtatatgagagttgagtagattccatgatctttccatggtttatctgaattatagctgtagccagatgtttgcctatattgaaacagaccagcgctttgactcggctggtcatgacctgttctgattggaattgtgagctggtcctcatagcttctcatagcatgcaggcattctctcccagaagacttttcccttccttcaatctcccccacagtagctgccttcctggtttacaccaaaaccccttttctacttacctggcctcacagtggtcaggaacaggactttgaccaggtattctaaagcatgtagtcacataaatgtatttttggatagctcaagaagaccttgcccaaagtggcggttgatgagattaattacaattaattatatgtgtatgagttagttttggtgtcctgtccttttctcatgccctggtctttcctatgaacagacaccttagaacctcgaggctgggattgcatggccctgctcagaagatgagtcacagagtccgggttagactgtggctaccccctcaggggatacaaatttctctatcagttaacacttaggacagcttctcccctttctttatctgtttgctgtttcctcctgtgtctaactgattcagttcaaaacctgtaattaaaagctagacatcccagctgtggttgcttcctgtccatgcctcttgtcctttgggcttgcctgcattccatgcttaaccaaaagattactttccacttgcaaatctgctaatgctaccaataaaatcgagtgctggtttcatatcattcctggaattgcactctctaaaactttatttctttatttctaatgcttggttttacttgaacaggtggtctgtatataacacatttgctatcctgtaaccgttttaattattgagtttgaatctgtgtcttgaaaggcctgtgtgctttcccaggcgtctgcctcctcacatggctgttcagtgtaccgtgttcaagtgagccagtcgaccactgttctgtttagaacttgtgcactgcaactttggctcttttgaccctcacccccagctttcagagctgcccgagtgtttccactgtaagccaagtgcaagcgctcactcttgtgtgtaggcggagttggatgccttgtaaccacatagccatgtgaggaggggacgcctttttcttcctgtaagctgtgtcaggaggcagtgtggtcaagcggaagtgtagttggctccaccttggcatctttccatgccagggtccctttctcagctttaatcaaaaacaaaagaaatgatggatggtgcatgcctagcacttgggaggctgaggcaaagaaaatagtggtgttttgaggctgaccatggcaagttcaaggccagtaggtgctatggtaagatcctatctcaaacctgtgtggggttgagaattggctttttttgtttgtttgtttttagaaacaggaaaatcccaaaaggatactatctttccagaagggaaggaaggatctttccttgtgtttgaacagaaaagagagccacccaaaatcccacatataataccattccaggctttgaccaccctgcctctgtatctgcatggagaagaaaagattaacctaagagttccttcctctcattagttccctgtctttccatgtgttgtctcttgtttttgccttcatccttcttccttatccttcctaccctaaaccttacagatagctcgagaggctgaggcagccgtgttccaccgcctgctgtttgaagagcttgcgcgagcctccagtcaatccacagaccccctggaggccatggccatgggcagcgtggaggcctcttataaatgtttagcagcagctttgatagttctgacggagtccggcaggtagggccctgagggcaggtatcattataagataaccagcttctcacacaactagggcccactgtgtgcctgagcctgggcatagccctctctcctgcaggaaggcagccaaggaggtcacgatagggcaggaccaaaggattccctagtgggtacagtggaagtcacaggcactggttcaggatggttcctgtggagtttctaatcttgctcagttcagaacgtgtagtgactcatcttctcctggctttttgtgcccagggacatgttccttcccagttgcctgtgactcctttcctccctctccatttgtgacaaagctctgacaaagccctgtcccccttcctcgtccctctggacggatgttgctcccctagattgcccgagaggcagaggctgccatctaccacttgcagttattcgaggaactccgccgcctggcgcccattaccagcgaccccacagaagctgccgccgtgggtgccgtggaggcctccttcaagtgctgcagtggggccattatcgtgctcaccaagtctggcaggtaggaggcggcagtggctccctggggatccccagagcaactctgggctgatttaagacccctggctgccaccaaaggactccaggcagcactcccaggtacaccagattaggtggctccagtgccagtcagtgcacggcctgcctcagggcctgaagcatatatacagttttgcttagttacggtgtggaagctgggcatgggtacatgcctttaatcctcccagcattcgggcagaagaggtgggcaaatgtctgcattcgtggacagtctggtctacaaagcaagttccaggacagagccctagctcacaaaaataactcagctggtaaatactacttgctacacaagttagaccatggaggagggagaaaggtaacttacaagcgtgcccaaggacatatacagtaacactttttctcatcccacttggagggagctcaatggataaagtgttggcaatctggatttggatctggcacctgtgggtttttgttgttgtttgggtttttgtttttttttttttttttaagtcacctggagaagtagttaggcctttaggagcactggggttttggtttttaagatttatttattatatatgaatacactgtccctatcttcagtcgcaccagaagagggtgattacagatgattgtgtaagccaccatgtggttgctgggagttgaactcaggacctctggaagagcagttagtgctcttaaccactgagccatctctccagccagcactgttctttcagaagcccaacacatacatgacagctcacactatctaactgcagtcctggggggaatctaatgccctcttctaacttccagccgtacatgtttatatgatgcatgtggtcaggatacccagacagctggagggaaaggaaggtgcccaacacacctgtgtgcctgtaatcccagcactcatggtacagaggtagaaagatcacaagataggtctaatctgagcaaattctgcaacagcctagatacagaaagccaaacacataaaaatcagtgtgggctgaggatgttgacctttgtgtgaacttggccatgaacattgtatgcagctggaagcaagggatggggtgctgaaaaatggggaccttaaaactaattttctgaagtcctgaagtttcgagagctccaagatacaagttctatgctaatcctggactttctctgaagagttcctggttccttcaaagcccatgtaggccagcatgaagcagggtattttctgctgtggaatgccacatcccctaataccttgtgtgtcctcaagatgcctagttaattcagtaagcctgaaacagatctgatagaagtaaacatatcatttgctatcggttgaggagagaggggctccctacattcccaaaggatcttgattggccagatattaaccatgcctggtcacacctagatttccaaaaaccataggnnnnagatttccaaaaaccatagaaattcagaggtttcttttagcaccaagttcagctgataagactcccaaaggagcagtatagtactgctctggcaaatactgtccactccagggtctctgcagataccagaagtcaggagctaggcacatggtgcttggattgtaaaagttgctggcagctacaggatggttctggtgagattaggccagagctgcctcactgcctgatggaagggttcacagtgtgggagggatcctgccagccgtggtcctatgggactgcccacactgagatcaggacaatgagttaaaaaggacaggacaggtctgagggggtggccaggcactgaaacagtaagttgtaagtgggcaaagcctgtggcctagaggtgaattagagggtgctgcctttggctgactgaaagggtctcgtgacacaatggccattcttcccaccttctcaggagtgctcaccaagtggcccggtaccgcccaagggctcctatcattgctgtgacacgcaatccccagacagcccgccaggcccatctgtaccgtggcatcttccctgtgctgtgtaaggatgccgtactggatgcctgggctgaggacgttgatcttcgtgtgaacttggccatgaatgttggtatgtagctggaagcaagggatggggtgctgggaaatggggagcctaaaactaccatttcctgaagccctcacccaattttgggcccaaggcaaaattaattgcctcattaggctcttgtaagattaattggagctactgccctgtggggtggggcacacaagctgttgtggtctttgttcctatataaggttggatatcaagagacaaggaagcagctgaccctgaacttgggcaaggctggccactctagagtctaaactgaatggtgtccaaaggtccctgcagtctgagttttgcctcagcccttgtttaagctaggagacttgcaccaccttgtggtaaaaagaagtaactggcaacctgctctcctacctgaattagaagcaatagggcatttgtacctgcctggttaggcttgtgtctgggctgggagaaagcaccaccttgtggtaggagagagtactggttgctcagttaggactgggtagggctttgcagttatccccaagtgttgtatatacctctactcaccaacctccttctcccctcaggcaaggcccgaggcttcttcaagaagggagatgtggtcattgtgctgactggatggcgccctggctctggcttcaccaacaccatgcgtgtagtgcctgtaccatgatgatcctctggagcttctcttctagcccctgtcccttcccctcccctatcctatccattaggccagcaacgcttgtagtgctcactctgggccatagtgtggcgctggtgggctgggacaccaggaaaaattaatgcctctgaaacatgcaatagagcccagctatttttcatggccctacttgagccaggggtgaaggaggaatgcaggattggaaaccctctgactttatcacagaagggcagcattatctctgtgttctttgctcctgtagaaagttttccagagaattcccagccctggcctggaatcaggagacagcaagaacagaggctgggggcccagggttcccatgtagatgacttttggccctgtccctgacttgctttcccaacagctttggcctctctcctcgtgcactccactgctgtccctgcagatgttccactctccacctcgtactctgcagcgtctccaggcctgttgctatagtgcccacctgaatgtcaataaacagcagcggaagca

SEQ ID NO: 51 - DNA, 13748 bp, Oryctolagus cuniculus
  source 1..13748: mol_type = genomic DNA
atgtcgaagtcccacagtgaagccgggtctgccttcatccagacgcagcagctgcacgcagccatggcggacacattcctggagcacatgtgccggctggacatcgactccgcgcccatcacggcccgcaacacgggcatcatctgtaccatcggtgagcctcggggaccgagtccgggaaggagcgcgcattgtgcgttctgctgggggtgagcgtccccagctcagaggcctaggcctatgccaccaagatgggcagagggctggccaagctcttaacctgggctttcctgactgtagggggctacagacctgtgcagggcgtggggagaggtgttcccctaaatcaagaaagcaggctagcacatcgacggccgtgagtgtgctctctgaaagacttgcgttatttgggggaggaaaccgaggaagcgtttctttgagtcactgacgctgtgccgcaatatgtaacaggccctgcctcccgatcggtggagacgctgaaggagatgatcaagtctgggatgaacgtggcgcgcatgaacttctcccacggaactcacgaggtgagcctcggctgcacattggccgcgggggcggctgtaggcgacagggaggagtgccttgttcacttagggtgggctccacagattccagtctgggtgtcccagccctaggctcctgccctgagctctctgcagcttggttctggcccagttcaggctgcagagagctgggcaggcgcccagaggtttccactctggcaggcgagtgccccgcccgtcagcactgtgcccccagcctcgccctctccagaggttctgctgcacggacagaaaaccagccggcgagtgtttgcgctggagctgttggccgtggcaggaagcggcgctctgagggtgctggggttccctgaggggccgcagtcgtggggcttcatttcggcattggtagaggagtaatgggcaggctgacgttgccctctcttgccctctgtcctttcctcctctgaggtcagtggtgagggtcactctctggcatttggaagctggtgttaagtttggactaaaaataagccctcgggcagtgcaaaccgcggggtgaattcttgctttctggaaacatgagacttgactctccatggtaaatgctgcagcccctaactctggggcccagccagggccagcaggcacctcgggcagcctgaggcgcatcctgggctctcaccaggaattgctgcgtgtgtcaccctgcccagagggccaaggcagcagtgaggatcagttcacctttgacccctgctgccggagtctgccgtgggccctgcgtgcgggtgaggagcagaaatagatttggaggcgctgacctttcagccgctgcgcgctccctccaccagtaaaacaacaccgccccgctcggaaatggcaagggctgcacctgtcgcacgagggcgtgggtctgtgcttctgattctcattcccagattaaaagtgggatgaaaaaggaaagaagcgcagtgcctctggtggtttagggtcaggcccaggtgagcttccagggtcagcatctgggaagcgggcctgaggtgctgaggtgtggactgacgtgaccgccttccccctcctcacctgacagaggcacggctgcttctgtgtctgggcttttgtgttgttttttgtttttgttttgttttgtttttttttttttttttaatttatttaattctatttgaaaaacaaacagattttcccactgatttattctccaaatgcctgcagcagccaggaggcaggactcggttcaggtctcccgcatgggtggcaggggcccgcacacttgagccccgtcacctgctgccatccaagcgtgacgagaaccaggactcaaccccaggccctccggtatgggatgcacacatcccaggtgctgtctccagtgctcagtcctacaccttgtctctggcagctctgaccgtgtccctagcccccgccctggcagctcatgtggaccatctgtgcttgctgaacatgcagttccacaacgccaggctggccagaactgactctgacataatctaaatttagagccttccacagggcagcagtaatggtggggtgggcaccactccgcagataataagcagcccagccccaccccagccagcaccctgtcaaccatttagaatgtttatgacataagattttacatccccaagaccaaaaatggcatgtttatatactttgagttgcaaaaagtattttctgtcactcaccaaaaaacttcaagttccataaaagtagaaattttacagacacatgacctgacataattttttagaattgtttgaaaggtagaggcagagcgacatctctgatcttcccaaatgtccatagcagctggagctgaaccaggccagagctgggagtccggaactcactctgggtctctgtacaaggcaggcagggacccaggcacttgaaccatcacctgctctgtccatgcactgtgttgtcaggaggctggatcggaagtgcacctgggactcaggcgtgtcttcagtggattcttgactgctgtgccacactcagccctgactttttttcatggaaatcaagatcagtatagagtgtgatgatcaggagagcttcaaaactcgttctagaaccagaagccgtggcaccggcaataaataaatctttaaaaaaaagttaaagaaaaagaatttgtaagaccatggcttgatgaaatagaagattgaaaaattaagctgagatctgattctttgtgggaaacagtaaatcagaaatctcaggaaggcctggtcaagaatacgaggatgggggccggcgctgcggctcactgggctaatcctccaccttgcggtggcggcacaccgggttctagtcccggttggggcgccggatcctgtcccggttgcccctcttccaggccagctctctgctgtggccagggagtgcagtggaggatggcccaaatgcttgggccctgcacctgcatgggagaccaggagaagcacctggctcctgccatcggatcagcgcggtgcgctggccacagcgcatcggccgcggcggccattggagggtgaaccaacggcaaaggaagacctttctctctctctctctctcactgtccgctctgcctgtcaagaaaaaaaaaaaaaaaagaatacgaggatggggagggaattgtggcgcagaggcttaagcctccactgggacccagacacctcatatcagagcactggtttgggtctcactggctctacttttttctttatttatttttaaagatttattttatttgtttgaaagagttacagagagaggtagaggcagaagaagagaggagtctttcatctgctggttcactctccagatggccacagtggctggagctgagctgatcccaagctgggagccaggagcttccgggtctcccacgtgggtgcaggggcccaagcacttgggccatcttcactgctttcccagatgcaccagcagagagctggatctgaagtagagcagctgggactcgaaccagtgcccataggggattctaacactgcaggccagaaatttaacccactgcaccgcagcgccagcccctggccccacttctgatccagtttcctgctggtgtgcctgggagtaatggcctcagtacttgggtcctggtcacccacatagagacccaggatagagtgaggttcctggcttcagcctggaccagccctagccattggagccacttggggagtgaaccaatgatgaaagatctcccactccctctcctctgttaaaggaatttcttgactggtggccaagtaaattttattgtgggaacatagagttattaaataattcctgtgtgcttgactcttctgagtttcatgttattttagtcctcagaacaaaaggtatttggtaatattcagcacccctccctccctccctgcctctctctctttctttctttaccggcagagttagagagagagacagagaaaggtctttcttttccattggttcacctccaaattggctgctgtggctggcgtgctgccccaatctgaagccaggagccaggtgcttctcctggtctcccatgcgggtacagggcccaagaacttgggccatcctccactgccctcctgggccacagcagagagctggacaggaagaggagcaaccaggactagaactcggcgccccaaccgggactagaactcagggtgccggtgccacaggcagaggattagcctagtgagctgtggcgccggccaatccgttccttttttacatactgcaggatcaaaaaacaccacaccagaactatctccagactgggtgtctggtcagaggttaagttgctccttggaatgtccacatctcatatctaagtgcattgttcaagtcccagcttctctgcttccaactgtttattgttagtgcatatcctgggaggcgacccatgcgggagacctggatggagttgtaggctcctgacaattgcaggcatttggggagtgaacccagcagctgcaagataatctctctctctgcctttgaaataaaaagtgaaaataaattaattataagtaaatgatttctattgcaaaccagcaatacgaagaatgtagaacctctgtggaatagaaagtggagccagtggtcgctccttgcagagagtgcctgagttagaagctcatggtggtcagacagctttgggcagtgtcgtccctggagacggctctggctcctggtaccttccagcgtcccaggcctgctggcagctgctgttgcccagacagtggtggttaaaggccaagcccatgaagtgctgagcgaggtgcgctgttatcagcagtgccgtattgattaccctatctgggagcccttttgacatcttatctcggctttgactgcctttttgattgaacagtactggcaagtgagggagaaagggctttggagagagacgggagccaggatgtgctgccagggcagttggtgcggagaggggcctgagagcaccttggcctgctcctctcctctcaggccacaccctgggaagggagggtgcagccagctctcatgacctacctgagctttcaggtgaagtccacaacctggtgttggaactgtggttggaatccgtgggagagggccctcgcctggccacctctgtcaggctctgcgctgattgcccggcggtctcacccacacctgccctggttcttctgccccagtcgtcttgccctgttagttgtcttaggggaaagagcacagcccgaaggtgctgctgcttgtgtggttctggacggcagtggcctctgctgagtgcgtgccgtggtggaggtgcggagaggctgcgggtgctcgggcggacggcggccgaggtgggcgatgcagctttggctcccagccccagggatgccagctggcccgagcgtgggaggacacttgtcctcttctccctcccccagtaccatgcggagaccatcaagaacgtgcgcacagccacggaaagctttgcctcagaccccatcctctaccggcccgtcgcggtggctctggacactaagggacccgagatccgaacggggctcatcaagggcgtgagtatccttgggacacggggagcccagggcgtgtctgtggtgtttgcctgggtgctgccatgttttagaattcaggtcaagtgtaaatgtggattccatggtaccacaaaaggatctttttttttttagtctaaaaactgacttctggctttgagaagcagtttctttgtgtgtcttggccgtctttaagtaacgcaacagaacgtgttggcctttgtctttggctgtttcctcggatgtgctaagttagagaagggcatctgttcaccaagggccaagctttgtgattcctctgagattcagacattgtgctggggaggcgggggcgctggctgccagctggactctcgctcactctcctgtccagagcggcacggcggaggtggagctgaagaagggagccacgctcaagatcacgctggacaacgcctacatggagaagtgcgacgagaacatcctgtggctggactacaagaacatttgcaaggtggtggacgtgggcagcaaggtctacgtggacgatggcctcatctccctgcaggtgaagcagaaaggtgtgtatcgccagcctgagcagcaccccggcctcaggacagcaaaggcagagagctcctccggcctcccgacttgcggggctcgtggaaggtagggaggtaggaagcttggttgtctggcggagagatggtggtatggtgagctctgtttatttgagaggtagattgacagaaaggggaagacagattttctgtctgctagttcactccccgggtgcccacggcatccaggaccaaaggcaggagccaggtctccttcgtggatgggccatcagctgtgcctccagctgtgtcagcagggagccggatcagcagctgaacgaggcgctccactatgggatgtaggcgtcccaagtggcggcctaacctgcaccacggcactcacccaaacgagtgagggagcgctggcagacaggctgtgggcggcggttgggcttgtttgcgtggccccggcccccaggtgtgcttccctggtcctccgcgggagggaggcacctgctgcttgctgctcccctgtcccctggggatgggccctggcggctctgcatcccctgctcgctgtgcccacaggtcctgacttcctggtgacggaggtggagaacggtggcttcttgggcagcaagaagggcgtgaacctccccgggctgcggtggacctgcctgccgtgtccgagaaggacatccaggatctgaagttcggcgtggagcaggacgtgacatggtgtttgcctctttcatcnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnaccgggggacccacaccagagagagcaggcctccggccacaggaaggggggctgcccccgttcctgaccggcgacccatggggaagcgcgagggtcggggtcttggtcttccctcagaagggctggcccgggcgatgagccactcgcccccagagggaagcctggggggtcttcttcaggtccccagggctgccccagcccagcctgaccacctccgccgtctcagtgtggccagctggcccctcctagagcagtagctcgtgaggggcagggcggcagcagccctggggccccacagcctggtgttctggccgtctccggctctgcctgggcgtccttcctaccacatgagctcggctctctgcctttgctgtcctgatgggccgggctgctggctcgggccggtgatgcgcgtgccctcttgcctccccggggcatgcctgtggtgtccttggctagatgaggccgccgaccttggggtgggaagcagccagcagcgtcggtgcccttcctgggaaatgctgactggagcacttgagggcctcacagggtcaggtggagaggcggggcggggcatcccagaaggtgtcgaggcccaggagccgggagcctggccacgtgcacgcatctgtgctgtgtcgggccctccgagttaggggtcgtggccggggcttcctgtctgtcaccatacttgggacccctgtggaatatgacttctgtttcgtccctgccccctctgtcaggtttgatgagatcctggaggccagcgacgggatcatggtggctcgtggtgatcttggcattgagattcctgcagagaaggtcttccttgcccagaagatgataattgggcgatgcaaccgtgctgggaagcctgtcatctgtgccacgcaggcatgtggccctccctggggaccccgtccctgtgcacccccagtccagcagggtctcacccgtgctctgcccctcccagatgctggagagcatgatcaagaagccccgccccacccgcgccgagggcagtgacgtggccaacgcggtcctggatggagccgactgcatcatgctgtctggagagacggccaagggcgactacccgctggaggccgtccgcatgcagcacctggtgagtgcccagggccccgcttccgcggagcctcactgcacgagtgaggcatgtgctgggcgtggggcgccccttctgccccgcctcccagctcctgtccggagaagtcaccccagcttccgagtccttcgcctgagtcctggggtactgctgccctcagcagatgcctgtcgccctcctcagaggtccaggggcttcagcacctttggcctgatctggaagacaccagggctcctgcccccagctgctctgtttggctttgactggactccctgcggggcctgtgtgcacccagcaagctgcctgtgagccggccttgcatggtgcctgcagcgggaccggcttctgtgctgtgggggtgcagctgcctcacttagtccagacgacccagaggggcttgggaaggtggccagccccggtggtcctcacactggagtcatctgcaggagtctggggctgggccatagccgcctgacctcttctgtaggtgggagcccagggctggttgtcaaggccgctcgggcttggtgcaccttgagccccagtcacctggagagtggctccctgtggtctctgacgcccctctgtctgtgtgctgtggtaagctcaagcgggagggtgatgaccttgtctgaagcacacggactacagaaagtgtctctgggcgaagtctccgctctctcaggacctcgtctgtaagctggattagatgagatctacgactgctggctttagttctgataagtccctggtacactgatgtaatcctgacagctcccagcatcaccagggctgtgggacaagaactcccaggacactgacccaaagccggccttctccctgcggttcccctgcccagtaggacttagaacgaggctctgggtcccagagttggttctgggttctgaattctgtgcctctttcccttaactggtctttcccatggacagacagcttagggcgttgcaggctctgggccaggatgtggctgccctgcaggtggtgcaggccactagccggcagccgcctctcagtgtccgtgctcggactgatgcactgacccgtaactcagggctaggccgcaccaggtatggccctggctcctgtccctgtccctgtccctcgggctgcatgcactcattccatcgttaactcggcggtgctccccgcgtctgcttccgctcggagatgaaatgaatggggcggtgctgctgacccttcctcgcccgttgatgactaagccactgggaactgcattgtaatctcgaacgcttccactcgctttctgattctcagtctcacctgagctggtggtctgtggtaactcagctctctcgcgcctgttttaatcagtagctgttaatcaagcgtcctgtgacgtccccgcatgctcccatgggtgtctcttcacatggctgttcagggcggccctcgcgagctggcctgtgctgccagctctcctgcgtggccttggctgctgacgcctgcctcccagccctgttgtctgtttccacggcgggcactggggagagcacccttgtgcctcaagccaagcagcctccatgtggcacaaacacccagacagccaagtgaggaggggcctgctccttcccgaaatctgtgtcacgaggcagcgtggtcaggtccagcctgggcaggtccccatgccacggcgctagagcccgcggccatactggcttccctccactcccagccccggtatcggagggtgaggtgtgaaattctccatgccccacagctggctggacctggaccctctgcctctgcctgacgacccgattaacccggggtgccttcctccctgattaatcccccgttctgtctttccgtgtcttgtcttccctgtctcctcctcctcctctcttccttgccctctcccctaacccttacagatagctcgcgaggctgaggcagccatgttccaccgcaagctgttcgaagaacttgcgcgagcctccagtcactccacagacctcatggaggccatggccatgggcagcgtggaggcttcctataagtgtttagcggcagctttgatagttctgacggagtctggcaggtagggcccgaggccaggtaacgctgcaggggaccctgcctcttgcgccagctgctccaggatgcgcggcctgggcccagagcctctgctcagctgcaggaagacagccagggtggtcaggacagggcaggaccggaggctctgcctcggtaggcagcggcgtctcgggcagccggtggaggaacgccctcggggagtctggctctgctgctcccgggctgcagccctcaggctcgtgttggccttgctgggccgtgtcccccacccgctgtctgtagactcatcccctcccctctgacgaagctctgacgaagctctgtcccctctcgtccccctggacggatgttgctcccctagattgcccgtgaggcagaggctgccatctaccacttgcaattatttgaggagctccgccgcctggcgcccattaccagcgaccccacagaagccgccgccgtgggtgccgtggaggcctccttcaagtgctgcagtggggccataatcgtgctgaccaagtctggcaggtaggaggcgccagcgagcgcctggcagtgccctgctgcgggacacccctgcaggaggggcctgggagcggtcctggcgcagtgcgcagatggtgctgagccaaggtaagcccctctgcccaccacctgcaccctggcagggaaggatgctcccaggcccccgggccaggaccagcagtgagcggagccgggttgtggactcgctgcgtaaggggtagggctgacgacacatacttgttctttaaaagctgctccttggaaacagaagcagggttgccttttctggttcttaggacctctttgcaaacttagtctggaaaatctgaatgctgttcccatctctgccagacacaccagtgggttctgggcccaggaaggtcaccccccctttcccctcattttgtggggctgtggtgccggggttggacttggcctgagttaccctccctcccctgtggccctgggagacagcgggtggggaggaggggctgtagctgggctgccgtgtgcgaccaggagaggtctgcgacgcccactcttgggctttcgggctgacctttgtagaacggccggcttagctctgcactattcttagcggcagtcttctgagatttcacagctctgaccccagcagctgcctcccctgaaggccaagcctgttcctggcacctgttctgtcttcgcagcttgctcccggggcctgctccttcgcgggcccctccccagctcctgcagggcccctggcacagggctcagcagagtccccacctccagggtcccctgacaggcggagctgcccccatgcccacctttctctcatcacgaagcccttacctgaatctacacaatcttaggagttaatctttggttaaaaaatcttttttacttatttgaaaggcagagttggagaaagggagacaggtcttccccggttcacgccccgaatggctgcagcggccagggctgggccaggctgaagccgtgcccgtgtgggatgcccatgtccctatgccacaatgctggcccccccgggaatttttttttttttttttaagatttatttatttatttgaaagagttacacagagaaagaagcagagagagagaggtcttccatcggctggttcactccccaattggccacaatggccagagctgtgccagttcaaagccaggagcttctgggtctcccacgtgggtgcaggggcccaaggacttggggcgtcttccactgcttccccaggccacagcagagagctggatcggaagtggagcagccgagactcaggcagtacccttatgggatgtcggcactgtatgtggcggcttcacctgctatgccacagcactggccctttgggaattcttaagatgcctccggatcttcaagtttgtctgagatctggagcccagttgctggggccgcttgggtggacgagcccggcagtggagggtccgtgtgttgctgggcccagggtctggcagctgtgctgcggctctgcagtcaggctgtgggccacacgctgctccgctgtgcttctgccggccccaagacgaggccctgcactagggctgccaggtggagggagcaggactccctctgccatagtggaaaagtccagggtgtggcaaggcagctgccagccctggccacgtggggtcagtggatgagatggggcggctccctgccctcagaggcaccagagagcaggtgtggaagtggccagagcttcaggctggcgtagtgagttggattagggggtgagctattgccaaaggcccgggggggggggggggctccagggacactgcttaatggtcgtcctgcttgtctcctgtcccaggtctgctcaccaagtggcccggtaccgcccgcgcgcccccatcattgccgtgacacggaaccaccagaccgcccgccaggcccacctgtaccgtgggatcttcccggtggtgtgtaaggatccggtccaggaggcctgggctgaggatgtcgacctccgggtgaacttggccatgaatgtcggtgcgtgctgggagcaagactagagacctgaggggcaggatacctggctactggcttctgggaccctgaagcctggggaacataacggggggaaccccaagaccagagaggccaagagacctagctgagcacccctggtccccactggggatgttccctgaatcccactgcttgccagtcctgagcagcgcccgggaggggaggggtgccatagctcgcagccacagtaaaacgcctcgttctgtggcagtgctctgggggttaaggtttatgtcatttatttgaagggcagagtgcctgagggggagggagggaggcaaagatcttgcatccactggttcagtcctcaaataggcgcaacagccagaaggccagagtcgggcccagagctccatccgggtcccccccatggatggcagaggcaaaggcatttgggccaccttaggatgccttccctggctcgttggtaggaagcaggagcagcctggacaggggatgccggcgtggagtggcagcttaccctgctgcaccacgctgttggccccgctgcccgggtgttgggagaggagaggagcagctggctctgaccttaggcagtcttaggtcgtgtccaagtccctgcccactttgtccccgctgtgaggctggcgccaccttgtggagggtggaagtccctcaccagcttctctcctgcaggcaaggcccgcggtttcttcaagaagggagatgtggtcattgtgctcaccggatggcgccctggctctggcttcaccaacaccatgcgcgtggtgcccgtgccgtga

SEQ ID NO: 52 - DNA, 17257 bp, Gallus gallus
  source 1..17257: mol_type = genomic DNA
agcggcggcggcgctcgggcaggacaggctttgggcacggcggcggaagcagcagcaggaggtaacggcggcctccggccccgcatagggagggcgttgaaggagggcggcgagcgggcagccgaggccgccgtcgtcatcgccggacccgacgttcaggcgggagtgttacgttcaggacagggcgctgagggggcgctgaggggagggggcggctccacgcggttggggtaacggcggcgctggcggagggatggatgatggatggatgatgggtgggtgggcagatgggtgcccgcagcgctccgcccgcccgggctgagtcagccgtcggcgccacggccacgtgctggggccccgctcggctcttcccgaggctcccggctctcccagccccgaggtcccattcatccccgcgtggccgcccaccccctccccccgccccgccgcgggccctgaagcgctcggggtgcgtgaatggggcttttgttggcgcacggcggccgcccgtcggggcggcgaggcggcgtgcagagcccgcttcgtgccgctccgggtcgcacagcgctctgtggggagcgggtggtgcgcggggccgggttccggcaccctcctcctccagggacggctccagagcacgcgctggcaccgggccgttgtgtgtgtgtgggggggtaaccggagaaccgcaccccaacgcggggccgaaatcgccgccgtcagtacgcggctccaccccgttcccggagcaacgctcggacagctgcgagccgtggaggagcggccgcgcccgcgcacctcgccgtgccccggggctgagcgccgcgaccacgtgctgccctgcgcggggtgggggcggggcggggcgccgcgctgtgcggtcccgcacgtacggcatggactgctgcgggggcttcctggggtacggctccagagcgccgagctctgattgctgccgttgtgcagcgaagctgtctgacctgtgccggcgcagcccgcagcgcggtgaggctggaaggtgtgcacgcttcaactgtactctctcgataagtttgccgtctgttctttaatcgaagcctgctccgtgcccttgtcctccgagaagggcagacgcagcatagcaacacgtagtgaaggtgcccaacgctcatcctccgcctgcaggctcagtggcccatcccggccggccccacgagggctgacgttgggaggccatcttgcagttgcataaggctgcggcgcaacctccctcccgcagtaggagtcatccttcccccccccccctccacccccctctctgcaaggggggttgccgtgttggcagcctgccgtgcgcccagctccatggctgtggggtggggatggcggtggggctgtgcctctttcctgccccggggtacagctggaatgcctcagctagaggttccgtactgcaactcacactgagtcaccccgctgccctgcaacggtcctgcctttctggaacgttcagcggccaagggaggaaccggccatcttgagcgtgccctgcctgaaatggggtgataccttggggggatttaagagttacgccccgagaaaggaaagcagctttgtgcagggaatcaggctttgcaaggtattactccccgcccccttcctggctcagctcagagtgccttcaggtgttcctgagaagctgatttaaaaaacaaaaagtgggaaatgccagacattcctcttagggttataaaaccgccctttgagcatcactccttgcagatgggatgcaggaggaagcacaggaatgtagggctccctgcagcccagcagacggtggctggggtttctttcttctgctgctgcgtttcttctctccccctcgggcaaggtcacagcatcgcggcgcctctcatctccttgaatttgctgaaaaatctcaggggattctgactcaaagcagttttaactctccttcctttttatacaatcgtaaagcctttggggcttgaaaatcacacttacaaatgacttgagtgggtggggagtttccacggggaggaagctgcctccaaagcagggaggctgagttgctgcccatctttctgggaacatatgagtcgcactcagtgcttgtccctccccacaccccattccccacagccacgcgggcccttttgctgctgtttgcagaggcagtttcccagcagaggccttttgcactgtccagcaggtgctccaggatgcgccagcagatgttagagggctgaaacaccgtgttcaggtctgagcagcgggcatccccgtaataaggcgcctttgttgaggcttttatgtgggcaggctttcttgtactgactcgagtgaatcagcagcttctggcagggctgaggtgaaggacgcctgccaggacaaattatggtttctgctggcccgtctttgcgctccatttcgcgcggcgatgaggcgatggtgagtcagggctgcctgcgagaggattggcacgtaggtggggggagcggccggcgggggacctccgtgctcatgctgttcccttctgctcagcccggctttagtggagctgctggtttattaatgccctgtgcagctggaggtgcagcgtgcgcggtgagctccatttttgcgacagatggagtgcatccctgcatggctcagtgtgccgatggcgttctgagcgtgagcggcccccatccaaccgtggggagcaatgcactcgaggtggtggcagcagcacagcaagaagctgagccttgcacaggcacaaagtcctgcctcacctgggtgtaaatgcagaaggaaggtgtgcagggaggggaacatgggcagctttgagctgtattgctgcttgaggacaaaattcctcaattcccccccctcagggagggggaggaggggggggattctcccattgcagcacacgtctgtgggtttcactcagctttcttatagggtcgggaccttccctgctgttctggggatgctttcatctcgtggaggcaagcacggagtttcttactcccagaatcagccagtcttgagccctgtggaggcttcctgccccctgcaaatgcaggggctgcagtagactgcatggtgtcttcgtttggaagagggcacttgaggacattcaaggattctttagccttattcgcgtttgtctgccccggtgttgtgttcaatcaactcaagtctgattatctgtgcaggcttaaaggccgggttaagcctaggtgcacttggtgtgctgaggtgtgtccccgcgtgcagcacgcctgcagtcacgctagcagccggctgcccggtccccaggctgctggcagagatgggctgagagcgggcagggagcgggcagcgcgctgcctgtgcactgaagcgcttgaattttgttgcgttgtgtagctgcaggggaagactggcaatgaagagaagctatgaagggaacttctgcatagaaggcaggtgagctttgggtggcatccgtggggaatgacatttaaatagaagtgtgtcttgcctgtatctaagactctcaagtaggaacataacgatagtctgacccagtagcagctttcttgttacctccatattcatcacgtgggtgtgttggtgtctctgcatggtgagcgagcaggggcaatccctaaacccggcaccgttttcttctcttgcagacaccgaactccagtaaccatgtcgaagcaccacgatgcagggaccgctttcatccagacccagcagctgcacgctgccatggcagacacctttctggagcacatgtgccgcctggacatcgactccgagccaaccattgccagaaacaccggcatcatctgcaccatcggtacgtggctcccagatcccaggaactgtggctcacgttaggctgtgaacgcctgcgttggtgaaactcattgtgggaatgtgggatggcttcagccttcatccgttgccagggttgtgtcttgtctgttgaaacaacacaactttcccatgcctgcctgcccagttttattgctaggggtaaatcgagcgatgtctgaactggttgggttcagatgtgctccctaagacaaggcaaaggagctgctttcgtgttgagcaagtctgtatccactgtgcttcacttatcccctccatgtgaaacagcctgacgtttgtgggttatctcattatttatatgaagttaatattcaaactgtttcctccttttttaggcccagcctcccgctctgtggacaagctgaaggaaatgattaaatctggaatgaatgttgcccgcctcaacttctcgcacggcacccacgaggtaagggtggggatggggctgacgctgccctgaacctgtaactgctcatcccttccttcccttctgtaagaggagaccaagggaaatcgagcttgctgcagagcagctgtggtagtgtaggaagacagaactttagctttatgtgtgggggaagtgtgccagctgtaaataccaagcctgcaagactgctgataagatttcagtgctttctggtagcagcttgagttgtttccacacccttctctgtacctacctctaccttgggagtcaggtagctgatatctgaggcccttcacttctttcccccctcatgggggagtgaacttgcatttcctttacaggactgctgctcctgagttacttgtgcacccctgcaggagtacacaactatcagttgttcaccctgcttcactgttggaggctttgcaagcggtcagtgaatgaccttgtctcaaaatataaatcatttgaagtaattactgtgtgcatttgaaagaattgagcttctggacttgcctggctagccagccgctcaggttatgttcccagtagtcatgaggctggttgcttgtcacttggggggcaataaacctctaagactaaggtcagaagaaatcaacactgcttgtggtggacgagccctttttgatataaatagatgagatcctcagcaatgctgcaggagcaaaggatcccattccagggttgcccagaatgtcatccttagcgttgaggctgagttcttacgctggctgtcctccagctttcaaaatgtcaaactgtaaccaaatgacagctgtcggtggctttacttactgaactgtgagccctgaggtgcgtaacagctcgacctctcaccctcatggcctttaaaggccacagggcactgcagaaatcaaaaggtctagtgtttagctgcgcacagccgttggaccatgactgtattagcctgtggtagcagcagtgtgcaggctacagctgaccaaaattccaatgcagaaagggccgtatggaccaacttcacccctcagtggtcatgtgctgctacacagccagcaggctcagcaaagagttggtggccaagcttgaaccttcttagctgtcttagcacagtgctgtggttggtgtgcttgttagctgaggtgaagggcacggaatgtggcttggctttgggtgtagtgcagctcaattgcgtatttggtttagatggaggttaggtgatggcagttggtcatgctgcaatctcctgcctggtttgactgccagctgtaaccaggcaagtctgagttcccttctggttcctcaagcactccgttgtccgcgacggttgcctggggttttcatgtgctgcgcttgtttcagttccagtgttcatcctaaagcacatcatggggctcgagttagcaactgcaaatagatgctagaccttgaagctctttcatggaggaagtctgctttacttaagcccctgtcagcattcctgtcacatacgtaattctcaaggaagtcacactccctaggagccggtcattgatctgtatcaagtcacaaaatgccccaggtgactgggagcagataagccccatgactctgctcagatatgtttcctgtgctgcccctgttacagctgtgatgtgtggaagggccaggtgcctcttcagtctccggttgtgcccttgatcagtaaacttaattttttctgctcttgagagttaactgtggtgtatttctggctgagggcatgcgcactgacctgagcacagcttgctgcagctgcaccttcctgatcccagggcaggtgatgtgagggcagccaggtgatccaggctgcatgctgtcagcccgtgccacagagaggggaaagggcatttcttatcttgaggagcctgccaagtattgcataactctgtcaagcttatctctagtgttgtttaaactagatagcaacctgacttgatgatcatctgaagcttgtttgagatgattgggttgggaatttccttgtgaaactgtcctgaaattggcagcgctaacaaaatgctcttgcacatgtggaagggttttggactgtggtgtgcagtgacagagccttggacttccggctgcagttgctttagctctcaaaataaaatgcaaatacgtaaagggataatatcctctgagaagatcccttatccctaggggagatttgagccagagttgtaaaagtgtccacccgtatgcctgacagcagagtccttgttttggtcatgcctgtaggactgacagagggggatgaggtcgaaaggctttggggcagtgtggcaccttcacttttaagctctgaggggaagaggacagcagccgtgtgcaactgtcacttagctgtgatgctttctcagtgttcagcccttccacacatgtcagtcctccagctgctgatgttttatcatgcttgagtagtgtttgggctatgtagcacaagcagtggagtagaagtgggctgaaagtttccctttcttttccagtatcatgagggcacaattaagaacgtgcgagaggccacagagagctttgcctctgacccgatcacctacagacctgtggctattgcactggacaccaagggacctgaaatccgaactggactcatcaagggagtaagttgctctttttatttacccccacccccagtgcctgagactagatagtgttgttcccatggcctgcttttcccaaacaaggattatcttatgagaacaaaataactgtttgctgtgtctggcatttgtagcaagagtctgcacagactgctgcaagctgggggacagctgggtgcttggttttttttctgaaatccttgttttgacatgcagagtggcacagcagaggtggagctcaagaagggcgcagctctcaaagtgacgctggacaatgccttcatggagaactgcgatgagaatgtgctgtgggtggactacaagaacctcatcaaagttatagatgtgggcagcaaaatctatgtggatgacggtctcatttccttgctggttaaggagaaaggtgtgtagcaaccctcacaggatttgctagctctgcctgttcattcttccctaagaatctttgtgggtttttttttcacctccagaatggttcattaagttctgtattaaagaaattagcactgagatctaagacttactactgctgtaaaatgttgaaagtttggagaaaaaaaaagttgctcagtttggggtaattagcttgagctgacattcactagtcatccaaaaccccctcttacaggtgaatatgtgggggacacgcttatgcttatttaactatgttaattcatacttccttctcagagttgagcaaattttacaacattaaaacccagtgttttatggttagaagctgaactagctttactggtaatgcaagggaaagttaattccccagtgtttccttcatggtggttcaaaaatctgctctagcctggaggtatctcctgtaaccccagagcgtgcaggtgaggtgctgggatattatgggatgctcatgtgcagaaatgcaacctgctagttcttggagggaggcagcccccgtctcctgcatggggggaaaagaggcatctttcccaaaagcagcacagtgggtttgtgctgtgctgccatgggtacattacaagctcacatgttatgggagtccaaggagagcagtgttgtgcttctgttgtgattgctgtaaacactgtggttctcaggcagctttggcaggagggggtgtgggagctggtggtggggagctcgccctgttatgctctgactgtgggaatacatctgtttcaggcaaggactttgtcatgactgaggttgagaacggtggcatgcttggtagtaagaagggagtgaacctcccaggtgctgcggtcgacctgcctgcagtctcagagaaggacattcaggacctgaaatttggcgtggagcagaatgtggacatggtgttcgcttccttcatccgcaaagctgctgatgtccatgctgtcaggaaggtgctaggggaaaagggaaagcacatcaagattatcagcaagattgagaatcacgagggtgtgcgcaggtgagctctgcacgagtggcactgcaagtccttcactctgtagcagcagaaggacacagccctttaatgctgtagatttctgaggagctacgtcactctttgtaatgagtgtgagttcagacttcagcgttctggatgcggactggtgagctaatgcatggaatcaccaaatctgtgacaaactattagactgaaatgtctcagatgaactcttgctcctcttaatctgtttatatatagcttaagaaactgtcctatggcttgaccttgttttcctacctcctactgtggtattacagggtaccaaaatggcacttggaaggtcagtgtgccgatagggtgtctgcgcatcaggctctagagtggtgcttcctttcagaaaaggggggacagagcagagggtccaccctaagaacaaagggagtaatgaatctctcctcttgttgtcaggtttgatgagatcatggaggccagcgatggcattatggtggcccgtggtgacctgggtattgagatccctgctgaaaaagtcttcctcgcacagaagatgatgattgggcgctgcaacagggctggcaaacccatcatttgtgccactcaggtgctcagaacatgaagctagcttgtataacggctgggtgtttgtgggtctggcagggtgtgagtgtcagcagctgtccttgcacgctgaggagattctgggggcaggtccttgatggcacttgctattgggtgagaagcaagagccaggaggtgttgcggtggctgaacaactcatcttcaggcaattcagggatttaggatcttcctctttaaggtttggctgctgaaggctctaccctttgaaaccttgtactatgcgagccttttgagctgtgaccaaacacattttggggaaaaataacttgcactttgcatctcagagcagcacatctgtgcctaacaaaatgattgctctgagttgctgtgtcccaggtcttttgaaagttgcacccacgctgctgcattagaaggaactgaaaaaacaatataatcttaccttgggagctgcaagtgaggggaggaagctgatgctctgcagctttagtacagatcacaaacaggcagtccttaggcactggcagggctgactttctagaagtacttgctctgggcacaggctgtgggaatgtgtgcttctggtagggtagtgcagactgactccaggcagtcattagttgagattctggatgtaccagccacatgttcccaaccagaacagcacgttagtagcaacctcagaaagggacgggaacatgcactgcaatgcatctccaagttgagttccttggaccaagccctggtgttgggaatttgaatcagaaagtgagaatacagtatctgtggtgctgtgatttaaatcccttcagaaaaggagtaagtgtagagtggggagaaaatgttggcttttcttgctttaattcagaggttgttagggagcaggcccctcttggccctgtaactacaggagaatgctaccagaggtcaggttatatcctgacttctaccttatgtgtgtattgaggggccttagtggaaaatcagccacttcttcctgctgtatcctgactcttggacttgtttatctgcagatgttggaaagcatgatcaagaaacctcgcccgacccgcgctgagggcagtgatgttgccaatgcagttctggatggagcagactgcatcatgctgtctggggagaccgccaagggagactacccactggaggctgtgcgcatgcagcacgctgtgagtagccatcctggtgcaagtggcagtgctctgccttccatctgagtccacacataaagatgtaaatacctggtgctaggtggcagcacctagtagcataaagcagcagttctttagttcaagctctcagcatgaggtttacttcacctagtgtggaaccttggactgaatgctgtgtgctactgtcaggtggagctgagagctgctttgcagtgctcagagacctaaaacctcaattataccacattcattttgatttaaggccacggtaaaggctttttggctgctaccttggtatgaatttctttgagtgccttgtgcattttagtgttttaaatgctttaaacttgcccaaggtcaggacgctgcttgttaagcagtgtatctctagataaaaagtgttagctgactcctctgggttaggggggggagggggtgttgggggttgggagagggtgaggagaaggcttgaggtgttgcatccagcagctgatgcatttggtggtgggaatgtttgttgcacgaatgctgacagcaccatgtttttacatgcctgaactgccagaaagctctgctctgcttcatgttaacccagcttgtgtttcagttcatcaaaaatcctgatgcaaggcatctagaagtactggatttccccacttgctagggctgtgaatcacgttagtttcacctgcagctcaggtgatacatctgaaggcactgagctagcgtttctgtttgcagtgcattatctaaacatcttttacctggtcagtatggagtgctgttttctcatgtggcaaataaagcatgaccttaactactgggcagtgactgctggcttacaagctgaattctaactgtggactcaaaatgttcttccttagtctttcagtcctacattgagggctgaatcttcctaccaaggtttggaaacttagtgccatgaatattgcaattttggcctgaaagcttagttaagtacagcacaacctgtctgagtttgtggctctgtaccccccctgcatctgcaaggaacttttcctgcagggctggggataatggtgttccttctttcaggcccccagagatgaactgtaagtttttacctgctgtgcagaggagcaaatccagatttatcttgtttaatgtacataggcacaggcagcaaggtcatcagcagcactgaaaatgagcagggcctgcacctttgcaccactgaaactctcccagggttttgctgggttgctgcgtggtttcacaaagcaatcactttccagtgagctttgcagcaatgagctttctgtttcctgactggcatctctgcagcaaaagaaagtgcttggagttcggcagcaagtttccaaatgttgggcaggctgattcctacttcttggagtgggcttccttccacttccagctgacgatctgcagttgtcaaatgctgacatggcccttctgtctaacaggactatgatgtgcatagctcttccacttcctttcctctcccttccaccctccagtctcttacctgcatgccaactacaaacacttaaactatttccatgcaagtgaatccagctgtgatgtgcagcgagtaattgctatgccctcctgaagtcattgtcagagcaggcacttgggcacacacactgtgactctttacagtggggaagtagccttcccactaaactcatgttcactctgcatgtagtgcagagctacctgcctctggcctgagcgctgaaggtgtgctgcctgcctgctggtgcctgcagagtttttaggtagagaagtatactccatattccatttgacctaacagtaagtgtgaagccagcagaaacaaaactgtgtgctttaggaccagtgtgttggctctggactcgtgttcctgtctgtcacgtgtcctgttatatttagacataagacacttaactactttggcttaaactgatatcagtggaagagggataattccagcatcacaaaaaataatctattagcaccagagtagcaaaatttctattggaagagggatccaagtacagaaattccatgacaatggcactgcagtctttgctttaacatcagcttgttgtgcagtgtgtggcggctgttcaactttgggagtaaggggatgaagtgtccttgagaagatcggcctttgcatgcttttctagtctctgcaagccttctttatcaggttgtcaaggacgagtgcagccctttgagatgggaattgtcttttaatgtgagtgattctcatgctactgggatgaataaatcatgaagcttgttgcacatgctaaggctgtgaccaggaggcctttaattgtgcacctctggcctgtttgagcctggcataaattcttcactgtacgtggtgagacagccctgtaaggaaggcttgcttctgcttctttcctctgttactgaaagagaagttggtactgcacttggaggtgaaacttctttggctcttagttgaactagatgaaacctggaagggagcacatctccctttccagggaagaactaccaagaagtgaaataagcagccacaagaaagtgcagtctgaggggctatgctgtaagccctgaaaagcgagctgtgtttggttgatctgagaagagtccaaaatacagcaagagtgctgccattaaaacacctgccggctggatggtgcggggcggccagtgcagctgtgctgtgacatgcagcattgcacctgggagccaaagagtgttttgcaccctgaccttgagtatcctcttgcatgcattactatgacaattaagcaaattaacgtgatctcatgttactctggtctacctttccttcttttctttcctcttttcttctctgcccttcttcctcgggcctgcagattgctcgtgaggctgaggccgcaatgttccatcgtcagcagtttgaagaaatcttacgccacagtgtacaccacagggagcctgctgatgccatggcagcaggcgcggtggaggcctcctttaagtgcttagcagcagctctgatagttatgaccgagtctggcaggtagggctcagagaagggagaggcttaggctgcgtacggtaaccgatgctcgctcccagcatgcgggttagatatgtgctgcttgccagtcactgcgtgaatgagaccacgcttaacattgcaggcattcagcaagcagcgtgcagctctggtatctgcagggcagggccgcaggtgggtaggagcagagcaagagtgcctggcagttgggcatgaatggcaattgagtgtgggctcagcagggtgaggtgccgccaactgggggaagaaaaacttggaggatcaaattgcttgttcattaaggtggtgctgaagccaattttggctcattggcaactcttctgaaagcctgaagctggcagcagctgtgtgacacctctgtgacagagcacctcaccctgccctgtgctgagtgactgacacgagtgttgtcttcccgaatgcgtgtcgctcttctagattgcccgtgaggcggaggccgccatctttcacaggcagctgtttgaggagctgcgtcgcctgacctccctgaactgtgatcccaccgaggccgctgctgttggcgctgtggaagcgtccttcaagtgctgcagcggggccattattgtcctcaccaagtctggaaggtaggaggccgcaggctccctcgggtcacgctggctccgtgaccgagttgggggaaggagacccacacagagtgccttgaaataaagctgtagcagcggagcccgggttctggcagcgacgattccttgggaggtgacagacatgtcaaacatctgcgcatacgcagcaccgggttcccgagcctgccagcgtggcatcggcaccaggccctgcctcttcctgttctgcacagcacctctgccagcagcgctggctggaagagttggaggggaaaagtaaactgctgcaacttccaacttcttagcaagaagtcctcaagttagcagtagagttaaaagttcagcagatgcagaatcttttcctttcaatcaaggctttgtcctcttgaaggagctgtagtttggctgctaccctgctgaacctattgggtgccagcccagctcccctaagcacaagaggggtgaagagaaaatgcttaactagaactgtcaggcagggggagtgaatgcgttgcaagttcttacgggcttcctgaggccttcccgtgagtagtcctgcagaagaagcaggcatccagcttctgcactgccagctgagcccagcactgtggtttcaggtgaggtggtgatgcagactgagactatgtagggagggatttctgaccagccgtgctggtaatgctgctcttcctgttgagtgggagcgaagcacagggctgcagcctggctgtgagcagcttttccatctagagaagccatgctcaaagcgtgttggaccataagtctcaagctactttctggaagagaagctgagtatcttacctggagctggcaacagaattagtgctgcaacggaaacacgaccgtgtgcgtgctgggcatggcctacctcaagagcccttgagtgaacccttaacctaaacttacagcttagaatgatttgaagcagccactttgtgcagagatgaggaacgactacatgagcactgagttgtggatacccttctaaagtttctttgtgaaaggttccagtcttacacttgacctttttggaaagatttgaactctggtatttgaatatttttagctgtggattctgttacagccttggagtacctgtgtctgttagagctgctgggaggtttggggaggattgtctgagagctggcacgtgtgactgggggcttctgaaccaggttcttgctgagcttggctccagaaagcaaggtttgcaaaagttggcgaggctgctggcatgcgtgtcagaacgacggagctgtaaatggcatggctgtgcagcttctattcatttatttatttttgcttctacaagcagagcccaagggaggggaggctgtagttgccacccagcgcatgctgcctgctgtatggaggcactggtgtctctggtcactgcatgtgggtggcagcctccttcagagcaacccggtactccctgctctaagaccaaatgaaggcacttgcttcagtgttgctttcagcagtgcttctgcttctggggaggtgggaggatgagcacgtgggattaaatgttggctgctcctctgggacttaaacccattggtgagctgtggtcagacccatttttgggggtgactgcctttccctgacatggattctgccctctggaagcatcctcctcacagtgctgctgacacaggcttgttccacctcacaaagtgggggttgacaagaatggtggaaacttgcagggaccctgctgagattccaaggagagcttgcagaggacagaataaatggcagctagccaccttcacatagcaacagagacaaataaatgcttgtgtttggggccaaagcatgccaatggcactcaggggtgtgacagtctcccccacaagactggttagagtctgttgccttctgctcttgctcagccccattgctaaatagagaaatgagcgttgagctctgctggtgggactcggctcttgcaaaatactgtgccagcatcccctggtcccaacctggcacacttccagaagtggcagagctctccgggatgacgggctgagcctcgtgctgcaggcaggggtgcccctctggttgtgcctgcgctgcagtgccgtgcctcaccctgtgcctgttgtccttcttgcaggtctgcacacctggtgtcccggtaccgcccgcgggctcccatcatcgccgtcacccgcaatgaccaaacagcacgccaggcacacctgtaccgcggcgtcttccccgtgctgtgcaagcagccggcccacgatgcctgggcagaggatgtggatctccgtgtgaacctgggcatgaatgtcggtgagtgctgcgctcctgccttgctctgggggctgcggtggggccgcagcaggacctgcagcacggtgttggcgctgctgttgggatgcgggggaccgaggttgcataagcgcaccgcatctaagggggaaatgggacggtggttttgtcacagcagccttgaaatagctgcctgcagaaaaccaggtttgctttaataagcgagagaaggggggtgtgaggagacctgagtgctgacgctttttcctccttttccccaggcaaagcccgtggattcttcaagaccggggacctggtgatcgtgctgacgggctggcgccccggctccggctacaccaacaccatgcgggtggtgcccgtgccatgagctgccggcgcccccgcctcttccacccccgcgccccttccccatgcattaggccagcagtcgcttgcgatgttctcctcgtccctagcgtggatttaggttgctcaacaccaccgagtgcagcagcgggggaagagaccttaaatcactcagaatacttgaagtgtgtagttgtttccagacctctctccctgtagttagccatgtcagagcggggccctgtgctccccacgtccccctagcttagcactgccctcagggtgtggctgtgcctctccttgccccggctgtggggtgatggggaaggagtgggcgcttatggtaacggttctgctgctgcacaccgcagctcccccaggttggtgccctctgtccatgaggggttataggttgtcccctctgggcttacactccaaaggtgtgctccacggttcctacaagaatcccaaaccagggtgctgctcgttgtccccctgggtgcaggtggagtcctgcgggttgttggggtgcaggatgggtgggggacgcacgcttcatctccatgcagccacctgcccttctatggtgtctgtctgtgttgtcgcctttcctgagagcgttccttttgtctgggcgccctgtttgctccggtgggtgtattgggttgtggagcaccactgtgcacgtcaataaagagctgagcacccctgc

SEQ ID NO: 53 - DNA, 60974 bp, Sus scrofa
  source 1..60974: mol_type = genomic DNA
cagagtggctcagcgcggccgtgacggaacgacgaggcgtgcgggtgcgcgacctctcccgcccggccccggcccccgccccttggcctgccgctccgcccccgcggggcgtcccgggccgctcgtcccctcagcggcggccgcgcgcggcccggggtcgccgtgagtactcgcgaggccagcttgtggggcggccgccacggggacggggcggggcggccgagccgcgggaagggtgcggggccagggtggaggaagtgaagggtttggacccggtggctctgagaggccttctgagtttaacaggcttcgcttatggccgggcggctggcgacacagatcgggctcagagcttcttccccgggaggcaggtgttgggtgtcttcggggtctccgggggaaaccggagaagagctctaagtgcttccccaagggaggcctcagtgagggcctgttctggaagcacgagtggcggccaatgttgggggctcaccccggcctcttcttccgccgggtacacgagatagcgctgggaagcgggcatcgccgggcggtgggttctcccccgcactctttgttccatggcctaggctttttgaaaggagcacttcgctgcagagcggagtcaccttctttcccaggaccgccaagccgggatcaaacgttgagatcggaggcttctttgactagatgcaaagttgtcacgcggagaagcgcttttggacctaggccaggacatttgtgatccggacttgatgcttatttacttttggcctattcttatgcaaattttttgttagctgctctctagcgctgacacccttctctgggcattcttcatacagtcagtatttgacttggcttttcttgaaagggttcccttgattacctcgtttagataaatcaaataggtctttcttactagttcattaacagctcccttaattgggttgtaatctcatcactggaagagtggatgtcattagaatctgacaaatgtgctatagtccccagttccactgtacgctattatacttctgaccctaagcaagttgcttgacgtccctgcacctgcatactctatctgtagtgtgaaaagtttaaaataggttatatacagcctagtttgaaaaagatggtagtctctaaattgtttcttcactggacaggaaaaattctctctgaactacttttttctccttgtggtaacagcagaaaccaagcagcaacagcccttggagagagactaagctttcttgacctctacagtggcacagtggcaaagagcttgagaaagttggcacttctgggctacaccattgtcatccaggacattaatgcctccctctttgttcctctgagaccgtccagatcagcacctgtttattttcaaatctcacaaaccccaccccagatcttcatagtttggttctcagctgctcttgaaggtgaatggagtcttgctttttaaaatcatttttgtatggttttgtctatgtgtagatgtgtttaggttcttgtatttatctcccctttggcttaggtcacagttcattcattcattcactcattcaattagctatgttatgagggactgacagtacagacagtgaaaatgctcaaagcctctgccaccgtagagcttacattctgatgggaggtaaatacaataaacaaacattttgtcatagagtggtaagtgctgtaggaaaaaataaagcaaggaaagggtttggagagtgctagtgtgttgtgtattgtgattttactgttttatgtaggatagtcaaggtgatcttcaagcagagatctgaaagagtaaaggagcaagcccagtgaatatgggtgaaaagcattccaagaagagtggcaagcaggcacaaagaccctgtggtgggaatatgcttgatatgttggaagaacagcaagttagagagtttcagtgatactcttcatttcacatttagagttgataggagactccttaaaaagtatgaaagtagcctctgggtttgtttgtttttctttttagaatgaaaatttccaactatatacaaaagtagagagtttacaaacccttgtgtacttatcactcagcttcgtttgtaatcaacattttatgattttcatctgaacatttcatctgttccccattttatttgtgctggaatagtattttaaagcaaatcttggcgttcccatcgtggtgcagtggaaacgaatccgactaggaaccatgaggttgtgggttctatccctgcccttgctcagtgggttaaggatctggtgttgccgtgagctgtggtgtagttcgaagacatggctcggatctggcgtggctgtggctgtggtgtaggccagtggttacagctccaattggacccctagcctgggaacctcaatatgctgcaggtgtggccttaaaaagacaataaaaaaggaaatatttaaattaagatttgaatatttaaagatatcaagcatttcatctctaagaacttcagctgcagtttccttgtgcattttgtttgtaatgggtcaggcagcactgttggctgaaggactgctatgttgtttgccctggcattgtgctttaggtccccattctgggtcagttaccaggtttaaattggtatccgaggaacagaccacaagcccttggaacacacaaggatttagaacagtggtctcagattttatcattcatcagaataacttggagggcttgttaaaacatcccatctctgaagtttctagttcagtaggtttcagtatgaggtctgagaatttactcttttttttttttttttttgccttttaggaacacacctggagcccatggaagctccctggctaggggtcgaattggagctccagcttctggcctatgccactgccacagcaacatgggatcccagccatgtctgcaacctacactgcggctcatggcaacaccagatccttaatttactgagcgtggccaggaattgaacccacatcctcatggatactagttgggtttgtttctgctgagccacaaaaggaactccatgaatttacatttttaacaagttcccaggtgatattgattctgcagatccacagaaccaaggatctagtaacctaagattcaatagaaccatgttacagaccaagaacatctctctttaaggttgggatggatgaaactgagataaatgtagagccaagaaggccaaggacagtggtttttagttttgtgaaagggctaggagttagtgtttttctgagcatttctttggatcattattacattaattggggagtgggttccaagatgagtccatggctttattttgactatgacaactcagactatgagtaccagagaatggtcagtgttgttgaggactttttaacttggctctgattgttatatgatgtctttgagggatgctagtgttaaataagagattatgactaaatcaccaggggtaaagactccaaaagagtctgcttctgatgggcgttggtactatgaagaatattctaaaatgaactgactagactgagtagcataagcgtctggttctgccaccatcacttgtggcgtcagaggtttgatgcagcattgtgaactagagcaaggacgtgattcaatcccagccttccagcttaattggaacagtagtatcttatgtttgtatagtgctttcatatttcattatctttaaagaactgtgcgtacatgcattcaagtatgtcttcttatgtgcaggtgcctacatgtgtataataaaaatatatgctaacgtgtacctgtgtatatgccccctagctgttaagtgaggatgaaatcagttgttttctactttccataggacagcgagtgacttattaccaccaccacaacagagcctgccttctccagcagatctccagtcagctcccgacatcatccccacccccttctcccttgtggcctcctaaatgcccatctcgttagcctgggttcatccggtggtatggaggggtgctgcctagcactgacctggaagtgtgtgtgaccctctgacccaatggtgagaactgattgcccacttcttcaactgatcactgggggcagatggaacagactgcagaccaccttttcctagtattttccttcccaccctttgggaaagtagagtggctatttgaagttagagagaaggggcatgggaacagtcttacttggtgtctacagagggagttttcttgggaaggaaggggagagttaaatacttaagggataaaaaaagggaattatgagacgaaagagtttaggtaggctggggagctgccttaggtaatgaccctagaagtggccagtatgtacttgaacctagatatcctagaaattaactctgacatcaaagtgctcctcaccctcagtcatcataccagtatttaaagaaggccaaaattccctgttgttttcttccatgccaccaagtcagtgatgggccatggatgtgaccagatttaggaagagactgagacccttccaaaagataaggtgaagggaacaaggggattagggcttcagttattctgttccaagagacctcccgttctttgtggaaatctgtttcccatcttaatttgactgtgttccctggaataacacggacccagcaaagcattgtacccttatttcttattaggacatcaaaggccagttctggaatgatgatgattcggagggagataatgaatcagaggaatttctctatggagttcaggtgaggtcctagcccccaaaacccatgttgcatgatctaaatgtgtgtttctctcttgatacattggaagtgaaaaagattgttttggagcagagataagatgaagacctaatgttggtgtggtctgaggattgtcatgttagagctaatttcatatctttgctcttgcctgggactcatacttttcccagggtaaactgcatcctctgtatgtggagcttactcatatgtttagttggaaataattattttctgactatttcctcgctagccagtgggagtaattttattcattagcatcagaattttaacagattattctgccagtgattggttaatgctgtcactgtccagtgcatgatcctgaacaggaatgttggacttttaaagtcagaaccaatctgatttaaattctgtactcttcagcatgttctgagttctcatagaaaagatacctaagtatcctggtcaaggtctgagagaagagttgagacttatggatgtgagctccttaagcaaagccctcagttggtctactgaaaattcttagagtggtgatggagatccccatcacctgtctctcagatccccagcaagagccaccaccaagagcaccttcatataagcagctcttgcttatatgtattgctgtagtttgattttcctgttcctgatgtttcttcatgttctattttcatctatttccttctccctgtatctttcctctctccactgtttttgctgctgactcaggggagctgtgcagctgacctctatcgacacccacagcttgatgcagacattgaagccgtgaaggagatctacagtgagaactctgtattcatcaggtgggacttgactttctgggatggcaagggaatcaaccacatctttccaagttgttaaaatgactagaggttttcttctttatggtgtgatgagttgggtgcggtctgtggatattgatgagctacgagcttaatgatagtgggagtggctcagaatatttaaggttggctgacaggaataaatagggaagtgcagccacttttcctagaagttccctcttatcctagactcctctaggctgtttcccctttaagataagaagggcttctggcctggagaagaccttgatcataaaagtgggatgaggctatagctaagagcctaaatacagagttgccttccacccttccctttcccttgtccccaggctcagttctctctccacttcagtctctgttcccttctttctcccttaatgagtctcacctatggtcacctgtatctctatagagaatatggaactatcgatgacgtggacattgacctccacatcaacatcagcttcctcgatgtaagtggtgctagtagctgccaacaacatgtctgttgggcttgtggattggagtttgacttgtaggcttagaatcttgtttctgagtactgtattcagagcacatcctcttcatccttgtctttccttgaccatcagcagacagtggtgtgtgcatgtgtgtcatatagggaaaatgtcaagtccattcctctctgttgccacactggaaactgtttcccttactcttttaggaggaagtttctacagcctggaaggtcctccggacagaacctattgtgttgaggctgcgattttctctctcccagtacctcgatggaccaggtaaaggcagtgacttcaggcaagtgggtgctttgaatgtggtctagtccaaaatcttaagttaactgaggtagagaacatcccttaaaggccctttttattaatgaaattgttttctctagtccctggatattatttattccttctttcccctcaacttctctaaggaaatatttgaaattggtcagttagttcccagattctaccttctgcctcgatgttttccttctccatcattggtgtttagagcatccttagaatctgaggaagctaatccactgaactggagctgtggactcccaagaggatagacacgcatatgtaggagctttctcttctctttgcagaaccatcgattgaagttttccagccatcgaataaggaaggatttgggttgggtcttcagttgaagaagtaagaacttttttttttttttttttttttttaatcttgttttaggggtgcacccacagcatatggagattcccagtctaagggtcaaattggagctgtagccgccagcctctgccacaggcacagcaacctgggatccgagcacatctgtgacctataccacagcacacggtaacactggatccttaaggcactgatcaaggccagggattgaacctgtatcttcatggatactagttggttttgttaactgctaagccatgatgggaactccaggaaaagtaggaactttgatctttctggttatgaactttttttttgcttttcagggccacactttcgacatatggaagtttccaggctaggggttgaattggagctacaggtattggcctatgctacagccacagcagcatgggatctgagctctgtctgtaacctgcaccacagctcatggcaatgccagatccttaaccccactgagcaaggccagggattgaacaggtgtcttatagatactggttgagtttgtttctgctgagccacaacagaaaccccttggttatgaacatttgaggggaagagatgtggatatgtggatggacagacagaaatctcaaatagattatttctttccaaaaatgtatttctgagatatgcccaagaaagagtaaaattaatttgggccaaatttctggttgtgtggggaagcttatgtttttggggtatctgcccttcagacttcttggaggtgagaagataggcttgaggaaaagcacaaccctggaatttgagtcatgctgatcacatttcctgtgtggtttactcagagtagggctctaggagctcagagagttttagctattgaatgtgtggggcaccttactaagtatttggtatacaaagagaagtgaattatagattctcccctcagtcacgttccaaatagagtccagtggatacatgttctaatacaagtgaatatgtaaggtttagaaaggacacagaggaagttataactcagtctggtggcaggtgaatgaatgaaagatgaaaggaggtaattaatggttttcaagaatgggtaagagttgcccatgaagacagtttctgagggtaacatttgcaagggcacagtcatgtgtaggaagcttaaagtagttatatattgctggtaaaatgcaaagggagtaggatctaaagctaaagaagtactaatgagtccaaatcagagaggatcttgaaattttattttgtaggcagtgggaagattgaagggttttaggcaagaaaaggacttgatcagttttagttttaaaaaaatcattttggtagcattgtgcaaagatagaaaggatgagacaagagaccaaaactatttttgtaggcgacatttgttggagtccatccatgagatgatgtaaacctgagctaggtatgactctaggtaagaagaaaggctagagagattatttgcagggtataaaattcataagacttgatgactgagttgggtatattatacatgacaagagccaaggagtagtggaatattactcatgcgtttccagcctggatgtatattgtaataccagtaatctaaataggggcttaagaagagtaagttcataggatgaagattaataattagttccattttaaacatgtagtttaaaggtaactttgggttattcaagtggagaagctcagtagagagatatatagatttgatacttaaaggagaggttcaggttgaagattcgtattagttataagggtcagaattgcagctatcatagaatatggtgctcagagattgctaagggagagcatgatagaatgagaagaaaatagaatcaagggcaaaacaccctttaacatccagacagaatgccaaaagtaggtagagggagagaaacactcaaggtaggacagagaagttggaaaagaacgggaagagaatagtgtcattgaaggcaaggaggagtgtttctacaaggagggccaaaatttctgtttgatttaacatcaaagtgagagcaattgtagcgcagtggtgaaggtagaaaccagactgtactaagtaaaaattcgtcattggcaataaaagagaaacagtaagtatgggttgagaaatttggttgcagataggagaggaaaagagaggtagctaggctgaggtgccaaaagttgaaactttttatagacttgggtaaaggaactgacaggaaagggggatggttgacagatcaaggtccctatagagggtctgagatctagggtacaaatagagcaatttgtccttaataggagggtcaccttgtcttcggagaatggagggatcaaggtaagggtagaaatggattttgtggtagggttgcaaaattttcagttttctttactgagaagaaaggtcattagttgtagggttgtaggtgaagttaggagcttaaaggagatgataggatttagaataacagtgtagaatgatatgagagagaactgatcaaagagaagtgaaaggattgacaagaccagctgagggcccagctcaagttggaaaccataaatttagggatgtaattttttgcccaacaaagtcagtacccctcgtgtcccttgtctttttcttcactgttcacaaggcagtgggtggtactcactatcccaccacttttgttccactctcagtatagttattcttggcaataaattgccaggttcacagccctggaagacacacctctaaaggcagaggatctgagaatttctctggtccattagatgaggcctgctagctagtgggtatatacccactgaggtaaagaggtactttaggacagtacatttccatgtgctaaggttttaacaggggggttctttagatccacattcccttgtaacaactagcttagtggtccctgggtctgaattttagattagttgccttttgtgatcctggtcccccaacccccagatcgggaaacatgtaagatgaagacaggatttcattaccatcttactgcaattttcttttctttctttctttttttttttttttttttttgtctttttgccttttctagggctgctccagcagcatatggaggttcccaggttaggggtcgaatcggagccatagccctaggcctatgccagagccacagcaacgggggatctgagctgcatctgcaacctacaccacagctcactgcgatgctgaatccttagcccactgagcaaggccagggatcgaacctgtaaccttgtggttcctagtcagatttgttaaccactgagccacgacgggaactccttactgcaattttcttatgtgttactagcctacatacctcatgttttctgtaatcacgcagatatgaatagaagtttgaatgattaaataaatgaaaagttcatttactgcagagaatcatttcacaaggcatcacagtcctatgtcttgatcacttccctgcctgggcttccttaagcctgttcctctttgattccttaggatcctgggtatgtttacatcccaacaatggaaacatctcagcaatgatttcctcaagacccagcaggaaaagaggcatagctggttcaaggcaagtggtaccatcaagaagttccgagctggcctcagcatcttctcacccatccccaagtaagtgttcctccataagctgattatctcttatcaccttaatctcctctaagcccctacctcaccttgattatgtaaacttacatattctttcctctcctcaggtcctctgaattctgtgtctttaattattaacaccatccacatggtcctgtcccctatgtgtctttacccttttactcctggcccagtagtcataccttgtcctttatgtaaaacccagtacctagctcatggtatgtatttggagaaatgtatgctaatctctccacctatcctgtctttgaccctcatccttattccctatgctaaggaggcagagtcagagcaccaaatccttgcttttctggaaggatacactgttttattatctaggttctagaaataaattctgctccattctttgaccccaggtctacattttcccctccctggtgccttctgatcttttcagaggcaaaaacaaacccagattgtagttttccttacacaattattgtcagtcaaacattaggctctttggggtccagctgcctcttgcctagtttaatcacatatatacaaatgcatgcgtgtgtgcacacacacacacagtaggtgaactaggcatcctctccttacctggcctagatgccgtagagattgacctaggctttgcctctgcctgcaacttcatgttctaatcttaagaacacacagataatgacataagatgtctggtgatctccttgttttgacaatttgatccaatgaaactttcctcagatatgagttgctctttgacttagcttgtttggtactttcttatctccaggcctcttccagactcatactgaatccccagtttcatcacttctctttactttctcttcaggtctcccagcttccctatcatacaggactccatgctgaaaggcaaactgggtgtaccagagcttcgggttggacgcctcatgaaccgttccatctcctgcaccatgaagaaccccaaagtggaggtgtttggctaccctcccagcccccaggcaggtctcctctgcccccagcacgcaggcctccctcccccagcacggacctctcctttggtacatgggctgagttagggtttgacttgggtatcagaggatgggaggggaaatgtgggaatatgatttctcaagatgaggtggggatctggttgtcaggtatcaaaaaccatgacatgaatcatgagggagtagaatgaagcacatttagtggttgtgaactcatatttaaacagtctggcaagcttctctgatggtaggcaaagagtctggggttgggcagctcagggaaatgtcatgtgtaatattattaggttgtggttaggacttggtagtcagctgctatagagacctattccacgtactttggagagggttaaggacaggtgagcctttttcaagagcctttggccaaaatgtctgaaggtcttctctccagcttggggactacttagactgatcagagctagggcatagctactttaggagaattttaggtaatgggtatgagtgaggtctttctaggcaccagcttcataggaggctcatgggatttgtctttttaatctcatcaggtcagtggtcactgcaagaacatccccactctggaatatggattccttgttcaggtaatcaagtctaaagcttcatgcataagcctttcccgttttctgacccatctaattccccaatacctcccagcttttcagtcagttagcacacatttcctgaattgctctatttctagaactaagtaactgttgttcagataggaaaaaagaaggcacagttcccatcttcaagaagcttgtagcattactgggaagacaacactcacagactacttacaatctaacgtcaaatgaggcactgcaaattttaagtgcacaggtgccaagatgagagttaaaaatgaagctaattttcttagattcccacacatccttgctcatgtgatctttacatttcctcacaaactttccctgcttgaccaccactggtcgcttgcttctttttaaaacctttttactttctggacacagtttcacttttgcatctgcaattcctttgttcatgtcattccctccagtggccttcttccctgtgtctgcctgttgcaatcaaagtctctgatgccaccttttctataaaatcttcccttttacctgaaatatattagctggaaatatgtctcctctcactgaactcacataacactttttgttttatatggcactgatgtgtcatgtgccacatgctgccttggattgtagttaatttgtgtacataccacatctctcctgttggactatagtgtctgagaaaacttttgttactgagtaatcaaggagacctttcaccaagttaggtacatagtaggcattcagtaaatccacattaaaggaatgagtaaaagcccttgtatgtccatttactataccaaaagtttgtgaaatggaaacagtttgatttatacacaaagtatatacgtggttggaatagccatgttaagacagtgtgtaaaaaaatacgttagaatattgctgcatgagctctggatttgtgctgtctgcccagcatagggttttatctatgatattgatcagtttacaggcagattgttcttggtagtaaaaatggctcttctggccccctgacatctaatcatggactgaatactcgtttgccacacagacacagtctttaatgactacagttaaggttaatagtttgattagtacctgtaaatgtttatgaagtatttcaagattcatctcattactcttgatacctctttgaagtgaataatgtgcactttgtaaatgaggaaaccaaggctcaggttaactaaattgcccaaagttatatatgatattaagtagcggaggaggggagttcccattgtggctcagaggaaatgaatctgactagtatccatgaggatgcaggttcaatccctggccttgctcaagtaggttaaggatccagtgttgctgtgagctgtggtgtaggtcgcagatgctgctcgaatctggcattgctgtggctgtggtgtaggccagcggctacacctccgattctacctctagcctgggaacctccatatgccacgagtatggccctaaaaagaaaatagatcgatagattgatagataaagatcagtcaaaaaatcccatactaaagaaaaaatagtagaggagggatcttaaccaaagtcatctaccatgtctggtcttgtttttactataacattcagtcttagctctagagtcaggccatcagtttaactaagtgtagaacctaagaatagattctaccttacttacatgtctgggaggtaggagagtaacagaaagaagtttaaattatttcctgtatttgggtctgtacttctgaaagacttaatagaatctcttctccctccagatcatgaaatatgcagagcagaggattccaacattgaatgagtactgtgtagtgtgtgatgagcagcatgtctttcagaatgggtcaatgctcaaggtaaatgcctcaaatgtgaccctcttccttagacccatggctaagaccgcttcagacctcatctctattttcctttcttcctgggtctgtggtaaaggtagaattctgtctctactccttgagactttagaaatctcatcaatgctacttggaaagataaggattatcattctagagttgtctgaggtcaagattgtccctcactccttaccttcttattgcacacctcttaccaccaccatctcttaatatttccattcagccagctgtttgtactcgcgaactgtgtgttttctccttctacacattgggagtcatgtctggagctgcagaggaggtggccactggagcagaggtatgggggagagaaaagctgctggggacccaggatcaagaaagaccaatgaacctagcagtttcaccgcttggaatttaatgtccagaaatgctttcctatgtcaggatattgactgtaggattgtttataacagcttttttaaaaatggatacaggcccatctgaatgaagtccatttaagttatggtattttcttacactggaaatactatacagctgttatttatggatagaaaattacatataaaagaaaatacaaccgtatgtatagcacagttcttttttgttgtatttcctatataataactatttctctaggaaaactatctggaagagtatatatcaaacttagctgtagttacctgtgaggaatggaatttggggatgaggggatggtttctatctttttaaaatcactcttttaaatattattcttattaggagtttatattttctaatgagaaaatgatacacatatttgcttttgaaaaaaatgctgagagcctccataaggtagacctatgctggctactgtgtgactcccagcactgggaaatcatttcgcttctcatctctttctctccttttccctaaggtggtggatctgctggtggccatgtgtagggcagctctggagtcccctagaaagagcatcatctttgagccttatccttctgtggtggaccccactgatcccaagactctggcctttaaccctaaggtatagttgcttgggaagaaagtcaggacaaaggaacagaaatgctaaagataatgggagactatgggctatcattctagtgtacacaacaggttggaataaggggaactcttaaaccctaaactttggttgttttagtgaataaataagtaaacataacttgcaatataacagaaacaataacgtctgagtttgtcagaagaaagggagaagagttaatattagttttgtcgttataggaaagaatatagactttgattcaagacacactatgattccgtttaggttccgttgcatcctagcttaatggtgttaggcaaatcactgaatatgtctgaacctcagtttccttataatgcctattataaaaataagagataacataaaatactatattagtaagggttctctagacaaacagaaccagtagttaatatatggagatatttattgtaagaaattggcttatgtgattatgaagactgagaataccaagatctgtggtgggcaagctggagattcagaagagctgacggtatagggtccaacccaaaagccagcaggttcaacatctaagaagagctcatttttctgtttcagtttgaaagcagggaaaaaaaaaaccaatgtctcagctctaggcagtcaggcaagaggagttccttcttactcagctgttgtgttctattcaggccttcagctgatttagatgaggcccatctgaagagggaggggtatccgctttcctcagtctaccctcaaacgtttatcccatctagaaatgccttaacagacacacccagaataatgtttgagcaaatgtttgggcagtccatggcccagtcaaattagcacaattaacagttgacaaaattaaccatcatgtgttcccagcagagtgcttcactattactcattgctgtgtatcaggcactataccaggttctttacatatgttaatcttcagaacatctctatgagaatagtgggattttgtgttttcacttacacagagatactgaggtcctaagaggcatataacttaccctattatgctcatttggtaaaagagaacatacatactattctagaacttcctgacactaaatatatgttctttccatgaaatatgctgtttcccaagggcctagtcttcttttgtacatgtcaccctcctttacatcacatctcactctcttcctcttttcttcacctggttcttctcttcccattgattccttgcagaagaagaattatgagcgacttcaaaaagctctggatagtgtgatgtccatccgggagatgacccaggtattctgccctttgcctgtcccctgtgttatgcctgccggctcatgtgccctattctgcttggaattcatgggagtggaatgaagatttagaaatgttactcaaatctctcactgtattttctttagtcttccctttttagccttcccagtctttgttctctgttttgttagctctgttacacctctttctctctcctatgtctctcttaacctttacctttattgctgatttattcgcaagttgttcatatttaatgtttttcccataattcttagggcatcagagaggtgagggaaagccctttgcccagggttgggagggtgaggaggggggcttggaggtaaggaactagatccaggggcctacatcttctatccttctaggggtggggcagccacatggagcagtgatcatcctttgactcaaggctgtccctcaccagacttccccacccctcgtctgcaatctctgcatgaatagacattcatttgtactcacattgacaggtgggcctcttctcagcaaatcctatatatcacaattcagattaacacataagatacacctaggagggataatttacttgtcggaagaattctttgctttatgtcaagttttaccacaggattccttagcctaatctttctgcgacattgattttgtttatttttttgatttatagtaaactttttagggctgtaggaattgcataaaatgagggagacccacctgcatgttgttttgtaagcatcttcagtcattttcatatttatttatttattagtctttgcaacctgactggggtccttgagggtgggaaccccatctcctcttgccttggtgtggccttcagtgcctaccttgccctgggctgatagtctttggaaaatgggagggctcccacctctcctgggagataaccttatggctgttgtcttacagggctcatatctggaaatcaagaagcagatggacaagctggatcccctggcccatcctctcctgcagtggtacgagtagaatggttcatccctcagcgggtgggggagtagggcatagtaccagatgtggagccttctcttcttggcatctttctcttagaggtggtttcagcatccttcccaaggttatatattttatggctgctttttcttggggctttattgatacacctggacttgacaagaactcagatcctgacaaagagcccacaggttctgacatgatgcctcaagttttctgtgggtgtttcctgtttcctagtaggcttttgagtgctgcttacatgtaggaattaatgctgttgaattcaattctttttttatgcatgcagagagatctgtctccctatttctactccctttccctatttcaagcccttccctgttgttgactggttgactggccagagccttttgaagcactatcattacccccgcttccttgcaaggttctggcccttctctactttcctctacccccctgctctctccctgattttgattccgttctctattcctgacagagctttccccttctagactactctctgtctggttagcctttgacattagtactttctcagtgcaacatgctctttcttagcatgaggaatggccaggcatagaccatccaggatggcatcacaggtactggcatgaagcaccaaaaaccaaaccatatccataggagataatgctcccttgctgagccattaatggtttactcctttgttctctcctgtcatctgagttcagcctacatacagctgagtctgtccctgaatcctcaggaaatttgggctaatttcttctgtttctttttccagcccaaagagtaacttttcatcttccccatctcggaggcttcaggcttcctctcaggataaccctgagccctagttctaaatttctaaatcccaagtgtcagttgtcacatcgtaatacagaccccacctttctagtttctttttttttggtagcttacctcttgttcatttctttttttgtttttactttttacgactgtacctatggcatatggaagttcctgagccagggatcaaatctgagctgcagctggggactgtgccataactatagcaacaccagatccgagctgcatatgcaacatatgccacagcttgtgacaatgctggacctgtaacccacaaagtgatgccagggatcaaacttatatcctcattcagacaatgtttggtccttaacccattgagccacagtggaaactctttgttcacttcctttcattgtattctttagtctccctagatataggcagcccatttcccacctctctctttcttttatccattttttaaactaccttgtaactagctagtctctggtcagtaacttcactttttttaagtttttatacacaaacactacccgtaggaatatccgtatagtgacctggacttaattcacacagccctggaatctttgcttgttctcgcagaggcagagcaagctgctactttgttcagagtggcctaaccacacacttattctatcctttttgtttttactctgttttgtcttaggatcatctctagcaacaggtcacacattgtcaaactacctctcagcagggtaagtgactgttctcccgctaaactctttaaatttctggtgagaggcagggaggggctccaaaccagctaaccacattagtcctagacctgcctggttgtcttgtccatcagtaatagtctatgcgcctaagtatatatcctttatttggggtcagtacaagcctgatttgctccctttccccagcccttccctccctgatctcgatttctgttctgttttcctgaccacccccccaccctgggcagcagctgaagttcatgcacacctcacaccagttcctcctgctgagcagccctcctgctaaggaggctcggttccggaccgccaagaagctctatggcagcacctttgccttccagtgaggagtgggggggggctgggcatgggtgctggatgaggcaaaaataatatagggggatgggagggtaggttctcttgaggaatacgagtttatgttacactatgtccatcaaccagtcagtgaacaaatacttattaaatgctattgtgataggtgttgtagagaatagagatacataagataaagtcattactctcaagctacttacagtgttgttgaggagaaatgatatttacatataaaaagatgtggttgaaagaacagattggcaggggtagatttgaaagctactttggaggaaatcactttatgcctaattgtaggaggagagagagggggaaaaaaagagagattaaagataatttcaaggggagtcagtggtacccttccttaggatagatgtgaagtcatgactggaagccatatttggaatcagtgataagcttgttgaattcaagatgattttgtttaatttgaaatagcaggataatagacaaatgaaaatattgaaccagcagttgaggatttaggatgggactagccaaagcacagaaaagagggtgggactgaatattagccacacacacacacatcttgcatgcctgtgatgacacccatgtgcatgtccattgctaggtgtatctgtgtccatcttcatgtcctgcgtgggtgagaatgggtgggagtggcagtcttggaagtgtttttgagaacctcataccatgaggagacatatattctaccctgccttgcagtgggtcccacattgagaactggcattcgatcctgcgcaatgggctggtcaatgcatcctacaccaaactgcaggtgaggctgtgccccttttccctttccactccctgccccctgccccactcagttttggggaagtctgccctagagtacccagctgttttttgcttcagcatcctcaaacctagcctcccgtgaggcttccctagggaggaagttgcagccacgtagaatgaatgtgactgttgtatatttgaccttaagctgtgctttttggtgttgtaggaatgggaatgtagctctaggcaggaatcagctgtaggctgattgcagccactgtgatcaattaacctgttagaacttttctatttatttaactgttcattcagagagttgatataggagacatatttctttgctctgcaagactctaaagaatggttattgtccctcatcctggctgctgtcctatattcctttttggggggtggtaatctgcatatgtcccaaggcctagggacaggagcatggattctgagggaatggattcaacggctctataactcctctccaaggcatcagtatctagtgtttcataggcaaagggttaacctgcatccacttttcagccaagcagcttttcaagagtaggtgcctaagattatctgaatgagtgagaagataaaatggggggacagagggctggataagccttgtggctgcctaatatacagccatgggagggcccaggctcatattgctcatcctggtgtttccctgcaagctgcatggagcagcctatggcaaaggcatctacctgagccccatctccagtatttcctttggatactcaggtaagaaataatctctggccacgttgactctatttacattgcatccattttgtgaatacataatcctatgctgtctcctccatcatggatacttggtctatgtcagttctctccccagtcatggatacataatcttctctactttctctccttagaaatttagattctttgcaaagtgaggccatgtctccctttctcttagatagtcctcttcttcatggggatggttttattcactggagaggcttggctctgagaatgggttatggaataactggtagatgaaactaggctcttttggagaacacatttttggtagtagcctgtcattacaggttgtgtgtgtgtacacatgcgtatcggtttatatatgaaagcttgagtcccttccattggcttatatctctggagacattgttcttctccctgtcaaccaccttttacaatttaaactgtctgtaattacacactattgtattagtcactttaaatataagagaaagcattttatttcaaagataggcatgaacaagtttcatagtttagttaggagatgagttaagctttgtccagttcagaaaatgtatctgacttagagctctgtctttgttccaggtttatgtctgagttccttttcagtcttcttttgttcttattctgtatcgtctgtgtataaaagatagccaccacgtagctgcatgtctaccgtatgaaggggacagtgctagatcctttatagattactatttagtattctccagtaattataactgttacaaagataaggaaacttgggtccagaaacgcaaaaccagaaaattccctaataataggataaaccaaacagtagaagaaaggagataatacaggtaagagccaaaatcagtgaaataaaaacaggtatcaataggagttcccactgtggcagagtgggattgacagtgtctctgtggtgccaggatgcaggtttgatcctctgcctggcacagtgggttgaaggatccggcattactgtagctctggcgtaggtcacagctgcagttcagatctgatccctggcccaggaactccacatgcctcggggtggtgaaaaaagaaaagccagatatattagataggtccaaaaaagccaaaagatgattcttttaaagaactaaccagacagataaacctggttagactgattaagaagaaaaagcacaattaataaaaagtacgattaaataatgaagaaaggaacataactatcgatgtagcagaattttaaaagttaatggtggaaatccatcagctttatgccagtgatttgaaacctttgtcaaaatggatgaatccctagaaaaatataacaacaaaactgattcaagaagaaatagtctgaatagctctaaaattataaaagacattgaaatagtagtttaaaattttctacaacaaaacagaaaaagttaccaacccctgataattttacctagtgagtcttaagtcaaactttcaaagaacatattatcccaatcttaaacaaaatcatccagagaatagaaagaaatggaattgcaaatcataataaattgataaatttaactttattaacactaaaatcttcatcagaagacaccttaagaaagtaaaaagttaagttacagagcaggagaaaacatttgcaacacatataaccgcaaaagattagtagtaaaaatgtgttaaaaaaaaattctttcaggagttcccattgtggctcaacgaaaatgaatgtgactagtatccacgaggatgcaggttcgattcctggcctctcagtgggttaaggatctggcattgccatgagctgtggtgtaggtcgcagacacatttggcgtggctgtggctgtggctatggctatggcttaggccaatggttacagctgcgatgagacccctagcctggggacctctatatgccatgggttaggccctaaaaagacccccccccccaaaaaaaaaatcacaaataataaaaagacaactcagttaaaaaataggcaaacaacatgaacagacatttcactgaagatgtgaccaaggaaacatgagcagctgctcagcttctattaatcaagtaagtaaaacaaaaccaaaatgacttaccatttagcatgtaagtggattggcaaaatttgagaatctgatgcctttgattgtagcagaggtgtagatcagcatgatctcttttattgcatatgggaatataaattggtacaaccactttggaaataatttggtggtaaatttcataaaagtgagaaaattttacccctgcaatccagcattttcatttctgcatatgtccacccaaaggtgtatacatgtatgttcagagtaatactgtttgcgacagcaaatcttagaaacaatgcaaattctcatcatcaggagaatggatagagtttccgttatggctcagtggtgacgaacctaactagtatctatgaggatactggttcagtccttggtctcgctcagtggattaaggatccagtgtttccatgaactatggtgtaggttgcagatgcagtttggatctggcattgctatggatgtggtagctacagctccgatttgacccctagcctgggaacttcaatatgctgcaggtacggccctaaaaagtcaaaaagagagagagaatgggtagatacattttaatatattcttgcatcgggctgtcattcagcagtgaaaatgagccaacaatggcattcaacaacttgggagaaattagcttaggcctgtaggtattcattgtgctattaaataaaagtgtgtaatgaaccaaaggtcatgagtgatccatgaagggtaagtgctcagtgaatatttattgaatgtgtgaatgagtcttactccatgtgcttagggtccaaagtgtgggtggggttggaatagtcttttggctttgtctaggttcttagtttttatctttgtccctgcctgagcttccctctaattaagctcttgttctaagtagtgaggttcctctgcctcccgctacccacagagtttctttttgtctattttctccctattctctctctctcttctctcccattggcctttatttattactgagtgcctgaatgactttagttatcatttgaagccagaaatctctattgactttttaattttcttctttatttactgcttatgtaaaatatcatatggcaccattcttcatcctctccaaaggtcaatttgattatatatacaaaatgacatatgcttttgctgttggagttacaggtcatcttgttcaatctcttattactttacagataagaaagcagatacacagaaaaaaatgactttgtcggggtcatacggcttgaagtagcagagctagaacccagatccagttctcttaacctctaggccagttacaggactgacagggtaggctttgcttgaatgactgagtttccgagggccccatagatatgtgagagtctctaccactgggtgggatgtttggtaatgtccagacttattttagtcactctagcatttggccagctctgtctttgttgtatgtttccttctgctcctttctgctctgattttctttattcttcctcagggtatctgtaggttagtgtcaccgggtaccctctggacatcattataaatagagagcatccatttaactagcggaaactgatgacttggccgtagttgcaggttaaaatgaggcacagtgggagatggtaaactttgtctgtttatttagtcatgtgtagaaacttctcacaagtatatactctggacaccaagtctttagggactgtctttgcctccaggttctcagtgtgcattggaaaactcacagaagatgattgggtcactgcggacaggacagtgtgcaacaggattgaaatgggacaagatttagattatatcataaggaccatctctaagactagtcgtagaaatcagagtgttctctggggtagaacctaagtgggaatttcacttaggaccgtgcactgaaatactcaaatctaaaaatgtcgtaacgtttggacacagagctaaacctagtcaagactaatggagaagttggaaacaggggtggtaggtatcctgtaggaggaaattggaaattcaagtcttgaggctctcattgccctgaattttgtcagggacttcagtatgagtgcatgatgaattgacagcctgagcagtgaagaatgtcagtccagtggttatcatttctacctctataccatgactatcacaaaaccaagacttgcagcagaagttagttggagttagaacgttccagggtgggctgatcagataagggcccctgcttatgacctgcaatgatggcatcattttccatcctcacttatcttcctttcagtgtcatggtctcatatgcacatacacagactcatcccaactgtcataaggctgtgcctgtgatgaagctgaatacagagactgaagtcctaatcataggccagaaagagttggcactctttcatatctaaacaggaagaattggaatgagtctatagttataaacagggctattcagtgaggggtaaggagggtggaggatttatggatctagatagggcgaaaatgagtgaattggcacctctcccttaagtctgggaagactttctgcaggaagcaggactcttcaaggtagttactgttatggttacctgtggaaaatttttcatctcttaatgttgtgctttatccctttaccctgaatgactcttgtcagagctttttatgtaactttgaaactagagtagttctgatttgctctgcttgcttcctctccactcatttcactcagcctggcatttgacatgaggcattgatgtcaccattctcctgggccgctcattcctggctcatttcacgtgttacttcctatcttagaatccttcagtgatgccctgatgttcttaggataaagtccagttgccttaacatggcctacaaggtctgcttgattttatcctagcctacctcttccacatcatctttcaccacttctctaccattgactataccccatacaaactaaggcacaaggctttgcttttcctccaggccattcatgtcatattacctctgcttagaacacttccctaccttcatttaactaattaagtcttcctaatcctatacttctcagcctagctgtcaccaggaagccttcttcaaacccctaggctaggctggttcccctcctctgactccacacagtgttctgtatccttcccatcataacctttagcatgctgtgttgtaattatttgttcctttcctgtaatctctataaaactaaagtctgtgagggcagatacattgttttcttcgctgttgtattaccatgtatctctagcttagtgcctgacacatagtaggcacttcagtaaatatttgattaaggaatgggtgactgaactggtttgttgttctgtgacaggaatgggaaaaggacagcacaggatgccctccaaggatgagctggtccagagatataacagaatgaataccatcccccaggtattattcgttagttgcccttggtccactgaggcagaacccttcctctgggataagtttaggctacaggggtggatggggaaacagtggtagtggtgggatgggtggtttaaagatcattttgaaggaatcagatatgttttaagatgaacacatttccagagtcatttgtggcctcagtcataagtatgaaggcagagactgagtttaaatgcagtgacatgaaactgtaaagatggaagcctggactcatctagactgattatgggaaagcaaaaattttgggggggtgactgggagcccaggatatcccaatacccatccccaaccattacctactaaccagcctctctagggaaagtgtaacagtctacctggctctgctttcctcttccagacccgatccattcagtcaaggttcctgcagagtcggaatctaaactgtatagcactttgtgaaggtatgtagcttacccagcccctgtttttctgggccctctgtctttatttctggtgcccctttggcacatggatccaaccaaggataatgatgatgccatttcttccctgcctaaacattatatctgtaacctctggccctcttcttcccagtgattacatccaaggacctccagaagcatgggaacatctgggtgtgccctgtctctgaccatgtctgcacacggttcttctttgtgtaagtctaggaggttaccttggggactccttggaggagtgagggtgcatgatgggactgggagcatgacgacgtgtgactatggggaaagtctctttctgttatcagagacttgtccctggctacctgagtggtgctagcactgggacttgccccttggaggacagggatgggtatagatcagggtctgtttagaggaaaccagataagctgaatcttttccacactaaagagtaacaagaaagggtttgacttacggggtggtgtggccacacttctgaatgcagttccattggttgacattattctggtattgttctccctcttgcgctcacacttgccctatctttggcagatatgaggatggtcaggtgggcgatgccaacattaatactcaggaccccaagatacagaaggaaatcatgcgtgtgatcggaactcaggtttacacaaactgagggggccccagccctcgtaccacccccattcccccgggatccatccaccctaacaaaaagggctcaggagcagcagacagggctgccctgaggacaaggggccattatcaaggggcaggaaaagaataaaagcggcggccttcatggtggaaattgacccaggaactactccacatttggatggcccagattgactccctactcctgattgtcccagttgacttcaccctgtttgtaaataaaacaataaaatggaaggtgctgtggactggatatgatcactgtggtctgactagccttggcccagcacctgccccaagccccaatggaggtgggggtggggagagtggaacatcacgagggttgggactgtttatgttgtgggaatcagagctgaatctgtctagtatagtatactacagatctgtcaacattttaaccaacaaattagataaaggaaacttatcagttatgcagatgacataaactgaggatatagaataccactgatgactgttcttcacgatttaaaaggactaatggaggccaaaacccctcagatttcctatccttagaacctctactttctgcataaaacctctcccctctcctttgctgtatatatcaggagctgatccctacttgcctcctccttccagctgtttctttttgatagggagatgaggggagagctggcaggtacaggaagtgggttggtctctaattcaaaatctataaagaaatcctaaggaatcagcttcccttagtgaaatgaaatccaaattctccaaggctagcaatgggagggaagacagctgttcctaggagacctaattgtttcagtgaaagccatctccctgatgaaattcccactgccagccttctgctggcctttaggctatgctccatgtacctaaaatcctaaattagttacaagagaccagttggacattacaaaatattttatgaattattaatgaaaaataggaacataagcatagagacaacaaaaagcactgaacaagcctggtttttaagtatagatttttaattctgttataacatcttgatgttggactacctgtttcttttcattatcttcaccctacttcggaaataaccttctttgcctgggtgccttacctgctttgtagttatgactacaaggcctgaacttaacatggaagtaccattcttaaggtccatctcctaggagctgtttcttaaactgctttactccttgaatgagactgctgccattctgactctacatgtctcctaaggctctctcccccaatctggtttcaggtctaggagaatagctcttctctaagtaaatgaccccatcaggtgacccaccctaactgaaaatcctgcctcctgggaagacccttgcagctcatctgtgtagtatcagagatagctactaacattttttagttcttaggtagatcattaagtttataacactttggctttcttaccaagtgcttggtattgttgtaagctctctacttgtattgactaatacaattctcaaaacaagcctgcgaagtagatgtcattattcacattttgcagatgagaaaacaggcacagaggatgagtgatttgctcagattccacggctagtgactgatggggcaacctgactccagaattgctattctggttctatgctatatatcctttcaaacttcacaatttttgtttttttcatttaatcctgacaacaaagatggaagataggtgtttttagagatcataagtaatatgtgcaaggtctcacagttggttagtggccattgctgggtttttaaccagatttgctcaactccaggacttgagctcttaaccattcttttacttccccagcggttctcaaactgttggcactggtgttccaacagttgaacctacgtgcttggctactcaaatggtataacatttttgctcagttttcagaaattattaatagatttttctcagttgtaaccatttcttagtttttctgtatcagtaacatctgacagagcaagtactagcaaacggtggaatgagaagtaatgaacaggagttcccgttgtggctcagtggttaacgaatctgactaggaaccacgaggttgtgggttcgatccctggccttgctcagtgggttggggatccggtgttgccgtgagctgtggtgtgggttgcagacgtggcttggatcccatgttgctatggctctggcgtaggccagtgactacagctccaattagacccctagcctgggaacctccatgtgctgcggaagtggcccaagaaatggcaaaaagataaaaaaaagaagtaatgaacagatagtgtatgtttccagaaaaactcaaaatacctgagctgacctcatggaagtcacttttttaataggatatacacaagtcttttagtgccaataaaattataatgaaattaaatgtaccattttatagtgttctgcataacagcaacaggagttttttaatctgaaaagttagaaagctgtacatgaaaaaattcaggaattccctggtggctcagtgggttatagtctagcattgtcactgctgtggcacaagttcagtccttagcctcagaacttccacatgccgcaggcatggaaaaaaaaaattcaaaacatgtatggtataaaacaaatctaattaaaacccgcagcattcctataacaagaatgcttgcattgcccagaaagttgcacagggccctgaatttctggagaggagtcctcccacagcctggagtggaaattaaaggaccattgcccctttctcccacccagggctggctgccttagaggtgggaggtgaaacctgtgaaagccaagtctgtgccagagattgtccttcaccaaagccaagattgttgcaaatcctacacacagaggttcacctactcctggttcaccctttcagacctggacaggctttgttagtactaggtctgggaaaataactattttcttcaattagttctagggggagagtaaatagctatgatttctggattgagaacttaaaactcctggagtttaaacttccccatccctcttatggtaattttccatccaggcagggtgtggccatgattcagtttgtggtgaggatgtggccatccagagaggaggagccttctgttagattctgcctttcatcttggccagtggggagagggagggaagaggctggtgctgagtcatgactgggtaacttaccaggttctcacaagatggattctgagatggaggtttgggtgctgtctcagtttctggaatcaatttgggagtggcagggaggaagcttcaggggtcacacaattccttcagagccctcttcccccagaaagggtgggaataaagtggttcatctgaagtgacatcagtctctggagtataggaattttcccaagtgtttcagagccaccctgcagtgggcccaaagatgttacctttaagttaaagcattgcctacaaaaattgttttatgtagaaaagagatcaaaatttcctttgtcctagctctccttcctgggacaagtagatcaggctagctggtagccagtgagcatggagtggagactagaaatatggtgctaaggacaatgtgactttgatggaactacagcaggaggacatcctggggaaggaggcatttgatctcttgtttgctgatgcctttcttggtcttccagatcttaattactttggggtaacaaggcctgcatacaattccacttgcatcttagggagcctgagctctggctgtacttgtagggaaaaaggagagagcaccgcagtgggaaaaggtagaggcttggtggataggacaaacccaccctaaggaggctttgtcctgtaagcactagcagttctgggctatagaaaaattgctggaggtcttgttggctggggtaggacttcactggaatgaacaaggacttctcaaaaatcttctctccatctaagtgctgatgaggcatctccttaggagggggacctagatacacagtttgatccagaaaactttttttttttccactcttcctttcctctgaaagggtctttccatacatacattcatgtctccttcccttttcctcacaacctcttccctcactacccttaactccaaaaggccaactttctctttcttccaggatttgtctggaattaagatttttctgccacccttccacctacccctggtgggcctgaagagatcttgggaatctgaaggaaagcaaagaagaaattaaaaaggctacttgttctggctctggaaagggcatataaagtccaaagtgaggagttcctgtcatggctcagtggaaatgaacctgactagtatccatgaggatgcagattcaatctctggcctcactcagtgggttaaggatccggtgttgccgtgagctgtggtgtaggtcgcagacgtggatcggatctggccttgctgtggctgtggtgtagaccagcgactacagctccaattagacccctagcctgggaacctccacaggccatgggtgcggctctgggaaaaaaaaaaaaaaattccaaagtgataagcatttctaaacctgacattcaggccaatgggttgtcctgggaatttatattggcctgagaaaactaactgctgtttcaggcagaagccaagagttaacataacggtaaataaaacttgtaatcttgccctctttgtcagggccaaagtttttctgttggctccagagttgggtcagatattgtttgcttacacacctaccttgccagtgactccaaatgaccatcaagagttaaatcaggagttcccgtcgtggcgcagtggttaacgaatccgactaggaaccatgaggttgcgggttcggtccctgcccttgctcagtgggttaccgatccggcgttgccgtgagctgtggtgtaggttgcagacgcggctcggatcccgcgttgctgtggctctggcgtaggccggtggctacagctccgattcaacccctagcctgggaacctccatatgccgtgggagcggcccaagaaatagcaacaacaacaacaacaaaaaagacaaaagacaaaaaaaaaaataaaataaaaaaaaaaaaaaaagacttaaatcattaaggagttcctgctgtggcgcagcagaaacaaatccaaactagtaaccatgaggtggtgggtttgatccctggcctcactcagtgggttaaggatccagcgttaacatgagctgtggtgtaggtcgcagacatggcttggatcctgtgttgttgtggctgtggctgtggcctgcagctgtagctctgattcaacccctagcctgggaacttccatatgctgagggcacagccctaaaaggggaaaaaacaaaacaaaacaaacaaaaaaatcattaagctccagagtctgatttcctggttaagaatccaaacaggtaacagcaatctataccctcactcacacctctgtccagtgaacctagcagctccccctcctatctgccaggtctgtgagcatagtaggagggctggcttatggaggaggagcctagcttctagttgagtctgtgtccttccttagtttccctagtttcatgcagtcctagggttacctgttgctgagtctaaggaataaaggttcaagaggatggatcagagaatggcagagttacttgaattgagaaagatgttctggtacaagtcacaaggaaaaacctctaggactagtcaattctcaaagaggatgcctgctgggcactggtttttatactcattcacataatcatctaaccaaaccccacccaacaagtatgactttccttcagtttacctttaatgaactggactcacgttcaattacaagatacggtcatagggctagtgagtggaattgtaactaaacaccacagatcccaagcttgcttctgtactctgctccttctcatcaaaataatgacaaccaccttaaactctgtgacagattttcaccatgcatctaatgtgagcaaaattatgatggattggaggagtaggtcacaactaggaactaacccctagtctgggaattccatatgccacagggtggccaggggggaaaaaaaaatccaaaaaactggattagggtctgccagtttcatggacagcattttaaaaaaataactgttggatgatcagtatctgtaactgaagggcaatagtttgccatcagctctctaacttaatgcatatgcttttccatcctgggaatatacctagtttagatctttaaagtgcaacatagtctagcattagccagatttttgccactccattgtggtccaccactgttcttagttcatcattagctcaggggccacagctggtcatttataccattcctttctctggatagggagggatctgattgtccctggtgggacatgtggcttttttcccctctcaacttcatgtgctctcaccatctcaataaccaagcatttacttcctatctgcaaactaaggggatgggaaaagattacagaagaaattataaagtattttatctcaaatttaagttgtcactttctcctcgaagcttatctgagctaacaggaaggcataatgaacgcctgtgattcagctacagcgtgtctgcctgagcgagcaggtcacacccattggtctcattttccaggaagaggaaagaatctaagagaatagcattcacgatttttaacgaggcaacactgtatctgcttttaaaacttggctgaaagtaggcaagagttataggtgtttcgttgataactgcagccatcacttgttgcctaattactgggaaatcagaaaatgctttagagaaaaatgtaaaaaacctccaacaatattgatttaaatgcacaaaattgaaaacagaagtgcttattccagcagagattacgggatctaagtctaactgtatttcctgttggttgagttctgcacgaaaactttgtattcactttctactgtctgctggactccagtccccaccaaagcatttgcctggcccaacttgtcctttgttctagccctaagaacaaaagggcctcgtgctcctgtgctcccgctccacgcaaactccgccctctctatttctccctaccccagttccagtgtcggagccctttcgaaggcggcccattgtctccgtcccttcccacctctgataccctctcaggctcctcaacctgcagctcggcttggtctccagggtgcgcgctggggcgggcacccgggcggagtggggggcggggagtcgcaggacaggacttggggaatgctgtccagggacccataaatttgggtagaccagggtagctcagctggggtggcctgggccaaaggtcaagcgaagagcgctggtccaggatcccaaattggcggcaacaggtggccttgggcgccgggaggtctgggagggaggtcttcggcactccgagggcgggtcctgctcctcgccgcttccccattggtctctacggaatgtcaatgctcagtattttatgtaatgtgtccttccgccacggaatgtagtcctccccaaaagggcaacctgcctgcctgttctgcctcagcggcacttcctgcagaaggtgactgtgccccgcgaggacgcagaggggcggggcactgcgagaggggagccaggattgggtgtggggcggggcggcggagggattgcggcggccctgcagtggagataaccttgaggctgaggcagccgattcccgcattggagcgacgcgtcgtgagcctccagaggtctctgcgagcatcgcatcctaagccggaccacgtcggaggtgagcagggggagacactgggccatctgtccccactagggctccatctgcccggccagtgcggaatcttggcctgcctacgcacacagggcacccatatcggagacagcgcaggaacttgggcatcttggagtcacccggttggggtcggaagagaggaaaaaagctgcttgcgcctagggcctctttttcaaaaccctccagctcctgctttttcggaaagcagatgggttggccagctcctttcctgccgcagtccttcatcttcccccacccccattctggttccaacctctcctccttttccttcccttctgtttctggtcccatcctcatcttcttccccttattctcacctaatttggtcctttcctttcgaccctcctcgtttctcctttctctactctgctcccatgttcgcctcctccccttcccccttaatactcgcatctcgtcttcaacctgactgtgcccccttcattctgatgctttccttgaaactcctctagctccactccctggtccctcctccgttcaggttcggtcccctccacatgcctacccgcattcatgcctggttcctgcccctccccctcctctctggtcacgtcctctcctccgcatccctctcctccgcaccctccttcgcgccacccgcgtcctcgcccctcctcttctgttctagtcacgtccgcctgctttcccgcccccgccccttggcctcacatctagttctgtctcctccatcgcggttcctcactcagtcttgggcactcctgcagccacggacctgcgtcccggggtcactgtgcgaagtacagtggtttgcgggataggctcccgtgtgcggccacgggatgcagaagcggcgcccggcaccgctaaagggttacataacccttggccttctccactgcacaggaagaaagggtggctgccctgtggcgtgttcctgttttgtgcggtccttaataagatggggcgacagcagcgctgcactagctccaagagggttacaagactccatcccgccgcggcggggttgcacgtagcggcccaggacccgccccgccccgcgggatcaccaactactgaccttccggcccgttactggccaggcatcacccctggcctgataagactgtgcatgctgctcgggggcctcttaacattggaggcagtcacctggctcctggcctctgcttcacgttaacagatactcccgattgtagaacatggaggccacggggtggggccaggagcaggttatggggagaggaggaatttgtcaagagtttggtgcccagtactgcatccactcagtatgtaaagtgtgcaaagggctgtttggggaaggggtgtggaaaccaggatggctagaatggtcatagcccagggaagtcacaatctaattaggggacacagtgctaaagtctgtgaggaaggtacacactccctgtggggttgaggggggagggcggccggaggccttataaggtggtatcacattgggtttgaagaagcaggaaagcaccttccaatctgaaggttatgtaaagacccagtaatggaaaggtatgccttggcttcatatctgtgttcttcaaacccagaacattgattctaccccccaccccccagcccttgcacaagcagtgcctgatagttaaggtccagacatgatagtggcattgggaacttggaaataggcttttagagtcagatctaactcattaacatactgctatgggtaatggagggaattcagcctcagcaccacagtttcccacatataaattgtagcattttcctcctgatgaaaggattgctttgtacacaactacatggaaatatgttgactactcagtgcagtatctaatctcttgtgggatggctctaaatgtatacttttcttttctttttttttgctttttatactcctgtgcccggggtaaatggaggttcccaggctaggggtcaaatcagagctgtagctgctgacctaggccacagccacaccagacccgagctgtgtctgcgacctacaccatggctcatggcaatgctggatccttaacccactgagtgaagctaggaatcgaacctgcagcctcaaggatactaatcagattcgtttccactgagccacgatgggaactcctaaatgtattcttttggtcactgagtgcaattgcttaatccataactgagcatgagggttcttcttctccctgccatccccaaaccctcagagttaatctttgagagtcaggctctctaaataccagacatctcctggaaaccagtcccctcagggatcctggtacagtgtttacaaagtaactaataatgttacattgcaactccgcttgcaacagcggctctttggtctcctgaaaatggggacctctggacaaagaaaggcaaggagggggatgattttagcctttgatgtttggctacaagagtagaatcaaatttgcaactggggatggtaatctatcttcaccttccactcccacctactccaggtagggttctaggacctgcttagccagctggtgagtgtgagattaggattggaggcaaaggacagtagctccttgttggctgtgcaagggtgtggccacttcctgaggcacagaggacagtttaggtcaggactctgcagcattgttagcaaataatttgccctcaggctgcctgaggatgctgctgggaatgcagtggggctgttctgaggggaagagtgtaacccatcacctggtctgtgggggagaggggaaggctttggggacaatggctcaacctttgaatatatctgcttttaagagaactgtcttcattgggtgagtggggaaactgcactataggcttccacaggcttaaaaactcccctctcccctttcagtgagtcaaaaaatgaagcatgaatttccctcagttaagtatctcgactaagtggaaaaatgattagtgcaggtccccaggcgctataccgccctaatgcttaggtgcagcacggcagagccccatgcccaggtaatataatctgtgcacctgaattcctgagccttatgcagtgggcatggtaaccctttggggaataaaagttgtagtggatatgaataacaatgactttaagaatgtcctttaactcacttctgaattactggacttgagccaaaatatatggacattagtcttctttctaggacactgctctcttgttaagcactgcattaaaagcagatgattttagtgttgaattcacagtttaaccaagagcactattgggtctttaagaattgcaccctcagtgatttggcaagtgactccaagcagaactagcaccttttgggaggtgcttctttgccctgagtattgtgacataatgcgggggtcacaggaaaagccattttatgtcttaaaactgtttatttcttaatattgaggcagtgggagaaatgaggaaatggttgggagcaggaaggtagaaaagatgtaactcttgcattacgaacttgcactgcagtggttttaacttgcttcagcttttatgaaggcaagctagggtgtgttattttggctaagagttgagacttaggtgaaggtagtgggaaatgactcatctgacattatctttatttctgaatcattcaaaatatccacagtccaatcccagttaactggtacaccacaagaatgagtcatcccatgctagaagcaatagttttcttgtaactttgttgaggatcttgtgagataatataagtgatttatttcataaaagttctcataagaccttgagtcctcactttagcatcaattctggtatgaagaggagcagagctaaatgaagactactaccatactttctgggaactttttttttctttctttctttctttcttttttcttttaaccatgagccagccttagacgataaacaaataggctgattatctggggctttgttagagaaactgctttgggaaggtgatccagagtgggtggtgagtgcaatgtgcagcctctgtcagactttttggcctcctggtatgactgctcagcttaggaggggaggttggcagggccaccaagtggttcagctccgccccttgcagccatttttcctacattggtttgtgccagtctgcacgtaggagggaggccaggctgcagtacttctggtggcctctgggactcacctcccccactctgtccataggcagaactcctggggagctgtaagcctctctttcctgacgccagcaagctgtatttggaagtcagacaaggattctggtgggggagatgccaagagccgctatgatagagcattgagcttgctggtcatagcatttcaaagtaaggaccagctcagctgatccaggggggaaaaaaattctctgaagggagccccagcccttccaaacacttaatcaactggtagataatatttgtttaactttagacaactggtatctcctctgtcttgggtgatgagtttaaaataattagtccgctgagtcaggcttagtataggagacaaagaaaattgcgcattttggaggaatcatggagttttgatctagttaagaggcagaagagtaaaaacttggtaaacaaaaccatcctggtaatctagtatccctcttctgtttttacattagagagcagattaacacatggcacccatcagatgggcaaatgttttaaaagtctgacagtgtcaagtgttggcaaaggtgtagggaggcaagagctccctttactggttgattgtaagtggatataaccactttggagagcagttcggcaatatttagtaaagtagaaagtaaacttaccctgtgacccaggaattcatttctggatgtatagaaactcacgtctggactaggacacatgtactaggagagtattgattaatgaggaaaattttggtttaatgggggaaatgaccaactagtaagggaatgcatagatgaatggactagtcatacaatggactagtatagcagttcgtacaagttaactagatctgtataccaactggatagatcttaaatgtgctggaaaggcaagttgtaggatggtaccttataatacactacttagggtattttatataatataatggagttcagtatagatacagacaccctcaaatgaactggaaaaatacgaagttcatgacagtggttggttatcacagtttatgctggggtgtggaaggactggagggacctggggatggaggaaaatgaacttatttttaagagtctagagaaaatttggctaaatgttaatggtcagttctgggtggtagaaatacagatgttaatatttctttagttcaaagcaaaacaaaatagaagcaaacctacgacagtcactatagggaacaccaggcttactaactaggtcagtgctctcagagctggcactgctctttcttccttccatccccccttctcccaacagctgtgatgccgagcagttcctcctttaaatgctgagccagtgcctcccttttgctgaggccctcaggcatcctggtgttgtacctgacttggaagggctctaaatcagggttgcagatcccttgctgcttccagagtgtaaatatcttgccctcagacaccgccttataattttaagcctcataaggggcacaacacagcaaaaatttcatctaaacttcctcctttagactgaaaccagttctctgctcttagtgtctgtgatcaaaccaggaaatctttgatacctgaggacagcaagatttctgtgacttgggacttcctgccctggagttgagtggtcctgttgggggtgggcccatttagtatcagcttttatgacttattcaacctcagatttgaaatagggccttgggctcctgcttataaagtttaagtcgacttctagccctgaataaaagagcaagggaattttacttgaacctataggtgggctcattttatgtaacctagtaacaccagactttgctttttggtatggggcctgctgagggtagaatggacttaatagtctcatttcttaggctaaaaactgcatgtcctggccccactccatatgcaaatatactgtcatcattactctaactgtatgtgaggatgaacagccattcactcaagaccttactaggttctgtggctggcgcagcacccatgttgatcttagtgctgctgttgtcagattggggccagtcttgggaattatgttctgctccatcatctaggaacagagtccggcacattctgatgcaaactttctagtgaatgctgcagttctgaaggtgtggaaatcgggccttcatgctggcttggagacatggagctgtgggtagcaaattgatgttgcaacaaggaacagctgtggacatcaggtgccgtcatcattgggttagtctctgtggtgagatgggcaacaaagcagaattggtacacagagccctggctagtggaagattggcttaagtagatgcgggctcctaggccatgctggtttactagggcggaatgatttgggcccctataaaggaaagaattggttgagtgaagcatgacttttcccagcacaacctgccaacagtactggctctaagtgacatgctgggtatttttaattaattaatttatttaaaaaaaaattttttttggctgcacctgtggcatctggaagtttcaggccagggattgaatctgagcctcagctgtgacctatgctgcagctgtggtaatgctggatcccctgctctgggccagggattgaacccgagcctccgaagcaacccgagtcactgccgtcggattcttaatccactgcaccgtggcaggaatgctgggcattttttcttatatggttttcaccctttggtttctgaaaagtgaccaaaggattgtgtctgacaacaaaactagtttttgagaaccagcagccacttaagatgaaaggtgggaataggggccgctggagctgagctggctgcctgctctatcgttttggcccccaccaaggcctctgatggtgtctcatttctcacagaggagaaatggctctaaggaagatcctactgcaggggctcaggctcaagaataaggctccctggacagggcacagcatgtgctgccatcagtggatatgcccggcttccactgcgggcttgaggttggctctgcatgtctgtgcctcctgaggaggatgaaggaatgagtcagcctggcgcgggctgggccttgggtgctggcaggcactgtcctgacagctgatgttggggaggggagctctttgcagagacctgccttgagaaaccagggccaaggctgctgtcctggggctgggggagaccccagagggtggtgtaggtggaggaccttggagacaagcaggagagccctgactctccctgtaagggagactctggggtggagtctgaggggagggagacctgtgatgacctgggcatgagtcatgaggtctctggtttggtctgggttttaaggacatgttgagatcttgtaggagtttggactgacttgctgacaagccaggtttttttgattgctggtgtgtaaatccactcaaaggccaaacaaaatttcatttgttgtttttgtccttaatagcctgggtgagcaaaagatgagagcttttagacctcagatttaattcaagatgacgaaatagggttagtgtttgagtgcagtatagttgtatctgctctatgtccttgagacggcccttctgtaccagaacattctttttttttttttttggtctttttgtctttttgtattttctagggccgcacctgcagcatatggaggttcccaggctaggggtcaaataagagctgtagctgccggcctttgccagagccacagcaacgcgggatccaagccacatctgcaacctacaccacagctcacagcaacaccagatccttaacccactgagcaaggccaaggatcgaacccgcaacctcatggttcctcgtcggattcgttaaccactgcgccatgacaggaactcctaccagaacattcttatcacaaatattttcctctgatgcttgaggacagactacgtaagaaaggaagtttgctgggtttgggcacataacaggctttttggcagagaactgtcctatggactgtattaattgtcccccaacccccccacccctttgaggttctgaaagggatcaaggttgaccttcttaagtcagtgaaattatcctgcagtgcctttgtgacctagggcttaaggtgcattattccaccaaggccatgtttaaacggtattcctgaagtgcatagagcagcgatggactaatggaactgggaacttctcattgctttcgcttccaacacttttaccaggatgcttgtaaatctatcatttttggaagaggtacatttgtaatttatcttattatgaaagtatttcacattagtaagctttttttaataagagaagaaaaatttgcaatttacagtgatcctaccacccagataacatgctgatagtaatttggtaactatccttccagtcctgttctgtatgtttaatttagtgacccttcccctactctcccccaaaggggagggaccaggttcttctgagatttttttttttttttgtcttttttcgggccacacccacagcatatggaggttcccaggctaggggtcaaatcagagctgtagctgctggcatacaccacagccacagcaacgccagatctgagctacgtctgtgacctacatacaccacagctcacggcaacgccgggtccttaacccactaagcaaggccagggatcaaacccaagtcctcatggatactagtctggttcgttactgctgagccacaatgggaagtcctaaagagactttaaaggagagcagtctttggagtattaggggagtgataagcctaataccaaagagatggtcccacagaaggaccaataagaactcagttttgcctgcaaattttctgttaatcagtttcttggtattttcttgggaggcttctgtgttttactttataacatggctgaatctctagaagcagctattgccaactactacctggatacctggttattaaggtaatttctctattaccccagagagccttagttgcactcagcttttaccttgatccttgtacagctctgcagggcaaacctttctcagatctgagcagtctttgcaagtggggccaaggagccaccctgaaccaacaaaagagcattatatcaccggaagcccaagccctgagaacacaaggtatgaacagtaatcagtagccccagccaggtctttgcaagagaccctaatgtctcaggtagagcgaagatccaaccgatgagttttaagaagagtggggttggttactttagcctactcattgaggtcacaatacagcccccaggctggatggcttctcccactgggaggccacaccaaggcttgtgtgtctgtagttgaggacttgtagccactggctttcactagcttgcaggagattcactcacaactggtctcaaaacttcttgggctttacctgacttctaggaagtggcagagatcgacttagatgcaggctctgctcaagccacccaaagaggctacaaagttagggatggaggaggagaaaaaggcggggttaaatgtaggggtgccttgatttggagccctgagatttaggaggaggtgggacagggcagaagaagggcttggcttgtttttctgagcctgggtgaagcacccaggcacggggatttgagaaggagtagcccttggtctgagggtttgaggagagagatggtctgctgacgaggcgtgtcaagcttctggctcacatccttgcctgatggttcttcaaatgctgcccagaagcttctcaggtctctgtctataatcagctagctaaccggctgagtgagtttagttgtaagtaatgaagaaaagcaaaggttggggcagttgtcaggagggtgacctgtgctgattaattacctggaacagatggtctttgttactcggggtggtgtgcttgtcagacttagaaaggcaatcgggttggtgcatcagaagatctgctgtggagaggatttaacaagcaaccttcacttcctcgtgttgcaactgtcctgttaatgtaactgtagtaattttagaatcagatagtgccactgtggttatcgttggcttgatgcaaactgcttcgcatgtcctaaagtcctgaccaaaagttacaggatggggttgggtacagattcctgggactggtttttattgcctgtccgtggcctgggacctcctctccagcctagggtgaattgaggaatgtgcttatgtagtcctgtgggttagtgaaaaccttggactccagaactaggaagaatgcctggatggtggttatctccatgattaagaaagtcactttgggtttgggtgtagattgttaagaggcagtgtgggtgccctatcctgtagcaggtatttgtagctttgagacagctttttgtacaaagatctagtttgaccaaggtactaaactaggggtgagttgttgatatcactgggagattggttgcagcttccttcagtgcatttaaaatgatatttataggattagcccagggtgagggtggtatgcttacagtggatgcctgggaagtcccctgtgttggccatgctttttaacttgtttctttgtggtcttttccacttgctcacggtgacaagcctggggacctacctgaggagcacccacacagccagagcgtggcagggctccctgtggcctgtgcacagagcgtgcttgttgagttaatgtgtaagcaggaaacggcttcctgtgccaagctgtatattagccatcccaggcctctcagactgatccctctctctcttcagttcctctggacccaagacctcagaagccatgccgaagccccacagtgatgccgggaccgccttcattcagacccagcagctgcacgcggccatggctgacacgttcctggagcacatgtgccgcctggacattgactcgccacccatcacggcccggaacaccggcatcatctgtaccatcggtgagtgggggtgcccccctcccctggaaacaagggcttcatgagacagggatcttttctctcctggaaagactaaataaatgtagcaaacctgggtgctgagtgagatgctaaaaagatccttatgggtactgaggtcataggaatttacagtgaggtactggtatgctctacgcatgctctatgctttatgataaaggtgaaatttagagggtatcttccttagtggatctttgtaggaacatctcagaaacctacctgggaaagatcttgacatgctgtgtacaactgagactctgtagctggacatctgggtgggcttcagggagtgcagagagcgctggtctagtcttagctaggctttcctaaaagaattaggagttatttgggggaggaaaagtttaaggggagggaaaaaaatttttttttgcttcttatactgaagcaaacttaaagtgttattcttattttgaataattaatacttaatcactttaattttttttaacttaggcccagcttcccgatcagtggagacactgaaggagatgattaagtctggaatgaacgtggctcgtttgaacttctctcacggaactcatgaggtgagcctctgccggacagtttggccactggggcaccttgtgggcaacagggcgggagatgccatgttcatttagccacggtggaccaggcaagaaagattggtttacagccagtgggaaggttgtcttcaccagcccatcctcacgctcctttcccctctcctcctcctgccctctcaagtggccaagtctcctctagcaaacaagacctgctttgtcattggccatggttctcccccacagctgagctccattagccctcagtaaggaacgtgtgggagagtttctctggaaacccctctaagccatggggcttgacattcccaccccagcatttctctaacagcttgtgttgcagtgatgaaccaatggctcaccttgcaggttgattctggccaagtttagacccaagacaaactgggcaggtccaggagaagggtttccactgttgccagtgaaggactgagtctaatgctctaacaccgtctggtgtatcctaccccacttcatctgcattgtgttgccagcctttcccttcccagggattctggtacattgaaataaagaagccagcatgtttccctaggagcctggtaggaatatcttagctggggctgtttatgagcagacttgacctgtgggaccttacctggggtcgaggtagaagctattgggaagcactccctcatgcttctgttcccaagttcagaacaaatttattttgaggagtgtgggagctccctgagggacagtgccttttaaaaagaccttcagtttcaaccacagaccatgggctgatgagagggtaatggatgagataaatttttctcactcttaatctctgtatttcctattcaggataaagaaaatgctaccccataaggtcaagtggtgaggggttgccctctggcatttggcagttggtgttaagtttaaactaaaaataaaggcttagaaggtgcaatccaagagctgaattcctcctttctggaaacatgaggctcttcattggaggttccctaacccatatttcataagcagcctatggtctggcaggtatccagcctccaaatgtcagaagggcctgaccaagtgcagaggaaccgtgctcagcaggcccctcgggcaggttgatacgtctgtggtggcattctgtaattttccaaaggcagggagtcctcagcaggaagtcactgggcattcctgcaagtattagcttagtccacggagagagctgaagacatcagtgaaggtcagttcactattgattgctcctgctggcctgtgtgcaggggaaggagcagaaatagattttaagaagttgacctttagctaggctttatggcttcttccatccagtaaaataacaccacacagctctgaaatggcaagggccagtggtatctggtgcacctgctggaagggggctggctcagggattttggctttttttaactccaagaggcttttgtaggttctctgtgctttaactctcattccaagatgagacgggatttaaacaaaaacccatgcctgccttgtggatttattttgagatttttgaccaggatgaagtgggttgtccctgggatcggcattttagaaactgatctgaaattgttctaatattaattatataacgtaagcagtcttattttgacaagcagttggaacatccttttccagctcatgacagttttcagtattcttctaagcagaacattggaatggctttctctccctcactgtgacaatgggacctccagtgtctgcatctgatctttttgtggggttgttgttgctacagcatctgatcttttgacagctttgccatagcacctctaaaccccacttttggcaagatgtgttcctactgattgtgtggtcccattgtgccatgtgggccagaaatacagacttctgaacaaactaaatgtagaaccttccataaggacagtgttggtttgatggaattcactgccaccccgccccagtcccctcctcgtgcgtgccactctcctggacggtggtggttaaatgacaaggcccataaagtgctgagttgggagacttttatcagcagtgtcatttcaataactcttatctaagagcttttgacttcttgcctctgagtccctttttagtcaagtagttgtggcaaatggaaaaagaaaaaggaatgggagaaaaaattatgtattcatgtatgtgtaactgggtccccattctgtatagtgggggaaaaaaagtgtgttgagggaaataacaataaaaaaagaaaaagagaaagggcttcacaggtgagattaaaaccaggatttgttgcaagtgagaatagcaagggttagttagttcagagaaaggtgtgttcccaccgttgccttttgcactttgagactcgaggaagtgattatcagaaccaggtctgtaggacctctgcaatgagttatttagcttggaccactgttagcttgagtaaataactgagtttgaagtgcggtcataatcaatagaggaacagctcagaattaccaaatctgtcagtctgaccctgatggccaccagactggtattttccaaacataagtcctaggtcttttagcccaagtgtctgctttatttgctgagggtttaggtggcagtctaaaagttcctaagattggccctgagagcgggggagtgctgtggtagaggtgggtggaaggtgcttggtgagtgggcggcgctgagactagccgatggcgacaggggtccagagcgtccgtccacagctctttcctgagtgctcagcttcagctctggtgctccaccctggggcagggaaggtggcttatccttctccctcctttcccccagtaccatgcagagaccatcaagaacgtgcgtgcggctacagaaagctttgcttcagatcccattctctaccggccagtggctgtggccctggacactaaaggacctgagatccgaaccgggctcatcaagggcgtgagtatcctgggggaggggggaaggagctggggccccagagacatcctctgcagcctatcccacccggaagccttgtttctcagagctgaggtctatacatgagttttatccatcactgcaaaaaactttgatctactttgattagaaacaatacccagaaggaatgattttttttactctcaaaacttgcttccaagactctcctaacctttgggatcaacttgtctcttttcccctatcatgaaatagtatcactaaaaccttgtgggtttttcttgggtcatttccttgaaatatactgagttagacaatgggatcatttctttccaaagaaaaaagctttattgttacttccagctgagatttgagccttgctccctggaggaaagcttttcttctcctgccaggagggcctggcttctctggacctgtggcttcctcttgtctgcagagcggcactgctgaggtggagctcaagaaaggagccacgctcaagatcaccctggataatgcctacatggaaaagtgtgacgagaatgtcctgtggctggactataagaacatctgcaaggtggtggatgtgggcagcaaggtctacgtggatgatggacttatttctttgctggtgaagcagaaaggtatatatgggagccagggtctaactgtctagaggcagctcccatctcttttctttggcagaagataggaaggtggtgggttggctggcagcaagattgaatccatgcatcctcaggaattccttttataataaactccttatcctcaaaagcagctctgtcgtacctcttgggaagcagatgagaggatttcttttctccctgtccgcactacttgaggttttctgcttttctcctccatctccccagtgtggcatgtctgggactccagtatgcctgtgattgatagctcacacttcagtgccaggtactactctaagcatttaacatattaatttgtttaaattttaaaacaacacgaattccttggtggcacagcagattaaggatctgtcatcgtggctcaggtcgctcctgtggcacaggtttgattcctggtcccagaactttggcatgtctcaggtgcagccaaaagcaagcaaacaaacaccttatgagtagatattgttattatctctatttaactgaggtatggagaggttaactgacttaaccccaggttacacagctagtggagtgtggatgtgaaccctgcaatgtggctccagcacccaccacgtgctgtcacccagttctgtctcctggagagagcctgcttcctggggaagacgtggcccccatggccctgccaccccacctactccattcccacaggtcctgacttcctggtgacggaggtggagaacggcggcttcctgggcagcaagaaaggtgtgaaccttcctggagctgctgtggacctgcctgccgtgtccgagaaggacatccaggatctgaagtttggggtggagcaggacgtggatatggtgttcgcatctttcatccgtaaggcggccgacgtccatgaagtcaggaaggtcctgggagagaaaggaaagaacatcaagataatcagcaaaatcgagaatcacgagggagttcggaggttagtcctcctgcctctcccctgcctccttcagcccccggcccagctctttgaagagcaagatgtccctgtaaatacctgcctccctgcccatcaacttagaggctctcaagcttatttcagagccactgaatcagaacatcctaagagggggagtggaaagtgttggttttttcaagggggaaaaacaaaccaataacttaggtgattgggatgctggtatcttttttttttttttttagtgatttttatttttttccatcatagctggtttacagtgttctgtcagttgtctacatacagcatggtgacccagtcacacatacattcttttctcacattatcgtatgctccatcacaagtgactagacatagatcccagtgctatacagcaggatctcattgctcatccattccaaaggcaatagtttgcatctatgaaccccaaattcccagtccatcccactcccacctctcccccttggagccacaagcctgttctccatgtccatgattttctcttctgtggaagggttcatttatgaggatgctggtatcttgacattgttcctaggccccaggaccagcaccggagagcccagggccactgcttcccagtttgggagccatcacgctgtgctcctaacacagcctgtgtttgctagaattgggccacccacagggaaggcagcagactcttaggggtgctccctcccctgggagccacgtggaagctcagggcctggggttgacaccaaagcatgtggcctcctagtcttcactggccgtggcttgggatgggccagttctggcatggtatcagcttcctagggggaagcatgccattctcctccaggtccaggttgtcccagctcatctcctccacctccccattgctcaccaggtagagccccatgagcatagccagcttggtggcatcacttcctctttggttcctttctagaaacacagctcagtgctgtgaccccctgacctgcttgtgtgggttcagtatgtgtgtttgcccaccttagctttgtgtagatgtccttagtactgggaaatttggggtcctccctctgccatccagaaaagccatgggtacttgctgaccttgtctccaggccaagcaaagccatatccccttgccttcagaaagctggaggtgccccctgaaggagcagccctcaggtgtccttgggtggaagaggccatgaacctttgtgtcaggagcagtcaaccaccttgggttccctttgctccaaaagcggggactggactggaggtggtagagaaacaccttaagaggggcctcataagaaggagatggagaggaaatgggaccctgggactttggaatagacctcactcccttccatttcccccagtaaagtgtccacactggtgccgacgaatcccagaaagcagaactgtcgaccctctttgaggtactgaggtagctggaagacccaccacctccatgtacccatgatgacttcatgcatgggtgtttgtcttggtgcaaggacggctgagtcagagaatcccaggaaaacgtcctttgtatcaccattcttctggcccctattccatataacttctctctctttccaacttacctgtcagatttgatgagatcctagaagccagcgatggtatcatggtggctcgtggtgatctaggcattgagattcctgcagagaaggtcttccttgcccagaagatgatgattgggcggtgcaaccgagctgggaagcctgtcatctgtgccacgcaggcatgtgcctctcctggagcttacaaccttgcacggggaagcattggcggctaccgggtctctcccatctcttccctgctccacgtctcacataacacacacacataccccctttcccccacatccctacacaaagccattctgtctgcacatacatgctctctaggggggttagcctgcagtcgtttgtgtccggagacagtccaggccacgtctcacgagtactctgtcccctcccagatgctggagagcatgatcaagaagccccgtcccacccgggctgagggcagtgatgtggccaatgcagtcttggatggagctgactgcatcatgctgtctggagagacggccaaaggggactaccccctggaggctgttcgcatgcagcacctggtgagttctcagcctgccatcttcccgctcagctcagcttgggcttgggctgggatggaggtgtgctctggcactgagcaataccttttaggcttctatgcctacagggttttatctgtcaagtcacaagccagagtgaggagatgtgtgctgggggcatggagatgtcttattttttttctgttctaccttcctgcccacacccctctcccagttccttctgttctggaggtggcctccttcagcagacgtagcacaattcactcctgtcttcagggttgtgaattcttcctttggctccagtactgggctatctatctgtccctggacagcccagaggacctgagtacctctgccctattctggaaaaggccacaggctcctgctcccagctgttctttgtttggcttctgttttgactcgacacctgtcccaagccagctgcctccgcctgcaggaggagcctgggcctgcggcctgcactggagtctgttgcccacgctgagtcgactgagctggctttgcatggtgcctgcagcagcgcctgctttgctgagctgttgcactcgcctcagactacaaagtggggtttaagtggtgggtgacagggccacaaatagagacagttggagggatgactgattctggctggctctggggtgagagcatctggaggagccctgagggccactcgtccttttcccctaggacctagcttttcagggctctcccactaagctgtgtcccacagcctggctgcttctcagggcttgaggctgggttcctgtgtctgctcctgctctagagcttactgaagagttagtgcagggatcagctatcagagctgagaaagcaagtgaactttcagagcagtcccaggtcactgtcgatatagctcagtgaatgaacttgcctgttaacctgaaatggtgtttctttaagcttttatttgcttttcagccaacaaaagaccctgtgccctttgtgtgacaccctctcccctgcattccacccaaatttaccccccacacacataccccataggaacccctggactagataggatccttttttccttcactttctgtgtaattattaggctttggtagcttccaaagaagatatgactaaatcattttttacactaaatcgctttttcagtggtgaaacaagacatcttgactaaagaggctctctgggctgtccttggcctcctgggtgggtttgcccataacaaaattgatagagaccaatccctgtccctgggatgcgagatatgtttagttagaaccttagagttaaattttgctgccaaaattttcctgtgtagattgatgtaatcttcataacttcagtgagatagaagtcaccccttgttatagaggagggtccctgatgataggacttgagtctgaacccagatcatctggctctaaaacccgtgttctcctctgcaatatggtgcttcgtctagcaggagatcaggacaggtttccatgtgggggctgtgcagaggccagagacgaggcatcaggctctgggctttgaatccatgtatgtttttccatatccctgtctttcctttgaacagacaccttagaactgcaaggctgggactgaatagcactgctcaggagataagttatggagtctgggccaggcagtgcaggcctctcctgtccatgtcaccagttaacactggccagcagcttcccctcttaactgtccactgctttctcctatatctaaccgaggcagtcaatatcccataactaagggttaggcaataccaggtatggttctctttcctgtcacatcccctgtcctgtgggctgcatgccttccattcttgcagggagaaaacctttaataaccgagcaaattctgccactcatctgcttatcctatctgattaatgagatggcattaccaattgtcccttgtccatcattctctaaggtgctggtaattgcattataacctctaagcttacattttctttctatttcttggtctgagcaggtctgtgaataactcagtggctttctcctgggtgttttaatcacttgattttaatgaaatgtcttgttacacttacctgcatgcttccacaggcacctgtctcttcacatggctgttcagtgtgtcccctcacagctcaccgattttccacctgcctgtttagaacgtggtcaagccacgtggccttggctttgtctgacccagcttcccagcgcagctctgtgtgcttccatggcaggccattgcaaaggctcgtagcgtccctgtgcctgaagccaagcaagtgctccatgaatgcatggaggccggcaggaagcccggttggtaaccagacagccatgtgaggaaggaggcctgttccttcctgtgagctgtgtcatgaggcagcgtggtcaagtcctaccagggagccatgcaggcccagcctatgtatgtttccatgccaaggcgctagggtctgctgccatactgtctcccaccgccagtcaaaaagtccttccaaaatcaaaaggcccaatttcccttctgttggaagggaagcagcagcagtcagtgcttggaccacacctccctgagtgggcttccatcaccctgcctctttgcatctgcctaaaggacagacttagccaattaacctaaggtaccttcctctctgattaattccccattctgtctttccatgttgttgtctctcgttttttttttccccctcctccttccctcttccttgcctcccctcctccttaaaccttacagatagctcgtgaggctgaggcagccatgttccaccgcaagctgtttgaagaacttgtgcgagcctccagtcactccacagacctcatggaagccatggccatgggcagcgtggaggcttcttataagtgtttagcggcagctttgatagttctgacggagtctggcaggtagggccccgagagcaggtaactctgtaggataaccagcctcttgctccagctgctctagaagacagccagggcctagccctctgctcaggacctcttctctcatctgcaggaagccagccagggaggtcagggcacgacaggactgcagggtccttgagccccgtagggacataatgtcacaggcacctggtgaagggctggttcctggggagccttgatctcacttagccccacaccccaatgtttgggctcctcttggcctctgcacccagggacatgttcctcaccagctgtctgtgcgactcttcccctccctctcccgtgtgacatggctctgataaagctctgtccccctctcgtccatccggacggatgttgcccccctagattgcccgtgaggcagaggccgccatctaccatttgcaattatttgaggagctccgccgcctggcgcccattaccagcgaccctaccgaagctgccgccgtgggcgccgtggaggcctccttcaagtgctgcagtggggccataatcgtcctcaccaagtctggcaggtaggaggtggcagcggctccccaggaacgccccgctcagtggcacccttccttgggggtcctgggagcagtgcactgaatggtgctcagatggcactgagccaaggtaagacccctctgcctgcgccccggaccctgcagggaaggagctcccccagccccactggccaggctctgggagcgagcctagcccttgccctgggcctgaaggatgggaccccaggcagcacgcataggatgaccgtttgggtccaaatcaagtttatatcttcatgatgaatcagagacttaattgcataactacagatagatagagtgatgacaaatggttattcaacattaaaagctgttctcaaaaagtaggagataatcagctaccacttttgcttattctagttactacatgctctttaaaaagttcagtttggaaagttagtgctgcttgcttacatgctttggttggtgcactcatcccatctgtctttgagtcccaaagacaagtaattgggtgctgcccaggaaaggtcaccgtgctgtcttgctggcactgtgggactgggcagccacgccgaggcctctgcttgacctcagactcggggtcccttccaaattcgggacattgacttatcaactttgctctggggcctggcgatatgctcctcccagggcagctagggtttcctggaagaggagggtggagaggaaggaacaccgaggagcagcagatggggctgctgtgggaccatgacacaagatgcagatttcctcagcagagggctcagaataaacagttccaaccattagggttgacctttgtgaaaaggtccaacctttgctacttggctatttttaactccagtcttctgggatttcactcagtgggtcctgacttcagcacttgcttccctccgaaagccttgtatggaggccagcccttaagcagggcattctctgctgtgacatggcttaagtttccctgacacctgttgagtgccctcatagcttgccttctggggcccagttccccctgcaagcccttccaacccctaagacacatctgcagctacaagtgcatagtgtcagtgttgaaagaagtgccggcattagggttttaagggggggggccctcacccctccagaggagcctccacaggcaaagccttccctgctaagcactccaatctcctggtcttgactcctgggtagcacctatatttctaaacctcgcttgggttttacttgagctctacagcccagttgctgggactgtttcaggggtcatgtgaggtcgggagagggcacagcagaagggaaggcttgcgttataaagtctcagggggactggtgggctctgctcctgttttatgtagatgtcaagagtcaggtgctattcacatgctgcttggcttgtatgtagtgttgctggcagcaaaagaaatgggtgtggtgaggttgggcagggctgcctccctgtgccagaatggaaaaattcagggaaggggggttctgccaactatgggccctgtgggtaagtgggtgagcctgaccaggctgggtcagcatgatccccaggcccaggcctgtggggaggggtgcagcatagatggagagcacagctgtgaagaagagctatatgctgtggaagataaaggatgaggggctgcccagccttgggagcagatactgaactggtcagagccttaggctggcatggtgagttggattagagggtgagctcttgctaaaggccctgaggggcttcctgggacactgtttaatggtcttgcctgtccctcatctcaggtctgcacatcaggtggctaggtaccgcccccgagcccccatcattgctgtgacccggaatcaccagacagctcgccaggcccacctgtaccgcggcatcttccccgtggtgtgtaaggacccagtgcaggaggcctgggccgaggacgtggacctccgggtgaacttggccatgaatgttggtgcgtggctgggaagcaagggctagaggtgggggttggggaaggtgtccctctctaatgctgaatgttctctctgaatcctccatcccattccctgaagcctctgggctgagaccaggagggtctgatccacccattttctctcctcactctcttctagtctggggtccaaagcaagggatcctggagagctagggttgttcattctttagcatctgctcattattgagatttttcataaatttagccagagctcctgcattgtggggcagcccacatatggcttctgtccagtataagagcagagtgttgtgggatcagaggaagcagctatagctgtgaccttgagctgggctctccaccctctagagtagaaaatgaatgttgtccaaaggtccctgccagctctgctctgaatgataaggaaatatcagggctgtggggctggcgccaccttgtgggtataaggaagtaaaggcctgcctgtccttcttgctatttttgattgtggggccgtcttgcaggtgcataccttcctggcatgatttttactcacccgcctctctcctctttttccccaggcaaagcccgaggcttcttcaagaagggagatgtggtcattgtgctgaccgggtggcgccctggttccggcttcaccaacaccatgcgcgtagtgcctgtgccgtgatgcaccctgcagcccctactccagccccatcccatccccctccctcaatccatccattaggccagaaatgcttgtagtgctcacttggggccgtgtgtggcactggtgggctgggacccagggacacctctgtgaaacatggctgtttttaagaccctgcttgggtggggtagttcagagctggacctcccatcaagtatccccatccaagcaagggatgaaggaagggtgcaggcaggactggagtccccagagggcaacagctcctgcttctcttcctttgtgtactcctgtagttctgtagaaaatggatacccagagaactcccagccctggcctggaatcagcaaagagcaggggccttagggcatggggcatgaagcagtggttccagtttaagcagactctggccctggcccttacttacttctccaacccccttagcctccctcactccccctttgttgtgcactgtgcacttctgttccttcactccatttagctgccgctgcagacaaacactccaccctccacctcccatttccccgactactgcagccgcctccaggcctgttgctatagagtctacctgtatgtcaataaacaacagctgaagcacc

SEQ ID NO: 54 - DNA, 26664 bp, Bos taurus
  source 1..26664: mol_type = genomic DNA
ggcacttcctactgaaggtgactgtgcccggcgaggacgcggaggggcggggccccgagagccgggagccaggattggatgggaggcggggcggcggagggattgcggcgacccacagtcgggataaccttgaggctaaggcagcggatccccgcattggagcgacgcgtcgcgagccccaggacctgtgtgcagtagccgcgcatcccgagccggaccacgtcggaggtgagcgtggggagagactgggccatccgtccccaccggggctccgtctcccggggcagtgcggaatgctggcccgcctgcgcactcaggatggcgcaggatccgcggcatcgtgggctcacccggttgggatcagaaaagaggagaaaggctgcttgtgcctagggtctccttcaaatcccccagctcccgcttcttccgaaggcagatgggtcagccagcccctttctttccccagtccctgatctcccatccccattctggtcccattctcacctccctattcctccctcctctatctggtctcgctctcgtcttcctccctttattctcctaatttgatagtttcctttcatgcgtcagtctcccccatttccactctgctcctatgcccgtccctttcccatcatccttaatactcgcttcttgttttcaacctgtccatgtccccctcattctgatgctttctgtaacttctccttgaaagtccccaagctccgctcccctgtttctcctccattcagctgctgtccccttcttgtacctaccctcattcacgccctggctcccgcccctcccccgcctctctggtcacgtcgctccagcgcatccctctcctgcgcaccttccttcgcgccaccagcctcctcgcccctccccttctgttctagtcacgtccgcctgcactcacgtccccgcccctcggcctcgcatctcctgccccccttctccatcatagttccccagccctgggctggagttccggggtcagctcccgaagtgcagcggtctgcggatgggcaggctggctcccaggcgacccggagatggatgcagtggcggccggaggagcagttaggttacataacccttgatctaacccaatgcaaaggaagaaagggtggctgcccagtggcgcgttcctgttttgtgctgcccagaatacgatagtgcgaccgctgcgctccgcttgctccagggggccttgggggcggggggggggggtggagggcggtgcgcggtgtcccgggctccatcccgccgaggcggggctgcacgtagcggcgccggacccgccctgccgcgcccgctcctcggcatcacgaaacactgaccttcgggcccgttaccgaccaagcatcatctccggcccgccaagcctctggatgctgctcagaggcctcctcgagttcgaggcactcacctggctccctgcctctgcttcacgttacttaacagcgttgcttaacagatgcgcccgattgtagagcgcggaggccgcgggttggaggagagtagaaatatatcaagagcttggtgccacgtacatgcgctcattcaataaaaactcgtataaagagttgtttgggggagggctgtggagaccagaccaggatgactaaaatcgtcctagcctacggaactcagtctgattagcaaacgaatgtaaaagttaccactgtaagtgccgtgaaaaggtacacacgtgcttccacacgtggagggtacgcacttcctgtccgggtttaggaggcctcaccaggtgtcatcacacggggtttgatgaagcggaaacgcaccttccagtctgaagattatttgaagacccagtaatgggttggcatcccttggcttcttatctgtgttcttcaaacccagaacactgatccttccccgtacaaatgagcctgataactgaggtccgtgacggacagaggcagtgggaagatgaaaacaggctttcagaatcaggtttaactcattacatactgatatgaataatagagggaattcagactcaggacctcagtttcccatgtgaaaattgtaacttttttcatcttggttgttaaaagggtgggtcgctatatacacacttacatagatacatattgagtactgggtgcagtgtttaatctctggtgagatgcccccagatgtattcttttggtcacagtgagcccaatgacttcatccataattgaactcaagcactcctcttcttgctgccatcccaaaacacagactcagttcatctttgggagtcaggctctgtgtaaataccagagatctcctagaaaccagtccgctcagggatcctgctgcagtgttggcaaagtaactaacaatgttactttgctgctctccatttgcaacaggggctctttggtcctctagccaaaggaaggcaagaagagagatggctttcagcctcctgtattatagattttggctattaagattagaaacaaacttgcaaacctaagaccaaaatcttcaggttctattggcacctgctccaggtagcattctgggatttgctcaaccagctggtgtgagattaagataagaggcaaaggacagtagctccttgttggctgtatgcagggtgtggccactccctgggatatagagaaggccctaggtcaggactctagtcattgttagcaaataattcaccctcaggttgcctgaggatgctgttgggaatgcaggggggcctgttctgaggggaggagtatgtaacacctcaccagatctctggggggggtggggtgggggaggatgtagggaagatgactcaacctttgaatttttctgcttttaagagaactgtcttcgttgggtgagtggggaaactgcactgtggtctttgcaggcttaaaaatctcctctcccttttcagtgagtcaaataatgaagtatgcattcttcccaattaaaagtgcctcagctaggtgacaaatggatggtggaggtccccaggctctctggacaatgcttaaatgccacgtggcagagcctcaggccccagagatagtctgcacacctgtcctgagccttgtgcgatggatgtggcaacccttttgggaataagcattgtagggaagatgaataatgatgacttaaagattatgtccttgaactcatttctgcattactagaatggagccaaaatgtagggacattagtcatatataggacagctgctctcattttaagcactgcattagaagtaaataatttcagtgttcacagtttaaccaaaagcattattgggtctttaggaattgcagccccagtgacttggcaagtttggtagctgttactccaaacagagctaacacctaaggggaggtgcttatgtgccctgagtgttgtgacattaatgttgggtcacgggaaatgccattttatttcttaaaactgcttatttcttaatgttgaggcagtgaaagaaatggagaagtgtttgagagcaggaacgttgaaaaacatgcaactcttacgttacagacttgtaatgcaagtctacaggagtttttaaatatttatcttcaggttttatgaaggcaagtaagggtgtgttattttggctaaaagctaagactgaggtgaaggtggggggaaatgactcatttgacattaactttatttctgaatcatttggaatgtccacagtcaaatcccagttaactggtgcaccacaggaatgaggcatccatgggagcagagttgttttgcaacactattgagtgagggtcttgtgagatatgtaagtgatttatttcgtgaccagtcctatgggaccttgaatcatcactttaagcttcagttctgatacagatagaactaaatgaagactactctttgcgggggtggggggggtgttgctttgtgtcacagcttgtgggtccttagtttccccaccaggggtcgaacgtgggccctcagcattgaaagtgcaatgtcttaaccattggaccactggggaattcccaatgaagaccattcttaattctaccatactttttgaaaacttgctttttctctctctctctctttaaccacaaactagccaaggactagataaacgaataggctgattatttggggctttgttaaagaagctgttttgggggaggtgagccaggatgggtggtgagtggcagtgtgtgcagactctgctgaacctttggacctcctcctatgactgctccgcttaggaggggaggtgggcagggccaccaagtggtgcaaccccacccctgcagccgtttttcctatattggtttgtgctggtccgcacgtaggagggagagccaggcttcagtacttctggtggcctctgtgactcccctcccccacttctccctataggcaaaactcctggggacccgtaagccgctctttcctgacgccagcaagctgtttggaagtcagacaagggctttagtcggggagataccaagactagctatgacagggttgttgttcgtggcatttcagagtatggaccagctcggctgatggcagtttatattaaaaaaaaaaaatttttttttccttataaaaattatctgaaaggagccccagcccttccaaacatttaatcagccaacaggtaatatttaactgtaggtgactggtgtcacatcacccctttctcaggtgatgagtgcagtcatggcatatgagaccaagccatacaacgttttggaggagcactgaagttttagtctggttaagagtcaggaaagtaaagactcaaaagaaaactatcctgatatctgatatccctcttgttatttcgtcaaagagcagattaacacatcaccaatcagttgggcaaacgttgtaagagtctgacagtgtcaagtgttggcaaaggtgtgggaaggcaagaactcacttgactggttggttgtaaattaatgtaaccactttggagagcaattaagcactattttgtaaaacagaaagcaagcataccctgtggcccaggaatacacctctggtatagaagccacacctgcacaaagagacatgtactaggagaaatattatagtattgattaatgaggaaaaattgggtttagtgggaaaatggtcaaccagtgagggaatacataaatagtctagtcacacactgggctagtatagcagttcatatgaattaactagagctgtgtgccaactggatagatcttaaatatgctgcgaaagcaagttgtttgataatgcctttcagtgtattacttagcatattatagaagcagcagtatcatagtgttgaatagagatacagacacccttagatgaactataaggatagaaaattcatgaccttggttgggtattaggatgtgatgtgaaagggttggaggaaccacctggggatggaggacaaatgaattggatacaatggtaattttaagtctaaaaatctggattaagaacaatttttttctggccatatctcacagcatcttagttccccaaccaggtttgaacccagcccttggtagtgaaagtgtggagtcctaaccattggactgccagggaattccttggataatattaatggtcagttctgggtggcagaaatatagatgttaatgtttcttccttcagttcaaaacagaagcaaacctatggtggtcactgtagggagaccaggcatactaagtaggtcagtgctcgggagatctggcatgcctttctcctcccatcccctcctcttctccctgcagctatgattgattcagggcagctcctcctctaagtactgagccaaggcctcccttttctgaggctatccctcccctggtgttgtacctgacttggaaggatcctgaatcagggtcccaggtcccttggtgcttccagagtgtcagtcttacgccctgaagcactgtctcataattttaagtttagtaaggagcacaactcagcaaaagctttgtctccacttcctccttcagactgaaaccaggtctcttctctgggcatctgatcgaaccaggaaatcctcagcttttgaaatggtagcagtttctctgactcaggacttcctgccctggagttgagtggtccttataggagtgggctctttagcatcagcttttgtgccctattgcacactcagatttgaaataggaccttgggctcctaggtataatatttaagtcacttagttctaaccctgaataaaagagcaagggaattttacttaaacctacagtgggctcattttgttttgtgtaacatggtaacatcaggctttgctttctgttatgggacctgttgagagaatggacttaatattcccatctcttagctaagaactgcttgtcctggccccactccatacttgagagaatggacttaatattcccatctcttagctaagaactgcttgtcctggccccactccatacgcgtatatactgttatcattactctaactgtgtaggaggatgaagggcttctcaggtggctcagtgtaagggacccatctgccagttcaggagacaatcccgactccttggagtaggaagttgcaacgactctagtattcttgcctggagaattgaatggacagaggagcctggtgggctacagtccatggggtcgcaaaagagtaagatgcgaccgacccacaacaacaaataggatgaacagctgagattcattccggaccttactaggttttgtggctggcctggcacccattttgattttgtcgctgcctttgtcagataagggccagtcttgggaattatatttttctgctccatcgtctactaacagagctcctagaaagcagactgacctgaatgccttgtccagcatttttttaatatttatttatttcaccgggtcaggtcttagttacggtacatgggatctagttccctgaccaaggatcaaacctgggccctctgcattgggagctcggagtcttagccactgggctagcaggaaagtccctgtccaacatactgtgatgaaaactttctaatggttgttagtgtgccaaagatttgggaatcagacctccatgctggcttggaaacgtggagatgtagatcgcaaattggtgtcacaattgtgggcagcaggtgccatcatcattgagtcagcctgtgtagtgagattggcatcagagcacacataggttaaaggttggcttatgcagatgtgggctcacaggccaagctggctttctagggcagaatgatgtgggcttttgggaaactcgaatcggtggagtgaagcgtgagtccttcagcacaccctgccagcagtactggctctcagtgacgtgttgggtagtttttcttttatggtttctaccccctggtttctgaggagtgatccaagacttgtgtctggcatgaacagaactgtggcttcagaagaccaggagccacttggaaaggagaggtcggagctggggcccgtggagctgagctgccacttgtgcctgctctcagtcctttcaactgctctcaggcctctgatggtgtcttgtttctcacagaggagaaatgtctctgaggaggatgctacaacaggggctcgggctcaagattcaggttcccgtacccgggcatggcatttgctgccatcagtggagatgcccagcttccactgctggctggaggttggctctgcatgtctgtgcctcctgaggaggaggatgtaggaatgagccagactggcatgagctggacaacgtgtgctggcaggcactgtcccagtggctagcctgagggtgggggagctcttcccagaagctggccctgagaaaccagggctgaggctgggggagagtccagggggtgttttaggtggggatgggcagcttgtgctctggagaagaggctcatgggaggcttcagggcaaagcttgagggaatggaaacccagctggggacctgggcaggtgacaaggtctctggtttgctagtggactttaaggacatggtgagatctcataggaatctggacagatttgctgacaaaccagcttttttggattgctggtgggtacacacactcaaatagcaagtaaaatttcacttgttatttttgtacttagtagcctgggtgagcaaaagatcagagcttgtagacctcaggcttaatctgagataatgaaatagggctagtggttgagccgagtgtattaattgtatctgctgatagcatctcctgttgttgaggcagcccttctgtaccaggatatccttgtcacaaagattttgctctgatgcttgaaggcagactaaacaggaagataagtttgttggatttgggcatgtaactcagatttggggcacagagttgtcctatggaccatattagctttcctccccaccaacttggaagttctggatgaatcaaggttttacctgcccattgcttctgcaccatcagtacaaataccagcacattaaagaaggcaaataatgtctttgaaagctctgctctcacggaccatcctttggtaaccgctagagttactcagttagagtgactcaggtttcttttttcctcactgtgtcatgtggcttgtgggatcttagtttgccaaccagggattgaacctgggcccagggagtgaaagcgctgagtcctaaacactgaaccaccagagaattcctaaggttgatcgtcttagtcagtgaaactatcctgctgtgagtttatgatctagggcccaagatgcattattctacctctaccatgtttagaatattcctgaggtacatagagtggcagttgaataatggtacaaggaccttcacttttgttttaattccaacatttttaccaggacgcttgtaagtgtgtcatttttgaaagaggtacatttagaatttttcttatgaaagtagtatcagtaaactttttagtaaagagaagaaagaaatttgcttgtaatcctaccacacagataatatgctgataatattttggtaggtatccttccagtccagttatgtatgtttaatttagtgcctcccctcccaaagaaggggtggggggtaatagtgaaatattagtaaacgaaggtttaaagagacgttatagggcaggggataaagcattgcaaaggagccactgacaactcttttacctgagaattttcttgggtgacttctgtgtattaatttataacgtagctgaacctctaggaactaccactgcctggatacctggttattaaggtgatttctccattacccccaagagctttagttgcactcagcttttaccttaatccttgtacagctctgcagggcaaacctttctcagctccgagcagtcttttcaagtggggccaaggagccaccctgagtcaaaaaaagagcattatgtcaccggaagcccaagcccagagaacacaaggtacggcacgataatcagtggccccactcgggggtctttgcaaggagaccctaatgtctcaggtctagagccgatccaactaatgcctcttttgggggccacacacagcagtgggatcttactttcccagctgaggatcagactcatggtccttgcagtggaagtgtggagtcttaaccactggactgccagtgaggtccccaactgatgatttttaagaagagttactttaacccactcattgaggtcacactggatgactccatttgcctggaggcctcagtgagacctgtgtgttcccgtggccttggcgtgccaccaagggaaccatctcttcagtggtgttccctttacagccagacgcaccgcagtaatagtgggtgctactgtgtaagcccagcttccaccctgagtgcacggtgggaccttggctgcccaggccatcctgtcagtgattcagaagttctcagatttggtgggggagggggcatagctataactgcgatttgtcacagaggcattaataggtgctctgaaacatcatttggcaaacaccggactacctccaaggtcatggtgctaagagatatgaaaccactatgtcgtggaggaaaggatgttgttttttggtggagaggatgttgttttcatctcggtacctggtatgttgtggggagttaagagatacatatcgaaaggaacagtgtatatgccacgttggatagttggctggtcttagtttcttgtttttaaaggtgtagttggggatctatagctactggttttcagcagcgtgtaggagcctcacttaatacctggtcacagagcttcttgaagttcaccagactttttgaaagtgtcaggcgggtttagatgtgtgctatgccccagccacctggagaggccactcagtcaaggatgcaggaggggaaagagtaagggttacatggatggggtgccttggtttggagccctgagattttggaggaggtgggacagggcaaaagaaaggcttgttcttgtgcctcggctgcagcacttaggcacaggatttgagaaggagcagcccttggtctgaaaacctgcaaaaaggtttctgatgaggactgcaagtttctggctcacgtgcttacctgatgagtcttcagacactgcctggaagcttctcaagtctcttgtctataatcagctaactaactgactgagtttagttgtaaagtcagcaatgaggaaaaataaaggtctgaaccattgtgccctgggtcaggcttgtgacctgggctgattaattacctggaacagatggtctttgttactgtgagtggtgtgctttccagacttagatagggaatgggtggagggtcagaagatcagcctggaaaggatttgacaagcagtgttaatttcttcatagtgcaataattttagagccaggctaggactattgtggttatccttggtttgacgcaaattgctttgaatgtgcctaagtccttctgactgaaagtaccagatgtagttaattcctggggaaaaaaatattttgcattcttggttctggttggacttaacctgtccagggatgggtttttttgcctctcagtgggctggggcatactgtccagcttatggtgaatcgaggaatggacttatgtgtccctgtgggttagtgaaacctaggactccaggactaggaggctacctgaatagtggtctccacgaccaggaaagtcactttgggtttgggtggggactgtgaagagagagtatgggtaccacaccccatagcagatgtttatagctttgagacagcttttcacaaaaagatctagattgaccatggtactaagctaggggtgagttgtctgtatccctggaagattggcaagagattttgacaaaacttccttttgaggaaactcctgcagtgcatgtaaatggtgccatttacaggattagtaggggagttgtcaagatagacattatcccagtgtgagggaggtgtgtgcttaacatgtgcctgggaagtacattcattgggaatgtggttttgacttattctttggggtctttcctacttgctcatgataatgtgagcccatggcccgacttgagcacccacgtagccagagtgtggcggggctgcctgtggcttgtgcacagagcatgcttgttgagttaatgtgtaagcaggaagcagcttctgtgccaagctatatattagccattcaggcagatcacactctctccagttcctctggacccaagacatcagaagccatgtcgaagcaccacagcgacgccgggacggccttcattcagactcagcagctgcacgcagccatggccgacacattcctggagcacatgtgccgcctggacatcgactcaccgcccattacggcccgaaacaccggcatcatctgtaccatcggtgagcgcgggtgcccactccccctgaaacaagggcttcctgagacagtgatcttttctccccccaaaagactaaatgtagcaaacctgggttctgagcaagatgctacagagagattctcaggggaaatgacgtcatggacttggtcgctcagacctgtccaactctgtgacccatggactgtagcccatgaggcccctctgtccatgggatttgtcagacaggaatactggagtgggttgccatttccttcttcaaactgagttcataggaatttgcaggaaggaattcatatgctttatgtgttctgctaaaggtaaaatttagagggtgttttccatggcagttcttactgtctctgagcatctcagagacctaggaaagatcttgtggacttgctctgttcaaccaaaatgctgtagttggacatctgactgggcttcagggctggctacattttactttggttattaagagttctttaaactgaagcacacttgaagtgttaatcctcttttggataatgaacatttaatgactttcaaacttttgttttgttttttctgaactaggcccagcttcacgagcagtggagacattgaaggagatgattaagtctggaatgaatgtggctcgtttgaacttctctcatggaacccacgaggtgagccttgatggtgtggccccttgggcagcttgtaggaaacagggtgggcatgctgtgtttatttagccacagtggaccaggcaggaagaagtggggaatcaatgggaaggttgtcttcaccagcccatccctaccgttcttccccccagatccctgacatctcaagtggccaagtctccttaagcaaagatgacctgctttgtcattggccatggttctccccaacacctgtgctccgttagcctttgataaggaaagcatggaagggttttcctggaatcccctctaagccatggggcttgacacttctgcaccagggtttctataacagctcataattgcaatgatgaaccagtaagtcaccctgcaggttattctgcctattttcattttagacccatgataaatggggcaggtcccagagaagggtttccactattgcaagtgaaggactgggtcctggtttgtagtaccccacttcatcaacattgtgttcccagcctgtcccttcccaggagttctgggacattaaaataaagtcagcacacttgtctagtgatctgggaggagtatgttagcactggggctgtttatgagctgacttggcccctgggaccaggtgacagctattggaaagtgctccactgtgctgctattcccaagatcggaacaagctcattttgaggagtgtgtgggctgcctgaggaacagtgccatgggatttcatactggcatcaatagaagactagtggaggaggcaagctcttgatcctcttggactctgtatttcctgttcagaataaagaaaatgctgccctgtaaggtcacggggttaaggggttgccctctggcatttggaagttggtgataagtttaaactaaaaataaagcctcagtaaatgcagtccaagaactgtatgcctcctttctggaaacatgagagtgttcctcgtaggttccctaccccatgttccataaacagcttatagccttcaaatgtcacagggacctgaccaagttcagaggaactgcgctcagcaggcaccctggggcaggttaagaagcccacagatggcattcttaattccccaaaggtgaagagtcctccgcaacagtcactaggcattcctgcaggcctgtgcttagcccagagagagagctagagacaccagtgaaggtccattcacaattgatccctcctgctgacttgtgccagggtctgctgtgtgcaggggaaggagcagaaataagaaatagcttttaggaagttgacctttaaccaggctttatggcctcttccatctggtaaaataataccacccagctctgaaatggcagggggagagggtggtatctgcacctgttggagggggctggctcaggggagttggtgttttaaactacaagaagcatttggtgattatctgtgctttgactcttattcccagagttggaatttgttttctttttccacactgtatggcaggatcttagttccccaaccagggatcaaacaaacccagggcccctgccttggaggcacagaatcttaaccactggaccacctgggaagtcccttgtgggtttaatttgaagtttttgaccaggatgaagttatgttgcccctggaatcagttttggaaactgatctgaaatattagttatataatgtaaacagttatagtttgacaaacttccaccttttggccgtttttaatactctactaagcagaacattgggtttgctttctgtccttcactcagacagaggaacatctaccatctgatctctttttttggttttgtttatttggtttattgcatctggccttttgacagctttgccacagtgtctccagcccccgcttttggcagctcttgtgggcaagacgtgtcgtattctccggtgccactcacccggatggtggtggttgaaggatgaggcctacatcctgtgttatcagcagtgtcacactgatagcactaggaacttttgccatcttggctccgagtccctttttagtcaaacagtaatggctaatgggaaagagaaagggcttccagggtgagactgaagccgggatttgttgcaagtgagaaatagcaaaggtcagttagttcagagaagcgtattcctacctctccgtgttccgtttccttttctaatcagactggaggaagtgattgtcagaaccaggcctgcatgacttgagcaacatatttaacttgggctgcgtcagcttaagtaaataactgagtttgaattgcggttgtaatcgatagagaagagctcagaattgtcagatctgtcattctaactctggtggccagagcattattatccagatacaaggtcttttaccccagttgtctgctttgtttgctgagaatgtttgttgaatcctagaggctggagggggaggaaacaagcctcttttatgttgacatgtgttgccaccaaaaaccattaaagatatgggtgtaaatattctaaagttcctaagatttgcccttagagtggatgatggcggtggagaagtgggtgaagagctggtaggaatctgctgtatagcacagggagctcaacttggtgctcggggatgacaagaagtgagtagaggcgaggaggttgatgtaagcaggtagaggatgctcggtgatggaggcagtggtctagaatcctggtgaccagcttcagcgctaatcctagatcccgttgtgtgagatgacagatgtggagcttatcgtctcccctcttttcccctagtaccacgcagagaccatcaagaatgtacgtgaagccacggagagctttgcttcagaccccattctctatcggccagtggcagtggccctggacactaaaggacctgagatccgaactgggctcatcaagggcgtgagtatcttggggagaagcaggagagaaggcataggggaccagaaagcccagaagccacgtttctcaaagtttaggtctataaaggagagttttatccatcagtgtagaaagtcttcctttgacctgctttgctaagaaacagtaccagaaagaaacctcttttacactcaaaatctacttccaagtcttttagggagcagcttatctcttcctctttagtataactgaaacactgcaggttattcttgggttatttccttgaaatatactaagttagaaaaggctttattgttccttccaactgaaatttaatcctggttccctagaggaaaaagctgttctcctgccaggaaggcccagctttggacttgtggcttactctcttgtctgctgcagagcggcaccgctgaggtggagctgaagaagggagccacactgaagatcaccctggacaatgcctacatggaaaagtgtgacgagaacatcctgtggctggactacaagaacatttgcaaggtggtggatgtgggcagcaagatctacgtggatgatgggcttatttctctgctggtgaagcagaaaggtagggacagaagtcggtctgattgtttaaaaccagctcccatctctaatttccttgccagaagataggaaggttgtttggttggcagagagactgaatctatatagtctcaggacttcctttcttttttcaaataatctgtttatttatttatatttattttggctgcgttgggtcttagttgtagcacatggggtctttagttgcaacactggactctgtggcacatgggctcatttttttatggctcgagggcttagtggctgtgcagcctgtgggatcttagttcccccaccagggatcaaactcacttcctatgcattgcaaagtggattcttaaacactggaccaccagggaagtcccaggagttccttttataatgaactcctatcctcaaaaacagatttcactccagttcagttgtactcctggagagcaggtgagaagcgcttttacccatcctcactatttaaagatttgcttccctccttcatccacttaacatggtgtctataggactccagtgtgcctgggaatgatggctaacctttaagtgatctgcagtgttctaagctctttctttacatgtagatttgtttaaattttgaaacaacctaatgagtagatactatcatccctgtggagctgagacactgagaggctgatggacttgctcacagttgaattggagtgtgaaccctggcatgtggctccaccacccaccacgtgccccttcccagttctctcgactggggagagcctgcctcctggtggggacacctgcacccagggctcggcctccccaacctgttccattccatacaggtcctgacttcctggtgacggaggtggagaacggtggctccttgggcagcaagaagggtgtgaacctccctggggctgccgtggacctgcctgctgtgtcagaaaaggacatccaggatctaaagtttggggtggagcagaacgtggatatggtgtttgcgtctttcatccgcaaggcttctgatgtccatgaagtcaggaaggtcctgggagagaaaggaaagaacatcaagataatcagcaaaatcgagaatcatgagggagttcggaggtaagtcccctgcctccttcagccccaaccaagctttctgcagagcaatatgtagcctgccacctgcccattagaaataacgtctcaaacttatttcagacccactgactcagaatatcctatgacattggtgggcttgccagatggccctagtggtaaagaccccacctgccagtgtaggagttgtgagttctgtccttggactgagaagatcccctggtgaagggaatggcaacccactctagtattcttgtctggagtatcccatggacagaggagcctggtgggcaaccatccatggcgtcgcaaaagcagacaaaactgaagtgacttagtgcacacgcctgatactggtattggtatttcaggggggagaaaaaaggcttcacagatgattggggtgccagcaccttgacattgttccagggagcagcactggagagggacagggtcaccaccactcccgggctcaagaaccgtgtgtagtgttcctacactggctacgtttgctggaattgggggccaccaaggggaaagacagcaggctctcaggaggagggggctccttctcttggcaagcagtgggattgttggttggggcagctctggcgtggcatccacctccctggggaaaacatgccgtcttcctctggttctgcgtggtcccaatcctgctccactgcttccccagtgctcaccaggcagagccacacgagtgaggccagctgggtgacatctactcctgcttggcctccctactgacagcccaggcttacatgaggggtcaggtgtgtgcatgggaagagtgggaggagccgtgccgcccactgttgcagccttgccatccttagctttgtgtctgtgtccgttgtacggggaaattcagggccctccctctgccatcctgatgagccatgggcacctgctgactttggcttcaggccaggcaaggccatgttcttgtgccttcaacacactggggagggacctctgaaggagcagccttccagtgtccttgactggaagaggccacggacctttgtgtcaagagcagtcagccaccttgggttccatttctgccccagcggtgatgactgaaatgcagatggtagagacacctcaagagggacctcataagatgcaggtggacactgagaatggacttagggttaccagaggggaacgatgggaggaaggaatggtcagggagtttgggatggacctgtacacgcttctgtatttaaaatggatgacccacaaggactactgtagagcacagggaactctgctcggtgttacatggcaacctgggtgggaggagggtctaggggagagtgggtgcacatatgtgtatggctgagtcgctctgctgtgcatctgaaaccatcaagacatggttaatagctatacctcagtataaaataaaaagttaagggaaaaaagacagatggagaggaagtgaacccatgggattttggaggagacccccctggcccctccccttcccctcagtgaagtgtccatcctggtgctgaaggagtccagcgagcagaactgttggcccctttgggggactgagacagctggaacacccctgtgatgatgtcatgggtacttgtcctggtgcaagaatggccaagtcagagaatcccgggaaactttgtgccaccgtacgcgaggcccctgtttgtataacctctgtctctgcaacttgcctgtcaggtttgacgagatcctggaagccagtgatgggatcatggtggctcgtggtgatctgggcattgagatccctgcagagaaggtcttccttgcccagaagatgatgattggtcggtgcaaccgagctgggaagcccgtcatctgtgccacacaggtgtgtgcctctccctgagcgtgcatccttgtacaaggaagctctggaggctacctggtctcttcccatggaccctgccccacatcatacacaaacctattccccccacctgcccccacacacctacacaaagctggattctgtacatacatgctctccagggggtcaccttgtgtccaaagccctcccaggccacatctcacaaatactgtgtcccctcccagatgctggagagcatgatcaagaagcctcgccctacccgggcggagggcagtgacgtggccaatgccgtcttggatggagccgactgcatcatgctgtccggagagacggccaaaggggactaccccctggaagctgtccgcatgcagcacctggtaagttctcccgctgcgggtggaggcaaactccagcatatgggggcaccttgtagtgtcaccctctgtgccctcgatcttttattgtcctccaagtcacacgcagagtgaggaggtgtgtgctggagtcatgggggtgtcttttttgatgggtttttttttttgctcttctgccttcctgcccacacccatctcctagtcccttctgttctggaggcggcctccttcatcacacgtagcacagttcactcccatcttcggggtgtgaatccttcctttgcttgagtcctgggatatctgtactgcttccccctggacagtctagaggacctgagcccttcttccctgttctggaaaagtccaagagctcctgctcccagctggcctgtgtttggcttccattttgattccccacctgtcccagccagtgcctccccgacggggtcctctgggcctgcactgatttggctgtgagctggccttgcatggtacctgcagcagcgcctgctttgctgagctgttgcactcaccctagcagtggggtttaagggaggggtgacaggcccacaaatagaagcaggtggaaggctggctgatcccagctggccctggggtgagagtgtctggaggagccctagtggccgctcctcattctcctgtgggagctagcttttccagggtcttctgctggaccatgtcccacagcctggctgcctctgctctaaagcatcctaaagaggaacacaggcctggggaaacgagcgctacagatggactttcacaagggtcctaggtggctgttgataatgtatagctcagcaaactgtttctttaaccctcctgttcattttccagccagcaaaggaccccaggccctttgtgtgacatccctccaatgcatcctcccccaaaaaacctctggactagataccatcctttccttcttcactttctaagtgattaggctcaagtggcttccaaagaagacatgacagtcacagtggatactttctcaggggtgaaacaagacatcttgactacagatgttttctggtctgtccttggtgttctgggtggatttgcccctaatgaaatcagtagagaccagtgtctgcccttgggacagctgtgtttttctggctgatggccaccccctgtgtgacatgactgctgtcctgatgctcatgcactgcaggctcacggttcttctcacctgtcccagtgagtacttcctctctcctgggagatagtgaccttgaaccagtcccctctagagtggttgatgttagtgtttatgggtttccatatagaattatatacatatactgtaacgataactgctttgcttagtcatgtggccccacggcctctgagaccttgacacgttagactttagggactttgtgtctttgggcacatcattggacctctcaaggccttatctaagaagtgaattagatgggatatgtttaattacaccctttgcgttaaattgcactgctgttataaaatgtctgctaagaacctcatgcaggttcttaacctcagtgaggtcaatgtcaacccacttctaatggatgagggcacatgataatcgagcttgagtttggacccaggtcagctgactctgaagccagcattccctcttagaacacatctagcagatcaggacaggtgtcctgtggggcctgtcctggggtcagagactaaaccagactccgggctctgcaagcatgtctgtttttccaaacccaatcttttctttgaacaaacaccttagaactgtgagactgggattgcatggccctattcaggaggtgagttctcctggccgtgtcaccagttaacactggccaatagcttcccctcttaactgtccactgctttctcctgtaactgacgcaattgacgttcttaactatgggctaggtagtaccagctgtggttctctttcctgttcatgtgccctgtcctcctgggctgcatgcattccattcttgcaggaagaaaccctttaacctagcaaattcagccactcattggctcatcctgtcagattaatgagatggcgttactgattcttccctcacccatcattgtctaagacgctcataactgcattataaccctctaaagcttaagttttctaattcttggtgttacctgagcaagatggtctgaataacacagtggctttctcatgggtgttttaatcattcaattttaatcaaatgccttggtaagcttacctgtgtgcttccacaggcacctgactcttcacatggctgttcgttgtgccccttacaagcttgccagtttccagctgcccgcttagaatgtggtcaagctgcgtgtccttggctttgaccccagctttctagcgctgctcaatgtgcttctgccgcaggccactggagaggcaggcagcatctctgtgtctgaagccaagcaagcactccatgtgtgcatggaggaggccaggcacgaagccctgtggtaactaggcagccatgtgaggaggggggcctattccttcctgcgagctatgtcatgaggcagtgtggtcgagtcctgccagggagccgtggaggggccccagcctccgcatgtttctatgctgaggcgttagagcctgccgtcatattgtctccctcacccccaccgccagtcatggagacctaccaaaatcaagagatccttccagaatggaagaaccagtttcctctctgatggaggaggaacagtagccattgacgcttgaacttccctgagcaggctttcaccaccctgcctctttgcatctgcctgaaggagagacttagccaattaacctacagtaccttcctctcattaattccccgttctgtctttccatgttgttgtctcttgtttgtttccccccaccctccttcttccctcttccttgcctcccctcctctaaaccttacagatagctcgtgaggctgaggcagccatgttccaccgcaagctgtttgaagaacttgcgcgagcctcaagtcactccacagacctcatggaagccatggccatgggcagcgtggaggcttcatataagtgtttagcagcagctttgatagttctcacggagtctggcaggtagggccccgagggcaggtaactctgtaggataaacagccttttgctccagccactctagacgacagccagggcccagccctgagcctaggacgctcctctcgtctgcaggaagccagccagggaggtcagggcaggacagggccacaggatcctccggctcagaaggcacagccatgtcacaggcacctggtgaggggctggttcctgcaagtctcgatctcactcagctcagaacctccagtgatcgggctgttcttggccactgtacccagggacttgctcctccccagctgtctgtgcgactcttcccctccccgtcccgtgtgacacggctctgacagctctgtccccctcgtccctctggacggatgttgcccctagattgcccgtgaggcagaggctgccatctaccatttgcaattgttcgaggagctccgtcgcctgtcacccattaccagcgaccccaccgaagctgccgctgtgggggccgtggaggcgtccttcaagtgctgcagtggggccataatcgtcctcaccaagtctggcaggtaggaggtggcagcaggtccctgggaatgccccgctcagcggcccctttccttgggggtcctgggagcgtgtactgcacggtgctcagatggcattgagccaaggtaagacccctctgcctgcaccccggaccctgcagggaaggagccccctccccctcccccacccagccccccaggcggcacttagtgggtccaaagcgagttttattaaccctcacaacgagctagacttaatcagataactacaggtagcctgaggacaagtggttatttgatattgaaaagctgttcttaaaaaggaaaggaccaccagttttacttctagctaagtgctctttaaaaaagtccagtttgcaaaatgagtttgcaaaaaaagtccagtttgctgacagtgctatttggttggtgtgcctgtcccatctgtctttgagtcccacagataagtgggtgctgcctaggaaaggtcactgtgctatcttgctggcactcgggccaggcaaccagtctgagacctctacttggcctctggcttgagtcccttccaaatccaggatctcaagttaataccccttgctccctgggtctgggggtatgctgctcctccctggggcagctagggtttcctggaggagggaggtggagaggagcagacaggcacccccgtacgccaaggagcagcagctgggactgcctttaggaccaggacacaagatgtgtgccgagtcccgcagtagagggcttagggtaaaaagctccagccgttagggttgacctttgtgaaaaggtccaacctttgctacttggctatttttagtccagtcttctggggtttcactctcagtgagtcctgacttcagcacttgcttccctcagaaaaccttgtatggaggccaggccttaagcagggcatcctctgctgtgacatggcttaagtttccctgacagctgttgcgtgtcctcacagcttgccttctgagcctcgggtcccctgccagcagccccctccaactcctgagacaccagctcctaccagtgtacagtgtcagtgttgaaggaagcatccagcctttgagtttaaaggggcccagtcccctccaggggatccttcataggcagagcctcccccgctgacactccatcccctagtactgccttctgggtagcacttatatttctaatcctcagcctcgcttagatactcaggtttgactctggggctctacagcccagttgctgggactatttcaggggcctgggggtcatgtggagtcaggagagggcacagcagtaggcaaggcttgtcttgtagatttcccaggcaggctggtggtctctgctcctggtttgggtttttttttttttttttttgcagatgtcaagtgtcaggtggtcttcacatgctgcttggcttgtagctgttgctggcagcaagagaaaaggatgtggtgggcttggacagggctgcttccctgtgccagaatgggaaagttcagggaatggtggggggtctgccaagtatgggccatgtggagtaagtgggtaagactgaccatgctgggtcagtatgatcctgcggccctggcctgtaggaggggtttagggtagcatagctctaaagaatatgctatggcagatacagggtgatgggacacctcagtcttgggagcaaatgttgaaatggtcagagcctcaggctggcagagttggattccagggtgagttgttgctgaaggccctgaggggactgtttaaccatcttgtctgcctaccccatctcaggtctgcacatcaggtggccagataccgcccccgggcccccatcattgctgtgactcggaatcaccagacagctcgccaggcccacctataccgcggcatcttccctgtggtgtgtaaggacccagtgcaggaggcctgggctgaggacgtggatctccgggtgaacttggccatgaatgttggtgcgtgtctgggagagaaagctcccggtggagggctggggaaggtgctagagcactggcttctctgagaccctattgaaagaggaacatgccagcctttgtgggcaagagctggcctcactaggaatgttctctggttccttcccagtccctggagtcctgggctacgctcagggcagggagaggacctttgacgtgtggggctgagcagcccaggttctcacctcacgttctttgggagtgggatccaaagcaaaggatcctggataatcacctttgtccattctttggcacttgctcattattgaggttttttttttcttccctataaaattaaccagatctcctacacttggggccagtacacatttagattatgtactcaaatggcctttgtcacagtataaaagtaggatgtcacaggatgagaggaaacagcgagctgtgaccttgggctgggctctccaccttcttgagtagaaaataaatgatgtccaaaggtccctgctacctctgccatctgagtgataaggaaataataccaaggcctccctcattccctttgcctgggctgtgagcctggcgccaccttgtggtgtaagaaagtaaaggcctgtcagcccttcctcagttgtggggcactccaatactttggccacctcatgtgaagagttgactcattggaaaagactctgatgctgggagggattgggggcaggaggagaaggggatgacagaggatgagatggctggatggcatcgactcgatggacatgagtttgagtgaactccaggagttggtgatggacagggaggcctggcgtgctgcgattcatgaggtcgcaaagagtcggacacgactgagtgactgaactgaactcccaagtgtataccttcttggcttaatttttcatttaccagctctcttctcttcttccaggaaaggcccgaggcttcttcaagaagggagacgtggtcattgtgctgaccgggtggcgccctggttccggcttcaccaacaccatgcgtgtagttcctgtgccatgatggactccgaagcccctcctccagcccctgtcccacccctcttccccaaaccatccgttaggccagcattgcttgtagtgctcacttggggctgtaatgtggcactggtgggctgggacaccagggaagaagatgaatacctctgtgaaacctggctggttttaagactttgcttgggtcgggtagttcagagctgggccactcatcacgtggctgcatccaagcaggggatgcaggagggatgcaggcaggactggaggctccagagcttcacacacagggcaacagctcctgcttcccttcctttgtatacccgattcagctcctgcagaaaatggatatccagagaattcccagccctggcccgtgatcaagagtaggggccttagggcatggggcagtggttccagtttaagtagactctggccctggcccttacttgcttctccaaccccctcagcctccctccccttgtgcaccgtatacttctcttccttcactccacccagccgatgctgcaaacactccaccccccatcttccatttcccccactactgcagctgcctccaggcttgttgctatagagcctacctgtatgtcaataaacaacagctgaagcacc

(19) United States
(12) Patent Application Publication
(10) Pub. No.: US 2025/0283068 A1
(43) Pub. Date: Sep. 11, 2025

(54) MODULATING LACTOGENIC ACTIVITY IN MAMMALIAN CELLS

(71) Applicant: GENENTECH, INC., South San Francisco, CA (US)

(72) Inventors: Shahram Misaghi, South San Francisco, CA (US); Masaru Ken Shiratori, South San Francisco, CA (US); Bradley Richard Snedecor, South San Francisco, CA (US); Michael W. Laird, South San Francisco, CA (US)

(73) Assignee: GENENTECH, INC., South San Francisco, CA (US)

(21) Appl. No.: 19/170,262

(22) Filed: Apr. 4, 2025

Related U.S. Application Data
(62) Division of application No. 17/036,075, filed on Sep. 29, 2020, now Pat. No. 12,286,619.
Application No. 17/036,075 is a continuation of application No. PCT/US2019/024774, filed on Mar. 29, 2019.
(60) Provisional application No. 62/649,963, filed on Mar. 29, 2018.

(51) Int. Cl. C12N 15/10; C12N 5/071; C12N 9/22; C12N 15/113

(52) U.S. Cl. CPC C12N 15/102; C12N 5/0682; C12N 9/22; C12N 15/113; C12N 2310/20

(57) ABSTRACT

The present disclosure relates to methods, cells and compositions for producing a product of interest, e.g., a recombinant protein. In particular, the present disclosure provides improved mammalian cells expressing the product of interests, where the cells (e.g., Chinese Hamster Ovary (CHO) cells) have modulated lactogenic activity. The present disclosure also relates to methods and compositions for modulating pyruvate kinase muscle (PKM) expression (e.g., PKM-1 expression) in a mammalian cell to thereby reduce or eliminate the lactogenic activity of the cell, as well compositions comprising a cell having reduced or eliminated lactogenic activity and methods of using the same.

### CROSS REFERENCE TO RELATED APPLICATIONS

This application is a divisional of U.S. application Ser. No. 17/036,075, filed Sep. 29, 2020, which is a continuation of International Patent Application No. PCT/US2019/024774, filed Mar. 29, 2019, which claims priority to U.S. Provisional Application No. 62/649,963, filed Mar. 29, 2018, the contents of each of which are hereby incorporated by reference in their entireties.

### SEQUENCE LISTING

The present application contains a Sequence Listing which has been submitted in xml format via EFS and is hereby incorporated by reference in its entirety. Said xml copy, created on May 16, 2025, is named 00B206_1532_SL.xml and is 379,842 bytes in size.

### 1. FIELD OF INVENTION

The present disclosure relates to methods and compositions for producing a product of interest, e.g., a recombinant protein. In particular, the present disclosure is directed to mammalian cells expressing a product of interest, where the cells (e.g., Chinese Hamster Ovary (CHO) cells) have modulated lactogenic activity. The present disclosure is also directed to methods and compositions for modulating pyruvate kinase muscle (PKM) expression (e.g., PKM-1 expression) in a mammalian cell to reduce or eliminate the lactogenic activity of the cell, as well compositions comprising one or more cells that have reduced or eliminated lactogenic activity and methods of using the same.

### 2. BACKGROUND

Chinese hamster ovary (CHO) cells have been widely used in the production of therapeutic proteins for clinical applications because of their capacity for proper protein folding, assembly and post translational application. Normally, CHO cells, like other immortalized cell lines, tend to consume glucose and generate lactate through aerobic glycolysis, a process known as the Warburg effect (Warburg, 1956, Science 123(3191):309-14). Accumulation of lactate in the production culture can adversely affect cell growth, viability and productivity. Such lactogenic behavior (i.e., lactate generating behavior) of CHO cells during manufacturing processes can cause a decline in viability and productivity and can alter the quality of the produced therapeutic proteins.

Several approaches targeting the process conditions have been developed to mitigate lactate generation in lactogenic CHO cell lines. For example, optimizing copper levels has been shown to be effective in preventing lactogenic behavior in some CHO cell lines (Luo et al., 2012, Biotechnol. Bioeng. 109(1):146-56; Xu et al., 2016, Bioprocess Biosyst. Eng. 39(11):1689-702). Another approach involving controlled nutrient feeding triggered by rising pH in culture (HIgh-end pH-controlled Delivery of Glucose, or HIPDOG) has also been shown to be effective in reducing or eliminating lactate accumulation in large scale CHO cultures (Gagnon et al., 2011, Biotechnol. Bioeng. 108(6):1328-37). However, these approaches have their limitations as the former approach cannot apply to all lactogenic CHO cell lines, and the latter can complicate the process of large-scale manufacturing. Furthermore, these approaches do not target the underlying mechanisms of lactogenic behavior in CHO cells.

Therefore, there is a need in the art for techniques for reducing lactate production in cell cultures.

### 3. SUMMARY

The present disclosure relates to methods, cells and compositions for producing a product of interest, e.g., a recombinant protein. In particular, the methods, cells and compositions described herein include improved mammalian cells expressing the product of interest, where the cells (e.g., Chinese Hamster Ovary (CHO) cells) have modulated lactogenic activity. The methods and compositions described herein modulate the lactogenic activity of mammalian cells, and thus reduce or eliminate the undesired effects associated with the lactogenic activity, e.g., reduced viability and productivity of the mammalian cells and altered quality of the produced products of interest.

This disclosure is further directed to methods and compositions for modulating pyruvate kinase muscle (PKM) expression (e.g., PKM-1 expression) in a mammalian cell to thereby reduce or eliminate the lactogenic activity of the cell, as well cells having reduced or eliminated lactogenic activity and methods of using the same.

In one aspect, the present disclosure relates to a mammalian cell having reduced or eliminated lactogenic activity, in which the expression of a pyruvate kinase muscle (PKM) polypeptide isoform, or isoforms, is knocked down or knocked out. In certain embodiments, the PKM polypeptide isoform knocked out or knocked down is the PKM-1 polypeptide isoform. In certain embodiments, the PKM polypeptide isoforms that are knocked out or knocked down are both the PKM-1 polypeptide isoform and the PKM-2 polypeptide isoform. In certain embodiments, the lactogenic activity of the mammalian cell is less than about 50%, e.g., less than about 20%, of the lactogenic activity of a reference cell. In certain embodiments, the reference cell is a cell that comprises one or more wild-type alleles of the PKM gene, e.g., both alleles of the PKM gene are wild-type or unmodified. In certain embodiments, the lactogenic activity of the mammalian cell is determined at day 14 or day 15 of a production phase. In certain embodiments, the mammalian cell produces less than about 1.0 g/L or less than about 2.0 g/L of lactate during a production phase, e.g., produces less than about 1.0 g/L or less than about 2.0 g/L of lactate during a production phase in a shake flask. In certain embodiments, the mammalian cell produces less than about 2.0 g/L of lactate during a production phase in a bioreactor. The present disclosure provides a mammalian cell comprising an allele of a PKM gene that comprises a nucleotide sequence selected from the group consisting of SEQ ID NOs: 39-41, or comprises the nucleotide sequences set forth in SEQ ID NOs: 37 and 38. The present disclosure further provides compositions comprising one or more cells, e.g., mammalian cells, disclosed herein.

In certain embodiments, the mammalian cell comprises a nucleic acid sequence encoding a product of interest. In certain embodiments, the nucleic acid sequence is integrated in the cellular genome of the mammalian cell at a targeted location. Alternatively and/or additionally, the nucleic acid encoding the product of interest is randomly integrated into the cellular genome of the mammalian cell. In certain embodiments, the mammalian cell is a CHO cell.

In certain embodiments, the product of interest comprises a protein, e.g., a recombinant protein. In certain embodiments, the product of interest comprises an antibody or an antigen-binding fragment thereof. For example, but not by way of limitation, the antibody is a multispecific antibody or an antigen-binding fragment thereof. In certain embodiments, the antibody consists of a single heavy chain sequence and a single light chain sequence or antigen-binding fragments thereof. In certain embodiments, the antibody is a chimeric antibody, a human antibody or a humanized antibody and/or a monoclonal antibody.

In another aspect, the present disclosure relates to a method for reducing or eliminating the lactogenic activity in a cell. In certain embodiments, the method includes knocking down or knocking out the expression of a pyruvate kinase muscle (PKM) polypeptide isoform. In certain embodiments, the method includes administering to the cell a genetic engineering system, in which the genetic engineering system knocks down or knocks out the expression of a pyruvate kinase muscle (PKM) polypeptide isoform. In certain embodiments, the genetic engineering system is selected from the group consisting of a CRISPR/Cas system, a zinc-finger nuclease (ZFN) system, a transcription activator-like effector nuclease (TALEN) system and a combination thereof. In certain embodiments, the method results in the cell having a lactogenic activity that is less than about 50%, e.g., less than about 20%, of the lactogenic activity of a reference cell. In certain embodiments, the reference cell is a cell that comprises one or more wild-type alleles of the PKM gene, e.g., both alleles of the PKM gene are wild-type or unmodified. In certain embodiments, the lactogenic activity of the cell is determined at day 14 or day 15 of a production phase. In certain embodiments, the cell produces less than about 1.0 g/L or less than about 2.0 g/L of lactate during a production phase, e.g., produces less than about 1.0 g/L or less than about 2.0 g/L of lactate during a production phase in a shake flask. In certain embodiments, the cell produces less than about 2.0 g/L of lactate during a production phase in a bioreactor.

In certain non-limiting embodiments, a genetic engineering system for use in the present disclosure is a CRISPR/Cas9 system that includes a Cas9 molecule, and one or more guide RNAs (gRNAs) comprising a targeting domain that is complementary to a target sequence of the PKM gene. In certain embodiments, the target sequence is selected from the group consisting of: a portion of the PKM gene, a 5′ intron region flanking exon 9 of the PKM gene, a 3′ intron region flanking exon 9 of the PKM gene, a 3′ intron region flanking exon 10 of the PKM gene, a region within exon 1 of the PKM gene, a region within exon 2 of the PKM gene, a region within exon 12 of the PKM gene and combinations thereof. In certain embodiments, the one or more gRNAs comprise a first gRNA comprising a target sequence that is complementary to a 5′ intron region flanking exon 9 of the PKM gene and a second gRNA comprising a target domain that is complementary to a 3′ intron region flanking exon 9 of the PKM gene. In certain embodiments, the one or more gRNAs comprise a first gRNA comprising a target sequence that is complementary to a region within exon 2 of the PKM gene and a second gRNA comprising a target domain that is complementary to a region within exon 12 of the PKM gene. For example, but not by way of limitation, the one or more gRNAs comprise a sequence selected from the group consisting of SEQ ID NOs: 33-34 and 42-43, and a combination thereof. In certain embodiments, the expression of the PKM polypeptide isoform is knocked out or knocked down, and the lactogenic activity in the cell is eliminated. In certain embodiments, the PKM polypeptide isoform is a PKM-1 polypeptide isoform or a combination of the PKM-1 and PKM-2 polypeptide isoforms.

In certain embodiments, a genetic engineering system for use in the present disclosure is a zinc-finger nuclease (ZFN) system or a transcription activator-like effector nuclease (TALEN) system. In certain non-limiting embodiments, the genetic engineering system includes an RNA selected from the group consisting of a short hairpin RNA (shRNA), a small interference RNA (siRNA) and a micro RNA (miRNA) and the RNA is complementary to an mRNA expressed by the PKM gene. In certain embodiments, the mRNA expressed by the PKM gene encodes a PKM-1 polypeptide isoform. In certain embodiments, the expression of PKM-1 polypeptide isoform is knocked down, and the lactogenic activity of the cell is reduced. In certain embodiments, the genetic engineering system further comprises a second RNA selected from the group consisting of a shRNA, an siRNA and a microRNA miRNA, wherein the second RNA is complementary to a portion of an mRNA expressed by the PKM gene that encodes the PKM-2 polypeptide isoform. In certain embodiments, the expression of PKM-1 and PKM-2 polypeptide isoforms are knocked out or knocked down, and the lactogenic activity of the cell is reduced.

In a further aspect, the present disclosure provides methods for producing a product of interest, e.g., from the cells disclosed herein. For example, a method of producing a product of interest comprises culturing mammalian cells to produce the product of interest, wherein the mammalian cells have reduced or eliminated lactogenic activity. In certain embodiments, the present disclosure provides a method of culturing a population of mammalian cells expressing a product of interest, wherein the mammalian cells have reduced or eliminated lactogenic activity. In certain embodiments, the reduction or elimination of lactogenic activity results from knocking down or knocking out the expression of a pyruvate kinase muscle (PKM) polypeptide isoform in the mammalian cells. In certain embodiments, the PKM polypeptide isoform is the PKM-1 polypeptide isoform. In certain embodiments, expression of the PKM-2 polypeptide isoform is also knocked down or knocked out. In certain embodiments, the method can further comprise isolating the product of interest from the cell culture.

### 4. BRIEF DESCRIPTION OF THE DRAWINGS

FIGS. 1A-1F. Lactate levels of mAb-1 cell line directly correlate with the PKM-1 levels. Percent cell viability (FIG. 1A), day 14 titer (FIG. 1B), and lactate levels (FIG. 1C) in mAb-1 cell line under low and high lactate processes. (FIG. 1D) PKM-1 and PKM-2 western blot analysis of mAb-1 cell lysates at different days during production under the low and high lactate processes. Actin was used as the loading control. Quantification of relative PKM-1 (FIG. 1E) and PKM-2 (FIG. 1F) levels, normalized against Actin.

FIGS. 2A-2F. Lactate levels of mAb-2 cell line directly correlate with the PKM-1 levels. Percent cell viability (FIG. 2A), day 14 titer (FIG. 2B), and lactate levels (FIG. 2C) in mAb-2 cell line under low and high lactate processes. (FIG. 2D) PKM-1 and PKM-2 western blot analysis of mAb-2 cell lysates at different days during production under the low and high lactate processes. Actin was used as the loading control. Quantification of relative PKM-1 (FIG. 2E) and PKM-2 (FIG. 2F) levels, normalized against Actin.

FIGS. 3A-3E. CHO cells do express PKL/R enzymes, but levels of these enzymes do not correlate with lactate levels in production culture. (FIG. 3A) Western blot analysis of intracellular expression of PKL/R proteins in two different CHO host cell lines (K1 and DHFR −/−) as well as human 293 cells. Actin is used as loading control. (FIGS. 3B and 3C) Intracellular protein levels of PKL/R at different days during the low and high lactate production processes of mAb-1 (FIG. 3B) and mAb-2 (FIG. 3C) cell lines. (FIGS. 3D and 3E) Quantification of relative PKL/R levels normalized against Actin in mAb-1 (FIG. 3D) and mAb-2 (FIG. 3E) cell lines.

FIGS. 4A-4C. Generation of PKM-1 knock out cell lines. (FIG. 4A) Schematic of exons 8-10 of PKM gene and gRNA and screening primers used to evaluate deletion of exon-9 in mAb-2 cell line. Two gRNAs flanking the exon 9 of PKM gene were co-transfected with Cas9 transgene to induce exon 9 deletion. Transfected cells were single cell cloned. PCR primers flanking the deletion region were used to screen cell lines with successful deletion of exon-9 in PKM allele(s). (FIG. 4B) Wild type (WT) mAb-2 cells and cell lines with targeted deletion of exon-9 via gRNAs and Cas9 transgene were analyzed by screening PCR primers. Upper band represents WT PKM allele(s) while lower band represents PKM allele(s) with exon 9 deletions. KO: knock out cell lines, HET: heterozygous cell lines. (FIG. 4C) Sequence comparison of PKM WT allele in contrast with exon-9 KO allele(s) in targeted cell lines. Note that KO-15 cell line bears larger than intended (by 122 bp) deletions in 5′ region of exon-9 in targeted PKM allele(s).

FIGS. 5A-5H. Abolishing or reducing PKM-1 expression averted lactogenic behaviors in mAb-2 cell line even under high-lactate process. Viable cell count (FIG. 5A), Percent cell viability (FIG. 5B), day 14 titer (FIG. 5C), day 14 specific productivity (FIG. 5D), and lactate levels (FIG. 5E) of WT, and PKM-1 HET and KO mAb-2 cell lines during a 14-day fed-batch production assay using AMBR15 bioreactors. Process control was the same as the high lactate process in FIG. 2. (FIG. 5F) Western blot analysis of cell lysates of indicated cell lines for PKM-1 and PKM-2 proteins. Actin is used as a loading control. (FIGS. 5G and 5H) Antibody product qualities including percent aggregation (FIG. 5G) and percent charge variant (FIG. 5H) of mAb-2 from indicated cell lines.

FIGS. 6A-6G. Lactogenic behavior is averted or reduced in PKM-1 KO or HET mAb-2 cell lines, using the high-lactate process, irrespective of cell age or post thaw. Viable cell count (FIG. 6A), percent cell viability (FIG. 6B), day 14 titer (FIG. 6C), day 14 specific productivity (FIG. 6D), and lactate levels (FIG. 6E) of indicated cell lines, at a young cell age, in a 14-day fed-batch production assay using AMBR15 bioreactors. (FIGS. 6F and 6G) Antibody product qualities including percent aggregation (FIG. 6F) and percent charge variant (FIG. 6G) of mAb-2 from indicated cell lines. Process control was the same as in FIG. 5. Error bars represent standard error from four individual experiments.

FIG. 7. MAb-3 producing pools derived from PKM and PKM-1 KO host cell lines had comparable or higher Qp and titer, but lower growth (in shake flask production).

FIGS. 8A-8B. MAb-3 producing pools derived from PKM and PKM-1 KO host cell lines generated lower lactate (FIG. 8A), but consumed more glucose (FIG. 8B) in shake flask production.

FIG. 9A. MAb-3 producing pools derived from PKM and PKM-1 KO host cell lines exhibited different amino acid synthesis/consumption rates in shake flask production.

FIG. 9B. Bars and pathways enclosed in rectangles show that PKM KO host cell lines accumulated 3-phosphoglycerate.

FIG. 9C. Bars and pathways enclosed in rectangles show that WT host cell lines accumulated pyruvate.

FIG. 9D. Bars and pathways enclosed in rectangles show that PKM-1 KO host cell lines accumulated the TCA cycle product, alpha-ketoglutarate (a-KG).

FIG. 9E. Bars and pathways enclosed in rectangles show that PKM-1 KO host cell lines accumulated the TCA cycle product, oxaloacetate.

FIG. 10. Host cell specific consumption or generation of glucose, lactate and amino acids.

FIG. 11. MAb-3 producing pools derived from PKM and PKM-1 KO host cell lines exhibited different glycosylation profiles in shake flask production. PKM KO host had decreased galactosylation, and PKM KO and PKM-1 KO host cell lines had slightly decreased fucosylation.

### 5. DETAILED DESCRIPTION

For clarity, but not by way of limitation, the detailed description of the presently disclosed subject matter is divided into the following subsections:
5.1 Definitions;
5.2 Modulating PKM expression;
5.3 Cells with reduced or eliminated lactogenic activity;
5.4 Cell culture methods;
5.5 Products; and
5.6 Exemplary embodiments.

### 5.1. DEFINITIONS

The terms used in this specification generally have their ordinary meanings in the art, within the context of this disclosure and in the specific context where each term is used. Certain terms are discussed below, or elsewhere in the specification, to provide additional guidance to the practitioner in describing the compositions and methods of the present disclosure and how to make and use them.

As used herein, the use of the word “a” or “an” when used in conjunction with the term “comprising” in the claims and/or the specification can mean “one,” but it is also consistent with the meaning of “one or more,” “at least one” and “one or more than one.”

The terms “comprise(s),” “include(s),” “having,” “has,” “can,” “contain(s)” and variants thereof, as used herein, are intended to be open-ended transitional phrases, terms or words that do not preclude the possibility of additional acts or structures. The present disclosure also contemplates other embodiments “comprising,” “consisting of” and “consisting essentially of,” the embodiments or elements presented herein, whether explicitly set forth or not.

As used herein, the term “lactogenic behavior” or “lactogenic activity” refers to the lactate producing activity of a cell, for example, by consuming glucose and generating lactate through aerobic glycolysis. In certain embodiments, the lactogenic activity of a cell can be measured by the level of accumulated lactate in the cell culture medium.

The term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value.

The terms “cell culture medium” and “culture medium” refer to a nutrient solution used for growing mammalian cells that typically provides at least one component from one or more of the following categories:
1) an energy source, usually in the form of a carbohydrate such as glucose;
2) all essential amino acids, and usually the basic set of twenty amino acids plus cysteine;
3) vitamins and/or other organic compounds required at low concentrations;
4) free fatty acids; and
5) trace elements, where trace elements are defined as inorganic compounds or naturally occurring elements that are typically required at very low concentrations, usually in the micromolar range.

The nutrient solution can optionally be supplemented with one or more components from any of the following categories:
1) hormones and other growth factors as, for example, insulin, transferrin, and epidermal growth factor;
2) salts and buffers as, for example, calcium, magnesium, and phosphate;
3) nucleosides and bases such as, for example, adenosine, thymidine, and hypoxanthine; and
4) protein and tissue hydrolysates.

“Culturing” a cell refers to contacting a cell with a cell culture medium under conditions suitable to the survival and/or growth and/or proliferation of the cell.

“Batch culture” refers to a culture in which all components for cell culturing (including the cells and all culture nutrients) are supplied to the culturing bioreactor at the start of the culturing process.

“Fed-batch cell culture,” as used herein refers to a batch culture wherein the cells and culture medium are supplied to the culturing bioreactor initially, and additional culture nutrients are fed, continuously or in discrete increments, to the culture during the culturing process, with or without periodic cell and/or product harvest before termination of culture.

“Perfusion culture,” sometimes referred to as continuous culture, is a culture by which the cells are restrained in the culture by, e.g., filtration, encapsulation, anchoring to microcarriers, etc., and the culture medium is continuously, step-wise or intermittently introduced (or any combination of these) and removed from the culturing bioreactor.

As used herein, the term “cell,” refers to animal cells, mammalian cells, cultured cells, host cells, recombinant cells and recombinant host cells. Such cells are generally cell lines obtained or derived from mammalian tissues which are able to grow and survive when placed in media containing appropriate nutrients and/or growth factors.

The terms “host cell,” “host cell line” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny does not need to be completely identical in nucleic acid content to a parent cell, but can contain mutations. Mutant progeny that have the same function or biological activity as screened or selected for in the originally transformed cell are included herein.

The term “mammalian host cell” or “mammalian cell” refers to cell lines derived from mammals that are capable of growth and survival when placed in either monolayer culture or in suspension culture in a medium containing the appropriate nutrients and growth factors. The necessary growth factors for a particular cell line are readily determined empirically without undue experimentation, as described for example in Mammalian Cell Culture (Mather, J. P. ed., Plenum Press, N.Y. 1984), and Barnes and Sato, (1980) Cell, 22:649. Typically, the cells are capable of expressing and secreting large quantities of a particular protein, e.g., glycoprotein, of interest into the culture medium. Examples of suitable mammalian host cells within the context of the present disclosure can include Chinese hamster ovary cells/-DHFR (CHO, Urlaub and Chasin, Proc. Natl. Acad. Sci. USA, 77:4216 1980); dp12.CHO cells (EP 307,247 published 15 Mar. 1989); CHO-K1 (ATCC, CCL-61); monkey kidney CV1 line transformed by SV40 (COS-7, ATCC CRL 1651); human embryonic kidney line (293 or 293 cells subcloned for growth in suspension culture, Graham et al., J. Gen Virol., 36:59 1977); baby hamster kidney cells (BHK, ATCC CCL 10); mouse sertoli cells (TM4, Mather, Biol. Reprod., 23:243-251 1980); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells (Mather et al., Annals N.Y. Acad. Sci., 383:44-68 1982); MRC 5 cells; FS4 cells; and a human hepatoma line (Hep G2). In certain embodiments, the mammalian cells include Chinese hamster ovary cells/-DHFR (CHO, Urlaub and Chasin, Proc. Natl. Acad. Sci. USA, 77:4216 1980); dp12.CHO cells (EP 307,247 published 15 Mar. 1989).

The term “peptone” within the context of the present disclosure is meant to refer to a media supplement that is essentially hydrolyzed animal protein. The source of this protein can be animal by-products from slaughter houses, purified gelatin, or plant material. The protein is typically hydrolyzed using acid, heat or various enzyme preparations.

“Growth phase” of the cell culture refers to the period of exponential cell growth (the log phase) where cells are generally rapidly dividing. The duration of time for which the cells are maintained at growth phase can vary based on the cell-type, the rate of growth of cells and/or the culture conditions, for example. In certain embodiments, during this phase, cells are cultured for a period of time, usually between 1-4 days, and under such conditions that cell growth is maximized. The determination of the growth cycle for the host cell can be determined for the particular host cell envisioned without undue experimentation. “Period of time and under such conditions that cell growth is maximized” and the like, refer to those culture conditions that, for a particular cell line, are determined to be optimal for cell growth and division. In certain embodiments, during the growth phase, cells are cultured in nutrient medium containing the necessary additives generally at about 30°-40° C. in a humidified, controlled atmosphere, such that optimal growth is achieved for the particular cell line. In certain embodiments, cells are maintained in the growth phase for a period of about between one and four days, usually between two to three days.

“Transition phase” of the cell culture refers to the period of time during which culture conditions for the production phase are engaged. During the transition phase environmental factors such as temperature of the cell culture, medium osmolality and the like are shifted from growth conditions to production conditions.

“Production phase” of the cell culture refers to the period of time during which cell growth is/has plateaued. The logarithmic cell growth typically decreases before or during this phase and protein production takes over. During the production phase, logarithmic cell growth has ended, and protein production is primary. During this period of time the medium is generally supplemented to support continued protein production and to achieve the desired glycoprotein product. Fed-batch and/or perfusion cell culture processes supplement the cell culture medium or provide fresh medium during this phase to achieve and/or maintain desired cell density, viability and/or recombinant protein product titer. A production phase can be conducted at large scale.

The term “expression” or “expresses” are used herein to refer to transcription and translation occurring within a host cell. The level of expression of a product gene in a host cell can be determined on the basis of either the amount of corresponding mRNA that is present in the cell or the amount of the protein encoded by the product gene that is produced by the cell. For example, mRNA transcribed from a product gene is desirably quantitated by northern hybridization. Sambrook et al., Molecular Cloning: A Laboratory Manual, pp. 7.3-7.57 (Cold Spring Harbor Laboratory Press, 1989). Protein encoded by a product gene can be quantitated either by assaying for the biological activity of the protein or by employing assays that are independent of such activity, such as western blotting or radioimmunoassay using antibodies that are capable of reacting with the protein. Sambrook et al., Molecular Cloning: A Laboratory Manual, pp. 18.1-18.88 (Cold Spring Harbor Laboratory Press, 1989).

As used herein, “polypeptide” refers generally to peptides and proteins having more than about ten amino acids. The polypeptides can be homologous to the host cell, or preferably, can be exogenous, meaning that they are heterologous, i.e., foreign, to the host cell being utilized, such as a human protein produced by a Chinese hamster ovary cell, or a yeast polypeptide produced by a mammalian cell. In certain embodiments, mammalian polypeptides (polypeptides that were originally derived from a mammalian organism) are used, more preferably those which are directly secreted into the medium.

The term “protein” is meant to refer to a sequence of amino acids for which the chain length is sufficient to produce the higher levels of tertiary and/or quaternary structure. This is to distinguish from “peptides” or other small molecular weight drugs that do not have such structure. Typically, the protein herein will have a molecular weight of at least about 15-20 kD, preferably at least about 20 kD. Examples of proteins encompassed within the definition herein include all mammalian proteins, in particular, therapeutic and diagnostic proteins, such as therapeutic and diagnostic antibodies, and, in general proteins that contain one or more disulfide bonds, including multi-chain polypeptides comprising one or more inter- and/or intrachain disulfide bonds.

The term “antibody” is used herein in the broadest sense and encompasses various antibody structures including, but not limited to, monoclonal antibodies, polyclonal antibodies, monospecific antibodies (e.g., antibodies consisting of a single heavy chain sequence and a single light chain sequence, including multimers of such pairings), multispecific antibodies (e.g., bispecific antibodies) and antibody fragments so long as they exhibit the desired antigen-binding activity.

An “antibody fragment,” “antigen-binding portion” of an antibody (or simply “antibody portion”) or “antigen-binding fragment” of an antibody, as used herein, refers to a molecule other than an intact antibody that comprises a portion of an intact antibody that binds the antigen to which the intact antibody binds. Examples of antibody fragments include, but are not limited to, Fv, Fab, Fab′, Fab′-SH, F(ab′)₂; diabodies; linear antibodies; single-chain antibody molecules (e.g., scFv, and scFab); single domain antibodies (dAbs); and multispecific antibodies formed from antibody fragments. For a review of certain antibody fragments, see Holliger and Hudson, Nature Biotechnology 23:1126-1136 (2005).

The term “chimeric” antibody refers to an antibody in which a portion of the heavy and/or light chain is derived from a particular source or species, while the remainder of the heavy and/or light chain is derived from a different source or species.

The “class” of an antibody refers to the type of constant domain or constant region possessed by its heavy chain. There are five major classes of antibodies: IgA, IgD, IgE, IgG and IgM, and several of these can be further divided into subclasses (isotypes), e.g., IgG₁, IgG₂, IgG₃, IgG₄, IgA₁, and IgA₂. In certain embodiments, the antibody is of the IgG₁ isotype. In certain embodiments, the antibody is of the IgG₂ isotype. The heavy chain constant domains that correspond to the different classes of immunoglobulins are called α, δ, ε, γ and μ, respectively. The light chain of an antibody can be assigned to one of two types, called kappa (κ) and lambda (λ), based on the amino acid sequence of its constant domain.

The term “titer” as used herein refers to the total amount of recombinantly expressed antibody produced by a cell culture divided by a given amount of medium volume. Titer is typically expressed in units of milligrams of antibody per milliliter or liter of medium (mg/ml or mg/L). In certain embodiments, titer is expressed in grams of antibody per liter of medium (g/L). Titer can be expressed or assessed in terms of a relative measurement, such as a percentage increase in titer as compared obtaining the protein product under different culture conditions.

The term “nucleic acid,” “nucleic acid molecule” or “polynucleotide” includes any compound and/or substance that comprises a polymer of nucleotides. Each nucleotide is composed of a base, specifically a purine- or pyrimidine base (i.e., cytosine (C), guanine (G), adenine (A), thymine (T) or uracil (U)), a sugar (i.e., deoxyribose or ribose), and a phosphate group. Often, the nucleic acid molecule is described by the sequence of bases, whereby said bases represent the primary structure (linear structure) of a nucleic acid molecule. The sequence of bases is typically represented from 5′ to 3′. Herein, the term nucleic acid molecule encompasses deoxyribonucleic acid (DNA) including, e.g., complementary DNA (cDNA) and genomic DNA, ribonucleic acid (RNA), in particular messenger RNA (mRNA), synthetic forms of DNA or RNA, and mixed polymers comprising two or more of these molecules. The nucleic acid molecule can be linear or circular. In addition, the term nucleic acid molecule includes both, sense and antisense strands, as well as single stranded and double stranded forms. Moreover, the herein described nucleic acid molecule can contain naturally occurring or non-naturally occurring nucleotides. Examples of non-naturally occurring nucleotides include modified nucleotide bases with derivatized sugars or phosphate backbone linkages or chemically modified residues. Nucleic acid molecules also encompass DNA and RNA molecules which are suitable as a vector for direct expression of an antibody of the disclosure in vitro and/or in vivo, e.g., in a host or patient. Such DNA (e.g., cDNA) or RNA (e.g., mRNA) vectors, can be unmodified or modified. For example, mRNA can be chemically modified to enhance the stability of the RNA vector and/or expression of the encoded molecule so that mRNA can be injected into a subject to generate the antibody in vivo (see, e.g., Stadler et al, Nature Medicine 2017, published online 12 Jun. 2017, doi:10.1038/nm.4356 or EP 2 101 823 B1).

As used herein, the term “vector” refers to a nucleic acid molecule capable of transporting another nucleic acid to which it has been linked.

The term “hybridoma” refers to a hybrid cell line produced by the fusion of an immortal cell line of immunologic origin and an antibody producing cell. The term encompasses progeny of heterohybrid myeloma fusions, which are the result of a fusion with human cells and a murine myeloma cell line subsequently fused with a plasma cell, commonly known as a trioma cell line. Furthermore, the term is meant to include any immortalized hybrid cell line which produces antibodies such as, for example, quadromas. See, e.g., Milstein et al., Nature, 537:3053 (1983).

A “human antibody” is one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues.

A “humanized” antibody refers to a chimeric antibody comprising amino acid residues from non-human CDRs and amino acid residues from human FRs. In certain aspects, a humanized antibody will comprise substantially all of at least one, and typically two, variable domains, in which all or substantially all of the CDRs correspond to those of a non-human antibody, and all or substantially all of the FRs correspond to those of a human antibody. A humanized antibody optionally can comprise at least a portion of an antibody constant region derived from a human antibody. A “humanized form” of an antibody, e.g., a non-human antibody, refers to an antibody that has undergone humanization.

The term “hypervariable region” or “HVR” as used herein refers to each of the regions of an antibody variable domain which are hypervariable in sequence and which determine antigen binding specificity, for example “complementarity determining regions” (“CDRs”).

Generally, antibodies comprise six CDRs: three in the VH (CDR-H1, CDR-H2, CDR-H3), and three in the VL (CDR-L1, CDR-L2, CDR-L3). Exemplary CDRs herein include:
(a) hypervariable loops occurring at amino acid residues 26-32 (L1), 50-52 (L2), 91-96 (L3), 26-32 (H1), 53-55 (H2), and 96-101 (H3) (Chothia and Lesk, J. Mol. Biol. 196:901-917 (1987));
(b) CDRs occurring at amino acid residues 24-34 (L1), 50-56 (L2), 89-97 (L3), 31-35b (H1), 50-65 (H2), and 95-102 (H3) (Kabat et al., Sequences of Proteins of Immunological Interest, 5th Ed. Public Health Service, National Institutes of Health, Bethesda, MD (1991)); and
(c) antigen contacts occurring at amino acid residues 27c-36 (L1), 46-55 (L2), 89-96 (L3), 30-35b (H1), 47-58 (H2), and 93-101 (H3) (MacCallum et al. J. Mol. Biol. 262: 732-745 (1996)).

Unless otherwise indicated, the CDRs are determined according to Kabat et al., supra. One of skill in the art will understand that the CDR designations can also be determined according to Chothia, supra, McCallum, supra, or any other scientifically accepted nomenclature system.

An “immunoconjugate” is an antibody conjugated to one or more heterologous molecule(s), including but not limited to a cytotoxic agent.

The term “monoclonal antibody” as used herein refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies in accordance with the presently disclosed subject matter can be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

The term “variable region” or “variable domain” refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three complementary determining regions (CDRs). (See, e.g., Kindt et al. Kuby Immunology, 6^{th} ed., W.H. Freeman and Co., page 91 (2007).) A single VH or VL domain can be sufficient to confer antigen-binding specificity. Furthermore, antibodies that bind a particular antigen can be isolated using a VH or VL domain from an antibody that binds the antigen to screen a library of complementary VL or VH domains, respectively. See, e.g., Portolano et al., J. Immunol. 150:880-887 (1993); Clarkson et al., Nature 352:624-628 (1991).

As used herein, the term “cell density” refers to the number of cells in a given volume of medium. In certain embodiments, a high cell density is desirable in that it can lead to higher protein productivity. Cell density can be monitored by any technique known in the art, including, but not limited to, extracting samples from a culture and analyzing the cells under a microscope, using a commercially available cell counting device or by using a commercially available suitable probe introduced into the bioreactor itself (or into a loop through which the medium and suspended cells are passed and then returned to the bioreactor).

As used herein, the term “seeding” refers to the addition or inoculation of growing cells into a culture medium at the beginning of the production phase. Further, as used herein, the term “seed train” refers to a continual passaging of cells in volumes of culture medium of about 20 L or less for the maintenance of the cell line.

As used herein, the term “recombinant cell” refers to cells which have some genetic modification from the original parent cells from which they are derived. Such genetic modification can be the result of an introduction of a heterologous gene for expression of the gene product, e.g., a recombinant protein.

As used herein, the term “recombinant protein” refers generally to peptides and proteins, including antibodies. Such recombinant proteins are “heterologous,” i.e., foreign to the host cell being utilized, such as an antibody produced by CHO cells.

As used herein, a “PKM polypeptide” refers to a polypeptide that is encoded by the PKM gene. A PKM polypeptide includes the PKM-1 polypeptide isoform and/or the PKM-2 polypeptide isoform.

### 5.2. MODULATING PKM EXPRESSION

Glycolysis is the process of glucose metabolism used by mammalian cells, e.g., CHO cells, to generate energy. Glycolysis can occur at high or low flux states. The cell response to glucose levels and the switch between these flux states can vary depending on the cell lines and the levels and the combinations of isozymes present in the glycolytic pathway (Mulukutla et al., 2014, PLoS One 9(6):e98756). Under normal culture conditions, CHO cells, like other immortalized cell lines, tend to consume glucose and generate lactate through aerobic glycolysis, a process known as the Warburg effect (Warburg, 1956, Science 123(3191):309-14). Accumulation of lactate in production cultures can adversely affect cellular growth, viability and productivity. Therefore, regulation of cellular energy flux and metabolism can be leveraged to avoid undesirable outcomes caused by lactate accumulation during the cell culture production phase (Mulukutla et al., 2010, Trends Biotechnol. 28(9):476-84; Luo et al., 2012, Biotechnol. Bioeng. 109(1):146-56; Ahn and Antoniewicz, 2012, Biotechnol. J. 7(1):61-74).

The final step of the glycolysis process involves conversion of phosphoenolpyruvate (PEP) to pyruvate, which is mediated by the pyruvate kinase (PK) enzymes. Four different isoforms of PK have been identified: PK liver (PKL), PK red blood cells (PKR), PK muscle 1 (PKM-1) and PK muscle 2 (PKM-2). The PK enzymes are expressed by two different genes, PKLR and PKM. Alternative exon splicing gives rise to different PK isoforms. The presence of both exons 1 and 2 in an mRNA transcript from the PKLR gene results in expression of PKR protein, whereas an mRNA transcript that starts with exon 2 results in the expression of PKL protein. Alternative splicing of exon 9 or 10 in the PKM gene transcript gives rise to PKM-1 or PKM-2, respectively. Specifically, PKM-1 includes exon 9 and excludes exon 10 of the PKM gene, and PKM-2 includes exon 10 and excludes exon 9 of the PKM gene. Tissue specific promoters, transcription factors, and alternative splicing regulate expression of these isoforms in different tissues and cell lines (Chaneton and Gottlieb, 2012, Trends. Biochem. Sci. 37(8):309-16; Israelsen and Vander Heiden, 2015, Semin Cell Dev Biol 43:43-51; Mazurek, 2011, Int. J. Biochem. Cell Biol 43(7):969-80; Harada et al., 1978, Biochim. Biophys. Acta. 524(2):327-39; Noguchi et al., 1986, J. Biol. Chem. 261(29):13807-12; Noguchi et al., 1987, J. Biol. Chem. 262(29):14366-71).

PKM-2 has been extensively studied due to its central role in cancer cell metabolism and tumor growth, hallmarked by high glucose consumption and lactate production. While the PKM-1 enzyme is constitutively active, PKM-2 activity is regulated by oligomerization, substrate binding and post-translational modifications (Christofk et al., 2008, Nature 452(7184):230-3; Chaneton and Gottlieb, 2012, Trends Biochem. Sci. 37(8):309-16; Israelsen and Vander Heiden, 2015, Semin. Cell Dev. Biol 43:43-51). For example, fructose 1,6-bisphosphate (FBP) reversibly binds to PKM-2, promoting its tetramerization and hence activation (Ashizawa et al., 1991, J. Biol. Chem. 266(25):16842-6; Dombrauckas et al., 2005, Biochemistry 44(27):9417-29). Phosphorylation of PKM-2 at tyrosine 105 or its binding to other phosphotyrosine proteins inactivates PKM-2 by blocking FBP binding and preventing PKM-2 tetramerization (Christofk et al., 2008, Nature 452(7184):181-6; Hitosugi et al., 2009, Sci. Signal 2(97):ra73). Increased levels of glycolysis, however, promote acetylation of PKM-2 at lysine 305 as part of a metabolic feedback loop, which targets PKM-2 for its degradation via chaperone-mediated autophagy (Lv et al., 2011, Mol. Cell 42(6):719-30) (Macintyre and Rathmell, 2011, Mol. Cell 42(6):713-4). Additionally, PKM-2 dimers have been shown to localize to the cell nucleus acting as protein kinases, utilizing PEP as a phosphate donor, to promote cell proliferation through phosphorylation of STAT3 and activation of MEK5 (Gao et al., 2012, Mol. Cell 45(5):598-609).

In accordance with one aspect, the present disclosure relates to methods for modulating lactogenic activity of a mammalian cell by modulating PKM expression, e.g., PKM polypeptide expression, in the cell. For example, but not by way of limitation, methods for modulating lactogenic activity of a mammalian cell include knocking out or knocking down PKM polypeptide expression in the cell. In certain embodiments, the expression of PKM-1 is knocked down or knocked out. In certain embodiments, the expression of PKM-2 is knocked down or knocked out. In certain embodiments, the expression of both PKM-1 and PKM-2 are knocked down or knocked out. As used herein, knocked out expression refers to the elimination of the expression of a PKM polypeptide, e.g., a PKM-1 polypeptide and/or a PKM-2 polypeptide, in the cell as compared to a reference cell. As used herein, knocked down expression refers to a reduction in the expression of a PKM polypeptide, e.g., a PKM-1 polypeptide and/or a PKM-2 polypeptide, in the cell as compared to a reference cell.

In certain embodiments, the reference cells are cells where the expression of a PKM polypeptide, e.g., PKM-1 and/or PKM-2, is not modulated, e.g., reduced. In certain embodiments, a reference cell is a cell that comprises at least one or both wild-type alleles of the PKM gene. For example, but not by way of limitation, a reference cell is a cell that has both wild-type PKM alleles. In certain embodiments, the reference cells are WT CHO cells.

In certain embodiments, the expression of a PKM polypeptide, e.g., PKM-1 and/or PKM-2, in a cell that has been modified to knock down expression of the PKM polypeptide is less than about 90%, less than about 80%, less than about 70%, less than about 60%, less than about 50%, less than about 40%, less than about 30%, less than about 20%, less than about 10%, less than about 5%, less than about 4%, less than about 3%, less than about 2% or less than about 1% of the PKM polypeptide expression of a reference cell, e.g., a WT CHO cell. In certain embodiments, the expression of a PKM-1 polypeptide in a cell that has been modified to knock down expression of the PKM-1 polypeptide is less than about 90%, less than about 80%, less than about 70%, less than about 60%, less than about 50%, less than about 40%, less than about 30%, less than about 20%, less than about 10%, less than about 5%, less than about 4%, less than about 3%, less than about 2%, or less than about 1% of the PKM-1 polypeptide expression of a reference cell, e.g., a WT CHO cell.

In certain embodiments, the expression of a PKM polypeptide, e.g., PKM-1 and/or PKM-2, in a cell that has been modified to knock down expression of the PKM polypeptide is at least about 90%, at least about 80%, at least about 70%, at least about 60%, at least about 50%, at least about 40%, at least about 30%, at least about 20%, at least about 10%, at least about 5%, at least about 4%, at least about 3%, at least about 2% or at least about 1% of the PKM polypeptide expression of a reference cell, e.g., a WT CHO cell. In certain embodiments, the expression of a PKM-1 polypeptide in a cell that has been modified to knock down expression of the PKM-1 polypeptide is at least about 90%, at least about 80%, at least about 70%, at least about 60%, at least about 50%, at least about 40%, at least about 30%, at least about 20%, at least about 10%, at least about 5%, at least about 4%, at least about 3%, at least about 2%, or at least about 1% of the PKM-1 polypeptide expression of a reference cell, e.g., a WT CHO cell.

In certain embodiments, the expression of a PKM polypeptide, e.g., PKM-1 and/or PKM-2, in a cell that has been modified to knock down expression of the PKM polypeptide is no more than about 90%, no more than about 80%, no more than about 70%, no more than about 60%, no more than about 50%, no more than about 40%, no more than about 30%, no more than about 20%, no more than about 10%, no more than about 5%, no more than about 4%, no more than about 3%, no more than about 2% or no more than about 1% of the PKM polypeptide expression of a reference cell, e.g., a WT CHO cell. In certain embodiments, the expression of a PKM polypeptide, e.g., PKM-1 and/or PKM-2, in a cell that has been modified to knock down expression of the PKM polypeptide is no more than about 40% of the PKM polypeptide expression of a reference cell, e.g., a WT CHO cell. In certain embodiments, the expression of a PKM-1 polypeptide in a cell that has been modified to knock down expression of the PKM-1 polypeptide is no more than about 90%, no more than about 80%, no more than about 70%, no more than about 60%, no more than about 50%, no more than about 40%, no more than about 30%, no more than about 20%, no more than about 10%, no more than about 5%, no more than about 4%, no more than about 3%, no more than about 2% or no more than about 1% of the PKM-1 polypeptide expression of a reference cell, e.g., a WT CHO cell.

In certain embodiments, the expression of a PKM polypeptide, e.g., PKM-1 and/or PKM-2, in a cell that has been modified to knock down expression of the PKM polypeptide is between about 1% and about 90%, between about 10% and about 90%, between about 20% and about 90%, between about 25% and about 90%, between about 30% and about 90%, between about 40% and about 90%, between about 50% and about 90%, between about 60% and about 90%, between about 70% and about 90%, between about 80% and about 90%, between about 85% and about 90%, between about 1% and about 80%, between about 10% and about 80%, between about 20% and about 80%, between about 30% and about 80%, between about 40% and about 80%, between about 50% and about 80%, between about 60% and about 80%, between about 70% and about 80%, between about 75% and about 80%, between about 1% and about 70%, between about 10% and about 70%, between about 20% and about 70%, between about 30% and about 70%, between about 40% and about 70%, between about 50% and about 70%, between about 60% and about 70%, between about 65% and about 70%, between about 1% and about 60%, between about 10% and about 60%, between about 20% and about 60%, between about 30% and about 60%, between about 40% and about 60%, between about 50% and about 60%, between about 55% and about 60%, between about 1% and about 50%, between about 10% and about 50%, between about 20% and about 50%, between about 30% and about 50%, between about 40% and about 50%, between about 45% and about 50%, between about 1% and about 40%, between about 10% and about 40%, between about 20% and about 40%, between about 30% and about 40%, between about 35% and about 40%, between about 1% and about 30%, between about 10% and about 30%, between about 20% and about 30%, between about 25% and about 30%, between about 1% and about 20%, between about 5% and about 20%, between about 10% and about 20%, between about 15% and about 20%, between about 1% and about 10%, between about 5% and about 10%, between about 5% and about 20%, between about 5% and about 30%, between about 5% and about 40% of the PKM-1 polypeptide expression of a reference cell, e.g., a WT CHO cell. In certain embodiments, the expression of a PKM-1 polypeptide in a cell that has been modified to knock down expression of the PKM-1 polypeptide is between about 1% and about 90%, between about 10% and about 90%, between about 20% and about 90%, between about 25% and about 90%, between about 30% and about 90%, between about 40% and about 90%, between about 50% and about 90%, between about 60% and about 90%, between about 70% and about 90%, between about 80% and about 90%, between about 85% and about 90%, between about 1% and about 80%, between about 10% and about 80%, between about 20% and about 80%, between about 30% and about 80%, between about 40% and about 80%, between about 50% and about 80%, between about 60% and about 80%, between about 70% and about 80%, between about 75% and about 80%, between about 1% and about 70%, between about 10% and about 70%, between about 20% and about 70%, between about 30% and about 70%, between about 40% and about 70%, between about 50% and about 70%, between about 60% and about 70%, between about 65% and about 70%, between about 1% and about 60%, between about 10% and about 60%, between about 20% and about 60%, between about 30% and about 60%, between about 40% and about 60%, between about 50% and about 60%, between about 55% and about 60%, between about 1% and about 50%, between about 10% and about 50%, between about 20% and about 50%, between about 30% and about 50%, between about 40% and about 50%, between about 45% and about 50%, between about 1% and about 40%, between about 10% and about 40%, between about 20% and about 40%, between about 30% and about 40%, between about 35% and about 40%, between about 1% and about 30%, between about 10% and about 30%, between about 20% and about 30%, between about 25% and about 30%, between about 1% and about 20%, between about 5% and about 20%, between about 10% and about 20%, between about 15% and about 20%, between about 1% and about 10%, between about 5% and about 10%, between about 5% and about 20%, between about 5% and about 30%, between about 5% and about 40% of the PKM-1 polypeptide expression of a reference cell, e.g., a WT CHO cell.

In certain embodiments, the expression of a PKM polypeptide, e.g., PKM-1 and/or PKM-2, in a cell that has been modified to knock down expression of the PKM polypeptide is between about 5% and about 40% of the PKM polypeptide expression of a reference cell, e.g., a WT CHO cell. In certain embodiments, the expression of a PKM-1 polypeptide in a cell that has been modified to knock down expression of the PKM-1 polypeptide is between about 5% and about 40% of the PKM-1 polypeptide expression of a reference cell, e.g., a WT CHO cell. The expression level of the PKM polypeptide, e.g., PKM-1 and/or PKM-2, in different reference cells (e.g., cells that comprise at least one or both wild-type alleles of the PKM gene) can vary. For example, seed train cells or low-lactate producing cells may produce low levels of PKM-1, whereas high-lactate producing cells may produce high levels of PKM-1.

Alternative splicing of exon 9 or 10 in the PKM gene transcript gives rise to PKM-1 or PKM-2, respectively. The PKM gene that is knocked down or knocked out can be a PKM gene from a human. In certain embodiments, the PKM gene can be from a non-human, e.g., a Rhesus Monkey, canine, green monkey, chicken, cattle, pig, mouse, Chinese hamster, rat or rabbit.

In certain embodiments, the PKM gene that is knocked down or knocked out is a Chinese hamster PKM gene. In certain embodiments, the Chinese hamster PKM gene sequence has a NCBI Reference Sequence ID NW_003613709.1 (range: 200602 . . . 223561) (SEQ ID NO: 44). In certain embodiments, the PKM gene sequence differs by 1, 2, 3, 4, 5, 6, 7, 8, 9, 10, 11, 12, 13, 14, 15, 16, 17, 18, 19, 20 or more nucleotides from the NW_003613709.1 sequence. In certain embodiments, the PKM gene sequence differs by 1%, 2%, 3%, 4%, 5%, 6%, 7%, 8%, 9%, 10%, 11%, 12%, 13%, 14%, 15%, 16%, 17%, 18%, 19%, 20% or more from the sequence set forth in SEQ ID NO: 44.

Additional non-limiting examples of PKM genes include the human PKM gene (e.g., NC_000015.10 (range 72199029 . . . 72231624) (SEQ ID NO: 45)), the Rhesus monkey PKM gene (e.g., NC_027899.1 (range 49211766 . . . 49245983) (SEQ ID NO: 46)), the green monkey PKM gene (e.g., NC_023667.1 (range 11224332 . . . 11255538) (SEQ ID NO: 47)), the canine PKM gene (e.g., NC_006612.3 (range 35712853 . . . 35737643) (SEQ ID NO: 48)), the mouse PKM gene (e.g., NC_000075.6 (range 59656368 . . . 59679375) (SEQ ID NO: 49)), the rat PKM gene (e.g., NC_005107.4 (range 64480963 . . . 64502957) (SEQ ID NO: 50)), the rabbit PKM gene (e.g., NC_013685.1 (range 304096 . . . 317843) (SEQ ID NO: 51)), the chicken PKM gene (e.g., NC_006097.4 (range 1506428 . . . 1523684) (SEQ ID NO: 52)), the pig PKM gene (e.g., NC_010449.5 (range 60971807 . . . 61032780) (SEQ ID NO: 53)) and the cattle PKM gene (e.g., AC_000167.1 (range 18965981 . . . 18992644) (SEQ ID NO: 54)). In certain embodiments, the PKM gene sequence differs by 1, 2, 3, 4, 5, 6, 7, 8, 9, 10, 11, 12, 13, 14, 15, 16, 17, 18, 19, 20 or more nucleotides from any one of the sequences set forth in SEQ ID NOs: 45-54. In certain embodiments, the PKM gene sequence differs by 1%, 2%, 3%, 4%, 5%, 6%, 7%, 8%, ^{9%}, 10%, 11%, 12%, 13%, 14%, 15%, 16%, 17%, 18%, 19%, 20% or more from any one of the sequences set forth in SEQ ID NOs: 45-54.

One skilled in the art would know that different mammalian cell lines, even those from the same species, e.g., two CHO distinct host cell lines, may not share identical PKM gene sequences. Small differences in the sequences of PKM gene, e.g., differences of 1, 2, 3, 4, 5, 6, 7, 8, 9, 10, 11, 12, 13, 14, 15, 16, 17, 18, 19 or 20 nucleotides, can exist between two mammalian cell lines from the same species.

### 5.2.1 Methods for Modulating PKM Expression

In certain embodiments, a genetic engineering system is employed to modulate (e.g., knock down or knock out) the expression of a PKM polypeptide (e.g., PKM-1 expression). Various genetic engineering systems known in the art can be used for the methods disclosed herein. Non-limiting examples of such systems include the CRISPR/Cas system, the zinc-finger nuclease (ZFN) system, the transcription activator-like effector nuclease (TALEN) system and the use of other tools for protein knockdown by gene silencing, such as small interfering RNAs (siRNAs), short hairpin RNA (shRNA), and microRNA (miRNA). Any CRISPR/Cas systems known in the art, including traditional, enhanced or modified Cas systems, as well as other bacterial based genome excising tools such as Cpf-1 can be used with the methods disclosed herein.

Any PKM inhibitors known in the art can also be used with the methods disclosed herein to modulate PKM activity, and thus modulate lactogenic activity of the cells disclosed herein. Non-limiting examples of PKM inhibitors include sodium monofluorophosphate, L-phenylalanine, creatine phosphate, Ca²⁺, flurophosphate and pyridoxal 5′-phosphate.

In certain embodiments, a portion of the PKM gene is deleted to modulate, e.g., knock down or knock out, expression of a PKM polypeptide. In certain embodiments, at least about 2%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85% or at least about 90% of the PKM gene is deleted. In certain embodiments, no more than about 2%, no more than about 5%, no more than about 10%, no more than about 15%, no more than about 20%, no more than about 25%, no more than about 30%, no more than about 35%, no more than about 40%, no more than about 45%, no more than about 50%, no more than about 55%, no more than about 60%, no more than about 65%, no more than about 70%, no more than about 75%, no more than about 80%, no more than about 85% or no more than about 90% of the PKM gene is deleted. In certain embodiments, between about 2% and about 90%, between about 10% and about 90%, between about 20% and about 90%, between about 25% and about 90%, between about 30% and about 90%, between about 40% and about 90%, between about 50% and about 90%, between about 60% and about 90%, between about 70% and about 90%, between about 80% and about 90%, between about 85% and about 90%, between about 2% and about 80%, between about 10% and about 80%, between about 20% and about 80%, between about 30% and about 80%, between about 40% and about 80%, between about 50% and about 80%, between about 60% and about 80%, between about 70% and about 80%, between about 75% and about 80%, between about 2% and about 70%, between about 10% and about 70%, between about 20% and about 70%, between about 30% and about 70%, between about 40% and about 70%, between about 50% and about 70%, between about 60% and about 70%, between about 65% and about 70%, between about 2% and about 60%, between about 10% and about 60%, between about 20% and about 60%, between about 30% and about 60%, between about 40% and about 60%, between about 50% and about 60%, between about 55% and about 60%, between about 2% and about 50%, between about 10% and about 50%, between about 20% and about 50%, between about 30% and about 50%, between about 40% and about 50%, between about 45% and about 50%, between about 2% and about 40%, between about 10% and about 40%, between about 20% and about 40%, between about 30% and about 40%, between about 35% and about 40%, between about 2% and about 30%, between about 10% and about 30%, between about 20% and about 30%, between about 25% and about 30%, between about 2% and about 20%, between about 5% and about 20%, between about 10% and about 20%, between about 15% and about 20%, between about 2% and about 10%, between about 5% and about 10%, or between about 2% and about 5% of the PKM gene is deleted.

In certain embodiments, at least one exon of the PKM gene is at least partially deleted. “Partially deleted,” as used herein, refers to at least about 2%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, no more than about 2%, no more than about 5%, no more than about 10%, no more than about 15%, no more than about 20%, no more than about 25%, no more than about 30%, no more than about 35%, no more than about 40%, no more than about 45%, no more than about 50%, no more than about 55%, no more than about 60%, no more than about 65%, no more than about 70%, no more than about 75%, no more than about 80%, no more than about 85%, no more than about 90%, no more than about 95%, between about 2% and about 90%, between about 10% and about 90%, between about 20% and about 90%, between about 25% and about 90%, between about 30% and about 90%, between about 40% and about 90%, between about 50% and about 90%, between about 60% and about 90%, between about 70% and about 90%, between about 80% and about 90%, between about 85% and about 90%, between about 2% and about 80%, between about 10% and about 80%, between about 20% and about 80%, between about 30% and about 80%, between about 40% and about 80%, between about 50% and about 80%, between about 60% and about 80%, between about 70% and about 80%, between about 75% and about 80%, between about 2% and about 70%, between about 10% and about 70%, between about 20% and about 70%, between about 30% and about 70%, between about 40% and about 70%, between about 50% and about 70%, between about 60% and about 70%, between about 65% and about 70%, between about 2% and about 60%, between about 10% and about 60%, between about 20% and about 60%, between about 30% and about 60%, between about 40% and about 60%, between about 50% and about 60%, between about 55% and about 60%, between about 2% and about 50%, between about 10% and about 50%, between about 20% and about 50%, between about 30% and about 50%, between about 40% and about 50%, between about 45% and about 50%, between about 2% and about 40%, between about 10% and about 40%, between about 20% and about 40%, between about 30% and about 40%, between about 35% and about 40%, between about 2% and about 30%, between about 10% and about 30%, between about 20% and about 30%, between about 25% and about 30%, between about 2% and about 20%, between about 5% and about 20%, between about 10% and about 20%, between about 15% and about 20%, between about 2% and about 10%, between about 5% and about 10%, or between about 2% and about 5% of a region, e.g., of the exon, is deleted. For example, but not by way of limitation, exon 9 of the PKM gene can be at least partially deleted or completely deleted. In certain embodiments, exon 10 of the PKM gene can be at least partially deleted or completely deleted. In certain embodiments, exons 9 and 10 of the PKM gene can be at least partially deleted or completely deleted. In certain embodiments, the region that encompasses exons 1-12 is at least partially deleted or completed deleted.

In certain non-limiting embodiments, a CRISPR/Cas9 system is employed to modulate the expression of a PKM polypeptide. A clustered regularly-interspaced short palindromic repeats (CRISPR) system is a genome editing tool discovered in prokaryotic cells. When utilized for genome editing, the system includes Cas9 (a protein able to modify DNA utilizing crRNA as its guide), CRISPR RNA (crRNA, contains the RNA used by Cas9 to guide it to the correct section of host DNA along with a region that binds to tracrRNA (generally in a hairpin loop form) forming an active complex with Cas9), and trans-activating crRNA (tracrRNA, binds to crRNA and forms an active complex with Cas9). The terms “guide RNA” and “gRNA” refer to any nucleic acid that promotes the specific association (or “targeting”) of an RNA-guided nuclease such as a Cas9 to a target sequence such as a genomic or episomal sequence in a cell. gRNAs can be unimolecular (comprising a single RNA molecule, and referred to alternatively as chimeric) or modular (comprising more than one, and typically two, separate RNA molecules, such as a crRNA and a tracrRNA, which are usually associated with one another, for instance by duplexing). CRISPR/Cas9 strategies can employ a vector to transfect the mammalian cell. The guide RNA (gRNA) can be designed for each application as this is the sequence that Cas9 uses to identify and directly bind to the target DNA in a cell. Multiple crRNAs and the tracrRNA can be packaged together to form a single-guide RNA (sgRNA). The sgRNA can be joined together with the Cas9 gene and made into a vector in order to be transfected into cells.

In certain embodiments, the CRISPR/Cas9 system for use in modulating expression of one or more PKM polypeptides comprises a Cas9 molecule and one or more gRNAs comprising a targeting domain that is complementary to a target sequence of the PKM gene. In certain embodiments, the target gene is a region of the PKM gene. The target sequence can be any exon or intron region within the PKM gene, e.g., the targeting of which eliminates or reduces the expression of PKM1 and/or PKM2 polypeptides. In certain embodiments, the target sequence can be a 5′ region flanking exon 1, a region within exon 1, a 5′ region flanking exon 2, a region within exon 2, a 5′ region flanking exon 9, a 3′ region flanking exon 9, a 3′ region flanking exon 10, a region within exon 12 and/or a 3′ region flanking exon 12 of the PKM gene. For example, but not by way of limitation, the target sequence is selected from the group consisting of a 5′ intron region flanking exon 9 of PKM gene, a 3′ intron region flanking exon 9 of PKM gene, a 3′ intron region flanking exon 10 of PKM gene and a combination thereof.

In certain embodiments, a 5′ intron region flanking exon 9 of the PKM gene and a 3′ intron region flanking exon 9 of the PKM gene are both targeted using a CRISPR/Cas9 system disclosed herein. For example, but not by way of limitation, the CRISPR/Cas9 system comprises a Cas9 molecule, a gRNA targeting a 5′ intron region flanking exon 9 of PKM gene and a gRNA targeting a 3′ intron region flanking exon 9 of the PKM gene, e.g., for generating cells that have PKM-1 knocked down or knocked out. In certain embodiments, the gRNA targeting a 5′ intron region flanking exon 9 of PKM gene comprises the sequence set forth in SEQ ID NO: 33. In certain embodiments, the gRNA targeting a 3′ intron region flanking exon 9 of PKM gene comprises the sequence set forth in SEQ ID NO: 34.

In certain embodiments, the target sequence can be a 5′ region flanking exon 1, a region within exon 1, a 5′ region flanking exon 2, a region within exon 2, a region within exon 12, a 3′ region flanking exon 12 or a combination thereof. For example, and not by way for limitation, a CRISPR/Cas9 system of the present disclosure can comprise a Cas9 molecule, a gRNA targeting a region within exon 1 of the PKM gene and a gRNA targeting a region within exon 12 of the PKM gene, e.g., for generating cells that have both PKM-1 and PKM-2 knocked down or knocked out. In certain embodiments, the CRISPR/Cas9 system of the present disclosure can comprise a Cas9 molecule, a gRNA targeting a region within exon 2 of the PKM gene and a gRNA targeting a region within exon 12 of the PKM gene, e.g., for generating cells that have both PKM-1 and PKM-2 knocked down or knocked out. In certain embodiments, the gRNA targeting a region within exon 2 of the PKM gene comprises the sequence set forth in SEQ ID NO: 42. In certain embodiments, the gRNA targeting a region within exon 12 of the PKM gene comprises the sequence set forth in SEQ ID NO: 43.

In certain embodiments, the gRNAs are administered to the cell in a single vector and the Cas9 molecule is administered to the cell in a second vector. In certain embodiments, the gRNAs and the Cas9 molecule are administered to the cell in a single vector. Alternatively, each of the gRNAs and Cas9 molecule can be administered by separate vectors. In certain embodiments, the CRISPR/Cas9 system can be delivered to the cell as a ribonucleoprotein complex (RNP) that comprises a Cas9 protein complexed with one or more gRNAs, e.g., delivered by electroporation (see, e.g., DeWitt et al., Methods 121-122:9-15 (2017) for additional methods of delivering RNPs to a cell). In certain embodiments, administering the CRISPR/Cas9 system to the cell results in the knock out or knock down of the expression of the PKM-1 polypeptide. In certain embodiments, administering the CRISPR/Cas9 system to the cell results in the knock out or knock down of the expression of both the PKM-1 and PKM-2 polypeptides.

In certain embodiments, the genetic engineering system is a ZFN system for modulating the expression of a PKM polypeptide in a mammalian cell. The ZFN can act as restriction enzyme, which is generated by combining a zinc finger DNA-binding domain with a DNA-cleavage domain. A zinc finger domain can be engineered to target specific DNA sequences which allows the zinc-finger nuclease to target desired sequences within genomes. The DNA-binding domains of individual ZFNs typically contain a plurality of individual zinc finger repeats and can each recognize a plurality of base pairs. The most common method to generate a new zinc-finger domain is to combine smaller zinc-finger “modules” of known specificity. The most common cleavage domain in ZFNs is the non-specific cleavage domain from the type IIs restriction endonuclease FokI. ZFN modulates the expression of proteins by producing double-strand breaks (DSBs) in the target DNA sequence, which will, in the absence of a homologous template, be repaired by non-homologous end-joining (NHEJ). Such repair can result in deletion or insertion of base-pairs, producing frame-shift and preventing the production of the harmful protein (Durai et al., Nucleic Acids Res.; 33 (18): 5978-90 (2005)). Multiple pairs of ZFNs can also be used to completely remove entire large segments of genomic sequence (Lee et al., Genome Res.; 20 (1): 81-9 (2010)). In certain embodiments, the target gene is part of the PKM gene. In certain embodiments, the target sequence is exon 9 of PKM gene. In certain embodiments, the target sequence is exon 9 and exon 10 of PKM gene.

In certain embodiments, the genetic engineering system is a TALEN system for modulating the expression of a PKM polypeptide in a mammalian cell. TALENs are restriction enzymes that can be engineered to cut specific sequences of DNA. TALEN systems operate on a similar principle as ZFNs. TALENs are generated by combining a transcription activator-like effectors DNA-binding domain with a DNA cleavage domain. Transcription activator-like effectors (TALEs) are composed of 33-34 amino acid repeating motifs with two variable positions that have a strong recognition for specific nucleotides. By assembling arrays of these TALEs, the TALE DNA-binding domain can be engineered to bind desired DNA sequence, and thereby guide the nuclease to cut at specific locations in genome (Boch et al., Nature Biotechnology; 29(2):135-6 (2011)). In certain embodiments, the target gene is part of the PKM gene. In certain embodiments, the target sequence is exon 9 of PKM gene. In certain embodiments, the target sequence is exon 9 and exon 10 of PKM gene.

In certain embodiments, the expression of PKM polypeptide can be knocked down using oligonucleotides that have complementary sequences to PKM nucleic acids (e.g., PKM mRNA, PKM-1 mRNA or PKM-2 mRNA). Non-limiting examples of such oligonucleotides include small interference RNA (siRNA), short hairpin RNA (shRNA), and micro RNA (miRNA). In certain embodiments, such oligonucleotides can be homologous to at least a portion of a PKM nucleic acid sequence, e.g., a PKM, a PKM-1 or a PKM-2 nucleic acid sequence, wherein the homology of the portion relative to the PKM nucleic acid sequence is at least about 75 or at least about 80 or at least about 85 or at least about 90 or at least about 95 or at least about 98 percent. In certain non-limiting embodiments, the complementary portion can constitute at least 10 nucleotides or at least 15 nucleotides or at least 20 nucleotides or at least 25 nucleotides or at least 30 nucleotides and the antisense nucleic acid, shRNA, mRNA or siRNA molecules can be up to 15 or up to 20 or up to 25 or up to 30 or up to 35 or up to 40 or up to 45 or up to 50 or up to 75 or up to 100 nucleotides in length. Antisense nucleic acid, shRNA, mRNA or siRNA molecules can comprise DNA or atypical or non-naturally occurring residues, for example, but not limited to, phosphorothioate residues.

The genetic engineering system disclosed herein can be delivered into the mammalian cell using a viral vector, e.g., retroviral vectors such as gamma-retroviral vectors, and lentiviral vectors. Combinations of retroviral vector and an appropriate packaging line are suitable, where the capsid proteins will be functional for infecting human cells. Various amphotropic virus-producing cell lines are known, including, but not limited to, PA12 (Miller, et al. (1985) Mol. Cell. Biol. 5:431-437); PA317 (Miller, et al. (1986) Mol. Cell. Biol. 6:2895-2902); and CRIP (Danos, et al. (1988) Proc. Natl. Acad. Sci. USA 85:6460-6464). Non-amphotropic particles are suitable too, e.g., particles pseudotyped with VSVG, RD 114 or GALV envelope and any other known in the art. Possible methods of transduction also include direct co-culture of the cells with producer cells, e.g., by the method of Bregni, et al. (1992) Blood 80:1418-1422, or culturing with viral supernatant alone or concentrated vector stocks with or without appropriate growth factors and polycations, e.g., by the method of Xu, et al. (1994) Exp. Hemat. 22:223-230; and Hughes, et al. (1992) J. Clin. Invest. 89:1817.

Other transducing viral vectors can be used to modify the mammalian cell disclosed herein. In certain embodiments, the chosen vector exhibits high efficiency of infection and stable integration and expression (see, e.g., Cayouette et al., Human Gene Therapy 8:423-430, 1997; Kido et al., Current Eye Research 15:833-844, 1996; Bloomer et al., Journal of Virology 71:6641-6649, 1997; Naldini et al., Science 272:263-267, 1996; and Miyoshi et al., Proc. Natl. Acad. Sci. U.S.A. 94:10319, 1997). Other viral vectors that can be used include, for example, adenoviral, lentiviral, and adena-associated viral vectors, vaccinia virus, a bovine papilloma virus, or a herpes virus, such as Epstein-Barr Virus (also see, for example, the vectors of Miller, Human Gene Therapy 15-14, 1990; Friedman, Science 244:1275-1281, 1989; Eglitis et al., BioTechniques 6:608-614, 1988; Tolstoshev et al., Current Opinion in Biotechnology 1:55-61, 1990; Sharp, The Lancet 337:1277-1278, 1991; Cornetta et al., Nucleic Acid Research and Molecular Biology 36:311-322, 1987; Anderson, Science 226:401-409, 1984; Moen, Blood Cells 17:407-416, 1991; Miller et al., Biotechnology 7:980-990, 1989; LeGal La Salle et al., Science 259:988-990, 1993; and Johnson, Chest 107:77S-83S, 1995). Retroviral vectors are particularly well developed and have been used in clinical settings (Rosenberg et al., N. Engl. J. Med 323:370, 1990; Anderson et al., U.S. Pat. No. 5,399,346).

Non-viral approaches can also be employed for genetic engineering of the mammalian cell disclosed herein. For example, a nucleic acid molecule can be introduced into the mammalian cell by administering the nucleic acid in the presence of lipofection (Feigner et al., Proc. Natl. Acad. Sci. U.S.A. 84:7413, 1987; Ono et al., Neuroscience Letters 17:259, 1990; Brigham et al., Am. J. Med. Sci. 298:278, 1989; Staubinger et al., Methods in Enzymology 101:512, 1983), asialoorosomucoid-polylysine conjugation (Wu et al., Journal of Biological Chemistry 263:14621, 1988; Wu et al., Journal of Biological Chemistry 264:16985, 1989), or by micro-injection under surgical conditions (Wolff et al., Science 247:1465, 1990). Other non-viral means for gene transfer include transfection in vitro using calcium phosphate, DEAE dextran, electroporation and protoplast fusion. Liposomes can also be potentially beneficial for delivery of nucleic acid molecules into a cell. Transplantation of normal genes into the affected tissues of a subject can also be accomplished by transferring a normal nucleic acid into a cultivatable cell type ex vivo (e.g., an autologous or heterologous primary cell or progeny thereof), after which the cell (or its descendants) are injected into a targeted tissue or are injected systemically.

### 5.3 CELLS WITH REDUCED OR ELIMINATED LACTOGENIC ACTIVITY

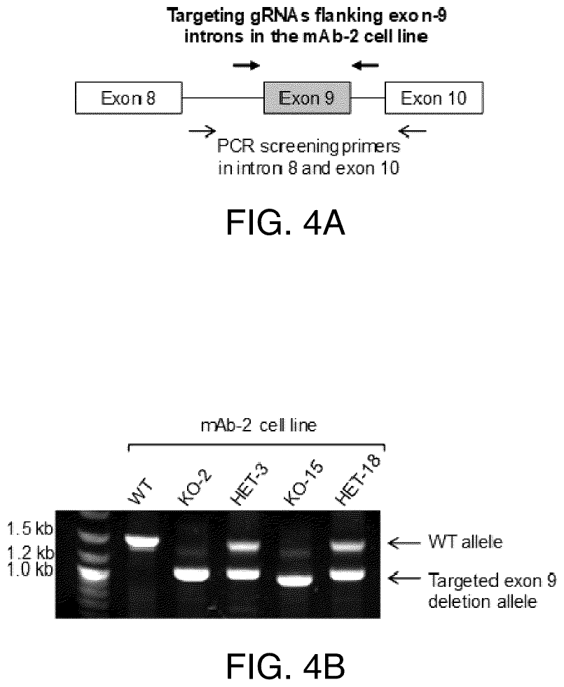

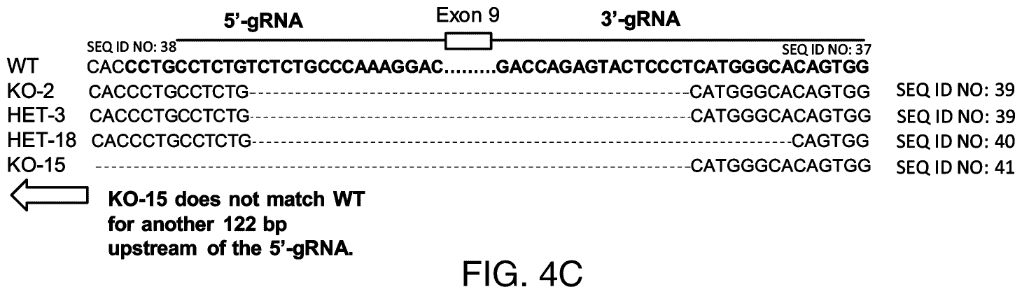

In one aspect, the present disclosure relates to cells or compositions comprising one or more cells, e.g., mammalian cells, having reduced or eliminated lactogenic activity and methods of using the same. The expression of a PKM polypeptide (e.g., PKM-1 expression) is knocked down or knocked out in the cell, which results in reduced or eliminated lactogenic activity of the cell. Non-limiting examples of the cells include CHO cells (e.g., DHFR CHO cells), dp12.CHO cells, CHO-K1 (ATCC, CCL-61), monkey kidney CV1 line transformed by SV40 (e.g., COS-7 ATCC CRL-1651), human embryonic kidney line (e.g., 293 or 293 cells subcloned for growth in suspension culture), baby hamster kidney cells (e.g., BHK, ATCC CCL 10), mouse sertoli cells (e.g. TM4), monkey kidney cells (e.g., CV1 ATCC CCL 70), African green monkey kidney cells (e.g., VERO-76, ATCC CRL-1587), human cervical carcinoma cells (e.g., HELA, ATCC CCL 2), canine kidney cells (e.g., MDCK, ATCC CCL 34), buffalo rat liver cells (e.g., BRL 3A, ATCC CRL 1442), human lung cells (e.g., W138, ATCC CCL 75), human liver cells (e.g., Hep G2, HB 8065), mouse mammary tumor (e.g., MMT 060562, ATCC CCL51), TRI cells, MRC 5 cells, FS4 cells, human hepatoma line (e.g., Hep G2), myeloma cell lines (e.g., Y0, NS0 and Sp2/0). In certain embodiments, the cells are CHO cells. Additional non-limiting examples of CHO host cells include CHO K1SV cells, CHO DG44 cells, a CHO DUKXB-11 cells, CHOK1S cells and CHO KIM cells. In certain embodiments, only one allele of the PKM gene is modified in a cell of the present disclosure. In certain embodiments, both alleles of the PKM gene are modified. In certain embodiments, a cell of the present disclosure comprises at least one allele of the PKM gene that comprises a sequence selected from the group consisting of SEQ ID NOs: 39-41, or comprises the nucleotide sequences set forth in SEQ ID NOs: 37 and 38 (see FIG. 4C). For example, but not by way of limitation, a cell of the present disclosure comprises an allele of a PKM gene that comprises a nucleotide sequence selected from the group consisting of SEQ ID NOs: 39-41, or comprises the nucleotide sequences set forth in SEQ ID NOs: 37 and 38. In certain embodiments, a cell of the present disclosure comprises at least one allele of the PKM gene that comprises a sequence selected from the group consisting of SEQ ID NOs: 39-41. In certain embodiments, the cell is a CHO cell. Non-limiting examples of the cell having reduced or eliminated lactogenic activity include HET-3, HET-18, KO-2 and KO-15, disclosed herein.

In certain embodiments, the expression of a PKM polypeptide (e.g., PKM-1 and/or PKM-2) is knocked out, which results in eliminating the lactogenic activity of the cell as compared to a reference cell. In certain embodiments, the expression of a PKM polypeptide is knocked down in the cell, which results in reduced lactogenic activity of the cell as compared to a reference cell. In certain embodiments, the reference cells are cells where the expression of a PKM polypeptide is not modulated. In certain embodiments, the reference cells are cells that have at least one wild-type PKM allele. In certain embodiments, the reference cells are cells having both wild-type PKM alleles. In certain embodiments, the reference cells are WT CHO cells.

In certain embodiments, the lactogenic activity of cells is indicated by the lactate concentration in the cell culture media during the culturing period of the cells. In certain embodiments, the lactogenic activity of cells is indicated by the lactate concentration in the cell culture media on day 2, day 3, day 4, day 5, day 6, day 7, day 8, day 9, day 10, day 11, day 12, day 13, day 14, day 15, day 16, day 17, day 18, day 19, day 20, day 25, day 30, day 35, day 40, day 45, day 50, day 55, day 60, day 65, day 70 or day 80 of the culturing period. In certain embodiments, the lactogenic activity of cells is indicated by the lactate concentration in the cell culture media more than 80 days after the start of the culturing. In certain embodiments, the lactogenic activity of the presently disclosed cells is indicated by the lactate concentration in the cell culture media during the production phase, e.g., on day 2, day 3, day 4, day 5, day 6, day 7, day 8, day 9, day 10, day 11, day 12, day 13, day 14, day 15, day 16, day 17, day 18, day 19 or day 20 of the production phase of a cell culture process. In certain embodiments, the lactate concentration is determined on day 6, day 7, day 10, day 11, day 14 and/or day 15 of the production phase. In certain embodiments, the lactate concentration is determined on day 7, day 10 and/or day 14 of the production phase.

Any methods known in the art for measuring lactogenic activity of a cell and/or for measuring lactate production by cells in culture can be used with the subject matter disclosed herein. Non-limiting exemplary methods include those disclosed in TeSlaa and Teitell, Methods Enzymol (2014) 542:91-114 and in Lehman et al., Med Sci Sports Exerc. (1991) 23(8):935-8, which are incorporated for reference in their entireties herein. For example, but not by way of limitation, such techniques include using commercial extracellular lactate kits, use of an extracellular bioanalyzer, measuring the extracellular acidification rate (ECAR), e.g., by using a Seahorse XF analyzer, measuring the activity of rate-limiting glycolytic enzymes and measuring lactate production by using tracers.

In certain embodiments, the lactogenic activity of the cells having reduced lactogenic activity (e.g., because of the modulation of the PKM gene in the cells to knock down or knock out a PKM polypeptide) is about 80%, about 70%, about 60%, about 50%, about 40%, about 30%, about 20%, about 10%, about 5%, about 4%, about 3%, about 2% or about 1% of the lactogenic activity of a reference cell. In certain embodiments, lactogenic activity of the cells having reduced lactogenic activity (e.g., because of the modulation of the PKM gene in the cells to knock down or knock out a PKM Polypeptide) is less than about 80%, about 70%, about 60%, about 50%, about 40%, about 30%, about 20%, about 10%, about 5%, about 4%, about 3%, about 2% or about 1% of the lactogenic activity of the reference cell. In certain embodiments, the lactogenic activity of the cell is less than about 50% of the lactogenic activity of a reference cell, e.g., as observed at day 14 or day 15 of the production phase of the cell culture. In certain embodiments, the lactogenic activity of the cell is less than about 20% of the lactogenic activity of a reference cell, e.g., as observed at day 14 or day 15 of the production phase of the cell culture. In certain embodiments, the lactogenic activity of the cell is less than about 10% of the lactogenic activity of a reference cell, e.g., as observed at day 14 or day 15 of the production phase of the cell culture. In certain embodiments, the reference cell is a cell that comprises at least one or both wild-type alleles of the PKM gene.

In certain embodiments, the lactate concentration in the cell culture media produced by cells of the present disclosure is less than about 15 g/L, less than about 14 g/L, less than about 13 g/L, less than about 12 g/L, less than about 101 g/L, less than about 10 g/L, less than about 9 g/L, less than about 8 g/L, less than about 7 g/L, less than about 6 g/L, less than about 5 g/L, less than about 4 g/L, less than about 3 g/L, less than about 2 g/L, less than about 1 g/L or about less than about 0.5 g/L, e.g., in the production phase of the cell culture (e.g., on day 2, day 3, day 4, day 5, day 6, day 7, day 8, day 9, day 10, day 11, day 12, day 13, day 14, day 15, day 16, day 17, day 18, day 19 or day 20 of the production phase). In certain embodiments, the lactate concentration in the cell culture media produced by cells of the present disclosure is less than about 5 g/L, less than about 2 g/L, or less than about 1 g/L. In certain embodiments, the lactate concentration in the cell culture media produced by cells of the present disclosure is less than about 5 g/L, less than about 2 g/L, or less than about 1 g/L on day 7, day 10, day 14 or day 15 of the production phase of the cell culture. In certain embodiments, the lactate concentration in the cell culture media produced by cells of the present disclosure is less than about 1 g/L, or less than about 2 g/L during the production phase of a shake flask culture. In certain embodiments, the lactate concentration in the cell culture media produced by cells of the present disclosure is less than about 2 g/L during the production phase of a cell culture in a bioreactor.

In certain embodiments, the cells with reduced or eliminated lactogenic activity as disclosed herein (e.g., cells generated by knocking out or knocking down of the expression of a PKM polypeptide) have comparable titer and/or specific productivity as cells with regular lactogenic activity (e.g., wildtype cells). In certain embodiments, the difference in titer and/or specific productivity between the cells with reduced or eliminated lactogenic activity and the cells with regular lactogenic activity is less than about 1%, less than about 5%, less than about 10%, less than about 15%, less than about 20%, less than about 25%, or less than about 30% of the cells with regular lactogenic activity (e.g., a reference cell). In certain embodiments, the cells with reduced or eliminated lactogenic activity have higher titer and/or specific productivity than the cells with regular lactogenic activity. In certain embodiments, the titer of the cells with reduced or eliminated lactogenic activity is about 1%, about 5%, about 10%, about 15%, about 20%, about 25%, about 30%, about 35%, about 40%, about 45%, or about 50% higher than the titer of the cells with regular lactogenic activity. In certain embodiments, the titer of the cells with reduced or eliminated lactogenic activity is more than about 50% higher than the titer of the cell with regular lactogenic activity. In certain embodiments, the specific productivity (Qp) of the cells with reduced or eliminated lactogenic activity is about 1%, about 5%, about 10%, about 15%, about 20%, about 25%, about 30%, about 35%, about 40%, about 45%, about 50%, about 55%, about 60%, about 65%, about 70%, about 75%, about 80%, about 85%, or about 90% higher than the specific productivity of the cells with regular lactogenic activity. In certain embodiments, the specific productivity of the cell with reduced or eliminated lactogenic activity is more than about 90% higher than the specific productivity of the cell with regular lactogenic activity.

The lactate concentration in the cell culture media can be measured by a chemistry analyzer or a lactate assay kit. Non-limiting examples of chemistry analyzer include Bioprofile 400 (Nova Biomedical), Piccolo Xpress Chemistry Analyzer, Excel—Semi-automated Chemistry Analyzer, Indiko Clinical and Specialty Chemistry System, and ACE Axcel® Clinical Chemistry System. Non-limiting examples of lactate assay kit include L-Lactate Assay Kit (Colorimetric) (ab65331, Abcam), BioVision Lactate Colorimetric/Fluorometric Assay Kit, PicoProbe™ Lactate Fluorometric Assay Kit, Lactate Colorimetric Assay Kit II, Cell Biolabs Lactate Assay Kits.

In certain embodiments, the cells disclosed herein express a product of interest. In certain embodiments, the product of interest is a recombinant protein. In certain embodiments, the product of interest is a monoclonal antibody. Additional non-limiting examples of products of interest are provided in Section 5.5. In certain embodiments, the cells disclosed herein can be used for production of commercially useful amounts of the product of interest.

In certain embodiments, the cells disclosed herein can comprise a nucleic acid that encodes a product of interest. In certain embodiments, the nucleic acid can be present in one or more vectors, e.g., expression vectors. One type of vector is a “plasmid,” which refers to a circular double stranded DNA loop into which additional DNA segments can be ligated. Another type of vector is a viral vector, where additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors, expression vectors, are capable of directing the expression of genes to which they are operably linked. In general, expression vectors of utility in recombinant DNA techniques are often in the form of plasmids (vectors). Additional non-limiting examples of expression vectors for use in the present disclosure include viral vectors (e.g., replication defective retroviruses, adenoviruses and adeno-associated viruses) that serve equivalent functions.

In certain embodiments, the nucleic acid encoding a product of interest can be introduced into a host cell, disclosed herein. In certain embodiments, the introduction of a nucleic acid into a cell can be carried out by any method known in the art including, but not limited to, transfection, electroporation, microinjection, infection with a viral or bacteriophage vector containing the nucleic acid sequences, cell fusion, chromosome-mediated gene transfer, microcell-mediated gene transfer, spheroplast fusion, etc. In certain embodiments, the host cell is eukaryotic, e.g., a Chinese Hamster Ovary (CHO) cell or lymphoid cell (e.g., Y0, NS0, Sp20 cell).

In certain embodiments, the nucleic acid encoding a product of interest can be randomly integrated into a host cell genome (“Random Integration” or “RI”). For example, but not by way of limitation, a nucleic acid encoding a product of interest can be randomly integrated into the genome of a cell that has been modulated to have knocked down or knocked out expression of a PKM polypeptide, e.g., PKM-1.

In certain embodiments, the nucleic acid encoding a product of interest can be integrated into a host cell genome in a targeted manner (“Targeted Integration” or “TI”). For example, but not by way of limitation, a nucleic acid encoding a product of interest can be integrated into the genome of a cell that has been modulated to have knocked down or knocked out expression of a PKM polypeptide, e.g., PKM-1, in a targeted manner. An “integration site” comprises a nucleic acid sequence within a host cell genome into which an exogenous nucleotide sequence is inserted. In certain embodiments, an integration site is between two adjacent nucleotides on the host cell genome. In certain embodiments, an integration site includes a stretch of nucleotide sequences. In certain embodiments, the integration site is located within a specific locus of the genome of the TI host cell. In certain embodiments, the integration site is within an endogenous gene of the TI host cell. Any integration site known in the art can be regulated and used with the subject matter disclosed herein. The targeted integration can be mediated by methods and systems known in the art. For example, but not by way of limitation, methods and systems disclosed in International Application No. PCT/US18/067070, filed Dec. 21, 2018, the content of which is incorporated herein by its entirely, can be used for targeted integration.

In certain embodiments, the nucleic acid encoding a product of interest can be integrated into a host cell genome using transposase-based integration. Transposase-based integration techniques are disclosed, for example, in Trubitsyna et al., Nucleic Acids Res. 45(10):e89 (2017), Li et al., PNAS 110(25):E2279-E2287 (2013) and WO 2004/009792, which are incorporated by reference herein in their entireties.

In certain embodiments, the nucleic acid encoding a product of interest can be randomly integrated into a host cell genome (“Random Integration” or “RI”). In certain embodiments, the random integration can be mediated by any method or systems known in the art. In certain embodiments, the random integration is mediated by MaxCyte STX® electroporation system.

In certain embodiments, targeted integration can be combined with random integration. In certain embodiments, the targeted integration can be followed by random integration. In certain embodiments, random integration can be followed by targeted integration. For example, but not by way of limitation, a nucleic acid encoding a product of interest can be randomly integrated into the genome of a cell that has been modulated to have knocked down or knocked out expression of a PKM polypeptide, e.g., PKM-1, and a nucleic acid encoding the same product of interest can be integrated in the genome of the cell in a targeted manner.

In certain embodiments, the host cell is a RI host cell. In certain embodiments, the host cell is a TI host cell.

### 5.4. Cell Culturing Methods

In one aspect, the present disclosure provides a method for producing a product of interest comprising culturing a cell disclosed herein. Suitable culture conditions for mammalian cells known in the art can be used for culturing the cells herein (J. Immunol. Methods (1983) 56:221-234) or can be easily determined by the skilled artisan (see, for example, Animal Cell Culture: A Practical Approach 2nd Ed., Rickwood, D. and Hames, B. D., eds. Oxford University Press, New York (1992)).

Mammalian cell culture can be prepared in a medium suitable for the particular cell being cultured. Commercially available media such as Ham's F10 (Sigma), Minimal Essential Medium (MEM, Sigma), RPMI-1640 (Sigma) and Dulbecco's Modified Eagle's Medium (DMEM, Sigma) are exemplary nutrient solutions. In addition, any of the media described in Ham and Wallace, (1979) Meth. Enz., 58:44; Barnes and Sato, (1980) Anal. Biochem., 102:255; U.S. Pat. Nos. 4,767,704; 4,657,866; 4,927,762; 5,122,469 or U.S. Pat. No. 4,560,655; International Publication Nos. WO 90/03430; and WO 87/00195; the disclosures of all of which are incorporated herein by reference, can be used as culture media. Any of these media can be supplemented as necessary with hormones and/or other growth factors (such as insulin, transferrin, or epidermal growth factor), salts (such as sodium chloride, calcium, magnesium, and phosphate), buffers (such as HEPES), nucleosides (such as adenosine and thymidine), antibiotics (such as gentamycin (gentamicin), trace elements (defined as inorganic compounds usually present at final concentrations in the micromolar range) lipids (such as linoleic or other fatty acids) and their suitable carriers, and glucose or an equivalent energy source. Any other necessary supplements can also be included at appropriate concentrations that would be known to those skilled in the art.

In certain embodiments, the mammalian cell that has been modified to reduce and/or eliminate the expression of a PKM polypeptide is a CHO cell. Any suitable medium can be used to culture the CHO cell. In certain embodiments, a suitable medium for culturing the CHO cell can contain a basal medium component such as a DMEM/HAM F-12 based formulation (for composition of DMEM and HAM F12 media, see culture media formulations in American Type Culture Collection Catalogue of Cell Lines and Hybridomas, Sixth Edition, 1988, pages 346-349) (the formulation of medium as described in U.S. Pat. No. 5,122,469 are particularly appropriate) with modified concentrations of some components such as amino acids, salts, sugar, and vitamins, and optionally containing glycine, hypoxanthine, and thymidine; recombinant human insulin, hydrolyzed peptone, such as Primatone HS or Primatone RL (Sheffield, England), or the equivalent; a cell protective agent, such as Pluronic F68 or the equivalent pluronic polyol; gentamycin; and trace elements.

In certain embodiments, the mammalian cell that has been modified to reduce and/or eliminate the expression of a PKM polypeptide is a cell that expresses a recombinant protein. The recombinant protein can be produced by growing cells which express the products of interest under a variety of cell culture conditions. For instance, cell culture procedures for the large or small-scale production of proteins are potentially useful within the context of the present disclosure. Procedures including, but not limited to, a fluidized bed bioreactor, hollow fiber bioreactor, roller bottle culture, shake flask culture, or stirred tank bioreactor system can be used, in the latter two systems, with or without microcarriers, and operated alternatively in a batch, fed-batch, or continuous mode.

In certain embodiments, the cell culture of the present disclosure is performed in a stirred tank bioreactor system and a fed batch culture procedure is employed. In the fed batch culture, the mammalian host cells and culture medium are supplied to a culturing vessel initially and additional culture nutrients are fed, continuously or in discrete increments, to the culture during culturing, with or without periodic cell and/or product harvest before termination of culture. The fed batch culture can include, for example, a semi-continuous fed batch culture, wherein periodically whole culture (including cells and medium) is removed and replaced by fresh medium. Fed batch culture is distinguished from simple batch culture in which all components for cell culturing (including the cells and all culture nutrients) are supplied to the culturing vessel at the start of the culturing process. Fed batch culture can be further distinguished from perfusion culturing insofar as the supernatant is not removed from the culturing vessel during the process (in perfusion culturing, the cells are restrained in the culture by, e.g., filtration, encapsulation, anchoring to microcarriers etc. and the culture medium is continuously or intermittently introduced and removed from the culturing vessel).

In certain embodiments, the cells of the culture can be propagated according to any scheme or routine that can be suitable for the specific host cell and the specific production plan contemplated. Therefore, the present disclosure contemplates a single step or multiple step culture procedure. In a single step culture, the host cells are inoculated into a culture environment and the processes of the instant disclosure are employed during a single production phase of the cell culture. Alternatively, a multi-stage culture is envisioned. In the multi-stage culture cells can be cultivated in a number of steps or phases. For instance, cells can be grown in a first step or growth phase culture wherein cells, possibly removed from storage, are inoculated into a medium suitable for promoting growth and high viability. The cells can be maintained in the growth phase for a suitable period of time by the addition of fresh medium to the host cell culture.

In certain embodiments, fed batch or continuous cell culture conditions are devised to enhance growth of the mammalian cells in the growth phase of the cell culture. In the growth phase cells are grown under conditions and for a period of time that is maximized for growth. Culture conditions, such as temperature, pH, dissolved oxygen (dO₂) and the like, are those used with the particular host and will be apparent to the ordinarily skilled artisan. Generally, the pH is adjusted to a level between about 6.5 and 7.5 using either an acid (e.g., CO₂) or a base (e.g., Na₂CO₃ or NaOH). A suitable temperature range for culturing mammalian cells such as CHO cells is between about 30° to 38° C. and a suitable dO₂is between 5-90% of air saturation.

At a particular stage the cells can be used to inoculate a production phase or step of the cell culture. Alternatively, as described above the production phase or step can be continuous with the inoculation or growth phase or step.

In certain embodiments, the culturing methods described in the present disclosure can further include harvesting the product from the cell culture, e.g., from the production phase of the cell culture. In certain embodiments, the product produced by the cell culture methods of the present disclosure can be harvested from the third bioreactor, e.g., production bioreactor. For example, but not by way of limitation, the disclosed methods can include harvesting the product at the completion of the production phase of the cell culture. Alternatively or additionally, the product can be harvested prior to the completion of the production phase. In certain embodiments, the product can be harvested from the cell culture once a particular cell density has been achieved. For example, but not by way of limitation, the cell density can be from about 2.0×10⁷ cells/mL to about 5.0×10⁷ cells/mL prior to harvesting.

In certain embodiments, harvesting the product from the cell culture can include one or more of centrifugation, filtration, acoustic wave separation, flocculation and cell removal technologies.

In certain embodiments, the product of interest can be secreted from the host cells or can be a membrane-bound, cytosolic or nuclear protein. In certain embodiments, soluble forms of the polypeptide can be purified from the conditioned cell culture media and membrane-bound forms of the polypeptide can be purified by preparing a total membrane fraction from the expressing cells and extracting the membranes with a nonionic detergent such as TRITON® X-100 (EMD Biosciences, San Diego, Calif.). In certain embodiments, cytosolic or nuclear proteins can be prepared by lysing the host cells (e.g., by mechanical force, sonication and/or detergent), removing the cell membrane fraction by centrifugation and retaining the supernatant.

### 5.5 PRODUCTS

The cells and/or methods of the present disclosure can be used to produce any product of interest that can be expressed by the cells disclosed herein. In certain embodiments, the cells and/or methods of the present disclosure can be used for the production of polypeptides, e.g., mammalian polypeptides. Non-limiting examples of such polypeptides include hormones, receptors, fusion proteins, regulatory factors, growth factors, complement system factors, enzymes, clotting factors, anti-clotting factors, kinases, cytokines, CD proteins, interleukins, therapeutic proteins, diagnostic proteins and antibodies. The cells and/or methods of the present disclosure are not specific to the molecule, e.g., antibody, that is being produced.

In certain embodiments, the methods of the present disclosure can be used for the production of antibodies, including therapeutic and diagnostic antibodies or antigen-binding fragments thereof. In certain embodiments, the antibody produced by cell and methods of the present disclosure can be, but are not limited to, monospecific antibodies (e.g., antibodies consisting of a single heavy chain sequence and a single light chain sequence, including multimers of such pairings), multispecific antibodies and antigen-binding fragments thereof. For example, but not by way of limitation, the multispecific antibody can be a bispecific antibody, a biepitopic antibody, a T-cell-dependent bispecific antibody (TDB), a Dual Acting FAb (DAF) or antigen-binding fragments thereof.

### 5.5.1 Multispecific Antibodies

In certain aspects, an antibody produced by cells and methods provided herein is a multispecific antibody, e.g., a bispecific antibody. “Multispecific antibodies” are monoclonal antibodies that have binding specificities for at least two different sites, i.e., different epitopes on different antigens (i.e., bispecific) or different epitopes on the same antigen (i.e., biepitopic). In certain aspects, the multispecific antibody has three or more binding specificities. Multispecific antibodies can be prepared as full length antibodies or antibody fragments as described herein.

Techniques for making multispecific antibodies include, but are not limited to, recombinant co-expression of two immunoglobulin heavy chain-light chain pairs having different specificities (see Milstein and Cuello, Nature 305: 537 (1983)) and “knob-in-hole” engineering (see, e.g., U.S. Pat. No. 5,731,168, and Atwell et al., J. Mol. Biol. 270:26 (1997)). Multispecific antibodies can also be made by engineering electrostatic steering effects for making antibody Fc-heterodimeric molecules (see, e.g., WO 2009/089004); cross-linking two or more antibodies or fragments (see, e.g., U.S. Pat. No. 4,676,980, and Brennan et al., Science, 229: 81 (1985)); using leucine zippers to produce bi-specific antibodies (see, e.g., Kostelny et al., J. Immunol., 148(5):1547-1553 (1992) and WO 2011/034605); using the common light chain technology for circumventing the light chain mis-pairing problem (see, e.g., WO 98/50431); using “diabody” technology for making bispecific antibody fragments (see, e.g., Hollinger et al., Proc. Natl. Acad. Sci. USA, 90:6444-6448 (1993)); and using single-chain Fv (sFv) dimers (see, e.g., Gruber et al., J. Immunol., 152:5368 (1994)); and preparing trispecific antibodies as described, e.g., in Tutt et al. J. Immunol. 147: 60 (1991).

Engineered antibodies with three or more antigen binding sites, including for example, “Octopus antibodies”, or DVD-Ig are also included herein (see, e.g., WO 2001/77342 and WO 2008/024715). Other non-limiting examples of multispecific antibodies with three or more antigen binding sites can be found in WO 2010/115589, WO 2010/112193, WO 2010/136172, WO 2010/145792 and WO 2013/026831. The bispecific antibody or antigen binding fragment thereof also includes a “Dual Acting FAb” or “DAF” (see, e.g., US 2008/0069820 and WO 2015/095539).

Multispecific antibodies can also be provided in an asymmetric form with a domain crossover in one or more binding arms of the same antigen specificity, i.e., by exchanging the VH/VL domains (see, e.g., WO 2009/080252 and WO 2015/150447), the CH1/CL domains (see, e.g., WO 2009/080253) or the complete Fab arms (see, e.g., WO 2009/080251, WO 2016/016299, also see Schaefer et al, PNAS, 108 (2011) 1187-1191, and Klein at al., MAbs 8 (2016) 1010-20). In certain embodiments, the multispecific antibody comprises a cross-Fab fragment. The term “cross-Fab fragment” or “xFab fragment” or “crossover Fab fragment” refers to a Fab fragment, wherein either the variable regions or the constant regions of the heavy and light chain are exchanged. A cross-Fab fragment comprises a polypeptide chain composed of the light chain variable region (VL) and the heavy chain constant region 1 (CH1), and a polypeptide chain composed of the heavy chain variable region (VH) and the light chain constant region (CL). Asymmetrical Fab arms can also be engineered by introducing charged or non-charged amino acid mutations into domain interfaces to direct correct Fab pairing. See, e.g., WO 2016/172485.

Various further molecular formats for multispecific antibodies are known in the art and are included herein (see, e.g., Spiess et al., Mol. Immunol. 67 (2015) 95-106).

In certain embodiments, particular type of multispecific antibodies, also included herein, are bispecific antibodies designed to simultaneously bind to a surface antigen on a target cell, e.g., a tumor cell, and to an activating, invariant component of the T cell receptor (TCR) complex, such as CD3, for retargeting of T cells to kill target cells.

Additional non-limiting examples of bispecific antibody formats that can be useful for this purpose include, but are not limited to, the so-called “BiTE” (bispecific T cell engager) molecules wherein two scFv molecules are fused by a flexible linker (see, e.g., WO 2004/106381, WO 2005/061547, WO 2007/042261, and WO 2008/119567, Nagorsen and Bauerle, Exp Cell Res 317, 1255-1260 (2011)); diabodies (Holliger et al., Prot. Eng. 9, 299-305 (1996)) and derivatives thereof, such as tandem diabodies (“TandAb”; Kipriyanov et al., J Mol Biol 293, 41-56 (1999)); “DART” (dual affinity retargeting) molecules which are based on the diabody format but feature a C-terminal disulfide bridge for additional stabilization (Johnson et al., J Mol Biol 399, 436-449 (2010)), and so-called triomabs, which are whole hybrid mouse/rat IgG molecules (reviewed in Seimetz et al., Cancer Treat. Rev. 36, 458-467 (2010)). Particular T cell bispecific antibody formats included herein are described in WO 2013/026833, WO 2013/026839, WO 2016/020309; Bacac et al., Oncoimmunology 5(8) (2016) e1203498.

### 5.5.2 Antibody Fragments

In certain aspects, an antibody produced by the cells and methods provided herein is an antibody fragment. For example, but not by way of limitation, the antibody fragment is a Fab, Fab′, Fab′-SH or F(ab′)₂ fragment, in particular a Fab fragment. Papain digestion of intact antibodies produces two identical antigen-binding fragments, called “Fab” fragments containing each the heavy- and light-chain variable domains (VH and VL, respectively) and also the constant domain of the light chain (CL) and the first constant domain of the heavy chain (CH1). The term “Fab fragment” thus refers to an antibody fragment comprising a light chain comprising a VL domain and a CL domain, and a heavy chain fragment comprising a VH domain and a CH1 domain. “Fab′ fragments” differ from Fab fragments by the addition of residues at the carboxy terminus of the CH1 domain including one or more cysteines from the antibody hinge region. Fab′-SH are Fab′ fragments in which the cysteine residue(s) of the constant domains bear a free thiol group. Pepsin treatment yields an F(ab′)₂ fragment that has two antigen-binding sites (two Fab fragments) and a part of the Fc region. For discussion of Fab and F(ab′)₂ fragments comprising salvage receptor binding epitope residues and having increased in vivo half-life, see U.S. Pat. No. 5,869,046.

In certain embodiments, the antibody fragment is a diabody, a triabody or a tetrabody. “Diabodies” are antibody fragments with two antigen-binding sites that can be bivalent or bispecific. See, for example, EP 404,097; WO 1993/01161; Hudson et al., Nat. Med. 9:129-134 (2003); and Hollinger et al., Proc. Natl. Acad. Sci. USA 90: 6444-6448 (1993). Triabodies and tetrabodies are also described in Hudson et al., Nat. Med. 9:129-134 (2003).

In a further aspect, the antibody fragment is a single chain Fab fragment. A “single chain Fab fragment” or “scFab” is a polypeptide consisting of an antibody heavy chain variable domain (VH), an antibody heavy chain constant domain 1 (CH1), an antibody light chain variable domain (VL), an antibody light chain constant domain (CL) and a linker, wherein said antibody domains and said linker have one of the following orders in N-terminal to C-terminal direction: a) VH-CH1-linker-VL-CL, b) VL-CL-linker-VH-CH1, c) VH-CL-linker-VL-CH1 or d) VL-CH1-linker-VH-CL. In particular, said linker is a polypeptide of at least 30 amino acids, preferably between 32 and 50 amino acids. Said single chain Fab fragments are stabilized via the natural disulfide bond between the CL domain and the CH1 domain. In addition, these single chain Fab fragments might be further stabilized by generation of interchain disulfide bonds via insertion of cysteine residues (e.g., position 44 in the variable heavy chain and position 100 in the variable light chain according to Kabat numbering).

In another aspect, the antibody fragment is single-chain variable fragment (scFv). A “single-chain variable fragment” or “scFv” is a fusion protein of the variable domains of the heavy (VH) and light chains (VL) of an antibody, connected by a linker. In particular, the linker is a short polypeptide of 10 to 25 amino acids and is usually rich in glycine for flexibility, as well as serine or threonine for solubility, and can either connect the N-terminus of the VH with the C-terminus of the VL, or vice versa. This protein retains the specificity of the original antibody, despite removal of the constant regions and the introduction of the linker. For a review of scFv fragments, see, e.g., Plückthun, in The Pharmacology of Monoclonal Antibodies, vol. 113, Rosenburg and Moore eds., (Springer-Verlag, New York), pp. 269-315 (1994); see also WO 93/16185; and U.S. Pat. Nos. 5,571,894 and 5,587,458.

In another aspect, the antibody fragment is a single-domain antibody. “Single-domain antibodies” are antibody fragments comprising all or a portion of the heavy chain variable domain or all or a portion of the light chain variable domain of an antibody. In certain aspects, a single-domain antibody is a human single-domain antibody (Domantis, Inc., Waltham, MA; see, e.g., U.S. Pat. No. 6,248,516 B1).

Antibody fragments can be made by various techniques, including but not limited to proteolytic digestion of an intact antibody.

### 5.5.3 Chimeric and Humanized Antibodies

In certain aspects, an antibody produced by the cells and methods provided herein is a chimeric antibody. Certain chimeric antibodies are described, e.g., in U.S. Pat. No. 4,816,567; and Morrison et al., Proc. Natl. Acad. Sci. USA, 81:6851-6855 (1984)). In one example, a chimeric antibody comprises a non-human variable region (e.g., a variable region derived from a mouse, rat, hamster, rabbit, or non-human primate, such as a monkey) and a human constant region. In a further example, a chimeric antibody is a “class switched” antibody in which the class or subclass has been changed from that of the parent antibody. Chimeric antibodies include antigen-binding fragments thereof.

In certain aspects, a chimeric antibody is a humanized antibody. Typically, a non-human antibody is humanized to reduce immunogenicity to humans, while retaining the specificity and affinity of the parental non-human antibody. Generally, a humanized antibody comprises one or more variable domains in which the CDRs (or portions thereof) are derived from a non-human antibody, and FRs (or portions thereof) are derived from human antibody sequences. A humanized antibody optionally will also comprise at least a portion of a human constant region. In certain embodiments, some FR residues in a humanized antibody are substituted with corresponding residues from a non-human antibody (e.g., the antibody from which the CDR residues are derived), e.g., to restore or improve antibody specificity or affinity.

Humanized antibodies and methods of making them are reviewed, e.g., in Almagro and Fransson, Front. Biosci. 13:1619-1633 (2008), and are further described, e.g., in Riechmann et al., Nature 332:323-329 (1988); Queen et al., Proc. Nat'l Acad. Sci. USA 86:10029-10033 (1989); U.S. Pat. Nos. 5,821,337, 7,527,791, 6,982,321, and 7,087,409; Kashmiri et al., Methods 36:25-34 (2005) (describing specificity determining region (SDR) grafting); Padlan, Mol. Immunol. 28:489-498 (1991) (describing “resurfacing”); Dall'Acqua et al., Methods 36:43-60 (2005) (describing “FR shuffling”); and Osbourn et al., Methods 36:61-68 (2005) and Klimka et al., Br. J. Cancer, 83:252-260 (2000) (describing the “guided selection” approach to FR shuffling).

Human framework regions that can be used for humanization include but are not limited to: framework regions selected using the “best-fit” method (see, e.g., Sims et al. J. Immunol. 151:2296 (1993)); framework regions derived from the consensus sequence of human antibodies of a particular subgroup of light or heavy chain variable regions (see, e.g., Carter et al. Proc. Natl. Acad. Sci. USA, 89:4285 (1992); and Presta et al. J. Immunol., 151:2623 (1993)); human mature (somatically mutated) framework regions or human germline framework regions (see, e.g., Almagro and Fransson, Front. Biosci. 13:1619-1633 (2008)); and framework regions derived from screening FR libraries (see, e.g., Baca et al., J Biol. Chem. 272:10678-10684 (1997) and Rosok et al., J. Biol. Chem. 271:22611-22618 (1996)).

### 5.5.4 Human Antibodies

In certain aspects, an antibody produced by the cells and methods provided herein is a human antibody. Human antibodies can be produced using various techniques known in the art. Human antibodies are described generally in van Dijk and van de Winkel, Curr. Opin. Pharmacol. 5: 368-74 (2001) and Lonberg, Curr. Opin. Immunol. 20:450-459 (2008).

Human antibodies can be prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. In such transgenic mice, the endogenous immunoglobulin loci have generally been inactivated. For review of methods for obtaining human antibodies from transgenic animals, see Lonberg, Nat. Biotech. 23:1117-1125 (2005). See also, e.g., U.S. Pat. Nos. 6,075,181 and 6,150,584 describing XENOMOUSE™ technology; U.S. Pat. No. 5,770,429 describing HUMAB® technology; U.S. Pat. No. 7,041,870 describing K-M MOUSE® technology, and U.S. Patent Application Publication No. US 2007/0061900, describing VELOCIMOUSE® technology). Human variable regions from intact antibodies generated by such animals can be further modified, e.g., by combining with a different human constant region.

Human antibodies can also be made by hybridoma-based methods. Human myeloma and mouse-human heteromyeloma cell lines for the production of human monoclonal antibodies have been described. (See, e.g., Kozbor J. Immunol., 133: 3001 (1984); Brodeur et al., Monoclonal Antibody Production Techniques and Applications, pp. 51-63 (Marcel Dekker, Inc., New York, 1987); and Boerner et al., J. Immunol., 147: 86 (1991).) Human antibodies generated via human B-cell hybridoma technology are also described in Li et al., Proc. Natl. Acad. Sci. USA, 103:3557-3562 (2006). Additional methods include those described, for example, in U.S. Pat. No. 7,189,826 (describing production of monoclonal human IgM antibodies from hybridoma cell lines) and Ni, Xiandai Mianyixue, 26(4):265-268 (2006) (describing human-human hybridomas). Human hybridoma technology (Trioma technology) is also described in Vollmers and Brandlein, Histology and Histopathology, 20(3):927-937 (2005) and Vollmers and Brandlein, Methods and Findings in Experimental and Clinical Pharmacology, 27(3):185-91 (2005).

### 5.5.5 Target Molecules

Non-limiting examples of molecules that can be targeted by an antibody produced by the cells and methods disclosed herein include soluble serum proteins and their receptors and other membrane bound proteins (e.g., adhesins). In certain embodiments, an antibody produced by the cells and methods disclosed herein is capable of binding to one, two or more cytokines, cytokine-related proteins, and cytokine receptors selected from the group consisting of 8MPI, 8MP2, 8MP38 (GDFIO), 8MP4, 8MP6, 8MP8, CSFI (M-CSF), CSF2 (GM-CSF), CSF3 (G-CSF), EPO, FGF1 (αFGF), FGF2 (βFGF), FGF3 (int-2), FGF4 (HST), FGF5, FGF6 (HST-2), FGF7 (KGF), FGF9, FGF10, FGF11, FGF12, FGF12B, FGF14, FGF16, FGF17, FGF19, FGF20, FGF21, FGF23, IGF1, IGF2, IFNA1, IFNA2, IFNA4, IFNA5, IFNA6, IFNA7, IFN81, IFNG, IFNWI, FEL1, FEL1 (EPSELON), FEL1 (ZETA), IL 1A, IL 1B, IL2, IL3, IL4, IL5, IL6, IL7, IL8, IL9, IL10, IL 11, IL 12A, IL 12B, IL 13, IL 14, IL 15, IL 16, IL 17, IL 17B, IL 18, IL 19, IL20, IL22, IL23, IL24, IL25, IL26, IL27, IL28A, IL28B, IL29, IL30, PDGFA, PDGFB, TGFA, TGFB1, TGFB2, TGFBb3, LTA (TNF-0), LTB, TNF (TNF-α), TNFSF4 (OX40 ligand), TNFSF5 (CD40 ligand), TNFSF6 (FasL), TNFSF7 (CD27 ligand), TNFSF8 (CD30 ligand), TNFSF9 (4-1 BB ligand), TNFSF10 (TRAIL), TNFSF 11 (TRANCE), TNFSF12 (APO3L), TNFSF13 (April), TNFSF13B, TNFSF14 (HVEM-L), TNFSF15 (VEGI), TNFSF18, HGF (VEGFD), VEGF, VEGFB, VEGFC, IL1R1, IL1R2, IL1RL1, IL1RL2, IL2RA, IL2RB, IL2RG, IL3RA, IL4R, IL5RA, IL6R, IL7R, IL8RA, IL8RB, IL9R, IL10RA, IL10RB, IL 11RA, IL12RB1, IL12RB2, IL13RA1, IL13RA2, IL15RA, IL17R, IL18R1, IL20RA, IL21R, IL22R, IL1HY1, IL1RAP, IL1RAPL1, IL1RAPL2, IL1RN, IL6ST, IL18BP, IL18RAP, IL22RA2, AIF1, HGF, LEP (leptin), PTN, and THPO.k

In certain embodiments, an antibody produced by cells and methods disclosed herein is capable of binding to a chemokine, chemokine receptor, or a chemokine-related protein selected from the group consisting of CCLI (1-309), CCL2 (MCP-1/MCAF), CCL3 (MIP-1a), CCL4 (MIP-Iβ), CCL5 (RANTES), CCL7 (MCP-3), CCL8 (mcp-2), CCL 11 (eotaxin), CCL 13 (MCP-4), CCL 15 (MIP-Iδ), CCL 16 (HCC-4), CCL 17 (TARC), CCL 18 (PARC), CCL 19 (MDP-3b), CCL20 (MIP-3a), CCL21 (SLC/exodus-2), CCL22 (MDC/STC-1), CCL23 (MPIF-1), CCL24 (MPIF-2/eotaxin-2), CCL25 (TECK), CCL26 (eotaxin-3), CCL27 (CTACK/ILC), CCL28, CXCLI (GROI), CXCL2 (GR02), CXCL3 (GR03), CXCL5 (ENA-78), CXCL6 (GCP-2), CXCL9 (MIG), CXCL 10 (IP 10), CXCL 11 (1-TAC), CXCL 12 (SDFI), CXCL 13, CXCL 14, CXCL 16, PF4 (CXCL4), PPBP (CXCL7), CX3CL 1 (SCYDI), SCYEI, XCLI (lymphotactin), XCL2 (SCM-Iβ), BLRI (MDR15), CCBP2 (D6/JAB61), CCRI (CKRI/HM145), CCR2 (mcp-IRB IRA), CCR3 (CKR3/CMKBR3), CCR4, CCR5 (CMKBR5/ChemR13), CCR6 (CMKBR6/CKR-L3/STRL22/DRY6), CCR7 (CKR7/EBII), CCR8 (CMKBR8/TER1/CKR-L1), CCR9 (GPR-9-6), CCRL1 (VSHK1), CCRL2 (L-CCR), XCR1 (GPR5/CCXCR1), CMKLR1, CMKOR1 (RDC1), CX3CR1 (V28), CXCR4, GPR2 (CCR10), GPR31, GPR81 (FKSG80), CXCR3 (GPR9/CKR-L2), CXCR6 (TYMSTR/STRL33/Bonzo), HM74, IL8RA (IL8Ra), IL8RB (IL8Rβ), LTB4R (GPR16), TCP10, CKLFSF2, CKLFSF3, CKLFSF4, CKLFSF5, CKLFSF6, CKLFSF7, CKLFSF8, BDNF, C5, C5R1, CSF3, GRCC10 (C10), EPO, FY (DARC), GDF5, HDF1, HDF1α, DL8, PRL, RGS3, RGS13, SDF2, SLIT2, TLR2, TLR4, TREM1, TREM2, and VHL.

In certain embodiments, an antibody produced by methods disclosed herein (e.g., a multispecific antibody such as a bispecific antibody) is capable of binding to one or more target molecules selected from the following: 0772P (CA125, MUC16) (i.e., ovarian cancer antigen), ABCF1; ACVR1; ACVR1B; ACVR2; ACVR2B; ACVRL1; ADORA2A; Aggrecan; AGR2; AICDA; AIF1; AIG1; AKAP1; AKAP2; AMH; AMHR2; amyloid beta; ANGPTL; ANGPT2; ANGPTL3; ANGPTL4; ANPEP; APC; APOC1; AR; ASLG659; ASPHDI (aspartate beta-hydroxylase domain containing 1; LOC253982); AZGP1 (zinc-a-glycoprotein); B7.1; B7.2; BAD; BAFF-R (B cell-activating factor receptor, BLyS receptor 3, BR3; BAG1; BAI1; BCL2; BCL6; BDNF; BLNK; BLRI (MDR15); BMP1; BMP2; BMP3B (GDF10); BMP4; BMP6; BMP8; BMPR1A; BMPR1B (bone morphogenic protein receptor-type IB); BMPR2; BPAG1 (plectin); BRCA1; Brevican; C19orf10 (IL27w); C3; C4A; C5; C5R1; CANT1; CASP1; CASP4; CAV1; CCBP2 (D6/JAB61); CCL1 (1-309); CCL11 (eotaxin); CCL13 (MCP-4); CCL15 (MIP1δ); CCL16 (HCC-4); CCL17 (TARC); CCL18 (PARC); CCL19 (MIP-3β); CCL2 (MCP-1); MCAF; CCL20 (MIP-3α); CCL21 (MTP-2); SLC; exodus-2; CCL22 (MDC/STC-1); CCL23 (MPIF-1); CCL24 (MPIF-2/eotaxin-2); CCL25 (TECK); CCL26 (eotaxin-3); CCL27 (CTACK/ILC); CCL28; CCL3 (MTP-Iα); CCL4 (MDP-Iβ); CCL5(RANTES); CCL7 (MCP-3); CCL8 (mcp-2); CCNA1; CCNA2; CCND1; CCNE1; CCNE2; CCR1 (CKRI/HM145); CCR2 (mcp-IRβ/RA);CCR3 (CKR/CMKBR3); CCR4; CCR5 (CMKBR5/ChemR13); CCR6 (CMKBR6/CKR-L3/STRL22/DRY6); CCR7 (CKBR7/EBI1); CCR8 (CMKBR8/TER1/CKR-L1); CCR9 (GPR-9-6); CCRL1 (VSHK1); CCRL2 (L-CCR); CD164; CD19; CD1C; CD20; CD200; CD22 (B-cell receptor CD22-B isoform); CD24; CD28; CD3; CD37; CD38; CD3E; CD3G; CD3Z; CD4; CD40; CD40L; CD44; CD45RB; CD52; CD69; CD72; CD74; CD79A (CD79a, immunoglobulin-associated alpha, a B cell-specific protein); CD79B; CDS; CD80; CD81; CD83; CD86; CDH1 (E-cadherin); CDH10; CDH12; CDH13; CDH18; CDH19; CDH20; CDH5; CDH7; CDH8; CDH9; CDK2; CDK3; CDK4; CDK5; CDK6; CDK7; CDK9; CDKN1A (p21/WAF1/Cip1); CDKN1B (p27/Kip1); CDKN1C; CDKN2A (P16INK4a); CDKN2B; CDKN2C; CDKN3; CEBPB; CER1; CHGA; CHGB; Chitinase; CHST10; CKLFSF2; CKLFSF3; CKLFSF4; CKLFSF5; CKLFSF6; CKLFSF7; CKLFSF8; CLDN3;CLDN7 (claudin-7); CLL-1 (CLEC12A, MICL, and DCAL2); CLN3; CLU (clusterin); CMKLR1; CMKOR1 (RDC1); CNR1; COL 18A1; COL1A1; COL4A3; COL6A1; complement factor D; CR2; CRP; CRIPTO (CR, CR1, CRGF, CRIPTO, TDGF1, teratocarcinoma-derived growth factor); CSFI (M-CSF); CSF2 (GM-CSF); CSF3 (GCSF); CTLA4; CTNNB1 (b-catenin); CTSB (cathepsin B); CX3CL1 (SCYDI); CX3CR1 (V28); CXCL1 (GRO1); CXCL10 (IP-10); CXCL11 (I-TAC/IP-9); CXCL12 (SDF1); CXCL13; CXCL14; CXCL16; CXCL2 (GRO2); CXCL3 (GRO3); CXCL5 (ENA-78/LIX); CXCL6 (GCP-2); CXCL9 (MIG); CXCR3 (GPR9/CKR-L2); CXCR4; CXCR5 (Burkitt's lymphoma receptor 1, a G protein-coupled receptor); CXCR6 (TYMSTR/STRL33/Bonzo); CYB5; CYC1; CYSLTR1; DAB2IP; DES; DKFZp451J0118; DNCLI; DPP4; E16 (LAT1, SLC7A5); E2F1; ECGF1; EDG1; EFNA1; EFNA3; EFNB2; EGF; EGFR; ELAC2; ENG; ENO1; ENO2; ENO3; EPHB4; EphB2R; EPO; ERBB2 (Her-2); EREG; ERK8; ESR1; ESR2; ETBR (Endothelin type B receptor); F3 (TF); FADD; FasL; FASN; FCER1A; FCER2; FCGR3A; FcRH1 (Fc receptor-like protein 1); FcRH2 (IFGP4, IRTA4, SPAP1A (SH2 domain containing phosphatase anchor protein 1a), SPAPIB, SPAPIC); FGF; FGF1 (aFGF); FGF10; FGF11; FGF12; FGF12B; FGF13; FGF14; FGF16; FGF17; FGF18; FGF19; FGF2 (bFGF); FGF20; FGF21; FGF22; FGF23; FGF3 (int-2); FGF4 (HST); FGF5; FGF6 (HST-2); FGF7 (KGF); FGF8; FGF9; FGFR; FGFR3; FIGF (VEGFD); FEL1 (EPSILON); FIL1 (ZETA); FLJ12584; FLJ25530; FLRTI (fibronectin); FLT1; FOS; FOSL1 (FRA-1); FY (DARC); GABRP (GABAa); GAGEBI; GAGECI; GALNAC4S-6ST; GATA3; GDF5; GDNF-Ral (GDNF family receptor alpha 1; GFRA1; GDNFR; GDNFRA; RETL1; TRNR1; RET1L; GDNFR-alpha1; GFR-ALPHA-1); GEDA; GFI1; GGT1; GM-CSF; GNASI; GNRHI; GPR2 (CCR10); GPR19 (G protein-coupled receptor 19; Mm.4787); GPR31; GPR44; GPR54 (KISS1 receptor; KISS1R; GPR54; HOT7T175; AXOR12); GPR81 (FKSG80); GPR172A (G protein-coupled receptor 172A; GPCR41; FLJ11856; D15Ertd747e);GRCCIO (C10); GRP; GSN (Gelsolin); GSTP1; HAVCR2; HDAC4; HDAC5; HDAC7A; HDAC9; HGF; HIF1A; HOPi; histamine and histamine receptors; HLA-A; HLA-DOB (Beta subunit of MHC class II molecule (Ia antigen); HLA-DRA; HM74; HMOXI; HUMCYT2A; ICEBERG; ICOSL; 1D2; IFN-a; IFNA1; IFNA2; IFNA4; IFNA5; IFNA6; IFNA7; IFNB1; IFNgamma; DFNW1; IGBP1; IGF1; IGF1R; IGF2; IGFBP2; IGFBP3; IGFBP6; IL-1; IL10; IL10RA; IL10RB; IL11; IL11RA; IL-12; IL12A; IL12B; IL12RB1; IL12RB2; IL13; IL13RA1; IL13RA2; IL14; IL15; IL15RA; IL16; IL17; IL17B; IL17C; IL17R; IL18; IL18BP; IL18R1; IL18RAP; IL19; IL1A; IL1B; IL1F10; IL1F5; IL1F6; IL1F7; IL1F8; IL1F9; IL1HY1; IL1R1; IL1R2; IL1RAP; IL1RAPL1; IL1RAPL2; IL1RL1; IL1RL2, IL1RN; IL2; IL20; IL20Ra; IL21 R; IL22; IL-22c; IL22R; IL22RA2; IL23; IL24; IL25; IL26; IL27; IL28A; IL28B; IL29; IL2RA; IL2RB; IL2RG; IL3; IL30; IL3RA; IL4; IL4R; IL5; IL5RA; IL6; IL6R; IL6ST (glycoprotein 130); influenza A; influenza B; EL7; EL7R; EL8; IL8RA; DL8RB; IL8RB; DL9; DL9R; DLK; INHA; INHBA; INSL3; INSL4; IRAK1; IRTA2 (Immunoglobulin superfamily receptor translocation associated 2); ERAK2; ITGA1; ITGA2; ITGA3; ITGA6 (a6 integrin); ITGAV; ITGB3; ITGB4 (b4 integrin); a407 and aEP7 integrin heterodimers; JAG1; JAK1; JAK3; JUN; K6HF; KAIl; KDR; KITLG; KLF5 (GC Box BP); KLF6; KLKIO; KLK12; KLK13; KLK14; KLK15; KLK3; KLK4; KLK5; KLK6; KLK9; KRT1; KRT19 (Keratin 19); KRT2A; KHTHB6 (hair-specific type H keratin); LAMAS; LEP (leptin); LGR5 (leucine-rich repeat-containing G protein-coupled receptor 5; GPR49, GPR67); Lingo-p75; Lingo-Troy; LPS; LTA (TNF-b); LTB; LTB4R (GPR16); LTB4R2; LTBR; LY64 (Lymphocyte antigen 64 (RP105), type I membrane protein of the leucine rich repeat (LRR) family); Ly6E (lymphocyte antigen 6 complex, locus E; Ly67,RIG-E,SCA-2,TSA-1); Ly6G6D (lymphocyte antigen 6 complex, locus G6D; Ly6-D, MEGT1); LY6K (lymphocyte antigen 6 complex, locus K; LY6K; HSJ001348; FLJ35226); MACMARCKS; MAG or OMgp; MAP2K7 (c-Jun); MDK; MDP; MIB1; midkine; MEF; MIP-2; MKI67; (Ki-67); MMP2; MMP9; MPF (MPF, MSLN, SMR, megakaryocyte potentiating factor, mesothelin); MS4A1; MSG783 (RNF124, hypothetical protein FLJ20315); MSMB; MT3 (metallothionectin-111); MTSS1; MUC1 (mucin); MYC; MY088; Napi3b (also known as NaPi2b) (NAPI-3B, NPTIIb, SLC34A2, solute carrier family 34 (sodium phosphate), member 2, type II sodium-dependent phosphate transporter 3b); NCA; NCK2; neurocan; NFKB1; NFKB2; NGFB (NGF); NGFR; NgR-Lingo; NgR-Nogo66 (Nogo); NgR-p75; NgR-Troy; NME1 (NM23A); NOX5; NPPB; NROB1; NROB2; NR1D1; NR1D2; NR1H2; NR1H3; NR1H4; NR112; NR113; NR2C1; NR2C2; NR2E1; NR2E3; NR2F1; NR2F2; NR2F6; NR3C1; NR3C2; NR4A1; NR4A2; NR4A3; NR5A1; NR5A2; NR6A1; NRP1; NRP2; NT5E; NTN4; ODZI; OPRD1; OX40; P2RX7; P2X5 (Purinergic receptor P2X ligand-gated ion channel 5); PAP; PART1; PATE; PAWR; PCA3; PCNA; PD-Li; PD-L2; PD-1; POGFA; POGFB; PECAMI; PF4 (CXCL4); PGF; PGR; phosphacan; PIAS2; PIK3CG; PLAU (uPA); PLG; PLXDCI; PMEL17 (silverhomolog; SILV; D12S53E; PMEL17; SI; SIL); PPBP (CXCL7); PPID; PRI; PRKCQ; PRKDI; PRL; PROC; PROK2; PSAP; PSCA hlg (2700050C12Rik, C530008016Rik, RIKEN cDNA 2700050C12, RIKEN cDNA 2700050C12 gene); PTAFR; PTEN; PTGS2 (COX-2); PTN; RAC2 (p21 Rac2); RARB; RET (ret proto-oncogene; MEN2A; HSCR1; MEN2B; MTC1; PTC; CDHF12; Hs.168114; RET51; RET-ELE1); RGSI; RGS13; RGS3; RNF110 (ZNF144); ROBO2; S100A2; SCGB1D2 (lipophilin B); SCGB2A1 (mammaglobin2); SCGB2A2 (mammaglobin 1); SCYEI (endothelial Monocyte-activating cytokine); SDF2; Sema 5b (FLJ10372, KIAA1445, Mm.42015, SEMASB, SEMAG, Semaphorin 5b Hlog, sema domain, seven thrombospondin repeats (type 1 and type 1-like), transmembrane Domain™ and short cytoplasmic domain, (semaphorin) 5B); SERPINAI; SERPINA3; SERPINB5 (maspin); SERPINEI(PAI-1); SERPDMFI; SHBG; SLA2; SLC2A2; SLC33A1; SLC43A1; SLIT2; SPPI; SPRRIB (Sprl); ST6GAL1; STABI; STAT6; STEAP (six transmembrane epithelial antigen of prostate); STEAP2 (HGNC_8639, IPCA-1, PCANAPI, STAMPI, STEAP2, STMP, prostate cancer associated gene 1, prostate cancer associated protein 1, six transmembrane epithelial antigen of prostate 2, six transmembrane prostate protein); TB4R2; TBX21; TCPIO; TOGFI; TEK; TENB2 (putative transmembrane proteoglycan); TGFA; TGFBI; TGFBII; TGFB2; TGFB3; TGFBI; TGFBRI; TGFBR2; TGFBR3; THIL; THBSI (thrombospondin-1); THBS2; THBS4; THPO; TIE (Tie-1); TMP3; tissue factor; TLR1; TLR2; TLR3; TLR4; TLR5; TLR6; TLR7; TLR8; TLR9; TLR10; TMEFF1 (transmembrane protein with EGF-like and two follistatin-like domains 1; Tomoregulin-1); TMEM46 (shisa homolog 2); TNF; TNF-a; TNFAEP2 (B94); TNFAIP3; TNFRSFIIA; TNFRSFIA; TNFRSFIB; TNFRSF21; TNFRSF5; TNFRSF6 (Fas); TNFRSF7; TNFRSF8; TNFRSF9; TNFSF10 (TRAIL); TNFSF11 (TRANCE); TNFSF12 (APO3L); TNFSF13 (April); TNFSF13B; TNFSF14 (HVEM-L); TNFSF15 (VEGI); TNFSF18; TNFSF4 (OX40 ligand); TNFSF5 (CD40 ligand); TNFSF6 (FasL); TNFSF7 (CD27 ligand); TNFSFS (CD30 ligand); TNFSF9 (4-1 BB ligand); TOLLIP; Toll-like receptors; TOP2A (topoisomerase Ea); TP53; TPM1; TPM2; TRADD; TMEM118 (ring finger protein, transmembrane 2; RNFT2; FLJ14627); TRAF1; TRAF2; TRAF3; TRAF4; TRAF5; TRAF6; TREM1; TREM2; TrpM4 (BR22450, FLJ20041, TRPM4, TRPM4B, transient receptor potential cation channel, subfamily M, member 4); TRPC6; TSLP; TWEAK; Tyrosinase (TYR; OCAIA; OCA1A; tyrosinase; SHEP3); VEGF; VEGFB; VEGFC; versican; VHL C5; VLA-4; XCL1 (lymphotactin); XCL2 (SCM-lb); XCRI(GPR5/CCXCRI); YY1; and ZFPM2.

In certain embodiments, an antibody produced by the cells and methods disclosed herein is capable of binding to CD proteins such as CD3, CD4, CD5, CD16, CD19, CD20, CD21 (CR2 (Complement receptor 2) or C3DR (C3d/Epstein Barr virus receptor) or Hs.73792); CD33; CD34; CD64; CD72 (B-cell differentiation antigen CD72, Lyb-2); CD79b (CD79B, CD790, IGb (immunoglobulin-associated beta), B29); CD200 members of the ErbB receptor family such as the EGF receptor, HER2, HER3, or HER4 receptor; cell adhesion molecules such as LFA-1, Mac1, p150.95, VLA-4, ICAM-1, VCAM, alpha4/beta7 integrin, and alphav/beta3 integrin including either alpha or beta subunits thereof (e.g., anti-CD11a, anti-CD18, or anti-CD11b antibodies); growth factors such as VEGF-A, VEGF-C; tissue factor (TF); alpha interferon (alphaIFN); TNFalpha, an interleukin, such as IL-1 beta, IL-3, IL-4, IL-5, IL-6, IL-8, IL-9, IL-13, IL 17 AF, IL-1S, IL-13R alpha1, IL13R alpha2, IL-4R, IL-5R, IL-9R, IgE; blood group antigens; flk2/flt3 receptor; obesity (OB) receptor; mpl receptor; CTLA-4; RANKL, RANK, RSV F protein, protein C etc.

In certain embodiments, the cells and methods provided herein can be used to produce an antibody (or a multispecific antibody, such as a bispecific antibody) that specifically binds to complement protein C5 (e.g., an anti-C5 agonist antibody that specifically binds to human C5). In certain embodiments, the anti-C5 antibody comprises 1, 2, 3, 4, 5 or 6 CDRs selected from (a) a heavy chain variable region CDR1 comprising the amino acid sequence of SSYYMA (SEQ ID NO:1); (b) a heavy chain variable region CDR2 comprising the amino acid sequence of AIFTGSGAEYKAEWAKG (SEQ ID NO:26); (c) a heavy chain variable region CDR3 comprising the amino acid sequence of DAGYDYPTHAMHY (SEQ ID NO: 27); (d) a light chain variable region CDR1 comprising the amino acid sequence of RASQGISSSLA (SEQ ID NO: 28); (e) a light chain variable region CDR2 comprising the amino acid sequence of GASETES (SEQ ID NO: 29); and (f) a light chain variable region CDR3 comprising the amino acid sequence of QNTKVGSSYGNT (SEQ ID NO: 30). For example, in certain embodiments, the anti-C5 antibody comprises a heavy chain variable domain (VH) sequence comprising one, two or three CDRs selected from: (a) a heavy chain variable region CDR1 comprising the amino acid sequence of (SSYYMA (SEQ ID NO: 1); (b) a heavy chain variable region CDR2 comprising the amino acid sequence of AIFTGSGAEYKAEWAKG (SEQ ID NO: 26); (c) a heavy chain variable region CDR3 comprising the amino acid sequence of DAGYDYPTHAMHY (SEQ ID NO: 27); and/or a light chain variable domain (VL) sequence comprising one, two or three CDRs selected from (d) a light chain variable region CDR1 comprising the amino acid sequence of RASQGISSSLA (SEQ ID NO: 28); (e) a light chain variable region CDR2 comprising the amino acid sequence of GASETES (SEQ ID NO: 29); and (f) a light chain variable region CDR3 comprising the amino acid sequence of QNTKVGSSYGNT (SEQ ID NO: 30). The sequences of CDR1, CDR2 and CDR3 of the heavy chain variable region and CDR1, CDR2 and CDR3 of the light chain variable region above are disclosed in US 2016/0176954 as SEQ ID NO: 117, SEQ ID NO: 118, SEQ ID NO: 121, SEQ ID NO: 122, SEQ ID NO: 123, and SEQ ID NO: 125, respectively. (See Tables 7 and 8 in US 2016/0176954.) In certain embodiments, the anti-C5 antibody comprises the VH and VL sequences QVQLVESGGG LVQPGRSLRL SCAASGFTVH SSYYMAWVRQ APGKGLEWVG AIFTGSGAEY KAEWAKGRVT ISKDTSKNQV VLTMTNMDPV DTATYYCASD AGYDYPTHAM HYWGQGTLVT VSS (SEQ ID NO: 31) and DIQMTQSPSS LSASVGDRVT ITCRASQGIS SSLAWYQQKP GKAPKLLIYG ASETESGVPS RFSGSGSGTD FTLTISSLQP EDFATYYCQN TKVGSSYGNT FGGGTKVEIK (SEQ ID NO: 32), respectively, including post-translational modifications of those sequences. The VH and VL sequences above are disclosed in US 2016/0176954 as SEQ ID NO: 106 and SEQ ID NO: 111, respectively. (See Tables 7 and 8 in US 2016/0176954.) In certain embodiments, the anti-C5 antibody is 305L015 (see US 2016/0176954).

In certain embodiments, an antibody produced by methods disclosed herein is capable of binding to OX40 (e.g., an anti-OX40 agonist antibody that specifically binds to human OX40). In certain embodiments, the anti-OX40 antibody comprises 1, 2, 3, 4, 5 or 6 CDRs selected from (a) a heavy chain variable region CDR1 comprising the amino acid sequence of DSYMS (SEQ ID NO: 2); (b) a heavy chain variable region CDR2 comprising the amino acid sequence of DMYPDNGDSSYNQKFRE (SEQ ID NO: 3); (c) a heavy chain variable region CDR3 comprising the amino acid sequence of APRWYFSV (SEQ ID NO: 4); (d) a light chain variable region CDR1 comprising the amino acid sequence of RASQDISNYLN (SEQ ID NO: 5); (e) a light chain variable region CDR2 comprising the amino acid sequence of YTSRLRS (SEQ ID NO: 6); and (f) a light chain variable region CDR3 comprising the amino acid sequence of QQGHTLPPT (SEQ ID NO: 7). For example, in certain embodiments, the anti-OX40 antibody comprises a heavy chain variable domain (VH) sequence comprising one, two or three CDRs selected from: (a) a heavy chain variable region CDR1 comprising the amino acid sequence of DSYMS (SEQ ID NO: 2); (b) a heavy chain variable region CDR2 comprising the amino acid sequence of DMYPDNGDSSYNQKFRE (SEQ ID NO: 3); and (c) a heavy chain variable region CDR3 comprising the amino acid sequence of APRWYFSV (SEQ ID NO: 4) and/or a light chain variable domain (VL) sequence comprising one, two or three CDRs selected from (a) a light chain variable region CDR1 comprising the amino acid sequence of RASQDISNYLN (SEQ ID NO: 5); (b) a light chain variable region CDR2 comprising the amino acid sequence of YTSRLRS (SEQ ID NO: 6); and (c) a light chain variable region CDR3 comprising the amino acid sequence of QQGHTLPPT (SEQ ID NO: 7). In certain embodiments, the anti-OX40 antibody comprises the VH and VL sequences EVQLVQSGAE VKKPGASVKV SCKASGYTFT DSYMSWVRQA PGQGLEWIGD MYPDNGDSSY NQKFRERVTI TRDTSTSTAY LELSSLRSED TAVYYCVLAP RWYFSVWGQG TLVTVSS (SEQ ID NO: 8) and DIQMTQSPSS LSASVGDRVT ITCRASQDIS NYLNWYQQKP GKAPKLLIYY TSRLRSGVPS RFSGSGSGTD FTLTISSLQP EDFATYYCQQ GHTLPPTFGQ GTKVEIK (SEQ ID NO: 9), respectively, including post-translational modifications of those sequences.

In certain embodiments, the anti-OX40 antibody comprises 1, 2, 3, 4, 5 or 6 CDRs selected from (a) a heavy chain variable region CDR1 comprising the amino acid sequence of NYLIE (SEQ ID NO: 10); (b) a heavy chain variable region CDR2 comprising the amino acid sequence of VINPGSGDTYYSEKFKG (SEQ ID NO: 11); (c) a heavy chain variable region CDR3 comprising the amino acid sequence of DRLDY (SEQ ID NO: 12); (d) a light chain variable region CDR1 comprising the amino acid sequence of HASQDISSYIV (SEQ ID NO: 13); (e) a light chain variable region CDR2 comprising the amino acid sequence of HGTNLED (SEQ ID NO: 14); and (f) a light chain variable region CDR3 comprising the amino acid sequence of VHYAQFPYT (SEQ ID NO: 15). For example, in certain embodiments, the anti-OX40 antibody comprises a heavy chain variable domain (VH) sequence comprising one, two or three CDRs selected from: (a) a heavy chain variable region CDR1 comprising the amino acid sequence of NYLIE (SEQ ID NO: 10); (b) a heavy chain variable region CDR2 comprising the amino acid sequence of VINPGSGDTYYSEKFKG (SEQ ID NO: 11); and (c) a heavy chain variable region CDR3 comprising the amino acid sequence of DRLDY (SEQ ID NO: 12) and/or a light chain variable domain (VL) sequence comprising one, two or three CDRs selected from (a) a light chain variable region CDR1 comprising the amino acid sequence of HASQDISSYIV (SEQ ID NO: 13); (b) a light chain variable region CDR2 comprising the amino acid sequence of HGTNLED (SEQ ID NO: 14); and (c) a light chain variable region CDR3 comprising the amino acid sequence of VHYAQFPYT (SEQ ID NO: 15). In certain embodiments, the anti-OX40 antibody comprises the VH and VL sequences EVQLVQSGAE VKKPGASVKV SCKASGYAFT NYLIEWVRQA PGQGLEWIGV INPGSGDTYY SEKFKGRVTI TRDTSTSTAY LELSSLRSED TAVYYCARDR LDYWGQGTLV TVSS (SEQ ID NO: 16) and DIQMTQSPSS LSASVGDRVT ITCHASQDIS SYIVWYQQKP GKAPKLLIYH GTNLEDGVPS RFSGSGSGTD FTLTISSLQP EDFATYYCVH YAQFPYTFGQ GTKVEIK (SEQ ID NO: 17), respectively, including post-translational modifications of those sequences.

Further details regarding anti-OX40 antibodies are provided in WO 2015/153513, which is incorporated herein by reference in its entirety.

In certain embodiments, an antibody produced by the cells and methods disclosed herein is capable of binding to influenza virus B hemagglutinin, i.e., “fluB” (e.g., an antibody that binds hemagglutinin from the Yamagata lineage of influenza B viruses, binds hemagglutinin from the Victoria lineage of influenza B viruses, binds hemagglutinin from ancestral lineages of influenza B virus, or binds hemagglutinin from the Yamagata lineage, the Victoria lineage, and ancestral lineages of influenza B virus, in vitro and/or in vivo). Further details regarding anti-FluB antibodies are described in WO 2015/148806, which is incorporated herein by reference in its entirety.

In certain embodiments, an antibody produced by the cells and methods disclosed herein is capable of binding to low density lipoprotein receptor-related protein (LRP)-1 or LRP-8 or transferrin receptor, and at least one target selected from the group consisting of beta-secretase (BACE1 or BACE2), alpha-secretase, gamma-secretase, tau-secretase, amyloid precursor protein (APP), death receptor 6 (DR6), amyloid beta peptide, alpha-synuclein, Parkin, Huntingtin, p75 NTR, CD40 and caspase-6.

In certain embodiments, an antibody produced by the cells and methods disclosed herein is a human IgG₂ antibody against CD40. In certain embodiments, the anti-CD40 antibody is RG7876.

In certain embodiments, the cells and methods of the present disclosure can be used to product a polypeptide. For example, but not by way of limitation, the polypeptide is a targeted immunocytokine. In certain embodiments, the targeted immunocytokine is a CEA-IL2v immunocytokine. In certain embodiments, the CEA-IL2v immunocytokine is RG7813. In certain embodiments, the targeted immunocytokine is a FAP-IL2v immunocytokine. In certain embodiments, the FAP-IL2v immunocytokine is RG7461.

In certain embodiments, the multispecific antibody (such as a bispecific antibody) produced by the cells or methods provided herein is capable of binding to CEA and at least one additional target molecule. In certain embodiments, the multispecific antibody (such as a bispecific antibody) produced according to methods provided herein is capable of binding to a tumor targeted cytokine and at least one additional target molecule. In certain embodiments, the multispecific antibody (such as a bispecific antibody) produced according to methods provided herein is fused to IL2v (i.e., an interleukin 2 variant) and binds an IL1-based immunocytokine and at least one additional target molecule. In certain embodiments, the multispecific antibody (such as a bispecific antibody) produced according to methods provided herein is a T-cell bispecific antibody (i.e., a bispecific T-cell engager or BiTE).

In certain embodiments, the multispecific antibody (such as a bispecific antibody) produced according to methods provided herein is capable of binding to at least two target molecules selected from: IL-1 alpha and IL-1 beta, IL-12 and IL-1S; IL-13 and IL-9; IL-13 and IL-4; IL-13 and IL-5; IL-5 and IL-4; IL-13 and IL-1beta; IL-13 and IL-25; IL-13 and TARC; IL-13 and MDC; IL-13 and MEF; IL-13 and TGF-˜; IL-13 and LHR agonist; IL-12 and TWEAK, IL-13 and CL25; IL-13 and SPRR2a; IL-13 and SPRR2b; IL-13 and ADAMS, IL-13 and PED2, IL17A and IL17F, CEA and CD3, CD3 and CD19, CD138 and CD20; CD138 and CD40; CD19 and CD20; CD20 and CD3; CD3S and CD13S; CD3S and CD20; CD3S and CD40; CD40 and CD20; CD-S and IL-6; CD20 and BR3, TNF alpha and TGF-beta, TNF alpha and IL-1 beta; TNF alpha and IL-2, TNF alpha and IL-3, TNF alpha and IL-4, TNF alpha and IL-5, TNF alpha and IL6, TNF alpha and IL8, TNF alpha and IL-9, TNF alpha and IL-10, TNF alpha and IL-11, TNF alpha and IL-12, TNF alpha and IL-13, TNF alpha and IL-14, TNF alpha and IL-15, TNF alpha and IL-16, TNF alpha and IL-17, TNF alpha and IL-18, TNF alpha and IL-19, TNF alpha and IL-20, TNF alpha and IL-23, TNF alpha and IFN alpha, TNF alpha and CD4, TNF alpha and VEGF, TNF alpha and MIF, TNF alpha and ICAM-1, TNF alpha and PGE4, TNF alpha and PEG2, TNF alpha and RANK ligand, TNF alpha and Te38, TNF alpha and BAFF,TNF alpha and CD22, TNF alpha and CTLA-4, TNF alpha and GP130, TNF a and IL-12p40, VEGF and Angiopoietin, VEGF and HER2, VEGF-A and HER2, VEGF-A and PDGF, HER1 and HER2, VEGFA and ANG2,VEGF-A and VEGF-C, VEGF-C and VEGF-D, HER2 and DR5,VEGF and IL-8, VEGF and MET, VEGFR and MET receptor, EGFR and MET, VEGFR and EGFR, HER2 and CD64, HER2 and CD3, HER2 and CD16, HER2 and HER3; EGFR (HER1) and HER2, EGFR and HER3, EGFR and HER4, IL-14 and IL-13, IL-13 and CD40L, IL4 and CD40L, TNFR1 and IL-1 R, TNFR1 and IL-6R and TNFR1 and IL-18R, EpCAM and CD3, MAPG and CD28, EGFR and CD64, CSPGs and RGM A; CTLA-4 and BTN02; IGF1 and IGF2; IGF1/2 and Erb2B; MAG and RGM A; NgR and RGM A; NogoA and RGM A; OMGp and RGM A; POL-1 and CTLA-4; and RGM A and RGM B.

In certain embodiments, the multispecific antibody (such as a bispecific antibody) produced according to methods provided herein is an anti-CEA/anti-CD3 bispecific antibody. In certain embodiments, the anti-CEA/anti-CD3 bispecific antibody is RG7802. In certain embodiments, the anti-CEA/anti-CD3 bispecific antibody comprises the amino acid sequences set forth in SE ID NOs: 18-21 are provided below:

(SEQ ID NO: 18)
DIQMTQSPSS LSASVGDRVT ITCKASAAVG TYVAWYQQKP
GKAPKLLIYS ASYRKRGVPS RFSGSGSGTD FTLTISSLQP
EDFATYYCHQ YYTYPLFTFG QGTKLEIKRT VAAPSVFIFP
PSDEQLKSGT ASVVCLLNNF YPREAKVQWK VDNALQSGNS
QESVTEQDSK DSTYSLSSTL TLSKADYEKH KVYACEVTHQ
GLSSPVTKSF NRGEC
(SEQ ID NO: 19)
QAVVTQEPSL TVSPGGTVTL TCGSSTGAVT TSNYANWVQE
KPGQAFRGLI GGTNKRAPGT PARFSGSLLG GKAALTLSGA
QPEDEAEYYC ALWYSNLWVF GGGTKLTVLS SASTKGPSVF
PLAPSSKSTS GGTAALGCLV KDYFPEPVTV SWNSGALTSG
VHTFPAVLQS SGLYSLSSVV TVPSSSLGTQ TYICNVNHKP
SNTKVDKKVE PKSC
(SEQ ID NO: 20)
QVQLVQSGAE VKKPGASVKV SCKASGYTFT EFGMNWVRQA
PGQGLEWMGW INTKTGEATY VEEFKGRVTF TTDTSTSTAY
MELRSLRSDD TAVYYCARWD FAYYVEAMDY WGQGTTVTVS
SASTKGPSVF PLAPSSKSTS GGTAALGCLV KDYFPEPVTV
SWNSGALTSG VHTFPAVLQS SGLYSLSSVV TVPSSSLGTQ
TYICNVNHKP SNTKVDKKVE PKSCDGGGGS GGGGSEVQLL
ESGGGLVQPG GSLRLSCAAS GFTFSTYAMN WVRQAPGKGL
EWVSRIRSKY NNYATYYADS VKGRFTISRD DSKNTLYLQM
NSLRAEDTAV YYCVRHGNFG NSYVSWFAYW GQGTLVTVSS
ASVAAPSVFI FPPSDEQLKS GTASVVCLLN NFYPREAKVQ
WKVDNALQSG NSQESVTEQD SKDSTYSLSS TLTLSKADYE
KHKVYACEVT HQGLSSPVTK SFNRGECDKT HTCPPCPAPE
AAGGPSVFLF PPKPKDTLMI SRTPEVTCVV VDVSHEDPEV
KFNWYVDGVE VHNAKTKPRE EQYNSTYRVV SVLTVLHQDW
LNGKEYKCKV SNKALGAPIE KTISKAKGQP REPQVYTLPP
CRDELTKNQV SLWCLVKGFY PSDIAVEWES NGQPENNYKT
TPPVLDSDGS FFLYSKLTVD KSRWQQGNVF SCSVMHEALH
NHYTQKSLSL SPGK
(SEQ ID NO: 21)
QVQLVQSGAE VKKPGASVKV SCKASGYTFT EFGMNWVRQA
PGQGLEWMG WINTKTGEATY VEEFKGRVTF TTDTSTSTAY
MELRSLRSDD TAVYYCARWD FAYYVEAMD YWGQGTTVTVS
SASTKGPSVF PLAPSSKSTS GGTAALGCLV KDYFPEPVTV
SWNSGALTS GVHTFPAVLQS SGLYSLSSVV TVPSSSLGTQ
TYICNVNHKP SNTKVDKKVE PKSCDKTHT CPPCPAPEAAG
GPSVFLFPPK PKDTLMISRT PEVTCVVVDV SHEDPEVKFN
WYVDGVEVH NAKTKPREEQY NSTYRVVSVL TVLHQDWLNG
KEYKCKVSNK ALGAPIEKTI SKAKGQPRE PQVCTLPPSRD
ELTKNQVSLS CAVKGFYPSD IAVEWESNGQ PENNYKTTPP
VLDSDGSFF LVSKLTVDKSR WQQGNVFSCS VMHEALHNHY
TQKSLSLSPG K

Further details regarding anti-CEA/anti-CD3 bispecific antibodies are provided in WO 2014/121712, which is incorporated herein by reference in its entirety.

In certain embodiments, a multispecific antibody (such as a bispecific antibody) produced by the cells and methods disclosed herein is an anti-VEGF/anti-angiopoietin bispecific antibody. In certain embodiments, the anti-VEGF/anti-angiopoietin bispecific antibody bispecific antibody is a Crossmab. In certain embodiments, the anti-VEGF/anti-angiopoietin bispecific antibody is RG7716. In certain embodiments, the anti-CEA/anti-CD3 bispecific antibody comprises the amino acid sequences set forth in SEQ ID NOs: 22-25 are provided below:

(SEQ ID NO: 22)
EVQLVESGGG LVQPGGSLRL SCAASGYDFT HYGMNWVRQA
PGKGLEWVGW INTYTGEPTY AADFKRRFTF SLDTSKSTAY
LQMNSLRAED TAVYYCAKYP YYYGTSHWYF DVWGQGTLVT
VSSASTKGPS VFPLAPSSKS TSGGTAALGC LVKDYFPEPV
TVSWNSGALT SGVHTFPAVL QSSGLYSLSS VVTVPSSSLG
TQTYICNVNH KPSNTKVDKK VEPKSCDKTH TCPPCPAPEA
AGGPSVFLFP PKPKDTLMAS RTPEVTCVVV DVSHEDPEVK
FNWYVDGVEV HNAKTKPREE QYNSTYRVVS VLTVLAQDWL
NGKEYKCKVS NKALGAPIEK TISKAKGQPR EPQVYTLPPC
RDELTKNQVS LWCLVKGFYP SDIAVEWESN GQPENNYKTT
PPVLDSDGSF FLYSKLTVDK SRWQQGNVFS CSVMHEALHN
AYTQKSLSLS PGK
(SEQ ID NO: 23)
QVQLVQSGAE VKKPGASVKV SCKASGYTFT GYYMHWVRQA
PGQGLEWMGW INPNSGGTNY AQKFQGRVTM TRDTSISTAY
MELSRLRSDD TAVYYCARSP NPYYYDSSGY YYPGAFDIWG
QGTMVTVSSA SVAAPSVFIF PPSDEQLKSG TASVVCLLNN
FYPREAKVQW KVDNALQSGN SQESVTEQDS KDSTYSLSST
LTLSKADYEK HKVYACEVTH QGLSSPVTKS FNRGECDKTH
TCPPCPAPEA AGGPSVFLFP PKPKDTLMAS RTPEVTCVVV
DVSHEDPEVK FNWYVDGVEV HNAKTKPREE QYNSTYRVVS
VLTVLAQDWL NGKEYKCKVS NKALGAPIEK TISKAKGQPR
EPQVCTLPPS RDELTKNQVS LSCAVKGFYP SDIAVEWESN
GQPENNYKTT PPVLDSDGSF FLVSKLTVDK SRWQQGNVFS
CSVMHEALHN AYTQKSLSLS PGK
(SEQ ID NO: 24)
DIQLTQSPSS LSASVGDRVT ITCSASQDIS NYLNWYQQKP
GKAPKVLIYF TSSLHSGVPS RFSGSGSGTD FTLTISSLQP
EDFATYYCQQ YSTVPWTFGQ GTKVEIKRTV AAPSVFIFPP
SDEQLKSGTA SVVCLLNNFY PREAKVQWKV DNALQSGNSQ
ESVTEQDSKD STYSLSSTLT LSKADYEKHK VYACEVTHQG
LSSPVTKSFN RGEC
(SEQ ID NO: 25)
SYVLTQPPSV SVAPGQTARI TCGGNNIGSK SVHWYQQKPG
QAPVLVVYDD SDRPSGIPER FSGSNSGNTA TLTISRVEAG
DEADYYCQVW DSSSDHWVFG GGTKLTVLSS ASTKGPSVFP
LAPSSKSTSG GTAALGCLVK DYFPEPVTVS WNSGALTSGV
HTFPAVLQSS GLYSLSSVVT VPSSSLGTQT YICNVNHKPS
NTKVDKKVEP KSC

In certain embodiments, the multispecific antibody (such as a bispecific antibody) produced by methods disclosed herein is an anti-Ang2/anti-VEGF bispecific antibody. In certain embodiments, the anti-Ang2/anti-VEGF bispecific antibody is RG7221. In certain embodiments, the anti-Ang2/anti-VEGF bispecific antibody is CAS Number 1448221-05-3.

Soluble antigens or fragments thereof, optionally conjugated to other molecules, can be used as immunogens for generating antibodies. For transmembrane molecules, such as receptors, fragments of these (e.g., the extracellular domain of a receptor) can be used as the immunogen. Alternatively, cells expressing the transmembrane molecule can be used as the immunogen. Such cells can be derived from a natural source (e.g., cancer cell lines) or can be cells which have been transformed by recombinant techniques to express the transmembrane molecule. Other antigens and forms thereof useful for preparing antibodies will be apparent to those in the art.

In certain embodiments, the polypeptide (e.g., antibodies) produced by the cells and methods disclosed herein is capable of binding to can be further conjugated to a chemical molecule such as a dye or cytotoxic agent such as a chemotherapeutic agent, a drug, a growth inhibitory agent, a toxin (e.g., an enzymatically active toxin of bacterial, fungal, plant, or animal origin, or fragments thereof), or a radioactive isotope (i.e., a radioconjugate). An immunoconjugate comprising an antibody or bispecific antibody produced using the methods described herein can contain the cytotoxic agent conjugated to a constant region of only one of the heavy chains or only one of the light chains.

### 5.5.6 Antibody Variants

In certain aspects, amino acid sequence variants of the antibodies provided herein are contemplated, e.g., the antibodies provided in Section 5.5.5. For example, it can be desirable to alter the binding affinity and/or other biological properties of the antibody. Amino acid sequence variants of an antibody can be prepared by introducing appropriate modifications into the nucleotide sequence encoding the antibody, or by peptide synthesis. Such modifications include, for example, deletions from, and/or insertions into and/or substitutions of residues within the amino acid sequences of the antibody. Any combination of deletion, insertion, and substitution can be made to arrive at the final construct, provided that the final construct possesses the desired characteristics, e.g., antigen-binding.

### 5.5.6.1 Substitution, Insertion, and Deletion Variants

In certain aspects, antibody variants having one or more amino acid substitutions are provided. Sites of interest for substitutional mutagenesis include the CDRs and FRs. Conservative substitutions are shown in Table 1 under the heading of “preferred substitutions”. More substantial changes are provided in Table 1 under the heading of “exemplary substitutions”, and as further described below in reference to amino acid side chain classes. Amino acid substitutions can be introduced into an antibody of interest and the products screened for a desired activity, e.g., retained/improved antigen binding, decreased immunogenicity, or improved ADCC or CDC.

TABLE 1

| Original | Exemplary | Preferred |
|---|---|---|
| Residue | Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; | Leu |
| | Norleucine | |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

Amino acids can be grouped according to common side-chain properties:
(1) hydrophobic: Norieucine, Met, Ala, Vai, Leu, le;
(2) neutrai hydrophiiic: Cys, Ser, Thr, Asn, Gin;
(3) acidic: Asp, Giu;
(4) basic: His, Lys, Arg;
(5) residues that influence chain orientation: Giy, Pro;
(6) aromatic: Trp, Tyr, Phe.

Non-conservative substitutions will entail exchanging a member of one of these classes for a member of another class.

One type of substitutional variant involves substituting one or more hypervariable region residues of a parent antibody (e.g., a humanized or human antibody). Generally, the resulting variant(s) selected for further study will have modifications (e.g., improvements) in certain biological properties (e.g., increased affinity, reduced immunogenicity) relative to the parent antibody and/or will have substantially retained certain biological properties of the parent antibody. An exemplary substitutional variant is an affinity matured antibody, which can be conveniently generated, e.g., using phage display-based affinity maturation techniques such as those described herein. Briefly, one or more. CDR residues are mutated and the variant antibodies displayed on phage and screened for a particular biological activity (e.g., binding affinity).

Alterations (e.g., substitutions) can be made in CDRs, e.g., to improve antibody affinity. Such alterations can be made in CDR “hotspots”, i.e., residues encoded by codons that undergo mutation at high frequency during the somatic maturation process (see, e.g., Chowdhury, Methods Mol. Biol. 207:179-196 (2008)), and/or residues that contact antigen, with the resulting variant VH or VL being tested for binding affinity. Affinity maturation by constructing and reselecting from secondary libraries has been described, e.g., in Hoogenboom et al. in Methods in Molecular Biology 178:1-37 (O'Brien et al., ed., Human Press, Totowa, NJ, (2001).) In some aspects of affinity maturation, diversity is introduced into the variable genes chosen for maturation by any of a variety of methods (e.g., error-prone PCR, chain shuffling, or oligonucleotide-directed mutagenesis). A secondary library is then created. The library is then screened to identify any antibody variants with the desired affinity. Another method to introduce diversity involves CDR-directed approaches, in which several CDR residues (e.g., 4-6 residues at a time) are randomized. CDR residues involved in antigen binding can be specifically identified, e.g., using alanine scanning mutagenesis or modeling. CDR-H3 and CDR-L3 in particular are often targeted.

In certain aspects, substitutions, insertions, or deletions can occur within one or more CDRs so long as such alterations do not substantially reduce the ability of the antibody to bind antigen. For example, conservative alterations (e.g., conservative substitutions as provided herein) that do not substantially reduce binding affinity can be made in the CDRs. Such alterations can, for example, be outside of antigen contacting residues in the CDRs. In certain variant VH and VL sequences provided above, each CDR either is unaltered, or contains no more than one, two or three amino acid substitutions.

A useful method for identification of residues or regions of an antibody that can be targeted for mutagenesis is called “alanine scanning mutagenesis” as described by Cunningham and Wells (1989) Science, 244:1081-1085. In this method, a residue or group of target residues (e.g., charged residues such as arg, asp, his, lys, and glu) are identified and replaced by a neutral or negatively charged amino acid (e.g., alanine or polyalanine) to determine whether the interaction of the antibody with antigen is affected. Further substitutions can be introduced at the amino acid locations demonstrating functional sensitivity to the initial substitutions. Alternatively, or additionally, a crystal structure of an antigen-antibody complex can be used to identify contact points between the antibody and antigen. Such contact residues and neighboring residues can be targeted or eliminated as candidates for substitution. Variants can be screened to determine whether they contain the desired properties.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of the antibody molecule include the fusion to the N- or C-terminus of the antibody to an enzyme (e.g., for ADEPT (antibody directed enzyme prodrug therapy)) or a polypeptide which increases the serum half-life of the antibody.

### 5.5.6.2 Glycosylation Variants

In certain aspects, an antibody provided herein is altered to increase or decrease the extent to which the antibody is glycosylated. Addition or deletion of glycosylation sites to an antibody can be conveniently accomplished by altering the amino acid sequence such that one or more glycosylation sites is created or removed.

Where the antibody comprises an Fc region, the oligosaccharide attached thereto can be altered. Native antibodies produced by mammalian cells typically comprise a branched, biantennary oligosaccharide that is generally attached by an N-linkage to Asn297 of the CH2 domain of the Fc region. See, e.g., Wright et al. TIBTECH 15:26-32 (1997). The oligosaccharide can include various carbohydrates, e.g., mannose, N-acetyl glucosamine (GlcNAc), galactose, and sialic acid, as well as a fucose attached to a GlcNAc in the “stem” of the biantennary oligosaccharide structure. In some aspects, modifications of the oligosaccharide in an antibody of the disclosure can be made in order to create antibody variants with certain improved properties.

In one aspect, antibody variants are provided having a non-fucosylated oligosaccharide, i.e. an oligosaccharide structure that lacks fucose attached (directly or indirectly) to an Fc region. Such non-fucosylated oligosaccharide (also referred to as “afucosylated” oligosaccharide) particularly is an N-linked oligosaccharide which lacks a fucose residue attached to the first GlcNAc in the stem of the biantennary oligosaccharide structure. In one aspect, antibody variants are provided having an increased proportion of non-fucosylated oligosaccharides in the Fc region as compared to a native or parent antibody. For example, the proportion of non-fucosylated oligosaccharides can be at least about 20%, at least about 40%, at least about 60%, at least about 80%, or even about 100% (i.e., no fucosylated oligosaccharides are present). The percentage of non-fucosylated oligosaccharides is the (average) amount of oligosaccharides lacking fucose residues, relative to the sum of all oligosaccharides attached to Asn 297 (e. g. complex, hybrid and high mannose structures) as measured by MALDI-TOF mass spectrometry, as described in WO 2006/082515, for example. Asn297 refers to the asparagine residue located at about position 297 in the Fc region (EU numbering of Fc region residues); however, Asn297 can also be located about ±3 amino acids upstream or downstream of position 297, i.e., between positions 294 and 300, due to minor sequence variations in antibodies. Such antibodies having an increased proportion of non-fucosylated oligosaccharides in the Fc region can have improved FcγRIIIa receptor binding and/or improved effector function, in particular improved ADCC function. See, e.g., US 2003/0157108; US 2004/0093621.

Examples of cell lines capable of producing antibodies with reduced fucosylation include Lec13 CHO cells deficient in protein fucosylation (Ripka et al. Arch. Biochem. Biophys. 249:533-545 (1986); US 2003/0157108; and WO 2004/056312, especially at Example 11), and knockout cell lines, such as alpha-1,6-fucosyltransferase gene, FUT8, knockout CHO cells (see, e.g., Yamane-Ohnuki et al. Biotech. Bioeng. 87:614-622 (2004); Kanda, Y. et al., Biotechnol. Bioeng., 94(4):680-688 (2006); and WO 2003/085107), or cells with reduced or abolished activity of a GDP-fucose synthesis or transporter protein (see, e.g., US2004259150, US2005031613, US2004132140, US2004110282).

In a further aspect, antibody variants are provided with bisected oligosaccharides, e.g., in which a biantennary oligosaccharide attached to the Fc region of the antibody is bisected by GlcNAc. Such antibody variants can have reduced fucosylation and/or improved ADCC function as described above. Examples of such antibody variants are described, e.g., in Umana et al., Nat Biotechnol 17, 176-180 (1999); Ferrara et al., Biotechn Bioeng 93, 851-861 (2006); WO 99/54342; WO 2004/065540, WO 2003/011878.

Antibody variants with at least one galactose residue in the oligosaccharide attached to the Fc region are also provided. Such antibody variants can have improved CDC function. Such antibody variants are described, e.g., in WO 1997/30087; WO 1998/58964; and WO 1999/22764.

### 5.5.6.3 Fc Region Variants

In certain aspects, one or more amino acid modifications can be introduced into the Fc region of an antibody provided herein, thereby generating an Fc region variant. The Fc region variant can comprise a human Fc region sequence (e.g., a human IgG₁, IgG₂, IgG₃ or IgG₄ Fc region) comprising an amino acid modification (e.g., a substitution) at one or more amino acid positions.

In certain aspects, the present disclosure contemplates an antibody variant that possesses some but not all effector functions, which make it a desirable candidate for applications in which the half life of the antibody in vivo is important yet certain effector functions (such as complement-dependent cytotoxicity (CDC) and antibody-dependent cell-mediated cytotoxicity (ADCC)) are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the antibody lacks FcγR binding (hence likely lacking ADCC activity), but retains FcRn binding ability. The primary cells for mediating ADCC, NK cells, express FcγRIII only, whereas monocytes express FcγRI, FcγRII and FcγRIII. FcR expression on hematopoietic cells is summarized in Table 3 on page 464 of Ravetch and Kinet, Annu. Rev. Immunol. 9:457-492 (1991). Non-limiting examples of in vitro assays to assess ADCC activity of a molecule of interest is described in U.S. Pat. No. 5,500,362 (see, e.g., Hellstrom, I. et al. Proc. Nat'l Acad. Sci. USA 83:7059-7063 (1986)) and Hellstrom, I et al., Proc. Nat'l Acad. Sci. USA 82:1499-1502 (1985); 5,821,337 (see Bruggemann, M. et al., J. Exp. Med. 166:1351-1361 (1987)). Alternatively, non-radioactive assays methods can be employed (see, for example, ACTI™ non-radioactive cytotoxicity assay for flow cytometry (CellTechnology, Inc. Mountain View, CA; and CytoTox 96® non-radioactive cytotoxicity assay (Promega, Madison, WI). Useful effector cells for such assays include peripheral blood mononuclear cells (PBMC) and Natural Killer (NK) cells. Alternatively, or additionally, ADCC activity of the molecule of interest can be assessed in vivo, e.g., in a animal model such as that disclosed in Clynes et al. Proc. Nat'l Acad. Sci. USA 95:652-656 (1998). C1q binding assays can also be carried out to confirm that the antibody is unable to bind C1q and hence lacks CDC activity. See, e.g., C1q and C3c binding ELISA in WO 2006/029879 and WO 2005/100402. To assess complement activation, a CDC assay can be performed (see, for example, Gazzano-Santoro et al., J. Immunol. Methods 202:163 (1996); Cragg, M. S. et al., Blood 101:1045-1052 (2003); and Cragg, M. S. and M. J. Glennie, Blood 103:2738-2743 (2004)). FcRn binding and in vivo clearance/half life determinations can also be performed using methods known in the art (see, e.g., Petkova, S. B. et al., Int'l. Immunol. 18(12):1759-1769 (2006); WO 2013/120929 A1).

Antibodies with reduced effector function include those with substitution of one or more of Fc region residues 238, 265, 269, 270, 297, 327 and 329 (U.S. Pat. No. 6,737,056). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. No. 7,332,581).

Certain antibody variants with improved or diminished binding to FcRs are described. (See, e.g., U.S. Pat. No. 6,737,056; WO 2004/056312, and Shields et al., J. Biol. Chem. 9(2): 6591-6604 (2001).)

In certain aspects, an antibody variant comprises an Fc region with one or more amino acid substitutions which improve ADCC, e.g., substitutions at positions 298, 333, and/or 334 of the Fc region (EU numbering of residues).

In certain aspects, an antibody variant comprises an Fc region with one or more amino acid substitutions which diminish FcγR binding, e.g., substitutions at positions 234 and 235 of the Fc region (EU numbering of residues). In one aspect, the substitutions are L234A and L235A (LALA). In certain aspects, the antibody variant further comprises D265A and/or P329G in an Fc region derived from a human IgG₁ Fc region. In one aspect, the substitutions are L234A, L235A and P329G (LALA-PG) in an Fc region derived from a human IgG₁ Fc region. (See, e.g., WO 2012/130831). In another aspect, the substitutions are L234A, L235A and D265A (LALA-DA) in an Fc region derived from a human IgG₁ Fc region.

In some aspects, alterations are made in the Fc region that result in altered (i.e., either improved or diminished) C1q binding and/or Complement Dependent Cytotoxicity (CDC), e.g., as described in U.S. Pat. No. 6,194,551, WO 99/51642, and Idusogie et al. J. Immunol. 164: 4178-4184 (2000).

Antibodies with increased half lives and improved binding to the neonatal Fc receptor (FcRn), which is responsible for the transfer of maternal IgGs to the fetus (Guyer et al., J Immunol. 117:587 (1976) and Kim et al., J. Immunol. 24:249 (1994)), are described in US2005/0014934 (Hinton et al.). Those antibodies comprise an Fc region with one or more substitutions therein which improve binding of the Fc region to FcRn. Such Fc variants include those with substitutions at one or more of Fc region residues: 238, 252, 254, 256, 265, 272, 286, 303, 305, 307, 311, 312, 317, 340, 356, 360, 362, 376, 378, 380, 382, 413, 424 or 434, e.g., substitution of Fc region residue 434 (See, e.g., U.S. Pat. No. 7,371,826; Dall'Acqua, W. F., et al. J. Biol. Chem. 281 (2006) 23514-23524).

Fc region residues critical to the mouse Fc-mouse FcRn interaction have been identified by site-directed mutagenesis (see e.g. Dall'Acqua, W. F., et al. J. Immunol 169 (2002) 5171-5180). Residues 1253, H310, H433, N434, and H435 (EU index numbering) are involved in the interaction (Medesan, C., et al., Eur. J. Immunol. 26 (1996) 2533; Firan, M., et al., Int. Immunol. 13 (2001) 993; Kim, J. K., et al., Eur. J. Immunol. 24 (1994) 542). Residues 1253, H310, and H435 were found to be critical for the interaction of human Fc with murine FcRn (Kim, J. K., et al., Eur. J. Immunol. 29 (1999) 2819). Studies of the human Fc-human FcRn complex have shown that residues I253, S254, H435, and Y436 are crucial for the interaction (Firan, M., et al., Int. Immunol. 13 (2001) 993; Shields, R. L., et al., J. Biol. Chem. 276 (2001) 6591-6604). In Yeung, Y. A., et al. (J. Immunol. 182 (2009) 7667-7671) various mutants of residues 248 to 259 and 301 to 317 and 376 to 382 and 424 to 437 have been reported and examined.

In certain aspects, an antibody variant comprises an Fc region with one or more amino acid substitutions, which reduce FcRn binding, e.g., substitutions at positions 253, and/or 310, and/or 435 of the Fc-region (EU numbering of residues). In certain aspects, the antibody variant comprises an Fc region with the amino acid substitutions at positions 253, 310 and 435. In one aspect, the substitutions are I253A, H310A and H435A in an Fc region derived from a human IgG1 Fc-region. See, e.g., Grevys, A., et al., J. Immunol. 194 (2015) 5497-5508.

In certain aspects, an antibody variant comprises an Fc region with one or more amino acid substitutions, which reduce FcRn binding, e.g., substitutions at positions 310, and/or 433, and/or 436 of the Fc region (EU numbering of residues). In certain aspects, the antibody variant comprises an Fc region with the amino acid substitutions at positions 310, 433 and 436. In one aspect, the substitutions are H310A, H433A and Y436A in an Fc region derived from a human IgG₁ Fc-region. (See, e.g., WO 2014/177460 A1).

In certain aspects, an antibody variant comprises an Fc region with one or more amino acid substitutions which increase FcRn binding, e.g., substitutions at positions 252, and/or 254, and/or 256 of the Fc region (EU numbering of residues). In certain aspects, the antibody variant comprises an Fc region with amino acid substitutions at positions 252, 254, and 256. In one aspect, the substitutions are M252Y, S254T and T256E in an Fc region derived from a human IgG₁ Fc-region. See also Duncan & Winter, Nature 322:738-40 (1988); U.S. Pat. Nos. 5,648,260; 5,624,821; and WO 94/29351 concerning other examples of Fc region variants.

The C-terminus of the heavy chain of the antibody as reported herein can be a complete C-terminus ending with the amino acid residues PGK. The C-terminus of the heavy chain can be a shortened C-terminus in which one or two of the C terminal amino acid residues have been removed. In one preferred aspect, the C-terminus of the heavy chain is a shortened C-terminus ending PG. In one aspect of all aspects as reported herein, an antibody comprising a heavy chain including a C-terminal CH3 domain as specified herein, comprises the C-terminal glycine-lysine dipeptide (G446 and K447, EU index numbering of amino acid positions). In one aspect of all aspects as reported herein, an antibody comprising a heavy chain including a C-terminal CH3 domain, as specified herein, comprises a C-terminal glycine residue (G446, EU index numbering of amino acid positions).

### 5.5.6.4 Cysteine Engineered Antibody Variants

In certain aspects, it can be desirable to create cysteine engineered antibodies, e.g., THIOMAB™ antibodies, in which one or more residues of an antibody are substituted with cysteine residues. In particular aspects, the substituted residues occur at accessible sites of the antibody. By substituting those residues with cysteine, reactive thiol groups are thereby positioned at accessible sites of the antibody and can be used to conjugate the antibody to other moieties, such as drug moieties or linker-drug moieties, to create an immunoconjugate, as described further herein. Cysteine engineered antibodies can be generated as described, e.g., in U.S. Pat. Nos. 7,521,541, 8,30,930, 7,855,275, 9,000,130, or WO 2016040856.

### 5.5.6.5 Antibody Derivatives

In certain aspects, an antibody provided herein can be further modified to contain additional nonproteinaceous moieties that are known in the art and readily available. The moieties suitable for derivatization of the antibody include but are not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1, 3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, propropylene glycol homopolymers, prolypropylene oxide/ethylene oxide co-polymers, polyoxyethylated polyols (e.g., glycerol), polyvinyl alcohol, and mixtures thereof. Polyethylene glycol propionaldehyde can have advantages in manufacturing due to its stability in water. The polymer can be of any molecular weight, and can be branched or unbranched. The number of polymers attached to the antibody can vary, and if more than one polymer are attached, they can be the same or different molecules. In general, the number and/or type of polymers used for derivatization can be determined based on considerations including, but not limited to, the particular properties or functions of the antibody to be improved, whether the antibody derivative will be used in a therapy under defined conditions, etc.

### 5.5.7 Immunoconjugates

The present disclosure also provides immunoconjugates comprising an antibody disclosed herein conjugated (chemically bonded) to one or more therapeutic agents such as cytotoxic agents, chemotherapeutic agents, drugs, growth inhibitory agents, toxins (e.g., protein toxins, enzymatically active toxins of bacterial, fungal, plant, or animal origin, or fragments thereof), or radioactive isotopes.

In one aspect, an immunoconjugate is an antibody-drug conjugate (ADC) in which an antibody is conjugated to one or more of the therapeutic agents mentioned above. The antibody is typically connected to one or more of the therapeutic agents using linkers. An overview of ADC technology including examples of therapeutic agents and drugs and linkers is set forth in Pharmacol Review 68:3-19 (2016).

In another aspect, an immunoconjugate comprises an antibody as described herein conjugated to an enzymatically active toxin or fragment thereof, including but not limited to diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), Momordica charantia inhibitor, curcin, crotin, Sapaonaria officinalis inhibitor, gelonin, mitogellin, restrictocin, phenomycin, enomycin, and the tricothecenes.

In another aspect, an immunoconjugate comprises an antibody as described herein conjugated to a radioactive atom to form a radioconjugate. A variety of radioactive isotopes are available for the production of radioconjugates. Examples include At²¹¹, I¹³¹, I¹²⁵, Y⁹⁰, Re¹⁸⁶, Re¹⁸⁸, Sm¹⁵³, Bi²¹², P³², Pb²¹² and radioactive isotopes of Lu. When the radioconjugate is used for detection, it can comprise a radioactive atom for scintigraphic studies, for example tc99m or I123, or a spin label for nuclear magnetic resonance (NMR) imaging (also known as magnetic resonance imaging, mri), such as iodine-123 again, iodine-131, indium-111, fluorine-19, carbon-13, nitrogen-15, oxygen-17, gadolinium, manganese or iron.

Conjugates of an antibody and cytotoxic agent can be made using a variety of bifunctional protein coupling agents such as N-succinimidyl-3-(2-pyridyldithio) propionate (SPDP), succinimidyl-4-(N-maleimidomethyl) cyclohexane-1-carboxylate (SMCC), iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis (p-azidobenzoyl) hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene). For example, a ricin immunotoxin can be prepared as described in Vitetta et al., Science 238:1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See WO 94/11026. The linker can be a “cleavable linker” facilitating release of a cytotoxic drug in the cell. For example, an acid-labile linker, peptidase-sensitive linker, photolabile linker, dimethyl linker or disulfide-containing linker (Chari et al., Cancer Res. 52:127-131 (1992); U.S. Pat. No. 5,208,020) can be used.

The immunuoconjugates or ADCs herein expressly contemplate, but are not limited to such conjugates prepared with cross-linker reagents including, but not limited to, BMPS, EMCS, GMBS, HBVS, LC-SMCC, MBS, MPBH, SBAP, SIA, SIAB, SMCC, SMPB, SMPH, sulfo-EMCS, sulfo-GMBS, sulfo-KMUS, sulfo-IBS, sulfo-SIAB, sulfo-SMCC, and sulfo-SMPB, and SVSB (succinimidyl-(4-vinylsulfone)benzoate) which are commercially available (e.g., from Pierce Biotechnology, Inc., Rockford, IL., U.S.A).

### 5.6 EXEMPLARY EMBODIMENTS

A. In certain non-limiting embodiments, the presently disclosed subject matter provides for a mammalian cell having reduced or eliminated lactogenic activity, wherein the expression of a pyruvate kinase muscle (PKM) polypeptide isoform is knocked down or knocked out, and wherein the PKM polypeptide isoform comprises a PKM-1 polypeptide isoform.

A1. The foregoing mammalian cell of A, wherein the expression of the PKM-2 polypeptide isoform is knocked down or knocked out.

A2. The foregoing mammalian cell of A or A1, wherein the cell is a CHO cell.

A3. The foregoing mammalian cell of any one of A-A2, comprising a nucleic acid sequence encoding a product of interest.

A4. The foregoing mammalian cell of A3, wherein the product of interest comprises a protein.

A5. The foregoing mammalian cell of A3 or A4, wherein the product of interest comprises a recombinant protein.

A6. The foregoing mammalian cell of any one of A3-A5, wherein the product of interest comprises an antibody or an antigen-binding fragment thereof.

A7. The foregoing mammalian cell of A6, wherein the antibody is a multispecific antibody or an antigen-binding fragment thereof.

A8. The foregoing mammalian cell of A6, wherein the antibody consists of a single heavy chain sequence and a single light chain sequence or antigen-binding fragments thereof.

A9. The foregoing mammalian cell of any one of A6-A8, wherein the antibody comprises a chimeric antibody, a human antibody or a humanized antibody.

A10. The foregoing mammalian cell of any one of A6-A9, wherein the antibody comprises a monoclonal antibody.

A11. The foregoing mammalian cell of any one of A3-A10, wherein the nucleic acid sequence is integrated in the cellular genome of the mammalian cell at a targeted location.

A12. The foregoing mammalian cell of A11, further comprising a nucleic acid encoding the product of interest that is randomly integrated in the cellular genome of the mammalian cell.

A13. The foregoing mammalian cell of any one of A-A12, wherein the lactogenic activity of the mammalian cells is less than about 50% of the lactogenic activity of a reference cell.

A14. The foregoing mammalian cell of A13, wherein the lactogenic activity of the mammalian cells is less than about 20% of the lactogenic activity of a reference cell.

A15. The foregoing mammalian cell of A13 or A14, wherein the reference cell is a cell that comprises wild-type alleles of the PKM gene.

A16. The foregoing mammalian cell of any one of A-A15, wherein the lactogenic activity of the mammalian cell is determined at day 14 or day 15 of a production phase.

A17. The foregoing mammalian cell of any one of A-A16, wherein the mammalian cell produces less than about 2.0 g/L of lactate during a production phase.

A18. The foregoing mammalian cell of any one of A-A16, wherein the mammalian cell produces less than about 2.0 g/L of lactate during a production phase in a shake flask.

A19. The foregoing mammalian cell of any one of A-A16, wherein the mammalian cell produces less than about 2.0 g/L of lactate during a production phase in a bioreactor.

B. In certain non-limiting embodiments, the presently disclosed subject matter provides for a mammalian cell comprising an allele of a PKM gene that comprises a nucleotide sequence selected from the group consisting of SEQ ID NOs: 39-41, or the nucleotide sequences set forth in SEQ ID NOs: 37 and 38.

C. In certain non-limiting embodiments, the presently disclosed subject matter provides for a composition comprising a mammalian cell of any of the foregoing mammalian cell of A-A19.

D. In certain non-limiting embodiments, the presently disclosed subject matter provides for a method for reducing or eliminating lactogenic activity in a cell, comprising knocking down or knocking out the expression of a pyruvate kinase muscle (PKM) polypeptide isoform.

E. In certain non-limiting embodiments, the presently disclosed subject matter provides for a method for reducing or eliminating lactogenic activity in a cell, comprising administering to the cell a genetic engineering system, wherein the genetic engineering system knocks down or knocks out the expression of a pyruvate kinase muscle (PKM) polypeptide isoform.

E1. The foregoing method of E, wherein the genetic engineering system is selected from the group consisting of a CRISPR/Cas system, a zinc-finger nuclease (ZFN) system, a transcription activator-like effector nuclease (TALEN) system and a combination thereof.

E2. The foregoing method of E or E1, wherein the genetic engineering system is a CRISPR/Cas9 system.

E3. The foregoing method of E2, wherein the CRISPR/Cas9 system comprises:
(a) a Cas9 molecule, and
(b) one or more guide RNAs (gRNAs) comprising a targeting sequence that is complementary to a target sequence in a PKM gene.

E4. The foregoing method of E3, wherein the target sequence is selected from the group consisting of: a portion of the PKM gene, a region within exon 1, a 5′ region flanking exon 2, a region within exon 2, a 5′ intron region flanking exon 9 of the PKM gene, a 3′ intron region flanking exon 9 of the PKM gene, a 3′ intron region flanking exon 10 of the PKM gene, a region within exon 1 of the PKM gene, a region within exon 12 of the PKM gene and combinations thereof.

E5. The foregoing method of any one of E3-E4, wherein the one or more gRNAs comprises a sequence selected from the group consisting of SEQ ID NOs: 33-34 and 42-43 and a combination thereof.

E6. The foregoing method of E3 or E4, wherein the one or more gRNAs comprises (1) a first gRNA comprising a target sequence that is complementary to a 5′ intron region flanking exon 9 of the PKM gene; and (2) a second gRNA comprising a target domain that is complementary to a 3′ intron region flanking exon 9 of the PKM gene.

E7. The foregoing method of any one of E3-E6, wherein the one or more gRNAs comprises a sequence selected from the group consisting of SEQ ID NOs: 33-34 and a combination thereof.

E8. The foregoing method of E3 or E4, wherein the one or more gRNAs comprises (1) a first gRNA comprising a target sequence that is complementary to a region within exon 2 of the PKM gene; and (2) a second gRNA comprising a target domain that is complementary to region within exon 12 of the PKM gene.

E9. The foregoing method of any one of E3-E5 and E8, wherein the one or more gRNAs comprises a sequence selected from the group consisting of SEQ ID NOs: 42-43 and a combination thereof.

E10. The foregoing method of any one of D and E-E9, wherein the expression of the PKM polypeptide isoform is knocked out, and the lactogenic activity in the cell is eliminated or reduced compared to the lactogenic activity of a reference cell.

E11. The foregoing method of any one of D and E-E9, wherein the expression of the PKM polypeptide isoform is knocked down, and the lactogenic activity in the cell is reduced compared to the lactogenic activity of a reference cell.

E12. The foregoing method of E10 or E11, wherein the lactogenic activity of the cell is less than about 50% of the lactogenic activity of the reference cell.

E13. The foregoing method of E10 or E11, wherein the lactogenic activity of the cell is less than about 20% of the lactogenic activity of the reference cell.

E14. The foregoing method of any one of D and E-E13, wherein the lactogenic activity of the cell is determined at day 14 or day 15 of a production phase.

E15. The foregoing method of any one of D and E-E14, wherein the cell produces less than about 2.0 g/L of lactate during a production phase.

E16. The foregoing method of any one of D and E-E14, wherein the cell produces less than about 2.0 g/L of lactate during a production phase in a shake flask.

E17. The foregoing method of any one of D and E-E14, wherein the cell produces less than about 2.0 g/L of lactate during a production phase in a bioreactor.

E18. The foregoing method of any one of E11-E17, wherein the reference cell is a cell that comprises wild-type alleles of the PKM gene.

E19. The foregoing method of any one of D and E-E18, wherein the PKM polypeptide isoform is the PKM-1 polypeptide isoform.

E20. The foregoing method of any one of D and E-E19, wherein the PKM polypeptide isoform is the PKM-1 polypeptide isoform and the PKM-2 polypeptide isoform.

E21. The foregoing method of E, wherein the genetic engineering system comprises an RNA selected from the group consisting of: a short hairpin RNA (shRNA), a small interference RNA (siRNA), and a microRNA (miRNA), wherein the RNA is complementary to a portion of an mRNA expressed by the PKM gene.

E22. The foregoing method of E21, wherein the mRNA expressed by the PKM gene encodes a PKM-1 polypeptide isoform.

E23. The foregoing method of E22, wherein the expression of the PKM-1 polypeptide isoform is knocked out or knocked down and the lactogenic activity of the cell is reduced as compared to the lactogenic activity of a reference cell.

E24. The foregoing method of any one of E21-E23, wherein the genetic engineering system further comprises a second RNA selected from the group consisting of: shRNA, an siRNA, and a microRNA miRNA, wherein the second RNA is complementary to a portion of an mRNA expressed by the PKM gene that encodes a PKM-2 polypeptide isoform.

E25. The foregoing method of E24, wherein the expression of the PKM-1 and PKM-2 polypeptide isoforms are knocked out or knocked down, and the lactogenic activity of the cell is reduced.

E26. The foregoing method of E, wherein the genetic engineering system is a zinc-finger nuclease (ZFN) system or a transcription activator-like effector nuclease (TALEN) system.

E27. The foregoing method of D and E-E26, wherein the cell is a mammalian cell.

E28. The foregoing method of E27, wherein the mammalian cell is a CHO cell.

E29. The foregoing method of D and E-E28, wherein the cell expresses a product of interest.

E30. The foregoing method of E29, wherein the product of interest expressed by the cells is encoded by a nucleic acid sequence.

E31. The foregoing method of E30, wherein the nucleic acid sequence is integrated in the cellular genome of the cell at a targeted location.

E32. The foregoing method of any one of E26-E31, wherein the product of interest expressed by the cells is further encoded by a nucleic acid sequence that is randomly integrated in the cellular genome of the mammalian cell.

E33. The foregoing method of E26-E31, wherein the product of interest comprises a protein.

E34. The foregoing method of E33, wherein the product of interest comprises a recombinant protein.

E35. The foregoing method of any one of E26-E33, wherein the product of interest comprises an antibody or an antigen-binding fragment thereof.

E36. The foregoing method of E35, wherein the antibody is a multispecific antibody or an antigen-binding fragment thereof.

E37. The foregoing method of E35, wherein the antibody consists of a single heavy chain sequence and a single light chain sequence or antigen-binding fragments thereof.

E38. The foregoing method of any one of E35-E37, wherein the antibody is a chimeric antibody, a human antibody or a humanized antibody.

E39. The foregoing method of any one of E35-E38, wherein the antibody is a monoclonal antibody.

F. In certain non-limiting embodiments, the presently disclosed subject matter provides a method of producing a product of interest comprising culturing mammalian cells expressing the product of interest, wherein the mammalian cells express the product of interest and have reduced or eliminated lactogenic activity.

G. In certain non-limiting embodiments, the presently disclosed subject matter provides a method of culturing a population of mammalian cells expressing a product of interest, wherein the mammalian cells have reduced or eliminated lactogenic activity.

G1. The foregoing method of F or G, wherein the reduction or elimination of lactogenic activity results from the knock out or knock down of the expression of a pyruvate kinase muscle (PKM) polypeptide isoform in the mammalian cells.

G2. The foregoing method of G1, wherein the PKM polypeptide isoform is the PKM-1 polypeptide isoform.

G3. The foregoing method of G1, wherein the PKM polypeptide isoform is the PKM-1 polypeptide isoform and the PKM-2 polypeptide isoform.

G4. The foregoing method of any one of F and G-G3, wherein the lactogenic activity of the mammalian cells is less than about 50% of the lactogenic activity of a reference cell.

G5. The foregoing method of any one of F and G-G3, wherein the lactogenic activity of the mammalian cells is less than about 20% of the lactogenic activity of a reference cell.

G6. The foregoing method of any one of F and G-G5, wherein the lactogenic activity of the mammalian cells is determined at day 14 or day 15 of a production phase.

G7. The foregoing method of any one of F and G-G6, wherein the mammalian cells produce less than about 2.0 g/L of lactate during a production phase.

G8. The foregoing method of any one of F and G-G6, wherein the mammalian cells produce less than about 2.0 g/L of lactate during a production phase in a shake flask.

G9. The foregoing method of any one of F and G-G6, wherein the mammalian cells produce less than about 2.0 g/L of lactate during a production phase in a bioreactor.

G10. The foregoing method of any one of F and G-G9, wherein the reference cell is a cell that comprises at least one or both wild-type alleles of the PKM gene.

G11. The foregoing method of any one of F and G-10, wherein the mammalian cells are CHO cells.

G12. The foregoing method of any one of F and G-G11, wherein the product of interest expressed by the mammalian cells is encoded by a nucleic acid sequence.

G13. The foregoing method of G12, wherein the nucleic acid sequence is integrated in the cellular genome of the mammalian cells at a targeted location.

G14. The foregoing method of any one of F and G-G13, wherein the product of interest expressed by the cells is further encoded by a nucleic acid sequence that is randomly integrated in the cellular genome of the mammalian cells.

G15. The foregoing method of any one of F and G-G14, wherein the product of interest comprises a protein.

G16. The foregoing method of any one of F and G-G15, wherein the product of interest comprises a recombinant protein.

G17. The foregoing method of any one of F and G-G16, wherein the product of interest comprises an antibody or an antigen-binding fragment thereof.

G18. The foregoing method of G17, wherein antibody is a multispecific antibody or an antigen-binding fragment thereof.

G19. The foregoing method of G17, wherein the antibody consists of a single heavy chain sequence and a single light chain sequence or antigen-binding fragments thereof.

G20. The foregoing method of any one of G17-G19, wherein the antibody is a chimeric antibody, a human antibody or a humanized antibody.

G21. The foregoing method of any one of G17-G20, wherein the antibody is a monoclonal antibody.

G22. The foregoing method of any one of F and G-G21, further comprising harvesting the product of interest.

### EXAMPLES

The following examples are merely illustrative of the presently disclosed subject matter and should not be considered as limitations in any way.

### Example 1: PKM-1 Expression Drove Lactogenic Behavior in CHO Cell Lines, Triggering Lower Viability and Productivity

In the process of cell line development (CLD), two lactogenic cell lines expressing different antibody molecules were identified. The lactogenic behaviors of these cell lines could be differentially mitigated through optimization of either nutrient feeds or culture pH, depending on the cell line. Analysis of various proteins involved in the glycolysis pathway revealed a direct correlation between the pyruvate kinase muscle-1 (PKM-1) isoform levels and lactogenic behavior.

CRISPR/Cas9 was used for targeted deletion of exon-9 to knockout PKM-1 expression. Knocking out PKM-1 expression completely abolished lactogenic behavior in the selected two cell lines, eliminating the needs for mitigation strategies. A single cell line was identified in which expression of both PKM-1 and PKM-2 genes were fully disrupted without any adverse effects on growth and viability. Further analysis of paternal and CHO cell lines revealed that they all expressed both PKL and PKR versions of pyruvate kinase, which, without being bound by theory, could enable them to tolerate complete lack of PKM expression. The unique mitigation strategies used to control the lactogenic behavior of these cell lines shifted their metabolic pathways, altering the pattern of PKM-1 transcription and expression, preventing or delaying the lactogenic behavior. PKM gene is knocked out entirely in order to analyze the behavior of the resulting cell lines in culture and in bioreactors.

In summary, the present disclosure delineates a direct correlation between lactogenic behavior and high PKM-1 levels in production cultures. Furthermore, elimination of PKM-1 expression was tolerated and reversed the lactogenic behavior in the selected CHO cell lines. Hence, permanent deletion of PKM-1, or the entire PKM gene for that matter, in CHO cells can be beneficial in reducing occurrences of lactogenic behavior during production in bioreactors.

### Materials and Methods

### Cell Lines

Cell lines secreting recombinant monoclonal antibody mAb-1 or mAb-2 were derived from a CHO-K1 host utilizing a glutamine synthetase (GS) selection marker. Seed trains were maintained in a proprietary DMEM/F12-based medium containing methionine sulfoximine (MSX) as a selection agent at 150 rpm shaking speed, 37° C., and 5% CO₂. Cells were passaged every 3 or 4 days.

### 2-L Bioreactor Production

2-L bioreactor production was performed in glass stirred-tank bioreactors (Applikon, Foster City, CA) with Finesse controllers (Applikon, Foster City, CA). All production cultures utilized a single inoculum train stage (N−1) for 3 days in the bioreactors before inoculating the production stage (N), lasting 14 days. The N−1 inoculation cultures were inoculated between 1.5 and 1.7 L of working volume to a target packed cell volume (PCV) of 0.17%. They were operated at 37° C., a pH set point of 7.0, a dissolved oxygen (DO) set point of 30%, and an agitation of 275 rpm. After three days, cells were transferred to inoculate the production (N) bioreactors to a volume between 1.4 and 1.7 L at a target PCV of 0.25%. All bioreactors were operated at 37° C. with a temperature shift to 35° C. at 72 hours. Cultures were run at a pH of 7.00 with a dead band of 0.03 using CO2 as acid control and 1 M sodium carbonate as the base control. The only exception to this pH operation was the mAb-2 pH level that was operated at a constant pH of 6.80 with the same dead band of 0.03. All cultures were set to operate at 350 rpm and a DO of 30% utilizing a combination of air and oxygen gas flows to keep DO constant. For the control process cultures of mAb-1, feeds occurred on day 3 of production (72 hours) and were 20% of the total volume of the culture at that time. For the enhanced feed process, feeds occurred on day 3 (72 hours) and on day 6 (144 hours) at 15% of the total volume culture for each feed.

### AMBR15 Operation

Production cultures in the AMBR15 system (Sartorius, Goettingen Germany) were operated at set points of a temperature of 37° C., a DO of 30%, a pH of 7.0, and an agitation rate of 1400 rpm. The N-1 inoculation trains were run for 4 days and then the production (N) stage was inoculated at 1 million cells/mL with a total volume of 13 mL with a temperature of 37° C., a DO of 30%, a pH of 7.0, and an agitation rate of 1400 rpm. For these cultures, a scaled down process of 2-L bioreactor was performed.

### Off-Line Sample Analyses

Supernatant and cell pellet samples were collected for submission to assays or Western blotting analysis. Samples were analyzed for viable cell concentration (VCC) and viability using the Vi-Cell XR (Beckman Coulter), and for pO₂, pH, pCO₂, Na⁺, glucose, and lactate using the Bioprofile 400 (Nova Biomedical). All samples from 2-L and AMBR bioreactors were analyzed on the BioProfile 400 within a few minutes after sampling to minimize off-gassing. The same Vi-Cell XR, BioProfile 400, and osmometer (Model 2020, Advanced Instruments) were used for all samples to eliminate instrument-to-instrument variability. Antibody titer was measured using high pressure liquid chromatography (HPLC) with a protein A column. Antibody product quality assays were conducted using cell culture supernatant samples purified by PhyTip (PhyNexus, San Jose, CA) protein A column. Antibody molecular size distribution was analyzed by size-exclusion chromatography (SEC). Protein charge heterogeneity was measured using imaged capillary isoelectric focusing (icIEF), and all charge heterogeneity samples were pretreated with carboxypeptidase B. All protein product quality assays were developed in-house, and detailed protocols have been published (Hopp et al., 2009, Biotechnol. Prog. 25(5):1427-32).

### Immunoblotting

Cell pellets were lysed in lysis buffer (10 mM Tris pH 8.0, 0.5% NP40, 150 mM NaCl, 5 mM MgCl₂, with protease inhibitors) and incubated on ice for 20 minutes. Samples were then spun down at 13,000 rpm for 10 minutes and the supernatant was transferred to a new tube to determine protein concentration using a Nanodrop 2000 (Thermo Scientific, Wilmington DE) using the absorbance at 280 nm. 15 μL of the lysate was combined with 5 μL of 4× running buffer (400 μL of 2× Invitrogen loading buffer+400 μL of 50% glycerol+200 μL of 20% SDS+100 μL beta-mercaptoethanol) and heated at 90° C. for 5 minutes. Equivalent amounts of protein were then loaded onto a 12 well 4-20% Tris-Glycine gel and run for 1.5 hours at 150 V using SDS running buffer. Afterward the proteins were transferred to nitrocellulose membrane using the iBlot2 from Thermo Fisher. After washing with 1×TBST buffer, blots were blocked in of 5% milk solution for a minimum of 1 hour followed by incubation with the primary antibodies overnight. The blots were then washed and incubated in the secondary antibody for a minimum of one hour and washed with TBST buffer again. ECL reagents were used followed by imaging of the blots using Bio-Rad imaging machine (Bio-Rad, Hercules, CA).

### PKM-1 Knockout

To knock out PKM-1, guide RNAs (gRNAs) targeting to both the 5′ and the 3′ intron regions flanking the exon 9 of PKM gene were cloned into a Genentech gRNA-expression vector. This construct was then co-transfected with Cas9 expression plasmid into mAb-2 expressing cells. Transfected cells were single cell cloned and deletion of exon 9 was confirmed by genomic DNA PCR. The following gRNA oligoes were most effective in deleting exon-9 (PKM-1) and the pools targeted with these oligoes were used to isolate PKM-1 knockout mAb-2 cell lines:

5′ gRNA:
(SEQ ID NO: 33)
GTCCTTTGGGCAGAGACAG
3′ gRNA:
(SEQ ID NO: 34)
GACCAGAGTACTCCCTCGT

### Sequence of Genomic DNA PCR Primers:

5′-primer:
(SEQ ID NO: 35)
CCAGATTTGGTGAGGACGAT
3′-primer:
(SEQ ID NO: 36)
AGCTGTGTTGTGAGGCATTG

### Results

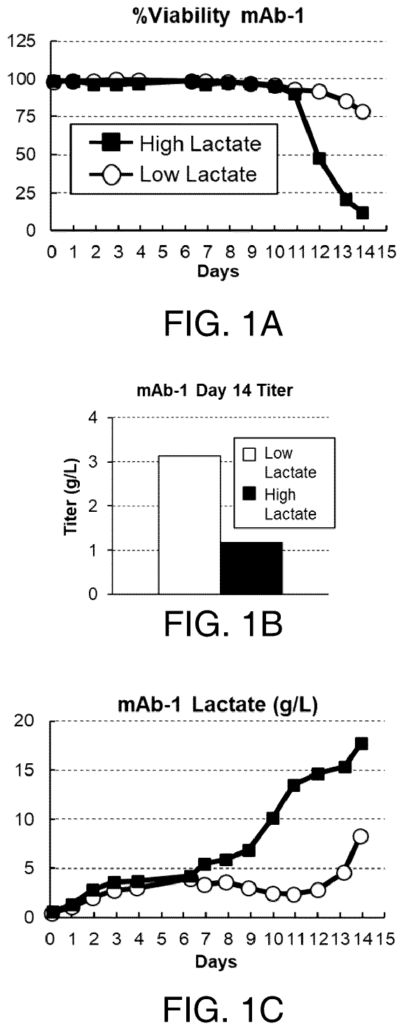

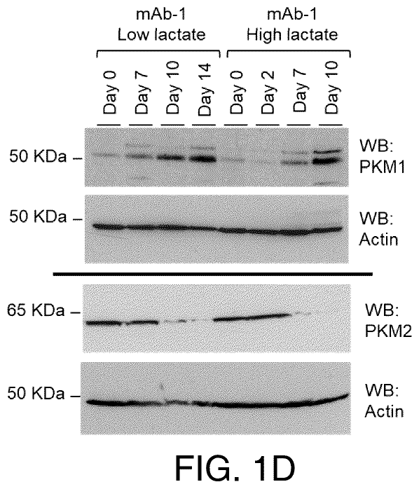

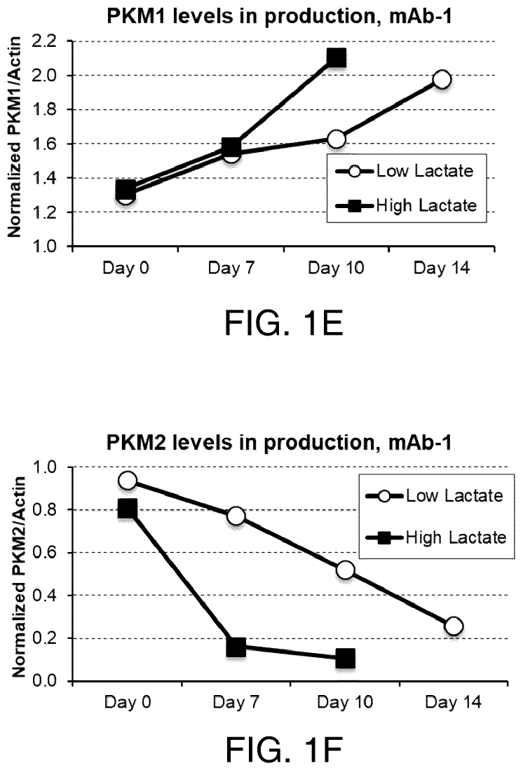

Increase in PKM-1 Levels During Production Correlates with CHO Cells Lactogenic Behavior A cultured seed train of the mAb-1 expressing cell line was used to source production cultures in bioreactors using standard or enhanced feeding strategies. Standard feeding triggered lactogenic behavior in mAb-1 cell line while an enhanced feeding strategy mitigated the lactogenic behavior. The high lactate conditions triggered a drop of cell viability after day 10 in the production culture (FIG. 1A), resulting in lower day 14 titers compared to low lactate conditions (FIG. 1B). While both culture conditions had comparable levels of lactate till day 7, the high lactate conditions showed a sharp increase in lactate accumulation from day 7 in production culture, reaching upwards of 15 g/L on day 14. For low lactate conditions, a delay of 3-4 days was observed before lactate levels started to trend higher (between days 12 to 14) reaching only 8 g/L by day 14 (FIG. 1C).

To identify enzyme(s) that might correlate with the observed lactogenic behavior, Western blot analysis was performed on a variety of proteins involved in cell growth signaling and glycolysis pathways. Since in high lactate conditions cell viability dropped sharply after day 10, samples from days 0, 2, 7 and 10 of this set were compared to samples from days 0, 7, 10, and 14 of the low lactate condition (FIGS. 1D-1F). Among all the different proteins analyzed (data not shown), only PKM-1 levels showed a direct correlation with lactogenic behavior of the mAb-1 expressing cell line. FIG. 1D-1F showed PKM-1 and PKM-2 protein levels in cell pellets. PKM-1 expression levels in high lactate samples started to divergently increase after day 7 in production culture relative to the low lactate samples. Interestingly, the increase in PKM-1 levels tightly trended with the increase in lactate accumulation since even under low lactate conditions the increase in PKM-1 levels towards the end of production culture (FIG. 1E, between days 10 and 14) trended with the higher lactate levels in culture (FIG. 1C). The levels of PKM-2, however, correlated inversely with lactogenic behavior in mAb-1 expressing cell line (FIG. 1F).

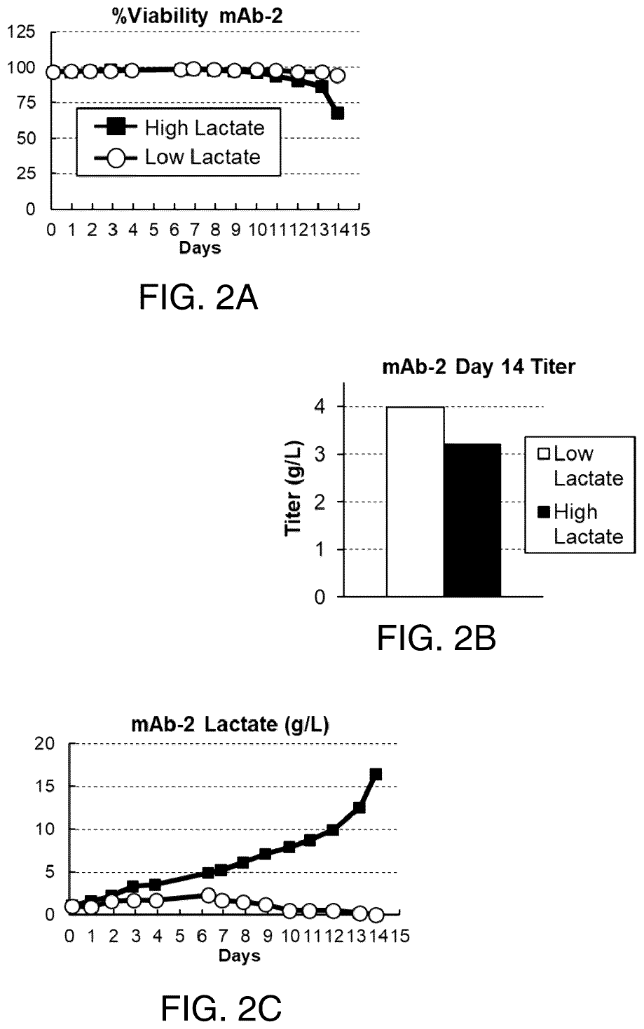

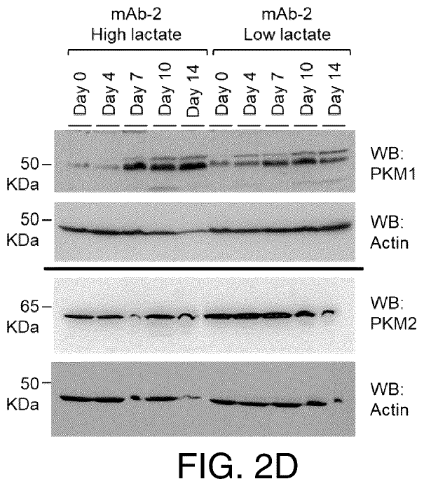

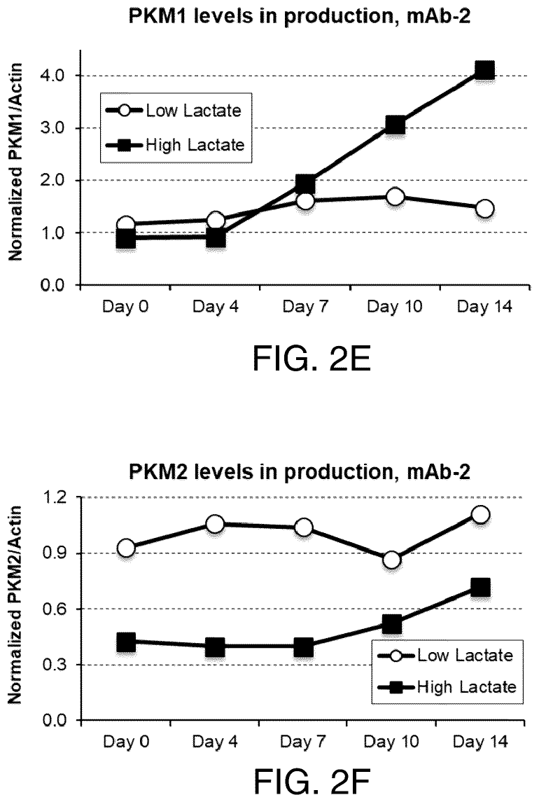

Similar bioreactor experiments were conducted for the mAb-2 expressing cell line. Such cell line was cultured in production media with conditions promoting lactogenic or non-lactogenic behaviors. Cell viability for mAb-2 cell line was comparable between high and low lactate conditions until days 13 and 14 of the production culture (FIG. 2A). This resulted in a titer reduction of approximately 25% for the high lactate compared to the low lactate condition (FIG. 2B). Throughout the production process, lactate accumulation in the media was low for the low lactate control arm of mAb-2 cell line experiment. However, under the high lactate conditions lactate levels started to diverge from day 7, reaching upwards of 15 g/L by day 14 of the production culture (FIG. 2C). Western blot analysis revealed that PKM-1 protein levels started to increase in the high lactate samples, relative to the low lactate ones, around day 7 in production and linearly increased until the end of the production culture (FIGS. 2D and 2E). This increase in PKM-1 levels correlated tightly with the increase in accumulated lactate (FIGS. 2C and 2E). Similar to mAb-1, overall levels of PKM-2 in low lactate mAb-2 samples were higher than those of high lactate samples as illustrated in FIG. 2D (lower panels) and FIG. 2F. Taken together, these data suggest a direct correlation between lactogenic behavior in mAb-1 and mAb-2 expressing cells and the PKM-1 levels in these cells.

Pkl/R Proteins are Expressed in CHO Cells but do not Correlate with Lactogenic Behavior

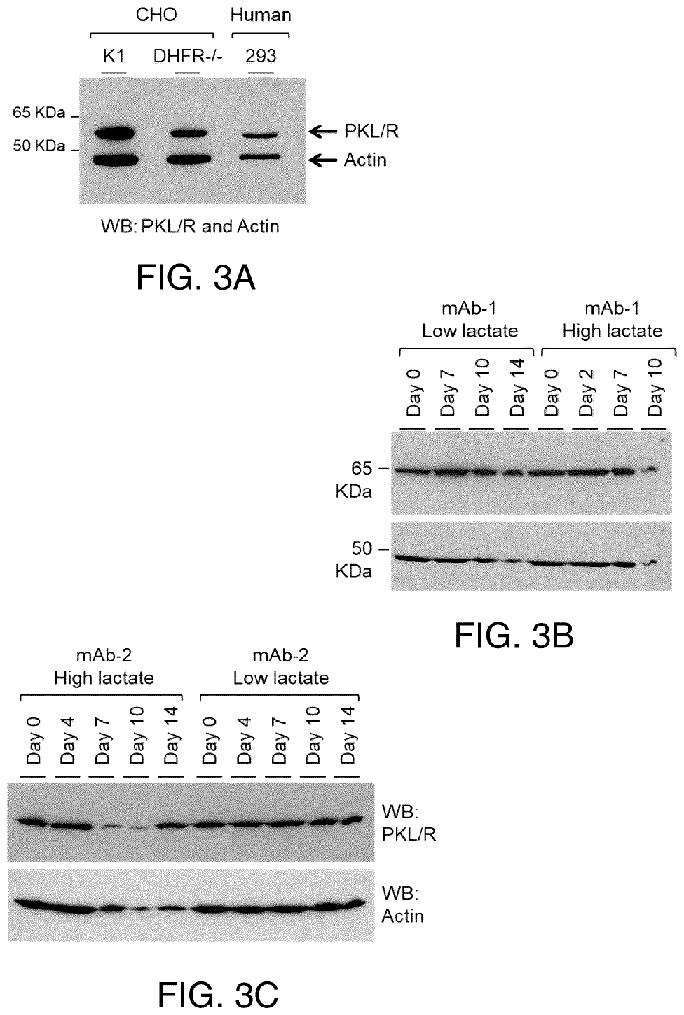

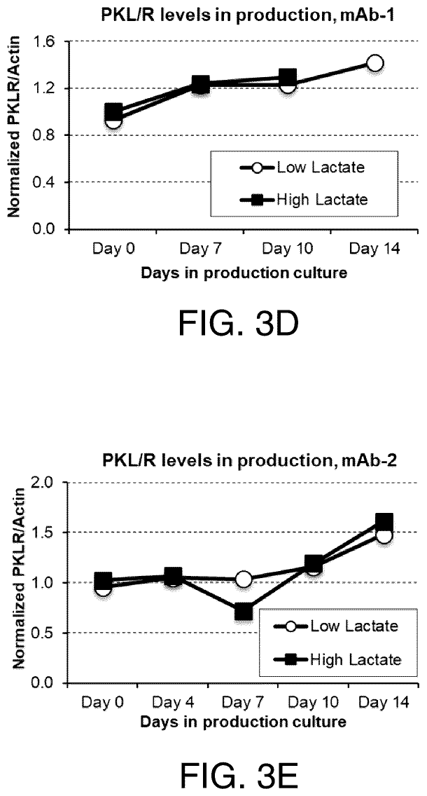

To evaluate whether other forms of PK (other than PKM) are expressed in the proprietary CHO host cells, two different CHO host cell lines (CHO-K1 and DHFR−/−) and a human (HEK 293) cell line were analyzed for expression of PKL and PKR (PKL/R) proteins. Both CHO cell lines as well as the control HEK 293 cell line showed expression of PKL/R enzyme (FIG. 3A), indicating that all isoforms of PK genes (including PKM-1, PKM-2, and PKL/R) are expressed in the selected CHO hosts. Whether expression of PKL/R was differentially regulated when comparing high versus low lactate conditions in mAb-1 and/or mAb-2 expressing cell lines was evaluated next. PKL/R expression levels were comparable (once the protein levels were normalized against actin as internal control) between high and low lactate conditions in both mAb-1 and mAb-2 cell lines, showing no correlation with lactogenic behavior in these cell lines (FIGS. 3B-3D). The lower levels of PKL/R in mAb-2 high lactate samples for days 7 and 10 in FIG. 3C, was due to lower overall protein loading as reflected by lower levels of actin (as loading control) in the same lanes. Besides PK isoforms, expression of several different proteins involved in the cell growth signaling and glycolysis pathways were also analyzed. However, no clear correlation between the expression of these proteins and the lactogenic behavior observed in mAb-1 or mAb-2 expressing cell lines was observed (data not shown). The fact that different PK isoforms were expressed in selected CHO host cells suggested that these cell lines might be able to tolerate targeted deletion of one or more isoforms of PK. The observed direct correlation between lactogenic behaviors of mAb-1 and mAb-2 cell lines with PKM-1 levels during production phase made this enzyme a good candidate for targeted deletion.

### Crispr/Cas9 Mediated Targeted Deletion of Exon-9 in PKM Gene

The PKM gene is responsible for expression of both PKM-1 and PKM-2 enzymes, which differ only in alternative inclusion of exon 9 or exon 10, respectively. Since the selected CHO cell lines were not sequenced and annotated, no information regarding PKM allele types, gene copy numbers, or sequence were available. Therefore, to disrupt expression of PKM-1 gene, guide RNA constructs targeting intron regions flanking exon 9 using available CHO sequences in the public databases were designed (Kent et al. 2002, Genome Res. 12(6):996-1006). The 5′ screening PCR primer(s) was designed to match the intron region between exons 8 and 9, and the 3′ screening PCR primer was designed to matched exon 10 (FIG. 4A). Exon sequence information was obtained based on amplifying and sequencing PKM cDNA from the selected CHO hosts' mRNAs. The mAb-2 expressing cell line was transfected with Cas9 and various gRNA constructs. After initial screening, the pool showing the most efficient targeting of exon 9 was subjected to single cell cloning. Twenty cell lines from this pool were screened for targeted deletion of exon-9, and two heterozygous (HET-3 and HET-18) and two knockout (KO-2 and KO-15) cell lines were identified for which the targeted allele's deletion were fully confirmed by PCR and sequencing (FIGS. 4B and 4C). Cultures for these cell lines were then expanded for further evaluation in bioreactors. Sequencing of the targeted allele(s) confirmed deletion of exon 9 in all selected cell lines (FIG. 4C). Three cell lines (KO-2, HET-3, and HET-18) had the deletion of exon 9 exactly at the 5′ and 3′ gRNA targeting sites (FIG. 4C). For KO-15 cell line, the PCR size of targeted allele(s) was smaller than those observed for other cell lines (FIG. 4B). Sequencing analysis confirmed that 122 base pairs (bp) upstream of 5′ gRNA was deleted in the KO-15 cell line and partially replaced with an unrelated insertion (FIG. 4C and data not shown). Deletion in this region could affect the integrity of the intron between exons 8 and 9. Irrespective of this, exon 9 was fully deleted in all selected cell lines according to the sequencing analysis.

Blocking or Decreasing PKM-1 Expression Averted or Reduced Lactogenic Behavior in mAb-2 Cell Line, Respectively.

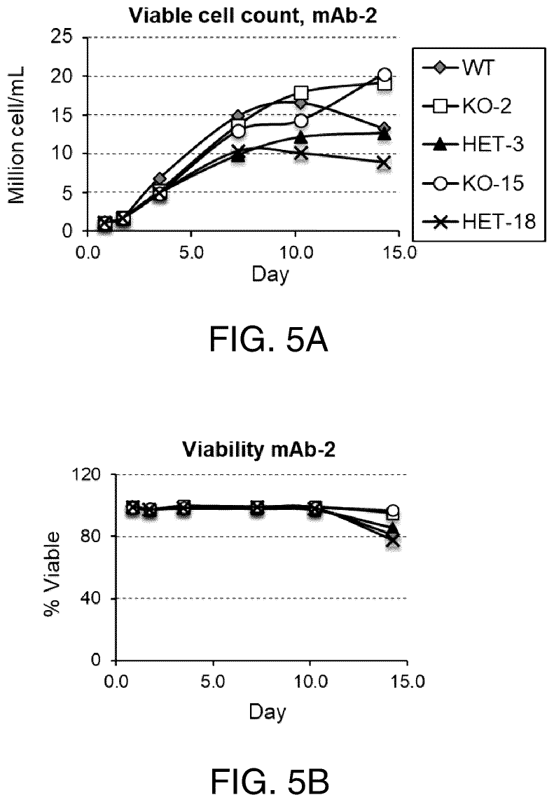

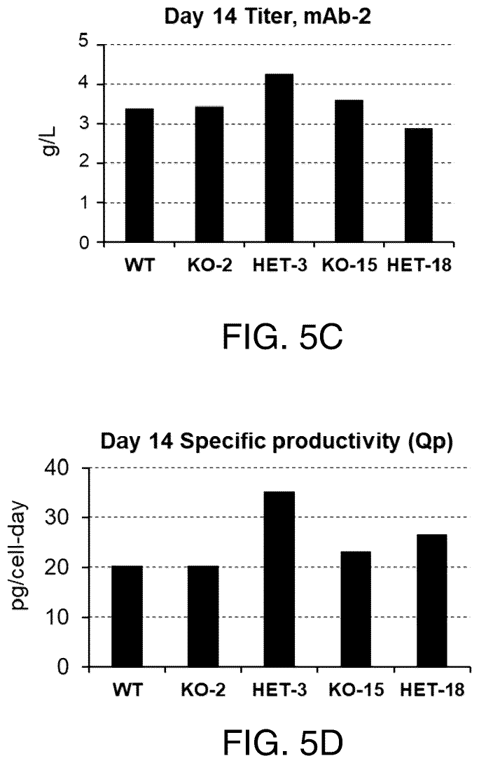

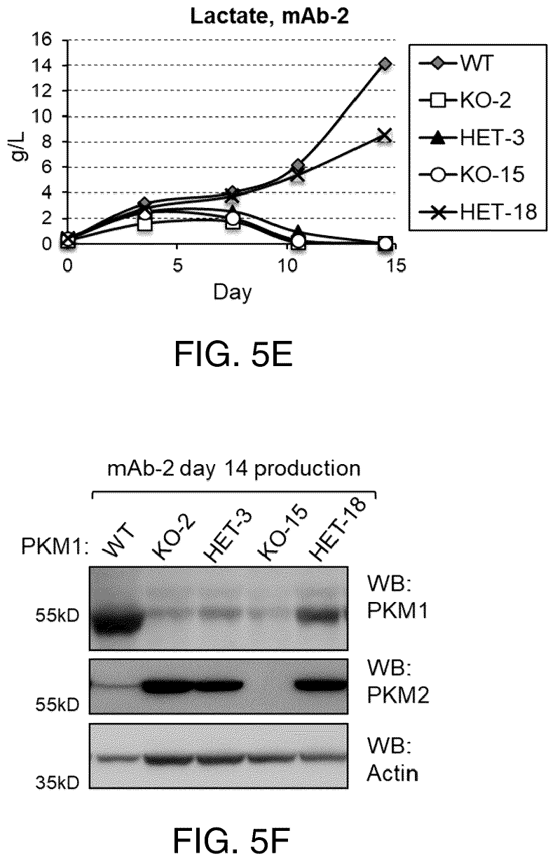

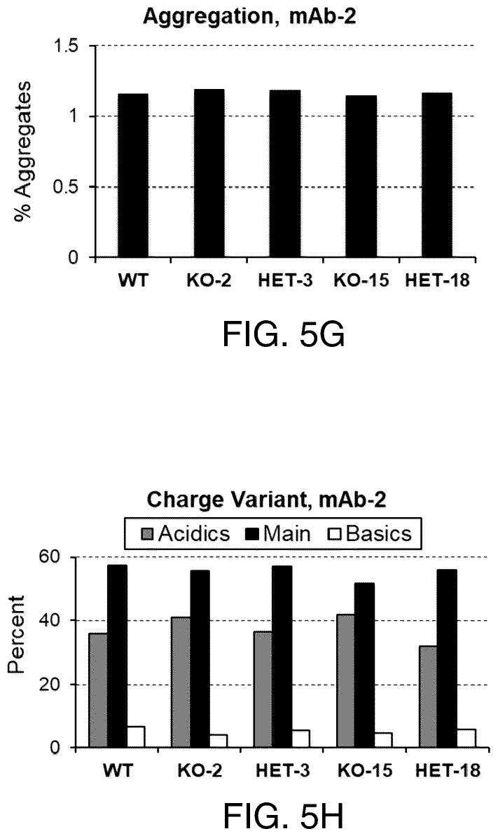

The four mAb-2 cell lines with deletion of exon-9 in some (HET) or all (KO) alleles and the WT mAb-2 line were all sourced to set up a 14-day production culture in AMBR bioreactors. Growth and viability of PKM-1 KO, HET and WT cell lines were shown in FIGS. 5A and 5B. The PKM-1 HET or KO mAb-2 lines had relatively similar titers and specific productivities to that of WT mAb-2 cell line (FIGS. 5C and 5D). PKM-1 KO (KO-2 and KO-15) and one of the HET mAB-2 cell lines (HET-3) had very low lactate levels compared to the WT mAb-2 cell line (FIG. 5E) when cultured under control (lactogenic) condition. The HET-18 line on the other hand displayed similar lactogenic behavior compared to the WT mAb-2 till day 10. After day 10 the rate of lactate accumulation in the WT mAb-2 line increased, approximately doubled relative to the HET-18 line by day 14 (FIG. 5E).

Western blot analysis of these cell lines showed that KO-2 and KO-15 lines were fully deficient in expression of PKM-1, and HET-3 cell line had lower PKM-1 expression compared to the HET-18 cell line (FIG. 5F). The fact that lactogenic behavior of PKM-1 HET and KO cell lines directly correlated with the PKM-1 protein expression in several cell lines suggested that the observed change in lactogenic behavior is not due only to other clonal differences. Since a band corresponding to the full length WT allele could be amplified from the genomic DNA of the HET cell lines (FIG. 4B), possible explanations for these observations could include, but not be limited to: differential expression of PKM-1 from different CHO PKM allele(s); targeting of the WT PKM allele(s) by only one gRNA construct, resulting in disruption of proper exon splicing; inversion of the gRNA targeted region, or 4-homogenitization mediated by allele recombination.

PKM-2 levels were low for the WT mAb-2 cell line (the relative difference was considered with the caveat that actin loading control was also lower for this sample), while it was higher for the HET-3, HET-18, and KO-2 cell lines (FIG. 5F). Interestingly, the KO-15 mAb-2 cell line was completely deficient in the expression of PKM-2 in addition to PKM-1 (FIG. 5F). This was likely due to the larger deletion(s) observed upstream of 5′ gRNA targeted region of the PKM allele(s), possibly affecting functionality of intron region between exons 8 and 9 (FIG. 4C). This suggested that the selected CHO cells could tolerate complete lack of both PKM-1 and PKM-2 enzymes, perhaps due to their ability to express PKL/R enzyme(s) (FIG. 3A).

Product quality attributes such as high molecular weight (aggregates) and charge variant species between WT and PKM-1 KO and HET mAb2 expressing cell lines were investigated. Although no significant differences in product aggregation levels between WT and PKM-1 KO and HET cell lines were observed (FIG. 5G), some variations were observed for mAb-2 product charge variant (FIG. 5H) profiles. Since there were no clear trends or correlations between lactate levels and product quality attributes (FIGS. 5G and 5H), the observed differences could be due to normal variations that could occur during cell line derivation. Additionally, the media and feed used in these experiments were optimized for the WT mAb-2 cell line, not the PKM-1 KO or HET cell lines. Since altering PKM levels could affect the overall metabolic profile of these cell lines, fine-tuning media and feed could be required in order to explore their full effects and influence on product quality attributes.

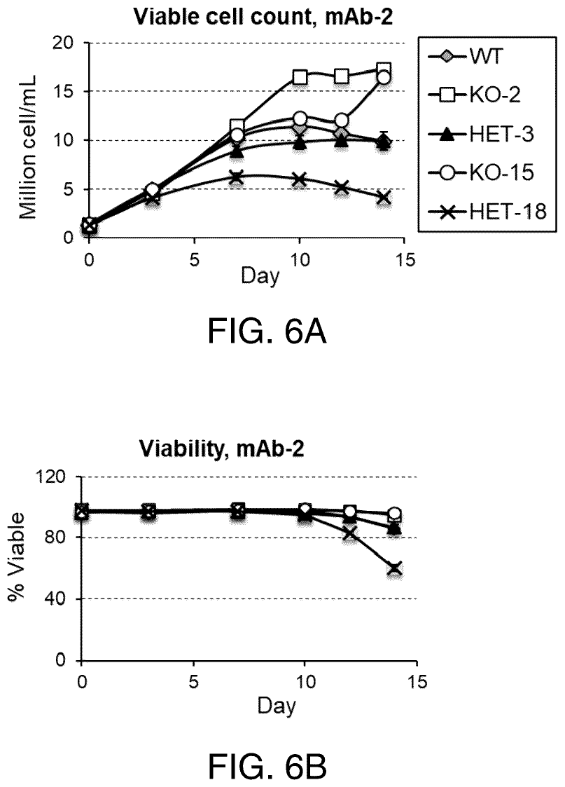

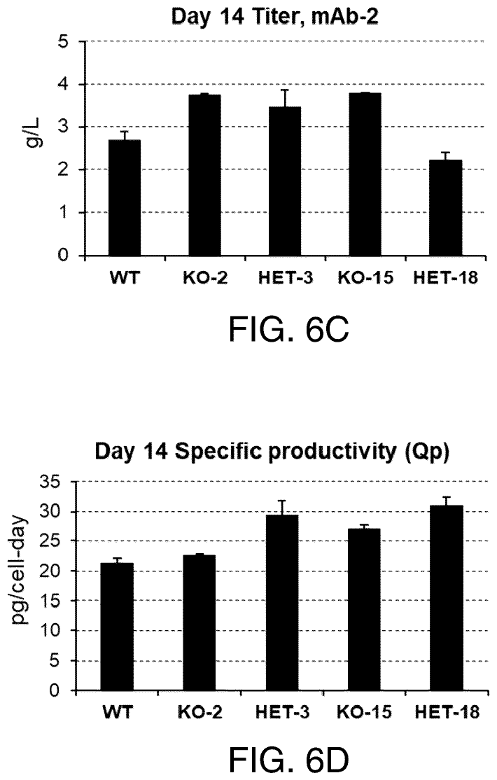

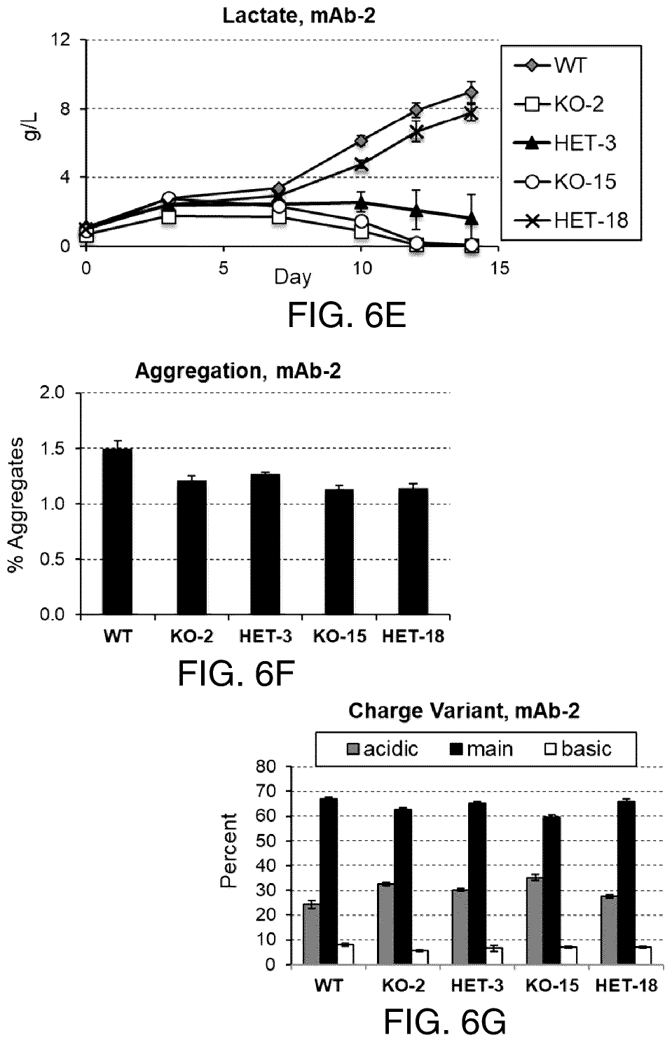

Since it is important to analyze the behavior of any cell line post cell banking and thaw, the PKM-1 KO and HET mAb-2 cell lines were banked and then thawed and maintained in culture for 2.5 weeks, along with the WT cell line as control. These cells were then sourced to set up production cultures, in quadruplet, for each cell line, in order to evaluate their performance post-thaw. The data showed that growth, viability, titer, specific productivity, and lactogenic profiles of PKM-1 KO and HET cell lines were comparable prior to, and post cell banking (FIGS. 5A-5D and 6A-6D). As was previously observed (data not shown), the freshly thawed WT mAb-2 cells were less lactogenic (10 g/L, FIG. 6E) relative to the older cells (15 g/L, FIG. 5E). However, this behavior was unique to the WT mAb-2 cell line, since cell age had no significant effects on the lactogenic behavior of mAb-2 PKM-1 KO and HET cell lines (FIGS. 5E and 6E). Note that for all the cell lines a gradual accumulation of lactate in the media was observed by day 7 of the production culture, after which PKM-1 KO and HET-3 cell lines consumed the lactate while a lactate run-away profile was observed for both WT and HET-18 cell lines (FIG. 6E). Although it was observed that all the younger cell lines (post-thaw) had relatively lower % acidic peaks (FIG. 6G) compared to the older cell lines (FIG. 5G), a trait that was perhaps related to the cell age, there were no major differences with regards to product quality between lactogenic (WT or PKM-1 HET-18) and non-lactogenic (PKM-1 KO or HET-3) cell lines (FIGS. 6F-6G).

### Discussion

Lactogenic behavior in CHO cell cultures could trigger culture acidification and high osmolarity as the result of base addition to control the culture pH, negatively impacting viability, VCC, and eventually production titer (Li et al., 2010, MAbs 2(5):466-79). Lactate generation results from aerobic glycolysis, a behavior that is commonly known as the Warburg effect (Warburg, 1956, Science 123(3191):309-14) and it is observed in many cultured and cancer cells. These cells utilize the glycolysis process to generate energy and regulate this process in response to various energy and metabolic fluxes (Mulukutla et al., 2014, PLoS One 9(6):e98756; Mulukutla et al. 2010, Trends Biotechnol. 28(9):476-84; Luo et al., 2012, Biotechnol. Bioeng. 109(1):146-56; Ahn and Antoniewicz, 2012, Biotechnol. J. 7(1):61-74). Although some lactogenic behaviors could be curbed by process engineering (Luo et al., 2012, Biotechnol. Bioeng. 109(1):146-56; Gagnon et al., 2011, Biotechnol. Bioeng. 108(6):1328-37), these approaches however did not reveal the root cause of the observed behavior. To have a better understanding of lactogenic behavior in CHO host, two lactogenic cell lines expressing mAb-1 or mAb-2 were identified for which lactogenic behavior could be curbed by either modifying feeding strategy or culture pH. These independently derived cell lines were utilized as tools where we analyzed a panel of proteins and enzymes involved in cell growth signaling or glycolysis pathways, in order to delineate their possible correlation with lactogenic behavior. These findings revealed a direct correlation between PKM-1 expression and lactogenic behavior in both mAb-1 and mAb-2 cell lines (FIGS. 1D-1F and 2D-2F).

The detected levels of PKM-1 in both mAb-1 and mAb-2 production cultures were low at early time points during production and increased in a manner correlated with increase in lactate levels in the culture. PKM-2 levels were inversely correlated with lactogenic behavior in these cell lines (FIGS. 1D-1F and 2D-2F). This could be due to the function of proteins involved in alternative splicing of PKM-1 or PKM-2 transcripts from PKM gene (Chaneton and Gottlieb, 2012, Trends Biochem. Sci. 37(8):309-16; Israelsen and Vander Heiden, 2015, Semin. Cell Dev. Biol. 43:43-51; Mazurek, 2011, Int. J. Biochem. Cell Biol. 43(7):969-80; Harada et al., 1978, Biochim. Biophys. Acta. 524(2):327-39; Noguchi et al., 1986, J. Biol. Chem. 261(29):13807-12; Noguchi et al., 1987, J. Biol. Chem. 262(29):14366-71). Of the two isoforms of PKM gene, PKM-2 is reported to act as a central switch for altering metabolic pathways, allowing cancer cells to survive and thrive under physiologically unfavorable conditions such as within a tumor environment (Christofk et al., 2008, Nature 452(7184):230-3; Chaneton and Gottlieb, 2012, Trends Biochem. Sci. 37(8):309-16; Israelsen and Vander Heiden, 2015, Semin. Cell Dev. Biol. 43:43-51). On the other hand, PKM-1 isoform is constitutively active once expressed. Without wishing to be bound by theory, the correlation of PKM-1 level with lactogenic behavior late in the production culture could be due to uncontrollable conversion of PEP to pyruvate by this enzyme, followed by LDH mediated conversion of pyruvate to lactate.

CRISPR mediated deletion of exon-9 and hence PKM-1 in the mAb-2 expressing cell line resulted in complete reversal of lactogenic behavior in PKM-1 KO cells under conditions where WT mAb-2 expressing cells displayed lactogenic behavior (FIG. 5E). The two PKM-1 HET mAb-2 cell lines displayed distinct behaviors. While the HET-3 KO cell line produced very little lactate (only a little more than its PKM-1 KO counterparts), the HET-18 cell line was lactogenic and generated approximately ⅔ as much lactate as the WT mAb-2 cells (FIG. 5E). A direct correlation between the lactogenic behavior of these PKM-1 HET cell lines and the higher levels of PKM-1 in these cells was also observed (FIG. 5F). Differences in PKM-1 levels observed in HET-18 compared to HET-3 cell lines could be due to: differential expression of PKM-1 from different PKM alleles, or partial targeting of PKM allele(s) by a gRNA construct(s) in HET-3 cell line, or inversion of targeted region within the targeted PKM allele(s).

A PKM-1 knockout cell line (KO-15) that was unable to express PKM-2 protein (FIG. 5F) was identified. The cell line has a 122-bp deletion and insertion of random bases at the intron upstream of 5′ gRNA targeting region (FIG. 3C). The KO-15 cell line can tolerate loss of both PKM-1 and PKM-2 enzymes because the CHO cell lines also express PKL/R (FIG. 3A), which can compensate for lack of PKM expression. The expression of PKL/R protein(s) in the WT and all the PKM-1 HET and KO cell lines were confirmed via Western blot analysis (data not shown). Nevertheless, lack of or reduced PKM-1 expression resulted in lower lactate generation in all the PKM-1 KO or HET cell lines, respectively, without an effect on titer or specific productivity (FIGS. 5C-5D and 6C-6D). Absence or attenuation of lactogenic behavior in these cell lines were reproducible and consistent in post-thaw younger cell ages and among all replicates (FIGS. 5A-5E and 6A-6E).

Controlling lactogenic behavior during production culture is important for obtaining optimal titer and product quality from manufacturing runs, therefore, knocking out PKM-1 expression from hosts can be advantageous in dealing with lactogenic behavior of CHO cells. It is important to confirm and monitor the expression of other PK enzymes in CHO cells prior to targeting PKM-1 or the entire PKM gene for deletion, because PKL/R enzymes can compensate PKM ablation. Knocking out of the entire PKM gene can be tolerated in CHO hosts, and the PKM KO CHO hosts are capable of expressing antibodies or products of interest with comparable titer and product quality to that of WT host. These PKM KO hosts can reduce lactogenic behavior and have a better growth profile than the WT host as observed in KO-2 and KO-15 cell lines (FIGS. 5A and 6A).

### Example 2: Production of mAb-3 in PKM Knockout and PKM-1 Knockout CHO Cell Lines

The PKM gene or PKM-1 gene was knocked out in a CHO-K1M cell line, generating one PKM KO host cell line and four PKM-1 KO host cell lines. A transgene encoding mAb-3 was introduced into the WT CHO-K1M, PKM KO, and PKM-1 KO host cell lines by target integration, generating three mAb-3 expressing WT pools from the same host cell line, three mAb-3 expressing PKM KO pools from the same host cell line, and four mAb-3 expressing PKM-1 KO pools from four different host cell lines.

The following experiments were performed as disclosed in Example 1 except that the gRNAs for generating the PKM KO host are different and provided below. The gRNAs used to generate the PKM-1 KO host cells are the same as used in Example 1.

The gRNAs for generating the PKM KO host cells had the following sequences:

5′-gRNA:
(SEQ ID NO: 42, targeting a region
within exon 2 of the PKM gene)
CCCATCACGGCCCGCAACAC
3′-gRNA:
(SEQ ID NO: 43, targeting a region
within exon 12 of the PKM gene)
CTTCTTCAAGACGGGGGATG

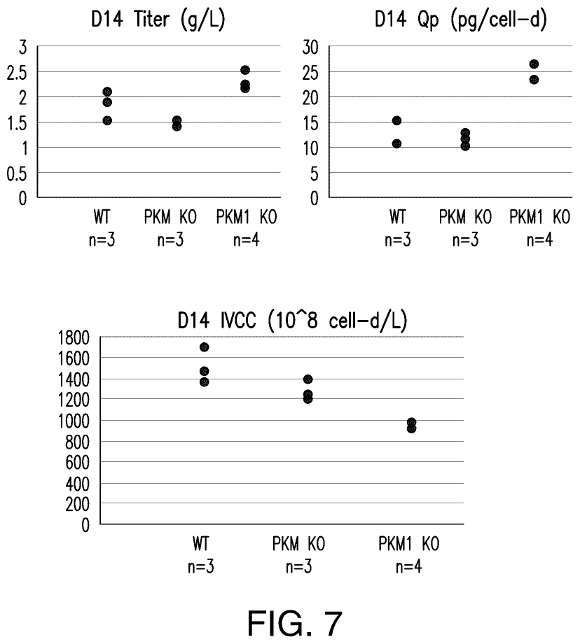

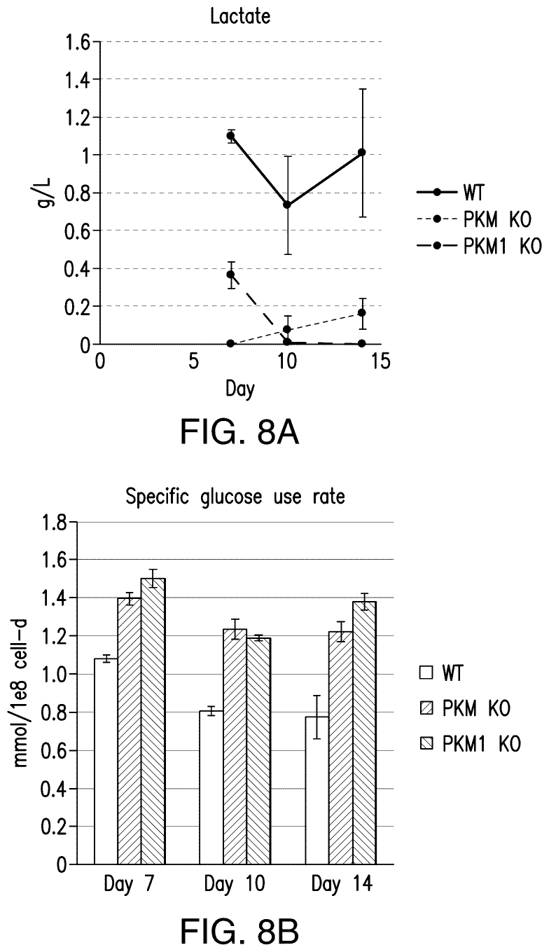

MAb-3 producing pools derived from PKM and PKM-1 KO host cell lines had comparable or higher Qp and titers than WT pools, but lower growth (represented by integral viable cell concentration (IVCC)) in shake flask production (FIG. 7). MAb-3 producing pools derived from PKM and PKM1 KO host cell lines also generated lower lactate than WT host cell lines (FIG. 8A), but consumed more glucose (FIG. 8B) in shake flask production. As shown in FIG. 8A, WT host cells produced about 1.0 g/L of lactate by day 15 of production in a shake flask. During shake flask production, a lactate concentration between 1-2.5 g/L is considered high. In contrast, the PKM-1 KO host cells produced very little lactate and the PKM KO host cells produced less than 0.2 g/L of lactate by day 15, resulting in a reduction in the production of lactate of greater than an 80% when PKM-1 or both PKM-1 and PKM-2 are knocked out.

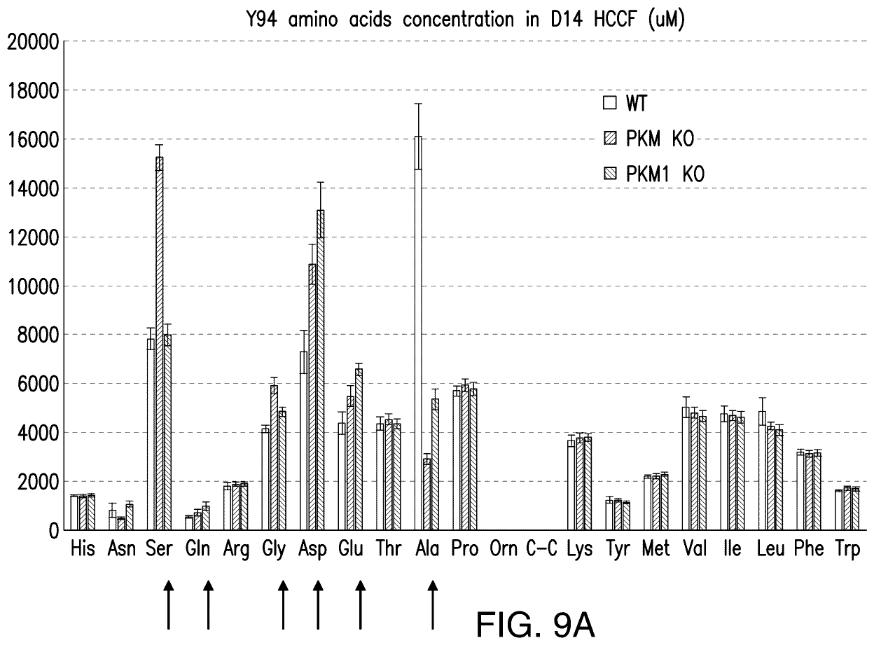

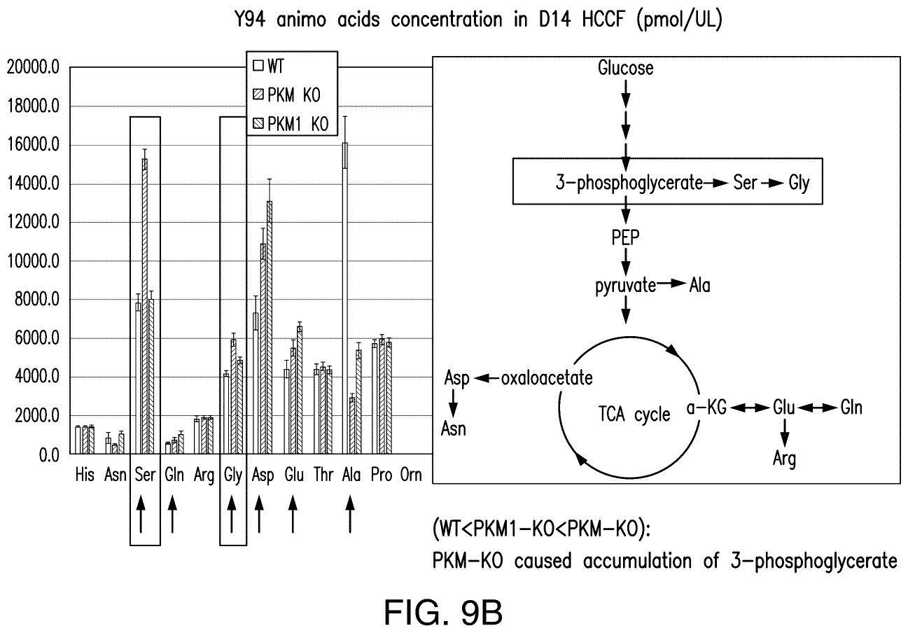

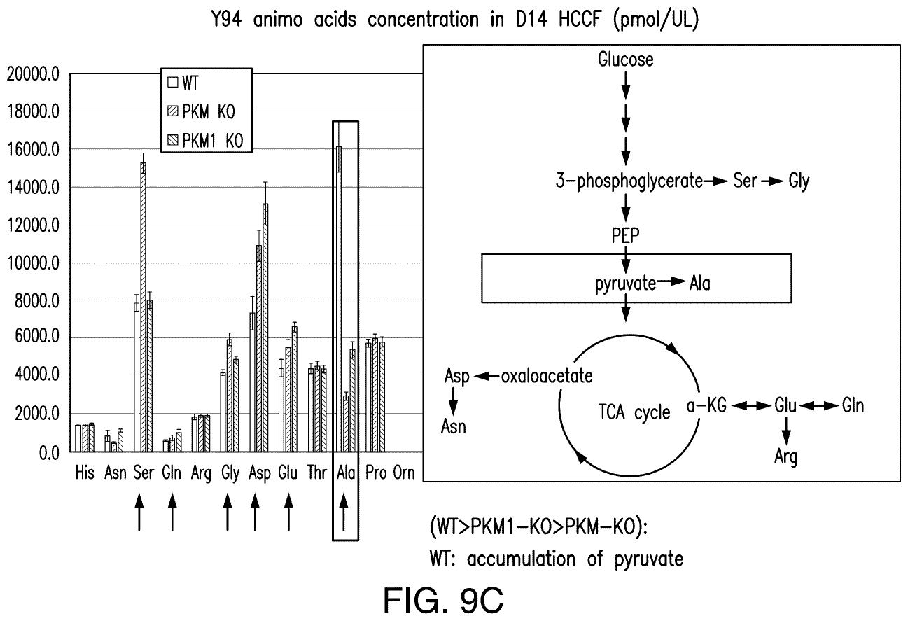

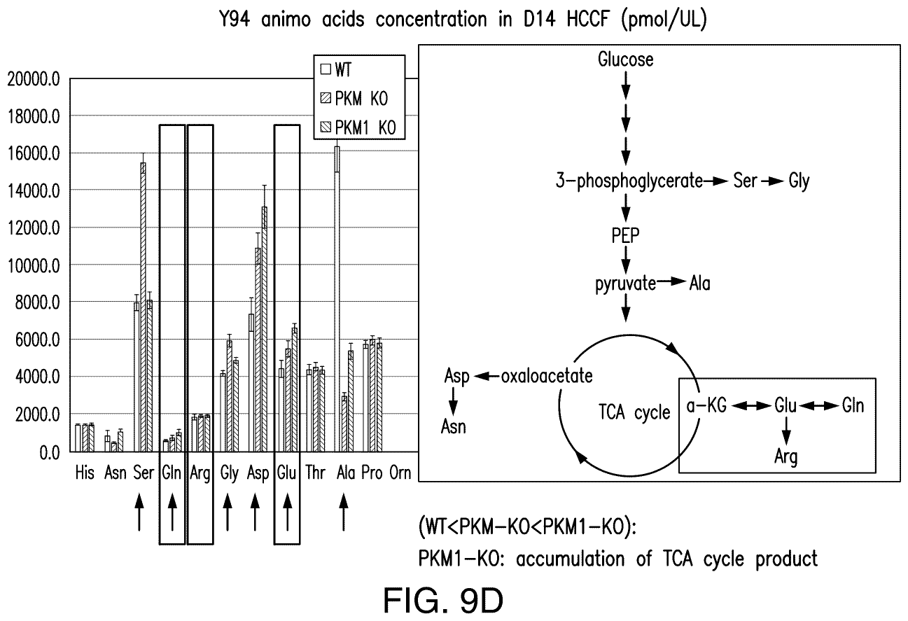

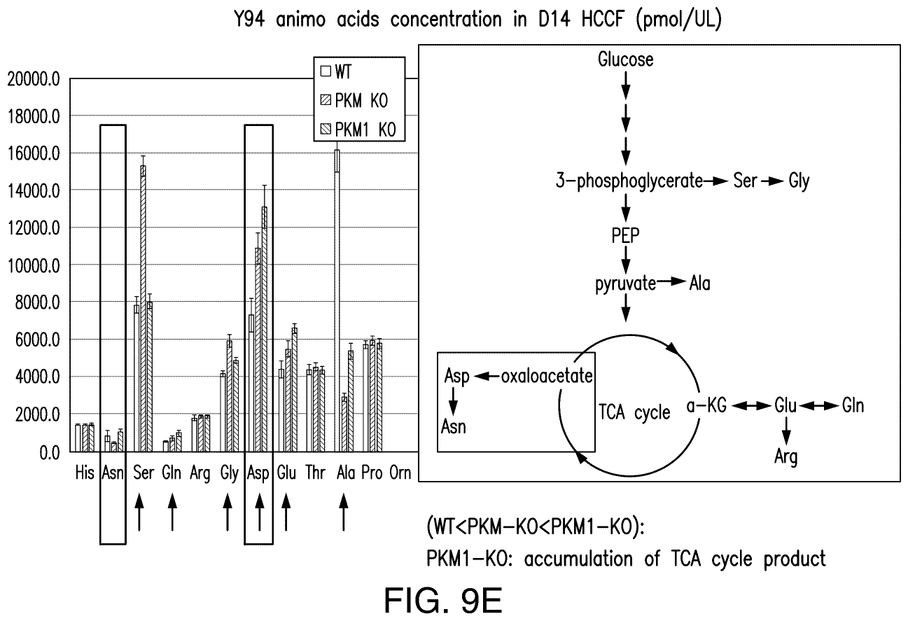

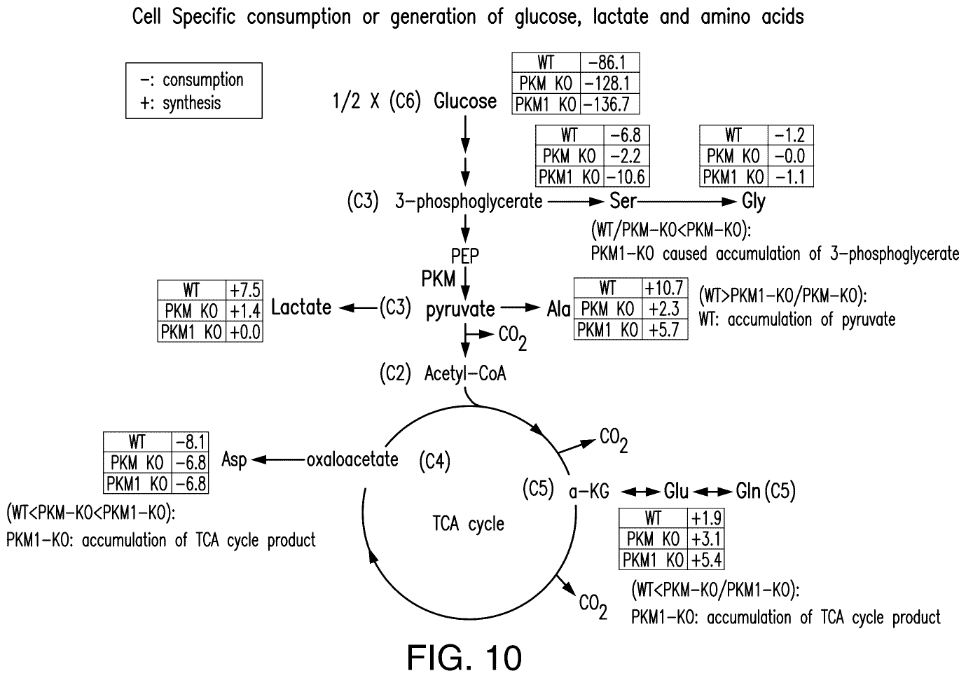

MAb-3 producing pools derived from PKM/PKM-1 KO host cell lines had different amino acid synthesis/consumption rates in shake flask production (FIG. 9A and FIG. 10). For example, PKM KO host cell lines accumulated 3-phosphoglycerate, which led to the increased production of serine and glycine as compared to WT and PKM-1 host cells (FIG. 9B). WT host cell lines accumulated pyruvate compared to PKM KO and PKM-1 host cells (FIG. 9C). Without being limited to a particular theory, the accumulation of pyruvate in WT host cells can lead to the accumulation of lactate and increased production of alanine (FIG. 10). PKM-1 KO host cell lines accumulated TCA cycle products such as oxaloacetate, which led to the accumulation of aspartic acid and asparagine (FIG. 9D), and alpha-ketoglutarate (a-KG), which led to the accumulation of glutamic acid, arginine and glutamine (FIG. 9E). Without being limited to a particular theory, these results suggest that PKM-1 and PKM-2 can function to regulate the level of pyruvate production so pyruvate preferentially enters the TCA cycle. A summary of the above findings is shown in FIG. 10. Based on these data, the cell culture media and conditions used to culture the PKM-1 and PKM KO cells can be adjusted to compensate for the amount of glucose that is consumed and/or the amount of amino acids that is consumed or synthesized.

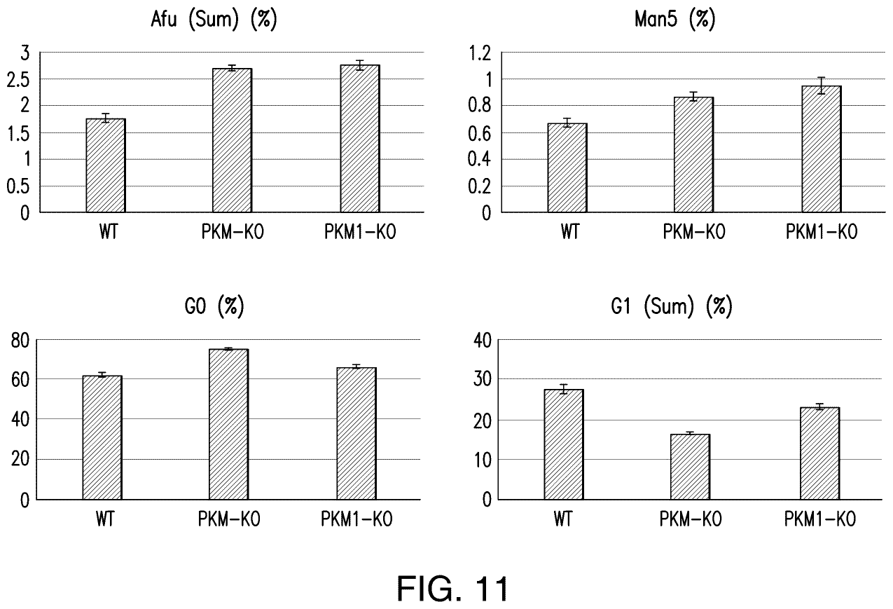

MAb-3 producing pools derived from PKM/PKM1 KO host cell lines had different glycosylation profiles in shake flask production. PKM KO host had decreased galactosylation, and PKM KO and PKM-1 KO host cell lines had slightly decreased fucosylation (FIG. 11). Without being limited to a particular theory, these changes in glycosylation will likely not affect the activity of the antibody generated.

The contents of all figures and all references, patents and published patent applications and Accession numbers cited throughout this application are expressly incorporated herein by reference.

What is claimed is:

1. A method for reducing or eliminating lactogenic activity in a mammalian cell, comprising knocking down or knocking out the expression of a pyruvate kinase muscle (PKM) polypeptide isoform in said mammalian cell; wherein the PKM polypeptide isoform comprises a PKM-1 polypeptide isoform or a PKM-2 polypeptide isoform.

2. The method of claim 1, wherein a genetic engineering system is administered to the mammalian cell for knocking down or knocking out the expression of the pyruvate kinase muscle (PKM) polypeptide isoform.

3. The method of claim 2, wherein the genetic engineering system is: (i) a CRISPR/Cas system; (ii) a zinc-finger nuclease (ZFN) system; (iii) a transcription activator-like effector nuclease (TALEN) system; (iv) an RNA selected from the group consisting of a short hairpin RNA (shRNA), a small interference RNA (siRNA), and a microRNA (miRNA), wherein the RNA is complementary to a portion of an mRNA expressed by the PKM gene; or (v) a combination thereof.

4. The method of claim 3, wherein the genetic engineering system is a CRISPR/Cas9 system comprising: (i) a Cas9 molecule; and (ii) one or more guide RNAs (gRNAs) comprising a targeting sequence that is complementary to a target sequence in a PKM gene in said mammalian cell.

5. The method of claim 4, wherein the target sequence is selected from the group consisting of: a portion of the PKM gene; a 5′ intron region flanking exon 9 of the PKM gene; a 3′ intron region flanking exon 9 of the PKM gene; a 3′ intron region flanking exon 10 of the PKM gene; a region within exon 1 of the PKM gene; a region within exon 2 of the PKM gene; a region within exon 12 of the PKM gene; and a combination of any of the foregoing.

6. The method of claim 1, wherein the mammalian cell expresses a product of interest.

7. The method of claim 6, wherein the product of interest is a recombinant protein.

8. The method of claim 7, wherein the recombinant protein is an antibody or an antigen-binding fragment thereof.

9. The method of claim 8, wherein the antibody is a multispecific antibody or an antigen-binding fragment thereof.

10. The method of claim 8, wherein the amino acid sequence of the antibody consists of a single heavy chain sequence and a single light chain sequence or antigen-binding fragments thereof.

11. The method of claim 1, wherein the product of interest is encoded by an exogenous nucleic acid sequence.

12. The method of claim 10, wherein the exogenous nucleic acid sequence is integrated in the cellular genome of the mammalian cell at a targeted location.

13. The method of claim 1, wherein the lactogenic activity of the mammalian cell is less than about 50% of the lactogenic activity of a reference cell.

14. The method of claim 1, wherein the mammalian cell is a CHO cell.

15. The method of claim 1, wherein the mammalian cell produces less than about 2.0 g/L of lactate during a production phase.

16. The method of claim 1, wherein the PKM polypeptide isoform comprises a PKM-2 polypeptide isoform, wherein the expression of the PKM-2 polypeptide isoform is knocked down or knocked out.
